SEQUENCE LISTING (39 sequences)

SEQ ID NO: 1 - DNA, 60196 bp, micromonospora carbonacea subspecies aurantiaca
gtgccagttccgacacaggaggcccccttgcggaacagcccgccgccagcccattcgcagctcgtcctgagcgaggtcacgaagcactacgccgagcgggtcgtcctggaccgcgtttcgctcaccgtcaagccgggggagcgggtcggcgtcatcggcgagaacgggtcggggaagtcgaccctgctgcggctcgtcgcggggctggagacgccggacaacggcgagttgaccgtctcggcgcccgggggcatcggctatctcgcccagcggcttcggctgccggccggcggcagcaccgtacgggatgtggtggaccacacgctcgccgacctgcgagacctggaggcgcggttgcgcgccgccgaggcggacctggccaccgccacgcccgagcagttggacgcctacggcacgctgctcactgtgttcgaggcccgcggcggctaccaggccgacgcccgggtggacgccgccctgcacggtctcggcctggccgagctcgaccgcgatcgcgacgtcgacacgctctccggcggggaacggtcccggctcgcgctcgccgcgaccctggccgccgcgccggaactgctgctgctcgacgagcccaccaacgacctcgacatcgaggccgtggagtggctggaggatcacctgcggtcgcaccggggcaccgtcgtcgtggtcactcacgaccgggtgttcctggagtcggtcacgtccaccatcctcgaggtcgacaccgacacccgggccgtgcaccggtacggcgacggctatgccagctacctgcgggccaaggccgccctccgggagagccgggagcgcgcgtacgcggaatgggtggccgaggtcgagcggcagtcccaactcgcggagcgggccgggacgatgctccggtcgatctcccgcaagggaccggctgcgttcagcggggccggtgcccaccgctcccggtcgtcgtcgacggcgacgtcacgcaaggcccgcaacgccaacgagcggcttcgccggctgcgggagaatccggtaccgcgacccgccgacccgttgcgcttcaccgcgtcggtcgccccggatgccacggacgccgatacccgccgcgtcgagttgaccgacgtccgggtgggccgccgcctgcacgtgcccgagctgaccatcggacccgccgaacggttgctggtgaccggacccaacggcgcgggtaagagcaccctgatgcgggtgctcgccggggaactcgtgcccgacggcggaacggtgcggctgccggctcggatcggccacctgcgtcaggacgtgacggtcgggcagcccgggcgctctctgctggagacgtacgcgtcgggtcggccggggcatcccgaggagtacgcggaggagttgctcgcccgcggtctgttccggcccgatgacctgcgcatgccggtcgggacgctctccgtcgggcagcgccgccggatcgacctggcccggctggtcgcccgcccggccgacctgctgctgttggacgagcccaccaaccacttcgcgcccctgctcgtggaggagctggaacaggcgctggacggctacgccggagcgctggtcgtggtgacgcacgaccggcggatgcggagcaccttcaccggggctcggctggaactgcaccagggcgtggccaccggggcgagccgggcctgacgagccgcccggggtgccgtggcgcgcccgggacggtggggatctcagccggggtcggcacccggcaccgccgtgagggcggtcgtggacaccgctgcgagggtggtcgtgacctcggcgcacacagcgtcgagctgatcgttgagatagaagtgcccgcccgggaacgtgcggaccatcgtggccgctgcggtcacctcggcccacgccgcggcctcgtcggtggtgacgtgggtgtcggcggccccggcgagtacggtgaccgggcaacgcagcctgggccctggccggtattcgtaggcggcggcggcccggtagtcgttgcggatggcggggaggagcatgtccagcagttccctgtcgtccaggaggctggaatcggtgccctggagccggcggatctcgtcgatcagctcgtcgtcaaaccggtagaaccggtcccgccgcccgacggacgggctacggcggccggaggcgaagaggtgcacgagccgatcggcgtcggccggtgggagccggcgggcggcctcgaaggccaccgtggcgcccatgctgtgaccgaagaaggccaccggtcggtccgcccaggcgagcagtgcgggcaggagcccgtccaccagggcgtcgacggactcgatcaagggttcgccgcggcggtcctgccggcccgggtactggaccgccagcacgtccacgtcggcggcgagccggcgggcgaacggcaggtacgcgctggccgcgcccccggcgtgcgggaagcagaacagccggacggcggggtcgttgacgggccggtagcggcgtagccacagctcggacggatcggcggacggggacatggtgatctgcgctcctcggtctgctcgacgttccggtgtcggtccccacccccgcgccgaagacggccatgatgtcgcgcacggcggccgtcaccggctcgacgtcttacttcgggtgccgtccgtcgcgtaccacctggacggggaggcgtcgcgcggtgagctggtcggcgtcgtagaactcgaccccgacgtggtcgatccggaactcggtgaactggtcgagcgtctggttgaggaagaccttcgcctccagcctggccaggaacgcgcccaggcagtggtggatgccgtggccgaacgccaggtgcttgttcgactcgcgtcggatgtcgaaggtgtccgggtccgtgaacacctcggtgtcgcggttcgcggaggcgatccaggcgatcaccatctggcccttgcgcatggggtggccgaggatgtcggtgtcctcgttcaggatccggaagatgcagttgaacggggaccggtagcgcagcgtctcctcgatcacgcccggcacgaggctgcggtcggcgcggaccgcggcctgtgcctgcgggtgctcctccagcaccaggaacaggttgctgagcagcgtggcgctggagatgtgcccggcggtgagcagcagcgcgacgatgttgacgacttcctcgtcggtcagcttgcgcccgtcgacctccgccgcacagaggccgctgatcaggtcgtccttcggttcggcgcgcttgtgggcgatctgggcgtacaggaattcggaccactcctcgatggcggggcccaccgtctcggtgaagtcgtccgggaggttgggatactccagcccttcgttgctgaggatgatgtccacccactcgcggaacttctcgtgatccttggtgggaatgccgagcagctcggcgatgaccgtcaccggcagcgggtacgcgaggtcgctggcgatgtcgatccggtcctggtcgcgtacctggtcgagcacgtcggcggtgatctgcccgatccgcagctccatctgggcgatccggcggggggtgaacgcctggctcaccagcttgcgcagcggcgcgtgccgcggcgggtcgatgccgccgatggtgccggggcccatcagcagggccagctccgacggtacgggaaagaccgaggtgaagtccgacgagaagatcagcgggttggtggtcacggtctggtagtcccggtaggagaacacgtgccaggcctgacgggtctcgtcccaggagacgggccagttcttccgcatgtacgcgaaccagtccagcagcccctgggcgtcggcgcccttgggcaggtcgatcggtcccgccggggcgttcggggtctgcgtcatggtgtgctcatctcctcggtggtctcggccgtcgggccgaagggaaagagaaccttggttcgcgagggcgtccggtcggggaggggatcttccgggctggcgctgtcacctgcggcctgctcggtcgcctcgccggcattgacggttgtgctgggcggcgagtcagcgctgtggcggcgggcagggcgggccctgcacttctccggggcgtcgtaatcttcggtccgaatcgtgatggccgcaaggccggacctgacatagtgctgtctgcaacgctcggagcacccgttttatcagttgattgcggtcatttttgtcgacgatcagggcggttctatatcgagacttgacatagtcttctacggattcgtgacaatgatcatcgatcggtgttggctgaatcgacgaaaggggcgtgctgttcgagggggcgttgccaagatcaatgcaaaaccgcatccttgatcaatgcggaaccgcaccctgcctggaagagagctgccatggagcatccagtaacggccgggtcctgcaggttctaccccttcagtgaccgtaccgacctgaatatcgatcccacgtacggcgaactgcgctcgaaagagccggtcgcccgcgtccgcatgccctacggcggggacgcctggctggtcacccggcacgccgacgccaagaaggccctctctgacccccgactcagcattgcagccggagccgggcgggacgtgccgcgcgcctccccccgtctccaggaacccgacggtctgatgggtcttccccccgacgcgcacgcccgactgcgcaggctcgtcgccacggcgttcacgccgaagcgcgtacgggacatcgccccgcgcgtcgtccagctcgccgacaagcttctcgacgacgtggtcgaaaccgggccgccggccgacctcgtgcagcagctcgcgcttcccctgccggtgatgatcatctgcgagatgatgggcatcgggtacgacgagcagcacctgttccgtgccttcagcgatgccctgatgtcctccacccgatacacggccgaccaggtcgaccgcgcggtagaggacttcgtcgagtacctcggcggcctcctcgcgcagcgccgtgcacaccgcaccgacgacctcctcggcgccctggtcgaggcgcgagacgacggcgatcggctgaccgaggacgaactcgtcatgctcaccggcggcctgctcgtcggcggccacgagacgaccgccagccagatcgcctcgcagatcttcctcctgctgcgcgaccggaccaggtacgagcaactccatgcccgtccggagttgatccccacggcagtcgaggaactgctgcgggtggccccgctctgggcctcggtcggccccacccgcatcgccaccgaggacctggaactcaacgggacgaccatccgggccggcgacgccgtcgtcttctcgctggcgtccgccaatcaggacgacgacgtcttcgcgaatgccgcagacgtcgtgctcgaccgcgacccgaatccgcacatcgccttcgggcacgggccccattactgcatcggggcgtcactggccagactggaaatacaggccgccatcggcgccttggccaggcggcttcccggtctccgcctggccgtcgaggaaaacgaacttgattggaacaagggaatgatggtacgcagcctcgtgtcccttccggtgacgtggtgacccggcccgggcgccggatcaggtgacgaacggatcagtagcgcattggctcggccggcccggagctgacatggcctggtgaagccgaaaaccatcggccgccgccgctgcaagcgcccgctgggacgatgcgagttgtgggcgcagacgcgtgcagcgcagccgtccccgccggaccgcggatgggcttcccagcatcgttcttcgacccaggagacctcatgaccgtgcagagtgacgtgttgcgccaccgcgatatcgccgtcatcgggatgtcctgccggcttcccggcgcgccgagcatcgaggaattctgggacctgctgtgcagcgggcggagcgcggtcgaccgccagcccgacggcggttggcgggcggtgatcgatgggaagggagaatccgacgccgcgttcttcggcatgtccccgcgccaggccgccgcggtcgacccgcaacagcgcctgatgctcgaactcggctgggaggcactggagaacgcccgcatccggcccgccgacctgaagggctccgacactggcgtcttcgtggggctcaccgccgacgactacgccaccttgctgcgccgctccggcacgcccatcagcgggcacaccgcgacaggcctgaaccgtagcctcacggccaaccgtctctcgtacctgctgggtctgcgcggccccagcttcaccgtggactccgcgcagtcgtcatccctggtcgccgttcacctggcgtgcgaaagcctgctgcggggcgagagcgcggtcgccgtcgtcggcggggtgagcctcatcctggcagaggagagcaccgccgccatggcgcgtatgggggcactctctcctgacgggcgttgcttcaccttcgacgcccgggccaacggctacgtccgtggcgagggtggcgtggccatggtcctcaagccgctgatccgcgcgatcgaggacggcgaccaggtgcactgcgtcatccggggctgtgccgtcaacaacgacggcggtggccccagcctcacccatcccgaccgggaggcccaggaggcattgctgcgccgggcgtacgagcgggcgggggtggcccccgaacacgtcgactacgtcgagctgcacggcaccgggacgaaggccggcgaccccgtcgaggcggcggccctcggggcggtgctgggtgtcgcccgcggctgcgacaacccactcgcggtcggatcggtcaagaccaacgtcggccacctggagggggcggccggcatcacgggcctgctgaaggcggtgctgtgcgtacgtgagggggtgctgccgccgagcctcaacttccgtacgccgaacccggacatccgcctcgacgagctgaacctccgggttcagacggaactgcagccgtggccgggcgacgggacgggccgcccgcgtgtcgccggagtgagttccttcggcatgggcggtacgaatgcgcatctgattctcgagcaggctccggtggcggctgaggaaacggctgttaccgatgccggtgtcggttcggttcgggtggttccggtggtggtgtcgggtcgttcggtgggggctttgcgggcgtatgcgggtcggttgcgtgaggtgtgcgcggggttgtctgacggtggtggctccggtggtggttctggtctggtggatgtgggttggtcgttggtgtcgtcgcggtcggtgttcgagcatcgggcggtcgtgttcggtgggggtgtcgccgaggtggtggcgggtttggatgcggtggcttctggggcggtgagttcgggttcggtggtggtgggttcggtggcgtcgggtgttgctggtggtggtggtcgggtggtgtttgtgtttccgggtcagggttggcagtgggtgggtatgggtgcggctctgttggacgagtcggaggtgtttgctgagtcgatggtggagtgtgggcgggcgttgtcggggtttgtggattgggatttgttggaagtggtccgcggtggtgggggtgacggatcgtttggtcgggttgatgtggtgcagccggtgtcgtgggcggtgatggtgtcgttggcgcggttgtggatgtcggtgggtgtggtgccggatgcggtggtgggtcattcgcagggtgaggttgctgcgccggtggtggggggtgtgttgagtgtggctgatggggcgcgggtggtggcgttgcggtcgcgggtgatcggtgaggtgttggcgggtggtggtgcgatggtgtcggtggggttgccggtggcggttgtgttggatcggttggcggggtggggtggtcggttgggtgtggcggcggtgaatggtccgtcgttgacggtggtgtcgggggatgtggatgctgctgtggggtttgttggtgagtgtgagcgggatggggtgtgggtgcggcgggtggcggtggattatgcgtcgcattcggcgcatgtggaggcggtggaggggatgctgtcggggttgttgggtggtttgtgtccggggcggggtgtggtgccgttttattcgtcggtggtgggtggtgtggttgatggggtgggtttggatggtgggtattggtatcggaatctgcgtgagcgggtgttgttttcggatgtggtggggcggcttgttggggatgggttttcggggtttgtggagtgttcggggcatccggtgttggcgggtggggtgttggagtcggtggcggtggtggatccggatgtgcggccggtggtggtggggtcgctgcgccgtgatgatggtgggtggggccggtttttgacgtcggtgggtgaggcgttcgtcggcgggatgagtgttgactggaagggtgtgttcgcgggggcgggcgcgcggttggttgacctgccgacgtatccgttccaacgacgccactactgggcaccgaacaccgacggcgcgccagctccgatcctcgatgatcacgcggaggcggagaacgaaccagccgaatccgagccagggattcgggccgagcttctgacgttggccgagcccgagcaactgaaccgactcttggcgaccgttcgcgccagcaccgccgtcgttctgggcctcgactcggcgcaggcggtcgatccggagcgcacgttcaaggagcatggattcgaatcggtcaccgccgtcgagctctgtaaccacctgcaacgcggcactgggctgcgggttcccgcctcgcttgtatacaaccatcccaccccgatggccgctgcccggaagctgcaggaagaaattcagggccggcaaccggagaacgtccggcaggtcacctccgctgctgctgtggatgatccggtggtggtggtggggatgggttgtcgttttccgggtggggtggtgtgtgcggagggtttgtgggatttggtgttggggggtggggatgcggtgtcggggtttccggtggatcggggttgggatgtggaggggttgtttgatccggtgcggggtgtggtggggaagtcgtatgtgcgggagggggggtttgtgtatgacgcggggatgttcgatgcggagttttttggtgtgtcgccgcgtgaggcggtggcgatggatccgcagcagcgtttgtttttggaggtgtcgtgggaggcgttggagcgtgcggggattgatccgttgggtttgcggggttcgcggacgggtgtgtatgtgggggtgatgggtcaggagtatgggccgcggttggtggagtcgggtggtgggtttgagggttatttgttgacggggacgtcgccgagtgtggtgtcgggtcgtgtttcgtatgtgttggggttggagggtccgtcgatttcggttgatacggcgtgttcgtcgtcgttggtggcgttgcatttggcgtgtcaggggttgcggttgggtgagtgtgatgtggcgttggcgggtggggtgacggtgattgcggcgccggggttgtttgtggagttttctcggcagggtgggttgtcgggtgatgggcggtgtcgggcgtttgcgggtggtgcggatgggacggggtggggggagggtgcgggggtggtggtgttggagcggttgtcggtggcgcgggagcgtggtcatcgggtgttggcggtggtgcggggttctgcggtgaatcaggatggtgggtcgaatggtttgacggcgccgtcgggggtggcgcagcgtcgggtgattggtgcggcgttggtggcggcgggtttgggtgtgtcggatgtggatgtggtggaggcgcatgggacggggactcggttgggtgatccgattgaggctgaggcgttgttggggtcgtatgggcggggtcgtgtgggtggggcgttgttgttgggttcggtgaagtcgaatattggtcatacgcaggcggctgcgggtgtggcgggtgtgatcaagatggtgatggcgttgcgggcgggggtggtgccggcgacgttgcatgtggatgtgccgtcgccgttggtggattggtcttcgggtggggtggagttggtgacggaggcgcgggattggccggtggtgggtcgtgtgcgtcgtgcgggtgtgtcggcgtttggggtgtcggggacgaatgcgcatctgattttggagcaggcccccgaattcgacgatccggttgttaccgacaccgacaccgatgctggtgtgggtaggggtctatcggtggttccggtggtggtttcgggtcgttcgacggcggctttgcgcgcttatgcgggccggttgcgtgaggtgtgcgcgggtctttccgatggtgccggtctggtgaatgtgggttggtcgttggtgtcgtcgcggtcggtgttcgagcatcgggcggtcgtgtttggtgggggtgtcgccgaggtggtggcgggtttggatgcggtggtttccggggcggtggcttcgggttcggtggtggtgggttcggtggcgtcgggtgttgctggtggtggtggtcgggtggtgtttgtgtttccgggtcagggttggcagtgggtgggtatgggtgcggcgctgctggacgagtcggaggtgtttgctgagtcgatggtggagtgtggtcgggcgttgtcggggtttgtggattgggatttgttggaggtggtgcggggtggggcgggtgagggggtgtggggtcgggttgatgtggtgcagccggtgtcgtgggcggtgatggtgtcgttggcgcggttgtggatgtcggtgggtgtggtgccggatgcggtggtgggtcattcgcagggtgaggttgctgcggcggtggtggggggtgtgttgagtgtggctgatggggcgcgggtggtggcgttgcggtcgcgggtaattggtgaggtgttggccggtggtggtgcgatggtgtcggtcggactgccgatcgtggatgcgcaggaacggttggcggggtggggtggtcggttgggtgtggcggcggtgaatggtccgtcgttgacggtggtgtcgggggatgtggatgctgctgtggggtttgttggtgagtgtgagcgggatggggtgtgggtgcggcgggtggcggtggattatgcgtcgcattcggcgcatgtggaggcggtggaggggatgctgtcggggttgttgggtggtttgtgtccggggcggggtgtggtgccgttttattcgtcggtggtgggtggtgtggttgatggggtgggtttggatggtgggtattggtatcggaatctgcgtgagcgggtgttgttttcggatgtggtggggcggcttgttggggatgggttttcggggtttgtggagtgttcggggcatccggtgttggcgggtggggtgttggagtcggtggcggtggtggatccggatgtgcggccggtggtggtggggtcgctgcgccgtgatgatggtgggtggggccggtttttgacgtcggtgggtgaggcgttcgtcggcgggatgagtgttgactggaagggtgtgttcgcgggggcgggcgcgcggttggttgacctgccgacgtatccgttccaacgccgccactactgggcaccgactcccaccaaccccgccaccaaccccgccacgggcgacaccaccaccgccgacccggtgggtggcgtgcggtatcggatcacctggaaaccgttgccgacggacgacccccgacccctcaccaaccgctggctactcatcgccgacccggggaccgccggctcggagcttgccgcagacatcacagcagcgctcattcgcaggggcgccgaggtcgagttgctggccgtggacccgctcgcgggccgggcccggatcgccgaactgctcgccaccacgacggctgggccggtgccgctgtcgggcgccgtgtctcttctcgggcttgtgcaggacgcgcatcctcaacacccctccatcggaatgggcgtggtctcgtcgctggcgctggtgcaggccatcggtgacgcgggagccgagactcctttgtggagcgtcacgcagggggcggtcgctgtggtgccccaggaggcgccggatgtgttcggtgcgcaggtgtgggcgttcgggcgggtggccgccctggaactgccggaccgctggggcggcctggtcgaccttccgtccgtaccgaatgcccggatgctggaccagctcgccaacgccctcgccggagcggacggcgaggaccagatcgcggtacgcggctcggggatctacgggcgtcgggtgacgcgcgcggcgggcactgcgcgccgggaatggcgccctcgcgggaacatcctggtgaccggaggtacgggaagtctgggtggccgggtggcccggtggctcgctcgcaacggtgccgaacacctcgttctcaccagtcgtcggggtgccgacgccccgggggcggcagaactggaagctgatcttcgcgcgctcggtgtcgaggtgaccatggccgcctgcgatgtagcggaccgggctgcgctgtccgacgtcctggcggcgcatccgcccactgcggtcttccacaccgccggagtcctgcacgacggtgtgatcgacacgctcgccgccggacacatcgacgaggtcttccgtccgaagaccgctgccgcgctgctgctcgacgaactcacccagcaccaggagctggacgccttcgtcctcttctcatcggttaccggagtctggggcaacggcggccaggcggcgtacgcggcggcgaacgcatcgctggacgccctggcggagcgacgtcgtgccgcaggtcttcccgccacctccatagcttggggactgtggggcggcggtggcatggcggaggggatcggcgagcagaacctgaaccgccgtggcatcacggccttggacccggagctcggcatcgccgctctgcagcaggccctcgaccgcgatgacgtgtctgtcaccgtcgccgacgtcgactggacggttttcgctccgcgtcttgccgacctgcgctcggggcggctcttcgacggggtgcccgaggccaggagcgcgctcgatgcccggaaagtggacaccgagtcgccgagcgccggccttgcgcagcgggtggcggggatgcccgacgcggaacggcagcgggtcctcctcgaaacggtgcgggcggcggccgcggcggtcctgaggcacgagacggtggatgcggtcgcgcccacccgggccttcaaggacgccggcttcgactcgctcacggcgctcgaactgcgcaaccacctcaacagcacgaccggtctgagtctgcctccgacggtggtcttcgaccaccccaccccgtccacgttggcgaagttcctggagggcgtcctcgtcggcgcttctgccgaggaagtcccggtgactgccgcagccgtgcccgtcgacgagcctattgccatcgtcggcatggcctgccgctaccccggcggagccgacactcccgagaagctctgggacctcctgctggccggtgctgacgtcatcggcccagcccccgacgaccggggctgggacgtggactccttctttgatcccgtgccgggcgccgcggggaagtcgtatgcgcgggagggggggtttgtgtatgacgcggggatgttcgatgcggagttctttggtgtgtcgccgcgtgaggcggtggcgatggatccgcagcagcgcttgttgttggagacgtcgtgggaggcgttggagcgtgcgggaatcgatccggcgggtctgcggggtagccggaccggcgtgtactccggcctgacccaccaggagtatgccgcccgtctgcacgaggctccgcaggaactcgagggctatctgctcaccggcaagtcggtgagcgtcgcgtcgggtcgtgtttcgtatgtgttggggttggagggtccgtcgatttcggttgatacggcgtgttcgtcgtcgttggtggcgttgcatttggcgtgtcaggggttgcggttgggtgagtgtgatgtggcgttggcgggtggggtgacggtgattgcggcgccggggttgtttgtggagttttctcggcagggtgggttgtcgggtgatgggcggtgtcgggcgtttgcgggtggtgcggatgggacggggtggggggagggtgcgggggtggtggtgttggagcggttgtcggtggcgcgggagcgtggtcatcgggtgttggcggtggtgcggggttctgcggtgaatcaggatggtgggtcgaatggtttgacggcgccgtcgggggtggcgcagcgtcgggtgattggtgcggcgttggtggcggcgggtttgggtgtgtcggatgtggatgtggtggaggcgcatgggacggggactcggttgggtgatccgattgaggctgaggcgttgttggggtcgtatgggcggggtcgtgtgggtggggcgttgttgttgggttcggtgaagtcgaatattggtcatacgcaggcggctgcgggtgtggcgggtgtgatcaagatggtgatggcgttgcgggcgggggtggtgccggcgacgttgcatgtggatgtgccgtcgccgttggtggattggtcttcgggtggggtggagttggtgacggaggcgcgggattggccggtggtgggtcgtgtgcgtcgtgcgggtgtgtcggcgtttggggtgtcggggacgaatgcgcatctgattttggagcaggcccccgagttcgacgatcctgccgattccgattccgattccgattccgattccgatgccggtgtcgtggatggcggcgagggtggtgttggcaggagcttgtcggtggttccggtggtggtgtcgggtcgttcggtgggggctttgcgggcgtatgcgggtcggttgcgtgaggtgtgcgcggggttgtctgacggtggtggctccggtggtggttctggtttggtggatgtgggttggtcgttggtgtcgtcgcggtcggtgtttgagcatcgggcggtcgtgttcggtgggggtgtggaggaggttgttgctggtcttggtgcggtggcttctggggcggtggcttcgggttcggtggtggtgggttcggtggcgtcgggtgttgctggtggtggtggtcgggtggtgtttgtgtttccgggtcagggttggcagtgggtgggtatgggtgcggcgctgctggacgagtcggaggtgttcgccgagtcgatggtggagtgtggtcgggcgttgtcggggtttgtggattgggatttgttggaggtggtgcgcggcggggcgggtgagggggtgtggggtcgggttgatgtggtgcagccggtgtcgtgggcggtgatggtgtcgttggcgcggttgtggatgtcggtgggtgtggtgccggatgcggtggtgggtcattcgcagggtgaggttgctgcggcggtggtggggggtgtgttgagtgtggctgatggggcgcgggtggtggcgttgcggtcgcgggtgatcggtgaggtgttggccggtggtggtgcgatggtgtcggtcggactgccgatcgtggatgtgcaggaacggttggcggggtggggtggtcggttgggtgtggcggcggtgaatggtccgtcgttgacggtggtgtcgggggatgtggatgctgctgtggggtttgttggtgagtgtgagcgggatggggtgtgggtgcggcgggtggcggtggattatgcgtcgcattcggcgcatgtggaggcggtggaggggatgctgtcggggttgttgggtggtttgtgtccggggcggggtgtggtgccgttttattcgtcggtggtgggtggtgtggttgatggggtgggtttggatggtgggtattggtatcggaatctgcgtgagcgggtgttgttttcggatgtggtggggcggcttgttggggatgggttttcggggtttgtggagtgttcggggcatccggtgttggcgggtggggtgttggagtcggtggcggtggtggatccggatgtgcggccggtggtggtggggtcgctgcgccgtgatgatggtgggtggggccggtttctgacgtcggtgggtgaggcgttcgtcggcgggatgagtgttgactggaagggtgtgttcgcgggggcgggcgcgcggttggttgacctgccgacgtatccgttccaacgacgccactactgggcccagacctcgcccgctggcgtcgggacggccgcggcggcccggttcggcatggagtgggaggaccatcccctgctcggcggtgcgctgtcggtcgggggctccaggagcctgcttctggccgggcatctgtcgctcgcctcgcacgcctggctgaccgaccatgccgtctccggcaccgtgctgctgcccggtacggccttcgtggaactcgccctgcacgccgccgctgcggctggctgtccggaggtcgaggagctgcggctggaggctcccctggtggtgccggccaggggcggggtgcggctccaggtgctcgtggacgaccccgacgacggatccgaccgccgcgcggtaagcgtgttctcccgggacgatgcggcgccggccgagtccgcctggacgcggcacgcggtgggcgtcctggccgcgcggtcgcggcctgcaccggctgcgccctggcacaccgacgcctggccaccttcgggcacggagccggtcgacgtggccgacctgtatgagcggttcgcggcgctgggctacgagtacggggaggcgttcgccgggctccagggggtctggcggggggacggcgaggtgttcgccgaggtgcggctgcccgaccgggtcagcgcggaggccattcgcttcgggctgcatcccgcgctgctcgacgccgccctgcaggggtggttggcgggcgacctcgtcggcgtccccgagggcagtgtgctgctgcccttcgcctggcagggcgtcgtgctccacgccaccggcgccgacactctgcgggttcgcatcggccggtccggtgactcggccgtctgcctgcacgcggtggacccggccggtgctccggtcctctcgttggacgccctggccctgcgtccgctcgtccgggaacgcctcgggctgcccgccgatgccggagccggggcgttgtaccgggtcggctggcggcggcaggccgccgttgccggggcagccgaccggcggtgggcggtcgtggccccgaacggtgccgaggcggacggggccgccgagccgcaccggtggccggtcgccgccgtcgacgtgcacaccgacgtggactcgctgcgggcggccctggacgcgggcgcggaactgcccgccgtcgtcctcgccgacttccggagggccgccggctggagcgtcgacagttcgctggccgccggcccgtcgcccaacgacggcgcggtgggcgacggcgcggtgggcgacgcccgggccggggccgtccgggcggcgacccgggccgggctggatctgctgcaacgctggctggccgacgagcggttcatcgcggccaggctcgtggtggtcaccgaacgggccgtggccgccgggccggacgaggacgtgccgggcctcgtccacgcgggactgtggggcctgctccggtcggcccaatcggagcacccggaccgcttcgtgctggtggacgtcgacgcggacgacagctcgctcgcggcgctgccgtcggccctcgccatggacgcgccccaactggtggtgcgggccggtcagatcctgctgcccgagatcgagccggtgcggcccgtacccgagccggagcaggcggaacccgaaccgggggccgtcctggaccccgacggcacggtcctgctcaccggcgcgaccggcacgctcggcgggctgctcgcccggcacctggtgaccacccgtggtgcgcgccggctgctgctggtcagccgcagcggtccggacgcccccgatgccggccggctgaccgaggagctgaccgggctcggcgcccacgtgacgctggccgcctgcgacaccacggatcgcgccgcgctggccggcgtcctgggcggcatccccgccgagcatccgctgaccgccgtggtgcacgtggccggcgtactcgacgacggggcggtgcaggcgctcacccccgagcgggtcgacgcggtgctccggccgaaggtggacgcggcactgcacctgcacgaactgaccgcggggctgccgctggccgcgttcgtgctgttctccggggcggcggggatcctgggccggcccggccaggccaactacgcggcggcgaacaccttcctggacgccctggcgcagcaccgacgggcccggggcctgcccggcgtctccctcgcctggggcctgtgggggctggccagcgacatgacgggccacctgggcgagcaggacctgcggcggatgcggcgctccggcatcgccccgatgaccggcgaggagggcctcgcgctgttcgacctggccctcgacctggcccgggacgaaccggtgctcgtaccggcccgactggacccggcggcgctgcgccgggagtgggccgccaacggaccgggcgccgtcccggtcctgctgcggggtctggtgccggcggctccgctccgtcgcgcggccccgtcgggcgccgccggcggtgcgcccgtgcccgccgtcgccgcgccgcagcaggcggacgagctgcgcgggcaactggccgggaaggacgcgcaggcccaggtccggcagctgctggatctggtacgcgcccatgtcgccggggtgctcgccctccgggaagcggcggacgtggacccgggcagaccgttccgcgaggtcggattcgactcgttgaccgcagtcgaactgcgcaaccggctgggctcggcgaccggcctgcggttggcaccgagcctggtgttcgaccatccgaccccgtcggccgtggccgagcacctcgtggaccgcctcgccgccgagggggcggctgacgagggcgcggcggcactgaccgggctcgacgcagtggccgcggcgctcggcgggatgcggacggacgacgttcgccgggacatcgtccgcaggcggctggaggagatgctcgccctggtcggcgggccacggtccgggccggcaggtgacgggctggtggatgccacggtcgccgagcgactggactcggcttccgacgacgaactcttcgccctgatcgaggagcagctgtgaaccccgaccgaggagagggccggcaggtgaccgcgaacgaggaccggatgcgtgagtacctcaagcgggtcaccgccgagctggccgggacgcggcgacgcctgcgcgagctggaggacagcgcgcgtgagcccatcgcgatcgtgggcatgagctgccggttgccgggcggggtgagcacgcccgaggacctgtggcggctggtcgaggccggtaccgacgcgatctccggcttccccgacgaccggggctgggatgtcgggaggctctacgacccggatccggactcgaccggaacgagctacgtgcgcgagggcggcttcctctacgactgcgccgagttcgacccggagttcttcaccgtctcgccccgcgaggcgctggccatggacccgcagcagcggctgctgctggaggccgcctgggagaccttcgaacgggcggggatcgcccccgactcggcccgcggcacccgcaccggggtctacgtcggggtgatgtacgacgactacggcagccggctgtcggaggtgccgaaggacctggagggctacctggtcaacggcagcgcgggcagtgtcgcgtcgggccggatcgcgtacacgctggggttgcaggggccggcggtgacggtcgacacggcctgctcgtcgtcgctggtcgcgttgcacctggccgtgcaggcgctgcggtcgggcgagtgtgagctggccctggcgggcggggcgacggtgctcgccacgccgacgatgttcgtcgacttcgcccggcagcgcggtctcgccgaggacggccgttgcaaggcgttcgcggacgccgccgacgggaccgggttcggcgagggcgtggggatgctgctggtggaacggctctcggacgcggtccgcaaccgtcgccaggtgctggccgtcgtgcggggcagcgcggtcaaccaggacggggcgagcaacggcctgaccgccccgaacggtacggcccagcaactggtcatccggcaggcgttgaccaacgcggggctggccgcggacgaggtggacgcggtggaggcacacggcaccggcacccggctgggcgatccgatcgaggcgcaggcgctgctggcgacgtacggccagggccggccggcggaccggccgctcctgctgggatccctgaagtccaacatcggccacacccaggccgccgcaggggtcgccggggtgatcaagaccgtgctggcgctgcgtcacgcgcggctgccccggaccctgcacgtcgatcgcccctcgacccgggtggactggtcgtcgggcgcggtgcggctgctgaccgaggggcggccctggcccgatcacggcgaccggccccgccgggccggggtctcctcgttcggcgcgagcggcaccaacgcgcacgtcatcctggagagcgcccccggtgcggcggcgggggcgaccggggcgacggacctctcggccccgccggcatccgtcgcccaccatccggccacggccacggccacggccccggcggcgacggtgcccactgcccacgaaccggcggggacggccggcgacgaccccgtctgggtcctgtccggccggaccgaggcggccctgcgcgagcaggcccggcggctacacgcccacctgacatcccgggcgcggcccgagcccgccgacgccgtggcccgcgcgctggcgcgctcccgcaccgcgttcgcgtaccgggccgccgtgctgggccgggacgacaccgcgcggctcgacggcctccacgcgctcgcggcgggtcgcagcgccgcggggctcgtcaccgggcgggccgtgccggagcggcgcgtggccttcctcttcaccgggcagggcagccagcgaccgggcgcgggccgggaactgtacgcccggcatcccgccttcgcacaggccctggacggcgtcctcgcggaactcgaccggcacctggaccggccgctgcgcgccgtcatgctcgccgagccgggcaccgaggcggcggcgctgctggacgacaccgcgtacacccagcccgccctgttcgcgctggaggtggcgctgttccggctggtcacgagctgggggctgcggcctgacgccctgctgggccactcggtcggggagatcaccgcggcgtacgtcgcgggcgtcctcaccctgccggacgccgcccggctggtggcggtgcgcggtcgactcatggcggacctgcgggccggcggtgcgatggccgcgctccaggccgccgagagcgaggtcgaccccctgttggcggggcgggagggcgaactgtcgatcgcagcggtcaacgggccgcaggcaaccgtgatcgcgggcgacgaggcggccgtcgaggagcaggtcgcgctgtggcgtgaccggggtcgccgggccaggcgactgcgggtcggccacgccttccactccgtacggatggacgggatgctcgccgagttcgagaaggcgatgggtgatctccgtgccggcgagccgacgatccccgtggtcgccaacgtcaggggggcgatcgcgtccggcaccgacctccgtacggccgggtactggatccggcacgcccgcgagccggtgcgtttcctcgacggcatgcgtgcgctgcgggccgagggcgtcgacacgttcgtggaactcggccccgacggagtgctcacggcgatggcgcgcgactgcctggcggatcccgccgacccggtggatctcgcggacgccgccgagcccgccggggccgcggagcccgaccgctccctgctgttcctgcccaccctgcgccgggaccgcgacgacgcagtggccgtgcgggaggccctggcatccgtccacgtgcacgggcttcccgtcgacccggtcgcgccgctcggcgacggcccgctcgccaccgacctgcccacctacccgttccagcggtcccgctactggctcgacccgcgtcccggggcacgcgacctgaccgccgtgggcctcgacgtggccgggcacccgctgctcgccgtcgccgtggacctgcccgacggcgccggcacggtctggagcggtcagctctgcgtgcggacgcatccgtggctcgccgaccacagcgtgtgggggcgcacggtggtgccggggaccgcgctgctggagatcatgcaccgagtgcgcgccgaggtgggctgcacccgggtcgcggaactgaccttcgaggcgccgatggtgctggccgacgacgggggcgtccgcgtgcgggtcgtcgtcgacggaccagacgccgacggggcccgccaggtccggatccactccgcaccggtggggcccgagcctccccactggacccggcacgcctcgggccgcgtcgacagcgccgcgccggggccggccgccggcccacccgcgtgggacgccggccctggcagcaactggccgcccgagggggcggagccggtgggcgtcgagagcgagtacgagcgcttcgccgacaacggcatcggatacggccccgccttccgagggctgcgcgccgcgtggcgtcgcgggaacgagacgttcgccgaggtccggctccccgaggggtacgccgccgaggcgggcgactacgccgtccatccggcactgctggacgcggccctgcacgcgatcgtcttcggtgaccagtttcccggtggggcacacgggatgctgccgttcgccttcaccgacgtgcgggtgttcagctccggcgccgaccggctccgggtgcgcatcgcgcccgccgatgccgactcggtctgcgtgaccgtcgccgacggcgacgggacgccggtcctcgccgcagccaccctggcgttgcgccgggtcgccgccgaccggatcgcggcgaccgtcaccggccaggcaccgctgtaccggttggagtggtccgccgtgcggcccgccccggtggccaccggggcgcggttcgccgtcgtcggcgcggacgccccgctgccgtccggtgcgctgggggccggggtgcccgtccaggcgtacccggacctgggcgcgctggccggcgcgttggccaccaacggggcaccgggccacgtgctcgtcgacttccgccgccgcgccgacggcccggcagggcggcagcccggtgacgtgggtgcacggacccgacgggcgctggccgtcgtccaggagtggctcgccgacgaccgtttcaccggctcacggctggtcgtgctcaccagcggagccgtggacgccggaacagccgtcaccgatccggccgccgccggggtgtggggcctgctgcgggtcgcccagaccgagcatccggaccggttcgtcctcgtggacaccgacgaccacccggattcgctgcgtgccctccccggggcgatcgttgcgggcgagccgcagctggcactgcgggccggcacggccagcgttccgggcctggtgcgggtgccggccggcaccggtgccgccccgccgtgggccgcagccggcaccgtcctcgtcaccgggggcaccggcatgctcggcggcgcggtggcccggcacctggtccgccggcacggggtccgccgcctgctgctggtcggccggcgcgggccggacgcacccggcgcggcggccctgacccgggaactggaggagctgggagcgtccgtccgcgtcgccgcctgcgacgtcggcgatcgtggcgcggtgacgcgcctgttggccggggttcccgccgcgcatccgctcaccgcggtggtgcactcggccggcctgcccgacgacggcgtgctgaccgcacagaccggcgagcgggtcgcggcggtgctccgcgccaaggcggacgcagcggtcaacctgcacgaactcacccggcatctcgacctcaccgccttcgtgctgttctcgtcggtagcggggacgatcggcagcgccgggcaggccgggtacgccgccgcgaacgccttcctcgacgcgttcgcgagctggcggcagggccaggggctgcccgccaccgccctggcgtgggggccgttggacggcgggatggccgccggcctcggcactgcggacgtggcacggctgcgccggtccgggctcgtgccgctcggcgtggacgacgcgctcgttctcttcgacgccgcctgctcccgaccggcggcggcgtaccaccccgtccgcctcgatccggcggtgctgcggtcccacgccgccgccgacagcgcggtgcccgccgtcctgctcggtccgagccgtgcgcacccgagggacggtacgccggggaagcctgccgaagccgccctcgccgcgctgctgaccggcaggtcggcggccgagcgtacggcgatcctgaccgacctggtgcggacggaggccgccgccgttctcgggcatggcgaggcggcgatgctgagcacgcagcgggccttccgcgacgccggcttcgactcgctcaccgccgtggacctccgcaaccggctcggcgcggccacgggcctcagcctgccggccgccgtcgtcttcgaccacccgaccccggcggccctggccgcctatctgcggaccgaactggaccgccggtcgcccaccgggcaacagttcccgacggacgccgccggtgttctggccatgctcgaccgcctgcgggacggaatcgcgacggtcgtcagggacgacgccgaccggacccgcgcagccgacctgttgcgtgtcctgctcgccgaggtcggcgggcccgggacgggcccgccccgcgacaccgacggcggctccggcggcgaggtcagcgaccgcctccggaccgcctccgacgaggaactgttcgacctgctcgacagcgatttccgactggcgtagcgccggccggagcactgcccgctcgaatcgaccgaccccgggaagacactcggatcacagggggaagcgccgtgtctgtcaacaacgaagacaagcttcgcgagtatctgcgtcgtgccatggcggatctccatgagtcccgcgagcggttgcggcagtacgagtccgctgctgctgtggatgatccggtggtggtggtggggatgggttgtcgttttccgggtggggtggtgtgtgcggagggtttgtgggatttggtgttggggggtggggatgcggtgtcggggtttccggtggatcggggttgggatgtggaggggttgtttgatccggtgcggggtgtggtggggaagtcgtatgtgcgggagggggggtttgtgtatgacgcggggatgttcgatgcggagttttttggtgtgtcgccgcgtgaggcggtggcgatggatccgcagcagcgtttgtttttggaggtgtcgtgggaggcgttggagcgtgcggggattgatccgttgggtttgcggggttcgcggacgggtgtgtatgtgggggtgatgggtcaggagtatgggccgcggttggtggagtcgggtggtgggtttgagggttatttgttgacggggacgtcgccgagtgtggtgtcgggtcgtgtttcgtatgtgttggggttggagggtccgtcgatttcggttgatacggcgtgttcgtcgtcgttggtggcgttgcatttggcgtgtcaggggttgcggttgggtgagtgtgatgtggcgttggcgggtggggtgacggtgattgcggcgccggggttgtttgtggagttttctcggcagggtgggttgtcgggtgatgggcggtgtcgggcgtttgcgggtggtgcggatgggacggggtggggggagggtgcgggggtggtggtgttggagcggttgtcggtggcgcgggagcgtggtcatcgggtgttggcggtggtgcggggttctgcggtgaatcaggatggtgggtcgaatggtttgacggcgccgtcgggggtggcgcagcgtcgggtgattggtgcggcgttggtggcggcgggtttgggtgtgtcggatgtggatgtggtggaggcgcatgggacggggactcggttgggtgatccgattgaggctgaggcgttgttggggtcgtatgggcggggtcgtgtgggtggggcgttgttgttgggttcggtgaagtcgaatattggtcatacgcaggcggctgcgggtgtggcgggtgtgatcaagatggtgatggcgttgcgggcgggggtggtgccggcgacgttgcatgtggatgtgccgtcgccgttggtggattggtcttcgggtggggtggagttggtgacggaggcgcgggattggccggtggtgggtcgtgtgcgtcgtgcgggtgtgtcggcgtttggggtgtcggggacgaatgcgcatctgattttggagcaggcccccgagttcgacgatcctgccgattccgattccgattccgattccgatgccggtgtcgtggatggcggcgagggtggtgttggcaggagcttgtcggtggttccggtggtggtgtcgggtcgttcggtgggggctttgcgggcgtatgcgggtcggttgcgtgaggtgtgcgcggggttgtctgacggtggtggctccggtggtggttctggtttggtggatgtgggttggtcgttggtgtcgtcgcggtcggtgtttgagcatcgggcggtcgtgttcggtgggggtgtggaggaggttgttgctggtcttggtgcggtggcttctggggcggtggcttcgggttcggtggtggtgggttcggtggcgtcgggtgttgctggtggtggtggtcgggtggtgtttgtgtttccgggtcagggttggcagtgggtgggtatgggtgcggcgctgctggacgagtcggaggtgttcgccgagtcgatggtggagtgtggtcgggcgttgtcggggtttgtggattgggatttgttggaggtggtgcgcggcggggcgggtgagggggtgtggggtcgggttgatgtggtgcagccggtgtcgtgggcggtgatggtgtcgttggcgcggttgtggatgtcggtgggtgtggtgccggatgcggtggtgggtcattcgcagggtgaggttgctgcggcggtggtggggggtgtgttgagtgtggctgatggggcgcgggtggtggcgttgcggtcgcgggtgatcggtgaggtgttggccggtggtggtgcgatggtgtcggtcggactgccgatcgtggatgtgcaggaacggttggcggggtggggtggtcggttgggtgtggcggcggtgaatggtccgtcgttgacggtggtgtcgggggatgtggatgctgctgtggggtttgttggtgagtgtgagcgggatggggtgtgggtgcggcgggtggcggtggattatgcgtcgcattcggcgcatgtggaggcggtggaggggatgctgtcggggttgttgggtggtttgtgtccggggcggggtgtggtgccgttttattcgtcggtggtgggtggtgtggttgatggggtgggtttggatggtgggtattggtatcggaatctgcgtgagcgggtgttgttttcggatgtggtggggcggcttgttggggatgggttttcggggtttgtggagtgttcggggcatccggtgttggcgggtggggtgttggagtcggtggcggtggtggatccggatgtgcggccggtggtggtggggtcgctgcgccgtgatgatggtgggtggggccggtttctgacgtcggtgggtgaggcgttcgtcggcgggatgagtgttgactggaagggtgtgttcgcgggggcgggcgcgcggttggttgacctgccgacgtatccgttccaacgccgccactactgggcaccgactcccaccaaccccgccaccaaccccgccaccaaccccgccaccaaccccgccacgggcgacaccaccaccgccgacccggcgggtgacctgcggtatcggatcacctggaaaccgttgccgaccgacgacccccgacccctcaccaaccgctggctgctgatggtgcccgaggcgctggccggtgacggggtggtggcgggcgtacggcaggcgctggccgcgcgtggcgcctccgtcgaactgctgaccgtcggcaccgccgaccgggccggccttgccgcgctcctgacctccgccgcccccggcgacccggaggcggccggcccggcgggcgtggtctccctgctggcgctcgccgagggcgcggacgcgcgccacccggccgtaccgctcggcctgaccgcctcgctcgccctgatccaggcattggcggacgcggggacgcaggcccgcctctgggcggtcacccggggggccgtcgccgtgtcctccggcgaggtgccggacgccgggcaggcccaggtgtgggggctcggccgggtcgcggccctcgaactgccggaccgatggggcgggctggtggacctgccggcgctcaccggggagcgtgccttcgcgcagctcgccgatgtcgtgggcggctcgaacggcgaggaccaggtcgccgtacgggcctccggcgtctacggtcgacgcctcgtgcgttcccgcgccaccgtcacgtccggcgactggccggcccggggcaccatcctcgtcgtcggggacaccggcccggtcgccgcgctcctggccggccgcctcctcggcgacggggcggcgcacgtggtgctcgccggcccggccgccgcgtccaccgtcgggctcaccggcggggccgaccgggtggccctgatcgactgcgacccgagcgaccgggacgcgctcgccgggctgctcggcgcgtaccggcccacgacgatcgtggtggctccgcccgccgtcgcgctcaccgccctcgccgagaccacgccggaggacttcgtcgccgccgtcgccgcgaagacgacgacggcagtgcacctcgacgcccttgcggcggaggcggaactggagctcgacgcgttcgtcgtcttctcctcggtctccggcacctggggcggcgcggggcacggcggctacgcggcgggcaccgcccggctggacgcgctggtcgaggagaggcgggcccgtggcctgcccgccacggcgatcgcgtggacgccgtgggccgacgcgaccacagccgccggcgggcaggcacccgatgccagcgccggcgggcacgaacccgacacgagggccgggggccccgaccgcgaactgctgcgccggggtggcctcaccccgttggacccgggggccgcgctggacgtgctgcgcggggcggtggcgcggggcgagggcctggtgaccgtggccgacgtcgactgggcgcggttcgtcgcctcgtacaccgcggcccggcccaccacgctcttcgacgaactgcccgagctgcgggcgacccgggaggcggagcacaccccggccgaggactcgtcggccggcggcgaactggtccgtgccctcagcggccggcccgcggccgatcagcaccggacgctgctgcggctggtccgtgcgcacgtcgcggccgtcctggggcacgacgaggccgaggcggccgatccggaccgggcgttccgggaactcggcttcacctcggtgacggcggtggacctgcggaaccggctgaacgcggccaccgggctgaacctgccggcgtccgtcgtcttcgaccatcccagcgcccgggtgctggccgcgtacctgcgtgccgagctgctcgggccggaggccgacgaggacacggcggaggccgtcgccccgccgtccgcgccggccggggcgggcgacgacgagccgatcgcggtgatcgggatggcctgtcggttcccgggcggggtcgacgcccccgacgacctgtgggatctgctggcgaagggccgcgacgccatctccaggttccccacgaaccggggctgggacgtcgacggcctgtacgacccggacccggaggcgcccggccgcacctacgtccgcgagggcggcttcctgcacgacgcgcccgacttcgatgccgcgttcttcgggatctcgccccgcgaggccctcgccatggatccgcagcagcgcctgctgctggagaccacgtgggagtccctggaacgggccgggttggacccgaccgcgttgcgcggcacccggaccggggtgttcgtggggaccaacggccagcactacatgccgctgctgcgagacggcgcggacgacttcgacggctacctcggcaccggcaactcggccagcgtcatgtccggccggctctcctacgtcttcggcctggagggcccggcggtgaccgtggacacggcctgctccgcctccctcgtggcgctgcacctcgcggtgcaggcgctgcgccggggcgagtgcacgctggccctggtcggcggggccacggtgatgtcgacgccggacatgctggtggagttctcccggcagcgggcgatgtcgccggacggccggtcgaaggcgttcgccgccgccgccgacggggtggcgctcagcgagggcgccgccatgatggtggtgcagcggctcgccgacgcggaggccgccgggcacgagatcctggccgtggtcaagggctcggccgtcaaccaggacggggccagcaacggcctcaccgccccgaacgggccctcccaggaacgggtcatccggcaggcgctggccgacgccggcctgcggccggaccaggtggacgcggtcgaggcgcacggcaccggcaccgccctgggcgaccccatcgaggcgcaggcgctgctcgccacgtacggccgggaccggccggcgggccggccactgtggctcggctcgctgaagtccaacatcggtcacacccaggccgccgccggcatcgccggggtgatgaaggtgatcctggcgctgcggcacgacacgctgccgcgcacgctgcacgtggaccggccgacgccccgggtggactgggcttccggggcggtgtcgttgctgaccgagccggtgccgtggccgcagggcgacgaaccccgccgggcggcggtgtcctcgttcgggatcagcggcaccaacgcccacgtgatcgtcgagcaggcgccgccggtggtgcgggaaccgatcgaccacgaggcggacgaggtcaccgtcccgctgttcctgtcggcccgggggagcgccgcgctctgcgcccaggcggcacggctgcgggcccggttgatcgaggaacccgacctggacatcgccgaggtcggctacacgctggcggccacccgggcccgcttcgagcaccgggccgtggtgatcggggagagccgcgcggaggtcggcgacgcgctcgccgcgctggcccggggcgaggagcacccgtcgctgctgcgggggcgggccggcgcgagcgaccgggtcgcgttcgtctttcccggccagggctcgcagtgggccgagatggccgacggcctgctcgaccgctccccggccttccgggcgagcgcgtcggcgtgcgacgaggcgctgcgggcgcacctcgactggtccgtgctggacgtgctgcgtcgcgtgccggacgcgcctgcgctgagccgggtcgacgtggtccagccggtgctgttcacgatgatggtgtcgctggcggcggcctggcgggcgctgggcgtgcacccgtccgccgtggtcggccactcgcagggtgagatcgcggcggcccacgtggcgggcggcctctcgctggacgacgcggcgcgcatcgtcgccctgcgcagccaggcgtggctgcggctggccgggcagggcgggatggtggcggtgtcgctccccgtcgacgcgctccgcgcccgcctggcgcggttcggcgaccggctgtccgtcgccgcggtcaacagccccggtacggcggcggtgagcggctaccccgacgcgctcgccgaactcgtcgacgagctgaccgccgagggcgtgcacgccaaggcgatcccgggggtggacacggccgggcactccgcgcaggtggaggtgctgaaggaccacctgatggccgccctcgccccggtgtcgccccgcagctcgcagatccccttctactcgaccgtcacgggcggcctgctggacaccgcgctgctggacgccgcctactggtaccgcaacatgcgcgacccggtggagttcgagcaggcgacccgggcgatgctcgcggacgggcacgaggggttcctggagcccagcccgcacccgatgctgtcggtgtcgttgcagggcaccgcggccgatgccggggtcgccgcgacggtgctggggacactgcggcgcggcaagggcggcgcccgctggttcggcatggcgctcgggctcgcccacgcccacgggatcgagatcgacgcgagtgtgctcttcggaaccgactcgcgccgggtcgacctgccgacgtacccgttccagcgcgagcgcttctggtatcacccgccggccgcgcgcggggacgtggcctccgccgggctcagcggtgccgaccatccgctgctgggcggggcggtcgagctgcctgaccggggcggccacgtgtatccggcccggctcggcgtccgacaccacccgtggctcggcgagcatgccctgctgggcgcggcgatcctgcccggggccgcgtacgcggaactcgccctgtgggccgggcggcgtgacggggccggccggatcgaggagctgaccctcgacgcgccgctggtggtggccgacgagtcggcggcgcaactgcggctcgtggtgggcccggcggacgcggaggggcgccggcagctcaccgtccactcgcgcgccgacggcgcggacgcggacaccgcgtggacccggcacgcgcagggcaccctcgtgccggccgacgccgacgccgccgggagcggggacccgggcgcgccctggccgccggccggggccgagcccgtcgaggtggcgggcctgtacgaccggttcgccgaccggggctaccagtacgggccgtcgttccggggggtccgggccgcctggcgggccggcgacacggtgtacgccgaggtggccctgcccgtcccgcagcccgggagcccgcgcttcggtgtccacccggcgctgctcgacgcggcgttccaggcgatgagcctcggcgcgttcttccccgaggacgggcaggtccggatgccgttcgccctgcggggcgtgtcgtcgtccggggtcggggccgaccggctgcgggtcaccatcagcccggccggtgccgaggcggtccggatcgcctgcgtcgacgagcggggcaacccggtcgtggtgatcgactccctggtggcgcgcgcggtgccggtggaggcgctcacccccggcacccccggcaccggggacggcgcgctgcaccacgtcgcctggaccgcccggccggaaccgggggtcgccgccgtgcagcgctgggcggtcgtgggcgcggccgatcccgggctggccgggggcctggaccgggcgggcggcctctgcggggcgtaccccgatctcgccggtctggtcgcggcggtggccgaaggggcggcgctgcccgacgtggtcgcggtgccggtcccgtcgggcgcgccggtcgggcccgacgcggtgcgcgccaccgtgctcggcgccctggacctgatccgggcctggctcgcggtcgagggccggctggggctggccaggctggcgttcgtcaccacctcggcggtggcggtcggcgacggcaccgagcacgtggacccggtgtcggccgccctgtgggggctggtgcgttccgcccagtccgaggagcccggccggttcgtcctcgtcgacctggacgccgacccggccagcgcctcggccctgcccgccgcgctcgccgccggtgagccgcaactggccgttcgcgccggggcggtgcacgtgccccggctggttcggcaccgaccccgcccggacggcccgctgacgcccccggccggtgccgcgtggcggctcgccgccggtgggcagggcaccctggagggcctggcgctggtcccggccccggacgccttggcgccgctggcccccgggcaggtccgggtcgcggtgcgcgccgccggagtgaacttccgggacaccctcatcgcgctcggcatgtacccgggcacgccggtgctgggtgccgagggggccggggtgatcaccgaggtcgcgccggacgtggccggcttcgcccccggcgaccgggtgctgggcatgtggaccggcggcctggggccggtggcggtcgccgacgcccggatgctcgcccgggttccgcgcggctggtcgtacgccgaggccgcgtcggtgccggccgtcttcctcacggcccactacgcgctcaccaggctcgccgggatccgcccggggcagtcgctgctggtgcacgcgggggccggcggcgtcggcatggcgaccctccaactggcccggcacctgggcgtggaggtctacgccacggcgagccggggcaagtgggacaccctgcgtggcctcggcctggacgacgcgcacatcgccgactcccgcagcctcgacttcgccggacggttcctggccgccaccggggggcgcggcgtcgacgtggtgctgaactcccttgccggggacttcgtggacgcgtccctgcggctgctgccgcgcggcggccacttcctggaactgggcaaggccgacgtccgcgaccccgaccggatcgcggccgaccacccgggggtcggctaccgggcgttcgacctcgtcgaggctggtccggagctggtcgggcagctgctcggcgagctgatggagctgttcgccgccggggtgctcagcccgctgccgttgaccgtgcgggacgtccggcgggcccgggaggcgttccgcctgatcagccaggcccggcacgtcggcaaggtggtgctgaccatgccgcccgcgttcggcgcgtacggcaccgtcctggtcaccggcggcaccgggacgctcggcggcgccgtcgcccggcacctggtcgcccggcacggcgtacggcacctggtgctcaccggccgcagcggcccggcggcggacggggcgtccgcgctcgtcgacgagctgaccgcgtccggcgcgtcggtgaccgtcgtcgcctgcgacgccgccgaccgggtcgcgctgcgccggctgctcgacggcattccggccgcgcacccgctcaccgccgtcgtgcacgctgccggcgtcctcgacgacgccaccatcaccgcgctgaccgccgggcaggtggacgcggtgctgcggcccaaggccgacgcggtgatcaacctgcacgagttgacccgggaccgggagctgtccgcgttcgtgctgttctcctcggcggcggccctgttcggcagcccggggcagggcaactactcggcggccaacgggttcgtcgacgcgttcgcccagtaccgccgcgcgcaggggctccacgcggtgtcgctggcctggggcctgtgggccgacagcagccggatggccgggcacctcgaccaggaggggatgcggcgccggatggcgcgcggcggcgtcctgccgctcaccaccgaccagggcctcgccctgttcgacgccgcgcagctggtggacgaggcgctccaggtgccgatccggctcaacgtcggcgcgttgcgggccgccgggagggtccccgcgctcctcgccgacctggtgccggcggcggcgtcgggggccccggccgccaccccgacccgggacgacgcggaccgcacgctcgccgaccggctcgccgggctgaccgtggccgaacagcgggagctggtgctggagagcgtgcgcggacacgcggcggccgtcctcggacacgccgacccgcaggccgtcgacgccgaccgggccttccgggaactcggcttcgactcgctgacggcggtggagctgcgcaatcggctggccaccgcgtccgggctgcgcctgccggcgacgctggtcttcgaccaccccaccccggaagcgttggcggagcacctgctcgccgggctcgcgcccgagcaggcccgggccgagttgccgttgctggccgagctgggccggctggaggcggccctggccgccaccgacggggccgccctcgacgggctggacgacctggtgcgccgggaggtcggcgtccggatcgcggcgctggccgccaggtggggcgcggccggcgacgacgtggccggcagcgacggcggcgggacggccgacgcgctcgagtccgctgacgacgacgagatcttcgcgttcatcgacgagcggttccgcgcctgacgaccccgcgtacgcgagggacggggtggacgggaccgacggtcaggagggacgaggcggcatgtcgaacgagcagaagctccgcgagtacctgcggttgaccaccaccgagctggccagggccaccgaccggctgcgcgcggtcgaggcgcgggcgcacgagccgatcgcgatcgtcggcatggcctgccggtaccccggcggggtcggctcaccggaggaactgtgggagctggtcgcctcgggcacggacgcgatctccccgttccccgacgaccacggctgggacggcgacgcgctgtacgacccggacccggaggcggcgggccgcacctactgccgcgagggcgggttcctcgccggggtcggcgacttcgacgccgcgttcttcggcatctcgccccgcgaggcgctggccatggacccgcagcagcgcctgctgctggagacgtcctgggaggcgctggagcgggccgggatccccccggactcgctgcgcggcagccgtaccggggtgtgcgtcggggcgtggcacggcggctacaccgacgtcgtcgggcagcccccggcggaactggagggccacctgctgaccggcggggtggtcagcttcacctcggggcggatctcgtacgcgctgggcctggaggggcccgcgttgacggtggacaccgcctgctcgtcctcgctggtggccctgcacctggcggtgcgggccctgcggcagggcgagtgcgacctggcgttggccggcggggcgacggtgctggccagcccggcggtgttcgtgcagttctcgcggcagcgggggctggccccggacggccggtgcaaggcgttcgccgactcggcggacgggttcgggccggccgagggggtcggcatgctggtcgtggagcggctgtcggacgccgtccgccacgggcgccgggtgctggccctggtcaccggcacggcggtcaaccaggacggggcgagcaacggcctcaccgcccccagcggcccggcgcaggagaaggtgctgcgccaggcgctcgtggacgcccgggtgacggccgccgacgtcgacgcggtcgaggcgcacggcaccggcacccggctcggcgacccgatcgaggtgcgggccctgatgaacgtgtacggtgccggccggcccgccgaccgtccgctctggctcggttcgctgaagtccaacatcggccacacccaggcggcggccggggtcggcggggtcatcaagacggtgctggcgatgcggcacggcgtcctgccgcccaccctgcacgtggacgccccgaccaccgaggtcgactggtccgccggccaggtggccctgctgcgggcagagacaccgtggccggacacgggtcgcccgcgccgcgccggggtctcctccttcggggtgagcggcaccaacgcgcacgtggtgctggagcaggcccctgggcccgccgccgccccggcgggtgacgccccgcccgccgagacccggcccgtcggcgacccgccgccggtcgtaccgctggtgttgtccgccaggtcgcagccggcgctggccgggcaggcccgccggctgcgcgacctgctggccgcagcgccggagaccgacctcgccagcgccggactcgccctggccaccgcgcggtcggtgttcgaccaccgggcggtggtgacggccgccgggcgaccgcaggcgctcgacgcgctcgacctgctggccggcggcgaacccggaccggcggtcacgaccggcgtcgccgcccccaccgggcgcaccgtgttcgtctttcccgggcaggggacgcactgggccggcatgggtgccgacctgctcgaccagtcaccggtgttcgccgagtcgatgcgacggtgcgagcaggcgctgtcggcgcacaccgactggaagctcggcgaggtgatccggggcgcggccggcagcccgccgctggaccgcgtggacgtgctccagcccgtctcctgggcggtgatggtgtcgctggcgcaggtgtggcggtcgctcggcgtcgagccggacgcggtggtcggccattcccagggcgagatcgccgccgcggtggtctgcggcgcgctgaccctgccggacgcggcccgggtggtcgcgctgcggtcccaggtcatcggtcgggtgctctccggtcgcggcggcatggcgtccgtccagctgccggcccgggaggtcgcggggcggctggccgcctgggcgggccggctcgacgtcgcggccgtcaacgggccacagtcgaccgtcgtgtccggtgccgccgacgcggtcaccgaactggtcgaggcgttcgcggccgaggacgtccgggtgcggcggatcccggtggactacgcgtcccactcgacgcaggtggaccggctgcgcgccgagctgctcaccgtcctgggcccggtcgacgcccgtccggcgcaggtgcccttctactcgacggtgcagggcgggcgcgtcgacactgccggcctggacgccggctactggtaccgcaacctgcgggggcaggtccgcttcgaggagaccgtgcgggtgctgctcgacgacgggcaccgcgccttcgtcgaggccgccgcgcacgccgtcctcgtacccgcgatccaggagctgggggacagcgccggcgtccgggtggtggccgtggggtcgctgcgccgggaggcgggcggcctggaccggctcctggcctcggcggccgaggcgttcacccagggggtggccgtggactggtcccgggctctggccggggccgcgcgcgtcgccgtggacctgcccacgtacgcgttccagcggcaacgctactggctggagcccgccgcgcaggcggactccggcccggccggggacggctggcgctaccgggtcggctggcggcggcttcagcgcaccggcgccgcgccggccgaccggtggctgctggtgaccggcccggagcagccggcggagctggtcgaggcggtgcgcgacgcgctcaccgcgcggggcgccgaggtgcgcctggtgaccgtcgagccgaccagcaccgaccgggccgcgtgcgcggcgttgctcaccgcggccggtgcgggcggggcgacccgggtgctgtcgctgctcggcaccgatcgtcgcccgcaccccgaccacccggccgtgtccgtcggcgccgccgcgacgttgctgctgacccaggccgtcgccgacgccctgccggccgcccggctgtgggtcgtcacccggggcgcggtctccgtcgggcccggcgagaccgccgacgagcgccaggcgcaggtctgggggttcggccgggtcgcggccctcgaactgccccgcacgtggggcgggctcgtcgacctgcccgccgacgcggacggcccggtgtgggaggcgttcgtggacgtgctggccggggacgaggaccaggtcgcgctgcgcggcccggtcgggtacggtcgccggctccggcgcgcccccgcgctacccgcgaagcggcggtaccggcccaggggcaccgtcctggtcaccggcggcaccggcgcgctcggcgcgcacgtggcccggcggttggccgccggcggggccgcgcacctcgtgctcaccagccggcgcggggccgacgcccccggtgcggccgggctggtcggggaactccgggcgctgggcgccgaggtgaccgtcgcggtctgcgacgtcgccgaccgggccgccgtggcggcgctgctcgccgggctgcccgccgacgcgccgctgagcgcggtcttccacaccgcgggcgtggcgcactcgatgccgatcggcgagaccgggctcaccgacgtcgccgaggtgttcgccgggaaggtcgccggagcccgccacctcgacgaactcacccgggggcacgacctggacgcgttcgtcctgtactcgtcgaacgcgggcgtgtggggcagcagcgggcagagcgcgtacggggcggccaacgcggccctcgacgcgctcgccgaacggcggcgcgccgccgggctgaccgccacctccgtcgcctggggcctgtggggctccgggggcatgggcgagggcgacgccgaggagtacctgagccgccggggcctgcggccgatgcctcccgagcgtggcgtggacgccctcctggccgccctggaccgggacgagaccttcgtcgccgtcgccgacgtggactggacgctgttcacggccgggttcaccgcgttccggcccagcccgctgctcggcgacctcccggaggcccgcgcgacgctggccgacgccggacccgcgggctccgacctgccggcctggcacgccgccgcgagccccgacgaacgccgccggggcctgctcgacctggtacgccggcaggtcgccgccgtcctcggccacccggggcccgagcacgtcggccccgacgccgcgttccgggagatcggattcgactcgctgaccgccgtcgacctggccaagcggctcagggcggcggtcggcgtgccgctgtccgccaccctcgtcttcgaccaccccaccgcgacggcggtcgccgagcacctggccgggctgctcggtcccgcgccggccggcggcgacccgcgcgaggccgaggtgcgccgggccctggccgacctgccgctggcccggctgcgggacgccggcctactggacggcctgcttgcgcttgcggggctggacgccgacgcggtgccggacgggcccgagccggctcccggcgacgccatcgacgaactcgatccagaggagctggtgcgccgggtgctggacaacgccagctcctgacccgttccctcttccccccgaggagcccgcccatggtcatgccccccgacaaggtgatcgaggcgctgcgtgtctccgtcaaggagacggagcggctgcgccggcagaaccacgagctgctcgccgccctgcacgggccgatcgccgtcgtgggcatggcctgccgctacccgggcggggtgtcctctccggaggacctgtggcggctggtcgagacgggcacggacgcgatcggcggcttccccaccgaccgtggctgggacgtcgacgccgtgtacgacccggatcctgagtcgcggaacaccacctactgccgggagggcgggttcctggccggggcaggagacttcgacgccgcgttcttcggggtgtcgccgcacgaggccgtggtcatggacccccagcagcggctgcttctggaggtgtcctgggaggcgctggagcggtccgggaccgacccgcacagcctgcgcggctcgcgcaccggggtctacgtcggtgcggcccaccaggggtacgcggtcgacgccggtcaggtgccggagggcgcggaggggttccggctgaccggcagcgccgacgccgtcctgtccggacggatctcgtacctgctcgggctggagggtccggccctgaccgtcgagacggcctgctcgtcctcgctggtggcggtgcacctcgcggtgcaggcgctgcgccggggcgagtgcgggctggcactggccggcggggtcgccgtgatgcccgacccggcggcattcgtggagttctcccggcagcggggcctcgcggcggacgggcgctgccgggcgttcggggcgggcgcggacggcaccggctgggcggagggcgtcggtgtgctggtcctgcaacggctctccgacgcggtgcgcgacggccgctgggtgctgggcgtgatccggggttcggccgtcaaccaggacggggccagcaacgggctgaccgccccgagcggccccgcccagcagcgggtcatccggcaggcgctgaccgacgcccggctcggcgccgaccagatcgacgcggtcgaggcgcacggcacgggcacccggctcggcgacccgatcgaggcgcaggcgctgatcgccgcctacggcgccgaccggaccccggaccggccgctctggctcggctcgttgaagtcgaacatcgggcacgcccaggcggcggccggcgtcggcggcctgatcaagatgctcctggcgatgcgggccgggacgctcccacccaccctgcacgccgacgtcccgaccccgctggtcgactggtccgccggtgtcgtccggctgtcgaccggggtggtgccctggcccgcgttgcccggggcgccccgcagggccgggatctccgcgttcggggtgagcggcaccaacgcgcacgtgatcgtcgagcagccgccgccggtcccggtcgacgacccggcgccacccacgaggaccctgccgctggtgccgtgggtgctctccggccggacggaggcggcgctgcgcgcccaggcggaccggttgcgtacgcacctggcggcgcaccccgacgcggacccgctggacgtgggattctccctggccaccagccgggccgcgctggagcaccgggccgtgctggtggccgccgaccgcgacggcctgctccgcctcgtcgacgcgctggccgccggcgagccggcggcgggcctgatccggggcacggtacgtcacgatcgccggaccgggttcctcttcgccgggcagggcggccagcgcgtcgggatggcgcgcgaactgtacgaggcgttccccgccttcgccgacgccctggaccagctcgccgcccggctggaccggcacctcgatcgtccgctgctgcgggtgctgttcgccgagccggggtcggacgacgcccggctgctcgacggcacccggtacgcgcaggccgccctcttcgccgtcgaggtggcgttgttccgactggtccacggctggggggtccggcccgacgtgctgctcggccactcggtgggcgagctggcggccgcgcacgtggccggcgtactcgacgtggacgacgcgtgcgagctggtcgcggcgcggggccggctgatgggggagctgccgtcgggcggcgcgatggtggcggtccgggccaccgaggaggaggtcgggcccctgctcgacgggcagcgggtcgcggtggcggcggtcaacggcccgcgctcggtcgtggtctccggcgacgaggaggcggtgctggccgtggccgcccggtgcgccgccctcggccaccggacgcgacgcctcaacgtcagccacgcgttccactccccgcacgtggaggcgatgctggagccgttccggcgggtggcgcggggcctgacgtaccatgccccgacgatcccggtggtgtcgaacgcgacgggccggctcgccaccgccgacgcgctgcgcgaccccggttactgggtccggcacgtccgccagcccgtccggttccgggacggggtgcgggccgcccgcgaccagggggccaccgccttcgtcgggctcggcccggacggggtgctgtgcgcgttggccgaggagtgcctcgggcccaccggcgacgtgctgctgctgccggtgctgcgccccggtcggccggagcccgccaccctgctggccgccctggccggggcgtacgccggcggcgcggaaatggactggtcccgggtgttcgcgggcaccggcgcgcgcagggtcgagctgcccacgtacgccttccagcaccggcgctactggctggcgccgggcccgccgtcggcccgccgcgacgacgcctggcggtaccggatcgcctggcggcccctgccgaccgtgcccgccgccgccgggaccgagacggtggccggggcgtggttgctggtggtccccgcccacgacggcgtcgcgtcgctcgccgacgccgccgagcgggccgtgcaccggggcggggccacggtcacccggctgacggtggacgccgccgacgtggaccgggacaccctcgccgccgtgctgaccgaggccgccgccgacgcggacggcgggccggacggggtgctctgcctgctgggcctcgacgaccgggcacatccccggtccgcctcggtgccccgcggggtgctggcgaccctgtccctcgcccaggccctgaccgacctgggggcctccgcgcggctgtggtgcgtgacccggggggcggtcgccgtgacgcccggcgagtccccgtcggtcgccggagcccagttgtggggcttcggccgcgtggccgcgctcgaactcccccggtcctggggcggcctggtggacctgccggtcgacccggacgaccgggactgggacctgctgcggcgcgcgctgcgcggcccggaggaccaggtcgcggtccggggggcggtcgggtacgcccggcggctggtccccgcgcccgcgccccgggccgagcgggcctggcgtccgcgcggcacggtcctggtgaccggcggtacgggcgcgctcggcgcgcacacggcccgctggctggcgcgcaacggcgccacgcacctcgtcctcaccagccgccggggcgggaacgcccccggggtcgccgcgctgcgggcggaactggtcacgctcggtgccgaggtgaccgtggtcgcctgcgacgtcgccgaccgggaggccgtggccggcctgctcgccgggattccccgcgccgctccgctcaccgccgtgttccacgcggcgggcgtgccccaggtgacgccgcttcacgagacgaccccggagttgttcgcgcaggtctgcgcaggcaaggtcgccggggcggtgcacctgcacgagttggccggtgacctggacgccttcgtcaccttcgcctccgccgccggggtgtggggcagcggcgggcagtgcgcgtacgctgcggccaacgccgccctcgacgcgctcgccgagcgtcgtcgcgccgcagggctgcccgcgacctccgtcgcctggggggtctggggcgggcccggcatgggggcgggcgcgggggaggagtacctgcgccgccggggcgtccgggcgatgcccccggcagccgccctcgccgccctcgggcggatcctggacgccgacgagaccggggtgacggtctccgacaccgagtggggccggttcgcgtccggcttcgccgccgcgcgtcccgccccgctgctcgccgagctgccgggcggggacgtcgatccggccggcccggcgcaccgggcgcagccgcccgtgccccgaccggccccggcagccaccgaccgccccgggctgctggcgctggtccgcgccgaggccgccggggtgctggggcacgacggtgccgacgacgttccggccgacgcggagttctccgccctcggcttcgactcgctcgccgccgtccagctgcgccgccggctcgccgaggccaccggcctgagcctctcggccccggttctgttcgaccaccgcacccctgacgcgctcgccgcgcacctgcacggcctgctcaccggcgcggcgggcgggccacccgcgccggccgccgggagcgccctggtcgagatgtaccggcgggccgtcgccaccggccgcgccgccgaggcggtggaggtgctcggcaccgtcgccacgttccggccggtgttccggtccccggacgaactgggcgagccaccggccctcgtcccgctcggcaccggggcggggggacccgcgctggtctgctgcgcgggcacggccgcggcgtccggcccccgcgagttcacggcgttcgccgccgcgctggccggtctccgggacgtcaccgtccttccgcagaccggcttcctgcccggcgagccgctgcccgccgggctggacgtgctgctcgacgcccaggccgacgccgtcctggcccactgcgccgggggacccttcgtcctggtcggccactcggccggggcgaacatggcgcacgcgctgacggtccgcctggaggcgcggggcgcggaccccgccgcgctggtgctgatggacatctacacgcccgccgccccgggggcgatgggggtgtggcgcgaggagatgctggcctgggtcgccgagcggtccgtcgtccccgtcgacgacacgcggctgaccgcgatgggcgcctatcaccggctgctcctggactgggcgccccggccgacccgggcacccgtgctgcacctgtatgccggtgaaccggcgggcgcctggccggatccccggcaggactggcgttcgcgcttcgacggcgcgcacaccagcgccgaggtgcccggcacccacttctcgatgatgaccgagcacgcccccgtcaccgccgcgaccgtgcacaagtggctcgacgaggtgtgcccgccccgcgttccgtgacccgtacgccgggtccgtcccggcgagtccgacgacagcaggagaggaagcgcatgatcacagtcccgcccgacggggatcccgcgacctgggcccgccggctgcaactgacccgcgccgcgcagtggttcgccggcaaccacggcgacccgtacgcgctgatcctgcgcgcggagaccgacgacccgaccccgtacgagcagcgggtggccgcccagccgctgttccgcagcgagcagttggacacctgggtgaccggggacgccgcgctggcccgggaggtgttgaccgacgaccggttcggctggctgacccgggctgggcagcggcccgccgagcggaccctgccgctggccggcacggcactggaccacgggccggaggcccggcgtcggctggacgcgctcgccgggttcggcgggccggtcctgcgggccgacgccgcaggggcgcgtacccgggtcgtggagaccaccgcggtcctgctcgacgggatcggggagcggttcgacctggccgtgctcgcccggcggctggtcgctgcggtgctggccgacctgctgggggtgcccgccgcgcggcggggccgcttcgccgaggcactcgccgccgccggccgtacgctggacagccggctgtgcccgcagaccgtggcgaccgctctcgccaccgtcgccgccaccgccgagctgaccgacctgctgggcgaggtgccgcccccgccgtcgctgtccccgtccgccgccggctccgggccgccgcgtccgtccgcagccggttcctggccgccgctgccggctgacgaccggacggccgccgcgctcgcgctggcggtcggcacggccgaaccggcgatcaccctgctctgcaacgcggtcggtgcgctgctcgaccgccccgggcagtgggccctgctcggtggggacctcgaccggtccgccgccgtcgtcgaggagaccctgcgctgccttccgccggtgcgcctggagagccgcgtcgcgcagcaggacgtcaccctgggcgggcagttcctcccggcggacagccacctggtcgtgctggtcgccatggcgaaccggggtccgcgcgcggcgaccgccccgagcccggacgcgttcgaccctggcgggtcgcgcgtcccggcccgcgacgtggtgggcctgccgcagcttgccggcgccgggccgctgatcagactcgtcgtcacgaccgccctgcggaccctcgccgaggcgctgcccacgctgcggcgggcgtccggcggcgtccggtggcgacgctcgcccgtcctgctcggccacgcccgctttcccgtcgcacgggcggagagcggcgaacagcggtccgacgaccgcccggcgctggaggaggcgatccgatgcgcgtcctgatgacgtccttcgcgcacaacacccactactacagcctggtgccgttggcctgggcgctgcgcgcggccggccacgaggtacgggtggcgagccagccctcgctcaccgacaccatcgtgcggtcggggctgaccgcggtgccggtcggcgacgaccaggcgatcatcgacctgctcgccgaggtcggcggcgacctggtgccgtaccagcggggactggacttcaccgaggcccgtcccgaagtgctgacctgggagtatctgctcgggcagcagaccatgctcaccgcgctgtgcttcgcgccgctcaacggcgtctccacgatggacgacatggtcgccctggcccggtcctggcagcccgagctggtgatctgggagccgttcacctacgccgggccggtcgcggcgcgggtcgtcggtgcgacgcacgcccggctgctctgggggccggacgtggtcggcaacgcccggcggctgttcaccgagagcctggcgcggcagccggatgagcagcgcgaggacccgatggccgagtggttgcgctgcaccctgcaccggtacggctgcgagctcggcgacgacgaggtggagaccctggtcaccggcgggtggaccatcgatcccaccgccgacagcacccggcttcccgtccccgggcgtcgggtggccatgcggtacaccccgtacaacagcccgtccgtggtgccggagtgggtggccaaggccgaccggccccgcgtctgcctcaccctcggcgtgtcgagccgggagacgtacggcagggacgtggtctccttccaggagctgctcggcgccctcggcgacctggacgtcgaggtcgtcgcgacgctcagcgacgcccagcgcgaggacctgggtgacctgccggacaacgtccgggtgtgcgacttcgtgccgctggacgtgctgctgccgacctgtgccgcgatcatccaccacggcggggcgggcacgtggtcgacggccatgctctacggggtgccgcagatcatgatcgcgtcgctgtgggacgccccgctcaaggcgcagcaggcggagcgactcggcacggggatctcgatcccgccggagcggctcgacgccccgacgctgcgggcggccgtcgtccggatcctcgacgacccgtcgatcgccgccgccgcccgccgtcagcgcgacgagctgcgtgccgcgccgtcgccggccgaggtggtccgcatcctggaacgcctcgtcgcggacgaccggcccggccggccggccggaaccgccaccgaccactcctgaaaggaacgatgtccatgatgtacgcggacgccatcgccgaggtctacgacctgatctaccagggcaagggcaaggactacgcggcggaggcggcggagctggaggcgctggcccgggcccgtcggccgcacgcccggacgctgctggacgtggcgtgcggcacggggctgcacctgcggcacctggcggggctcttcgacgacgtgggcggcatcgagctggcaccggacatgctgagcatcgcccagcagcgaaaccccggggcggccctgcacctcggcgacatgcggaccttcgacctggggcaccgctacgacgtcatcacctgcatgttcagttcggtgggccacctggccaccacggccgagctggacgcgacgttggcccggttcgccgcgcacctgtcccccgggggagtggcgatcgtcgagccgtggtggttcccggagaccttcacccccgggtacgtgggcgcgagcctggtggaggtcgacggccgtaccatctcgcgggtctcccattcggtgcgcgagggcggcgcgacccggatcaccgtgcactacctcgtggccagccccggcgggggagtccggcacttcgacgagagccacctgatcaccctcttcgaacggtccgactacgaacgtgccttcgcccgggcgggtttcacgacggagtacctgacgcccggcccgtccggccgcggtctgttcgtcggcgtccacccctgacgacccgttgccggtgcgcctcgacccgcgcccccgacccgctggaggaacagatgccagacacccccgagctgaaccggatactcgacgcgatcctcgcccaggagaccgacgcgcgggagctggcggccctgccgctgccctcctcctaccgggccgtgacggtgcacaaggacgagacggggatgttcctgggccttccccgccaggagaaggacccgcgcaagtcgctgcacacggaggaggtgccggtgcccgagctgggccccggggaggccctcgtcgcggtcctggccagctcggtcaactacaacacggtctggtcgtcgttgttcgagccgctgcccaccttcggcttcctggagcgctacggccggctctccgagctggcccggcggcacgacctgccgtaccacatcctcggctcggacctggccggcgtggtgctgagggtcgggcccggcgtcaaccgctggcggccgggtgacgaggtcgtggcgcactgcctctcggtggagctggagtccgccgacggccacggcgacaccatgctcgacccggaacagcggatctggggcttcgagaccaacttcggcggcctcgccgagatcgcgttggtcaaggcgaaccagctgatgcccaaacccgaccacctgacctgggaggaggccgccgcgccgggactggtcaactccaccgcctaccgccagctggtctccggcaacggggcccggatgaagcagggcgacaacgtcctcgtctggggggccagcggcggtctcggcgcgttcgccacccagctcgtgctggccggcggggccaatcccgtctgcgtggtctccagcccgcgcaaggccgacatctgccgtcggatgggcgccgaggccgtcatcgaccgggtcgccgaggactaccgcttctggtccgacgagcgcacccagaatccccgggagtggaagcgcttcggcgcacgcattcgggagctgaccggaggcgaggacgtcgacatcgtcttcgagcaccccggccgggagacgttcggcgcctcggtctacgtgacccgcaaaggaggcaccgtggtcacctgcgcctcgacgagcggtttcgagcacgtctacgacaaccgttacctgtggatgtccctgaagcgcatcgtcggcacgcacttcgccaattaccgggaggcgtgggaagccaaccggttggtggtcaagggcaagatccacccgacgctgtcgcgctgctacccgctggaggaggtcggccaggcggtctacgacgtccatcacaacctgcaccagggcaaggtcggcgtgctcgcgctcgcgccgcgcgaggggctcggggtccggaacccggagctgcgggaatgccatcttgccgcgatcaaccgcttccgggtgccggcctgacgggccgcctttgacgcccgggggcgcggcggctggcatgcgggcgaaccgggtgttaccgggcggaagcaattctcactgcgagtagttgcagggtgcaccggctactgtgaacatatcgatagtcttatgtagccatcgacccccctgaatcctctattcgttgtgtgcgaggtggttggacgcatgactggtaccagcattcccccgcgggaccacgaactccgattcttcgaacttctggccagggaggcacccttaccgcagtacgaggaactggtgcaccaggcgcaccgggacggagtggaccaggccacgctcgaccgggtgatgatcgccaagcgactcgcgttggagcttcgagaggtcatcgggaggcggtgtcagcggcaggcggagctggccgccctcgtcgacaccgcccgtgacctcgccggggcgacgaacctggaggccgggctgcagctggtggtgcggcggacccaactgctgctcgccggggacgtggcgttcgtcagcctcgtcgacgacgcgaccggcgaatcctacgtcgcctcggccgtcggggcggccaccgcgctgaccagcggctaccggctgccctggcgcgacgggctggtcgtggccgccgcaccgcgcgagccactctcctggacggcggaccacctcgccgacgagcgcctcgaacgacacccggccgccgacggcctggtccgcgcggaagggctgcacgcggtgctgtccgtggttctgagcgtcgagggccggcacctcggcaacctgcacgtcggccaccggcaggtccgccacttcgccccggacgaggtcgcgtcgctgcgcctgctcgccgatctcgcggcgacggcagtggagcggatcatgctgctcgacgacacgtgggccgaactcaagcaggcccagcaggaggcggccagggcccgagccgagctgaacgcggtccgcatggccgaccgcctgcaacccgaactcgtccagctcatcctcgacggcggcgaactcgacgacctggtgggcagcgccgtgcggcgactgggcggcgccctgcacgtgcgtgaccgggccaacggcgtgctggcggcggccggtgaaatccctgtcccgaacgagcgggaactggcccgagtgcggctgaacgcccacgccaccggccgacccggccgcctgaccaccggttcctgggtggtgcccctggcggcccgcgccggtgacctcggctgtgtgttgttccacgccgacgagccgtccgacgacgagcggatggcggccctgccggcggtcgcgcagaccgtggcgctgctgatgaccaggaacggcgggagccacggccagccgggcgacgggctcctggaggacctgctcggcccgtggccggacctggagcggggcgggaagcgccgtcggtacacacctgtcgagttcgaccggccctacgtcgtcgtggtcgcccgccccgagggcgccacctcgccccgggtgttcgaacgggcggtctccgtcgcccacggcctgaacggcatgaaggccatccgggacggccaggcggtgctgctgctgcccggtgacgacccgggggcccgggcccgggacgtgacgcgggaactgagcgggctgctcggcctaccggtcacggccggaggcgccggaccggtgcgcacggcggactcggtcagccgcacctaccaggaggcggcccggtgcgtcgacgccctggccgcgctggacgcgaaggggcgggcggcctgctcacgggacctgggcttcctcgggctgctggtcgccggcggccacgacgtcaccggtttcgtcgaccgggtcatcggacccgtgctgagctacgacgcgcgccggctcacgaatctcagggagaccctccagacctacttcgactcggcgggcagccgtacccgggcggcggagatgctgcatctgcatccgaacaccgtgtcccgccggctggaccgcatctcccagctgctcggccgggactggcggcagccggaccgggccctcgacacgcagctcgctctgcgcctgcaccggatccgtggcctgctctgccaggaacggggctacccgggcccatcgcaggagccggaccaacccgcgcggcctatccggcggcaccgccctccagcatccgcagggcgtgcgccacggacgccaaggtgacgtgccggtgccagccttggtatgaccgaccctcgaagtcttggatgccgacgtcgaggctgacctgggagaagtcggtctcgacccgccgggtgagcttgctcagccgcagcagtgggccgtacccggcgtcggtcatgttggtcagccacatctgccgtacgccgcgctcgtaggtctgccacttgccgagcagtgtcaggggcagcccgggcgcggcggcgcgcgccgcccccggcggggccggggcggacggaccgggcgggcgggcaccggacaggcccggccaatagacctgtagcggtgcgaccaggctcgtgcgccgtgcgccggggctggccgggtcgatccactccaccggacggcgctgggcccgcgtcaggctgagcaggtgctcggcggaggccgccgcgacccggttctcgcgcgggccgggcccggcggccagcagggtgcagccgctgttgatccgtagcaggaagggcagacccgccgtggtgaacgcctcgatcagcgggggcagcgccgagtgccgggcgtccattaccaccgggcgagggccgattccccaggccgcggccttcagcaccgcctgcaccgccgcgccgtcgctggtcgtgccgtcctcgtccgccggtacgctcgcgcgggcgcggttgtcctggagccaacccttaccgatggacaactgccagttgatgggcgcggcgacggtctccgaggccagccagaggccgtagctctgctggctgttgaccgtctcgcccagcgcgggcacgtaccggcgttccacgccgaccgagtgccggccggtcttcggcaccagcatcgaccgcaccacccaggcccggggcgacagcgtccggtccaggtggccggcgagcgcggcacggacggtctcccagtcccaggtggagcaactgatgaagtggtgcatgctctgtgccgccgccggatcgtcggcgatggcggccaggttgcgcatggtcttgcggccggaggcggtcagcagcccccggacgtacagttcgcccttgcgtcgctggtcggcgcggggcagcgaggccagcagggcggcgcagaaccgggacacggtgtccggatcggaccttgccgcggtcacctcctcgcggacgtcgagcgtcggcaccatcactcccctcctgcggcgggacgatgtgctgatcacggcagacccggccccccggtcccaccatcgcccggcgacgcctgccttgcccaggtgcgtcggaaacacacttggcgacgacggcgatcccgcacccaccgcagccccgccggtgcgtgtccgtggcgggcgggcgcgggccggcgacccggtgacgcccgcacacatcgcggcttcggccgcggcgaagtgtgtgaccggcgaacctcgcttcccgcgccgccatccggaagcctgcaagggaccggaagccttccaacgagattggcatccccccggcaaaggacccagatgacctccgcagcgcaccattccccgcatccggcgaaggccgacgccctgatggacgacgcccacgccgacatcggggccgatgccgaggccgacggtcgacggctcgaccgggccgccctgcggcgggtcgccgggctgtcgaccgagagggccgacgtcacggaggtcgagtaccggcaggtgcggctggagcgcgtcgtcctggtcggcgtgtggacctcgggcaccgccgacgaggccgaacggtccctcgccgagctggcggcactcgccgagaccgcgggagccgtggtgctcgacggggtgatccagcgccgcgaccggcccgacccggcgacgtacatcggctccggcaaggcgcgggagttgcgggacatcgtccaggaggtgggggccgacacggtgatctgcgacggtgagctgagcccggcccaactggtacgcctcgaagaggtcgtcgacgccaaggtggtggaccgcaccgcgctgatcctcgacatcttcgcccagcacgccacgtcccgcgaggggaaggcgcaggtggccctggcacagatgcaatacatgctgccgcggctgcgcggctggggccagtcgctctcccggcagatgggcggaggtgccggcggcggtggcatggccacccgggggcccggcgagaccaagatcgagaccgaccggcggcgcatccacgagaggatggcccggctccgacgggagatcgcggagatgaagtccggccgcgaactcaagcgccgcgatcggcggcgcaacagcgtcccgtcggtcgcgatcgccggttacaccaacgccggcaagtcctcgctgctcaaccggctcactggcgcgagcgtgctggtgcagaacgcgctgttcgccaccctcgacccgacggtgcgccgggccaccaccccgagcgggcgcagctacacgatcaccgacaccgtcggattcgtccggcacctgccgcaccacctggtggaggcgttccgctccaccctggaagaggtggccgaggccgacctcctgctgcacgtggtggacggcgcccaccccgccccgctggagcagctcgcctcggtgcgcgcggtcatccgggacgtggacgcggcgggagtgcccgaactcgtcgtgatcaacaaggccgacgccgccaccccggccgccctggccgcgttggcggaggccgagccgcaccacgtcgtcgtctcggcccgcaccggtcagggcatcgacacgcttcggcagttgctggaggccgcgctgccgcaccgggaggtccgggtcgacgtcctgatcccgtacgtcgcgggcagcctcgtggcccgggtgcacgccgacggcgaggtgctggccgaggagcacacggccgacggcaccctgctgcaggcgcgggtggcccccgacctggctgccgagctcagcgcgtacgccaggacctgagcgtcgccgccccccgggcggcatccggagctggcgaagctgtggcccgtagagggaggcaggcgatgaagcgagatctcggggatctggcactcttcggaggacacgccagcttcctccagcagatccacgtcgggcgccccaaccggatcgatcgggccaggctgttcgaccggctgtcctgggcgctcgacaacgagtggttgaccaacaacgggccgctggcacgggagttcgaggagcgggtcgccgacatggtcggggtcggcaactgcgtggcgacgtgcaacgccacggtggccctccagctgctcgcgcacgccaccgagctgaccggtgaggtgatcatgccatcgctcaccttcgccgcgaccgcacacgcggtgcgctggctcgggctggagccggtcttctgcgacatcgacccgcgcaccggatgcctcgaccacgtggcggtcgccgcggccatcacgccgcgcacgtcggcggtcttcggcgtccacctctggggccgcccctgcgacgtcaacgcgctggagaaggtgaccgccgacgcgggcctgcgcctgttcttcgacgccgcccacgccatcgggtgcacctcacagggccgcccggtggggcggttcggccacgccgaggtgttcagcttccacgcgacgaaggtcgtcaacgccttcgagggcggggcgatcgtcaccgacgacgacgacctcgcccaccgcgtccgctccctggcgaacttcggcttcggcctgcacagccccagcgcggccggcggcaccaacgcgaagatgagcgaggcgtccgccgccatggggctcacctcgctcgacgcgttccccgaggtggcccgccacaaccaggccaactacgagcagtactgcggtgagctggcccggattcccggcctcagcgtgatcgacttcgcccccgacgagcggcacaactaccagtacgtgatcgtcgagatcgacccggacgtcaccgggttgcaccgcgacctgctcgtcgacctgctccgggccgagaacgtcgtggcgcagcgctacttctcgccggcctgtcaccaattggagccctaccggtcccggcagcagttccagctgccgcacaccgagcggctctcggcgcgcgtcctggcgctgccgaccggctccgccatctcccgggaagacatccgcagggtgtgcaacatcgtgcggttggcggtctcccggggattcgaattgaccgctcggtggcagcagcagcccgggcccgacggacagagcgtggtggcacccggttgaccgaacggcaccggacggacgtgtgggagggcccgtgaccatggagatctccgcctcgaatcccgtggcgacctgcgctgtccccggcagcgacccgaccgcggcggcgcgcgtgctgtacgacgaggtcgccgggtcaggaatcgtgccgccggcagagatcggggccgccgcccaggggttggtggcattggcacgcatctacgggaccacaccttttctgccgcttgagcaggcccgccgcgaaatcggcctggaccgggccgggttcgggcggctgctggacctgttcgcccggattcccgggttgcgcaccgcagtggagaacggaccgtccggtcgctactggaccaacacggtgctcggcctcgaaagggccggcgtcttcgacgccgtgctcgaccggaggccggcgtttccgcatctcgtcgggctctacccgggccccacgtgcatgttccgctgtcacttctgcgtaagggtcaccggggcccgctaccaggcctcggcgctggacgacgggaacgccatgttcgcctctgtcatcgacgaggtccccgcgcacaaccgcgacgcggtgtacgtctccggtggcctcgagccactcaccaaccccgggctcggtgcactggtcagccgggcggccgagcggggatttcggatcatcctctacaccaactcgttcgccctcacggagcagaagctcaagggtgagcggggattgtggagcctgcacgccatccgcacgtcgctgtacgggttgaacgacgaggaataccgggcgaccaccggcaagcagggggccttcacccgggtacgggcgaacctcacgcggttccagcagctgcgtgccgagcggggcgagccggtgcggctcggcctcagctacatcgtcctgcccggccgcgccgggcggctgagcgcgctgatcgacttcgtcgccgagctcaacgaggcggcaccggaccgcccgctggactacatcaacctgcgggaggactacagcgggcggccggacgggaagctctccctggacgagcgcgccgagctccaggccgagctgcaccggttccgggagagggcaatgcagcggacgccgaccctgcacatcgactacggctacgccctgcacagcctgatgacgggaagcgacgtggagctcgtgcgtatccggccggagacgatgcgccctgcggcccacccgcaggtgtcggtgcaggtggatatcctcggtgatgtctacctctatcgggaggcggcgtttccgggcctggccggtgccgaccgctatcgcatcggcacggtatctcccggcacgacgttggcgcaggtggtggagacgttcgtgaccagcggcggatcggtggtcgcgaagcctggcgacgaatacttcctggacggattcgaccaggcggtgaccgcgcggctgaaccagatggagaccgacgtcgccgatggctggggagaccgacggggtttcctccgctgatggagatcgactggtgagagcgggtggccaacgccgaagaaagccagttgccggtggcccgcaccgccgtttcagtcgtcgggtatagtgcccgtcatggctgttgtgtgcttcatgaggctccgccgcgcatagcggcggaccatcgcttctcttgatgagtgtcgccgcccatcgggtcactgccggtgcggcgttccctgccgaccggctccgaacgatattcgcggagcacgcacatgccctacatccagcacgccgggcgacatgaattcggccagaatttcctggtcgaccgctcggtgatcgacgatttcgtcgaactcgtcgcccggaccgacggccctatcgtggagatcggcgccggcgacggtgcgctgaccctacccctgagccggcagggaagggagttgaccgcagtggagatcgactccaagcgttccaagcggctcagccggcagacacccgacaacgtcaccgtggtctgcgcggatgtcctgagcttccggttcccccagcatccgcacgtggtcgtcgggaacatccccttccacgtgaccacccccatcgtgcgggctctcctcgccgcggaccactggcacacggcggtgctgctggtgcagtgggaggtggcccgcaggcgggccggcgtcggcggcgcgacgctgctgaccgcgagctggtggccctggtacgacttcgaactgcactcccgggttccggcccgcgccttccggcctgtcccttccgtcgacggcgggctgttctccatggtccgtcgcgggaccccgctggtcgacgaccggaggggttaccaggaattcgtccggctggtgttcaccggcaaggggcacggattgccggagatccttcagcggaccgggcggatcgcccgcaaggaccagcaggactggcaacgggccaaccgggtggggccgcagcacctgcccaaggacctgaccgcccaccagtgggcctccctgtggcacctggtggcacccgcccggccggccggcccccgccgtccggcaccgcgccggccaggaagccccgcttcggcgcgccggcgctga

SEQ ID NO: 2 - protein, 560 aa, micromonospora carbonacea subspecies aurantiaca
VPVPTQEAPLRNSPPPAHSQLVLSEVTKHYAERVVLDRVSLTVKPGERVGVIGENGSGKSTLLRLVAGLETPDNGELTVSAPGGIGYLAQRLRLPAGGSTVRDVVDHTLADLRDLEARLRAAEADLATATPEQLDAYGTLLTVFEARGGYQADARVDAALHGLGLAELDRDRDVDTLSGGERSRLALAATLAAAPELLLLDEPTNDLDIEAVEWLEDHLRSHRGTVVVVTHDRVFLESVTSTILEVDTDTRAVHRYGDGYASYLRAKAALRESRERAYAEWVAEVERQSQLAERAGTMLRSISRKGPAAFSGAGAHRSRSSSTATSRKARNANERLRRLRENPVPRPADPLRFTASVAPDATDADTRRVELTDVRVGRRLHVPELTIGPAERLLVTGPNGAGKSTLMRVLAGELVPDGGTVRLPARIGHLRQDVTVGQPGRSLLETYASGRPGHPEEYAEELLARGLFRPDDLRMPVGTLSVGQRRRIDLARLVARPADLLLLDEPTNHFAPLLVEELEQALDGYAGALVVVTHDRRMRSTFTGARLELHQGVATGASRA

SEQ ID NO: 3 - DNA, 1683 bp, micromonospora carbonacea subspecies aurantiaca
gtgccagttccgacacaggaggcccccttgcggaacagcccgccgccagcccattcgcagctcgtcctgagcgaggtcacgaagcactacgccgagcgggtcgtcctggaccgcgtttcgctcaccgtcaagccgggggagcgggtcggcgtcatcggcgagaacgggtcggggaagtcgaccctgctgcggctcgtcgcggggctggagacgccggacaacggcgagttgaccgtctcggcgcccgggggcatcggctatctcgcccagcggcttcggctgccggccggcggcagcaccgtacgggatgtggtggaccacacgctcgccgacctgcgagacctggaggcgcggttgcgcgccgccgaggcggacctggccaccgccacgcccgagcagttggacgcctacggcacgctgctcactgtgttcgaggcccgcggcggctaccaggccgacgcccgggtggacgccgccctgcacggtctcggcctggccgagctcgaccgcgatcgcgacgtcgacacgctctccggcggggaacggtcccggctcgcgctcgccgcgaccctggccgccgcgccggaactgctgctgctcgacgagcccaccaacgacctcgacatcgaggccgtggagtggctggaggatcacctgcggtcgcaccggggcaccgtcgtcgtggtcactcacgaccgggtgttcctggagtcggtcacgtccaccatcctcgaggtcgacaccgacacccgggccgtgcaccggtacggcgacggctatgccagctacctgcgggccaaggccgccctccgggagagccgggagcgcgcgtacgcggaatgggtggccgaggtcgagcggcagtcccaactcgcggagcgggccgggacgatgctccggtcgatctcccgcaagggaccggctgcgttcagcggggccggtgcccaccgctcccggtcgtcgtcgacggcgacgtcacgcaaggcccgcaacgccaacgagcggcttcgccggctgcgggagaatccggtaccgcgacccgccgacccgttgcgcttcaccgcgtcggtcgccccggatgccacggacgccgatacccgccgcgtcgagttgaccgacgtccgggtgggccgccgcctgcacgtgcccgagctgaccatcggacccgccgaacggttgctggtgaccggacccaacggcgcgggtaagagcaccctgatgcgggtgctcgccggggaactcgtgcccgacggcggaacggtgcggctgccggctcggatcggccacctgcgtcaggacgtgacggtcgggcagcccgggcgctctctgctggagacgtacgcgtcgggtcggccggggcatcccgaggagtacgcggaggagttgctcgcccgcggtctgttccggcccgatgacctgcgcatgccggtcgggacgctctccgtcgggcagcgccgccggatcgacctggcccggctggtcgcccgcccggccgacctgctgctgttggacgagcccaccaaccacttcgcgcccctgctcgtggaggagctggaacaggcgctggacggctacgccggagcgctggtcgtggtgacgcacgaccggcggatgcggagcaccttcaccggggctcggctggaactgcaccagggcgtggccaccggggcgagccgggcctga

SEQ ID NO: 4 - protein, 264 aa, micromonospora carbonacea subspecies aurantiaca
MSPSADPSELWLRRYRPVNDPAVRLFCFPHAGGAASAYLPFARRLAADVDVLAVQYPGRQDRRGEPLIESVDALVDGLLPALLAWADRPVAFFGHSMGATVAFEAARRLPPADADRLVHLFASGRRSPSVGRRDRFYRFDDELIDEIRRLQGTDSSLLDDRELLDMLLPAIRNDYRAAAAYEYRPGPRLRCPVTVLAGAADTHVTTDEAAAWAEVTAAATMVRTFPGGHFYLNDQLDAVCAEVTTTLAAVSTTALTAVPGADPG

SEQ ID NO: 5 - DNA, 795 bp, micromonospora carbonacea subspecies aurantiaca
atgtccccgtccgccgatccgtccgagctgtggctacgccgctaccggcccgtcaacgaccccgccgtccggctgttctgcttcccgcacgccgggggcgcggccagcgcgtacctgccgttcgcccgccggctcgccgccgacgtggacgtgctggcggtccagtacccgggccggcaggaccgccgcggcgaacccttgatcgagtccgtcgacgccctggtggacgggctcctgcccgcactgctcgcctgggcggaccgaccggtggccttcttcggtcacagcatgggcgccacggtggccttcgaggccgcccgccggctcccaccggccgacgccgatcggctcgtgcacctcttcgcctccggccgccgtagcccgtccgtcgggcggcgggaccggttctaccggtttgacgacgagctgatcgacgagatccgccggctccagggcaccgattccagcctcctggacgacagggaactgctggacatgctcctccccgccatccgcaacgactaccgggccgccgccgcctacgaataccggccagggcccaggctgcgttgcccggtcaccgtactcgccggggccgccgacacccacgtcaccaccgacgaggccgcggcgtgggccgaggtgaccgcagcggccacgatggtccgcacgttcccgggcgggcacttctatctcaacgatcagctcgacgctgtgtgcgccgaggtcacgaccaccctcgcagcggtgtccacgaccgccctcacggcggtgccgggtgccgaccccggctga

SEQ ID NO: 6 - protein, 410 aa, micromonospora carbonacea subspecies aurantiaca
MTQTPNAPAGPIDLPKGADAQGLLDWFAYMRKNWPVSWDETRQAWHVFSYRDYQTVTTNPLIFSSDFTSVFPVPSELALLMGPGTIGGIDPPRHAPLRKLVSQAFTPRRIAQMELRIGQITADVLDQVRDQDRIDIASDLAYPLPVTVIAELLGIPTKDHEKFREWVDIILSNEGLEYPNLPDDFTETVGPAIEEWSEFLYAQIAHKRAEPKDDLISGLCAAEVDGRKLTDEEVVNIVALLLTAGHISSATLLSNLFLVLEEHPQAQAAVRADRSLVPGVIEETLRYRSPFNCIFRILNEDTDILGHPMRKGQMVIAWIASANRDTEVFTDPDTFDIRRESNKHLAFGHGIHHCLGAFLARLEAKVFLNQTLDQFTEFRIDHVGVEFYDADQLTARRLPVQVVRDGRHPK

SEQ ID NO: 7 - DNA, 1233 bp, micromonospora carbonacea subspecies aurantiaca
atgacgcagaccccgaacgccccggcgggaccgatcgacctgcccaagggcgccgacgcccaggggctgctggactggttcgcgtacatgcggaagaactggcccgtctcctgggacgagacccgtcaggcctggcacgtgttctcctaccgggactaccagaccgtgaccaccaacccgctgatcttctcgtcggacttcacctcggtctttcccgtaccgtcggagctggccctgctgatgggccccggcaccatcggcggcatcgacccgccgcggcacgcgccgctgcgcaagctggtgagccaggcgttcaccccccgccggatcgcccagatggagctgcggatcgggcagatcaccgccgacgtgctcgaccaggtacgcgaccaggaccggatcgacatcgccagcgacctcgcgtacccgctgccggtgacggtcatcgccgagctgctcggcattcccaccaaggatcacgagaagttccgcgagtgggtggacatcatcctcagcaacgaagggctggagtatcccaacctcccggacgacttcaccgagacggtgggccccgccatcgaggagtggtccgaattcctgtacgcccagatcgcccacaagcgcgccgaaccgaaggacgacctgatcagcggcctctgtgcggcggaggtcgacgggcgcaagctgaccgacgaggaagtcgtcaacatcgtcgcgctgctgctcaccgccgggcacatctccagcgccacgctgctcagcaacctgttcctggtgctggaggagcacccgcaggcacaggccgcggtccgcgccgaccgcagcctcgtgccgggcgtgatcgaggagacgctgcgctaccggtccccgttcaactgcatcttccggatcctgaacgaggacaccgacatcctcggccaccccatgcgcaagggccagatggtgatcgcctggatcgcctccgcgaaccgcgacaccgaggtgttcacggacccggacaccttcgacatccgacgcgagtcgaacaagcacctggcgttcggccacggcatccaccactgcctgggcgcgttcctggccaggctggaggcgaaggtcttcctcaaccagacgctcgaccagttcaccgagttccggatcgaccacgtcggggtcgagttctacgacgccgaccagctcaccgcgcgacgcctccccgtccaggtggtacgcgacggacggcacccgaagtaa

SEQ ID NO: 8 - protein, 402 aa, micromonospora carbonacea subspecies aurantiaca
MEHPVTAGSCRFYPFSDRTDLNIDPTYGELRSKEPVARVRMPYGGDAWLVTRHADAKKALSDPRLSIAAGAGRDVPRASPRLQEPDGLMGLPPDAHARLRRLVATAFTPKRVRDIAPRVVQLADKLLDDVVETGPPADLVQQLALPLPVMIICEMMGIGYDEQHLFRAFSDALMSSTRYTADQVDRAVEDFVEYLGGLLAQRRAHRTDDLLGALVEARDDGDRLTEDELVMLTGGLLVGGHETTASQIASQIFLLLRDRTRYEQLHARPELIPTAVEELLRVAPLWASVGPTRIATEDLELNGTTIRAGDAVVFSLASANQDDDVFANAADVVLDRDPNPHIAFGHGPHYCIGASLARLEIQAAIGALARRLPGLRLAVEENELDWNKGMMVRSLVSLPVTW

SEQ ID NO: 9 - DNA, 1209 bp, micromonospora carbonacea subspecies aurantiaca
atggagcatccagtaacggccgggtcctgcaggttctaccccttcagtgaccgtaccgacctgaatatcgatcccacgtacggcgaactgcgctcgaaagagccggtcgcccgcgtccgcatgccctacggcggggacgcctggctggtcacccggcacgccgacgccaagaaggccctctctgacccccgactcagcattgcagccggagccgggcgggacgtgccgcgcgcctccccccgtctccaggaacccgacggtctgatgggtcttccccccgacgcgcacgcccgactgcgcaggctcgtcgccacggcgttcacgccgaagcgcgtacgggacatcgccccgcgcgtcgtccagctcgccgacaagcttctcgacgacgtggtcgaaaccgggccgccggccgacctcgtgcagcagctcgcgcttcccctgccggtgatgatcatctgcgagatgatgggcatcgggtacgacgagcagcacctgttccgtgccttcagcgatgccctgatgtcctccacccgatacacggccgaccaggtcgaccgcgcggtagaggacttcgtcgagtacctcggcggcctcctcgcgcagcgccgtgcacaccgcaccgacgacctcctcggcgccctggtcgaggcgcgagacgacggcgatcggctgaccgaggacgaactcgtcatgctcaccggcggcctgctcgtcggcggccacgagacgaccgccagccagatcgcctcgcagatcttcctcctgctgcgcgaccggaccaggtacgagcaactccatgcccgtccggagttgatccccacggcagtcgaggaactgctgcgggtggccccgctctgggcctcggtcggccccacccgcatcgccaccgaggacctggaactcaacgggacgaccatccgggccggcgacgccgtcgtcttctcgctggcgtccgccaatcaggacgacgacgtcttcgcgaatgccgcagacgtcgtgctcgaccgcgacccgaatccgcacatcgccttcgggcacgggccccattactgcatcggggcgtcactggccagactggaaatacaggccgccatcggcgccttggccaggcggcttcccggtctccgcctggccgtcgaggaaaacgaacttgattggaacaagggaatgatggtacgcagcctcgtgtcccttccggtgacgtggtga

SEQ ID NO: 10 - protein, 4471 aa, micromonospora carbonacea subspecies aurantiaca
MRVVGADACSAAVPAGPRMGFPASFFDPGDLMTVQSDVLRHRDIAVIGMSCRLPGAPSIEEFWDLLCSGRSAVDRQPDGGWRAVIDGKGESDAAFFGMSPRQAAAVDPQQRLMLELGWEALENARIRPADLKGSDTGVFVGLTADDYATLLRRSGTPISGHTATGLNRSLTANRLSYLLGLRGPSFTVDSAQSSSLVAVHLACESLLRGESAVAVVGGVSLILAEESTAAMARMGALSPDGRCFTFDARANGYVRGEGGVAMVLKPLIRAIEDGDQVHCVIRGCAVNNDGGGPSLTHPDREAQEALLRRAYERAGVAPEHVDYVELHGTGTKAGDPVEAAALGAVLGVARGCDNPLAVGSVKTNVGHLEGAAGITGLLKAVLCVREGVLPPSLNFRTPNPDIRLDELNLRVQTELQPWPGDGTGRPRVAGVSSFGMGGTNAHLILEQAPVAAEETAVTDAGVGSVRVVPVVVSGRSVGALRAYAGRLREVCAGLSDGGGSGGGSGLVDVGWSLVSSRSVFEHRAVVFGGGVAEVVAGLDAVASGAVSSGSVVVGSVASGVAGGGGRVVFVFPGQGWQWVGMGAALLDESEVFAESMVECGRALSGFVDWDLLEVVRGGGGDGSFGRVDVVQPVSWAVMVSLARLWMSVGVVPDAVVGHSQGEVAAPVVGGVLSVADGARVVALRSRVIGEVLAGGGAMVSVGLPVAVVLDRLAGWGGRLGVAAVNGPSLTVVSGDVDAAVGFVGECERDGVWVRRVAVDYASHSAHVEAVEGMLSGLLGGLCPGRGVVPFYSSVVGGVVDGVGLDGGYWYRNLRERVLFSDVVGRLVGDGFSGFVECSGHPVLAGGVLESVAVVDPDVRPVVVGSLRRDDGGWGRFLTSVGEAFVGGMSVDWKGVFAGAGARLVDLPTYPFQRRHYWAPNTDGAPAPILDDHAEAENEPAESEPGIRAELLTLAEPEQLNRLLATVRASTAVVLGLDSAQAVDPERTFKEHGFESVTAVELCNHLQRGTGLRVPASLVYNHPTPMAAARKLQEEIQGRQPENVRQVTSAAAVDDPVVVVGMGCRFPGGVVCAEGLWDLVLGGGDAVSGFPVDRGWDVEGLFDPVRGVVGKSYVREGGFVYDAGMFDAEFFGVSPREAVAMDPQQRLFLEVSWEALERAGIDPLGLRGSRTGVYVGVMGQEYGPRLVESGGGFEGYLLTGTSPSVVSGRVSYVLGLEGPSISVDTACSSSLVALHLACQGLRLGECDVALAGGVTVIAAPGLFVEFSRQGGLSGDGRCRAFAGGADGTGWGEGAGVVVLERLSVARERGHRVLAVVRGSAVNQDGGSNGLTAPSGVAQRRVIGAALVAAGLGVSDVDVVEAHGTGTRLGDPIEAEALLGSYGRGRVGGALLLGSVKSNIGHTQAAAGVAGVIKMVMALRAGVVPATLHVDVPSPLVDWSSGGVELVTEARDWPVVGRVRRAGVSAFGVSGTNAHLILEQAPEFDDPVVTDTDTDAGVGRGLSVVPVVVSGRSTAALRAYAGRLREVCAGLSDGAGLVNVGWSLVSSRSVFEHRAVVFGGGVAEVVAGLDAVVSGAVASGSVVVGSVASGVAGGGGRVVFVFPGQGWQWVGMGAALLDESEVFAESMVECGRALSGFVDWDLLEVVRGGAGEGVWGRVDVVQPVSWAVMVSLARLWMSVGVVPDAVVGHSQGEVAAAVVGGVLSVADGARVVALRSRVIGEVLAGGGAMVSVGLPIVDAQERLAGWGGRLGVAAVNGPSLTVVSGDVDAAVGFVGECERDGVWVRRVAVDYASHSAHVEAVEGMLSGLLGGLCPGRGVVPFYSSVVGGVVDGVGLDGGYWYRNLRERVLFSDVVGRLVGDGFSGFVECSGHPVLAGGVLESVAVVDPDVRPVVVGSLRRDDGGWGRFLTSVGEAFVGGMSVDWKGVFAGAGARLVDLPTYPFQRRHYWAPTPTNPATNPATGDTTTADPVGGVRYRITWKPLPTDDPRPLTNRWLLIADPGTAGSELAADITAALIRRGAEVELLAVDPLAGRARIAELLATTTAGPVPLSGAVSLLGLVQDAHPQHPSIGMGVVSSLALVQAIGDAGAETPLWSVTQGAVAVVPQEAPDVFGAQVWAFGRVAALELPDRWGGLVDLPSVPNARMLDQLANALAGADGEDQIAVRGSGIYGRRVTRAAGTARREWRPRGNILVTGGTGSLGGRVARWLARNGAEHLVLTSRRGADAPGAAELEADLRALGVEVTMAACDVADRAALSDVLAAHPPTAVFHTAGVLHDGVIDTLAAGHIDEVFRPKTAAALLLDELTQHQELDAFVLFSSVTGVWGNGGQAAYAAANASLDALAERRRAAGLPATSIAWGLWGGGGMAEGIGEQNLNRRGITALDPELGIAALQQALDRDDVSVTVADVDWTVFAPRLADLRSGRLFDGVPEARSALDARKVDTESPSAGLAQRVAGMPDAERQRVLLETVRAAAAAVLRHETVDAVAPTRAFKDAGFDSLTALELRNHLNSTTGLSLPPTVVFDHPTPSTLAKFLEGVLVGASAEEVPVTAAAVPVDEPIAIVGMACRYPGGADTPEKLWDLLLAGADVIGPAPDDRGWDVDSFFDPVPGAAGKSYAREGGFVYDAGMFDAEFFGVSPREAVAMDPQQRLLLETSWEALERAGIDPAGLRGSRTGVYSGLTHQEYAARLHEAPQELEGYLLTGKSVSVASGRVSYVLGLEGPSISVDTACSSSLVALHLACQGLRLGECDVALAGGVTVIAAPGLFVEFSRQGGLSGDGRCRAFAGGADGTGWGEGAGVVVLERLSVARERGHRVLAVVRGSAVNQDGGSNGLTAPSGVAQRRVIGAALVAAGLGVSDVDVVEAHGTGTRLGDPIEAEALLGSYGRGRVGGALLLGSVKSNIGHTQAAAGVAGVIKMVMALRAGVVPATLHVDVPSPLVDWSSGGVELVTEARDWPVVGRVRRAGVSAFGVSGTNAHLILEQAPEFDDPADSDSDSDSDSDAGVVDGGEGGVGRSLSVVPVVVSGRSVGALRAYAGRLREVCAGLSDGGGSGGGSGLVDVGWSLVSSRSVFEHRAVVFGGGVEEVVAGLGAVASGAVASGSVVVGSVASGVAGGGGRVVFVFPGQGWQWVGMGAALLDESEVFAESMVECGRALSGFVDWDLLEVVRGGAGEGVWGRVDVVQPVSWAVMVSLARLWMSVGVVPDAVVGHSQGEVAAAVVGGVLSVADGARVVALRSRVIGEVLAGGGAMVSVGLPIVDVQERLAGWGGRLGVAAVNGPSLTVVSGDVDAAVGFVGECERDGVWVRRVAVDYASHSAHVEAVEGMLSGLLGGLCPGRGVVPFYSSVVGGVVDGVGLDGGYWYRNLRERVLFSDVVGRLVGDGFSGFVECSGHPVLAGGVLESVAVVDPDVRPVVVGSLRRDDGGWGRFLTSVGEAFVGGMSVDWKGVFAGAGARLVDLPTYPFQRRHYWAQTSPAGVGTAAAARFGMEWEDHPLLGGALSVGGSRSLLLAGHLSLASHAWLTDHAVSGTVLLPGTAFVELALHAAAAAGCPEVEELRLEAPLVVPARGGVRLQVLVDDPDDGSDRRAVSVFSRDDAAPAESAWTRHAVGVLAARSRPAPAAPWHTDAWPPSGTEPVDVADLYERFAALGYEYGEAFAGLQGVWRGDGEVFAEVRLPDRVSAEAIRFGLHPALLDAALQGWLAGDLVGVPEGSVLLPFAWQGVVLHATGADTLRVRIGRSGDSAVCLHAVDPAGAPVLSLDALALRPLVRERLGLPADAGAGALYRVGWRRQAAVAGAADRRWAVVAPNGAEADGAAEPHRWPVAAVDVHTDVDSLRAALDAGAELPAVVLADFRRAAGWSVDSSLAAGPSPNDGAVGDGAVGDARAGAVRAATRAGLDLLQRWLADERFIAARLVVVTERAVAAGPDEDVPGLVHAGLWGLLRSAQSEHPDRFVLVDVDADDSSLAALPSALAMDAPQLVVRAGQILLPEIEPVRPVPEPEQAEPEPGAVLDPDGTVLLTGATGTLGGLLARHLVTTRGARRLLLVSRSGPDAPDAGRLTEELTGLGAHVTLAACDTTDRAALAGVLGGIPAEHPLTAVVHVAGVLDDGAVQALTPERVDAVLRPKVDAALHLHELTAGLPLAAFVLFSGAAGILGRPGQANYAAANTFLDALAQHRRARGLPGVSLAWGLWGLASDMTGHLGEQDLRRMRRSGIAPMTGEEGLALFDLALDLARDEPVLVPARLDPAALRREWAANGPGAVPVLLRGLVPAAPLRRAAPSGAAGGAPVPAVAAPQQADELRGQLAGKDAQAQVRQLLDLVRAHVAGVLALREAADVDPGRPFREVGFDSLTAVELRNRLGSATGLRLAPSLVFDHPTPSAVAEHLVDRLAAEGAADEGAAALTGLDAVAAALGGMRTDDVRRDIVRRRLEEMLALVGGPRSGPAGDGLVDATVAERLDSASDDELFALIEEQL

SEQ ID NO: 11 - DNA, 13416 bp, micromonospora carbonacea subspecies aurantiaca
atgcgagttgtgggcgcagacgcgtgcagcgcagccgtccccgccggaccgcggatgggcttcccagcatcgttcttcgacccaggagacctcatgaccgtgcagagtgacgtgttgcgccaccgcgatatcgccgtcatcgggatgtcctgccggcttcccggcgcgccgagcatcgaggaattctgggacctgctgtgcagcgggcggagcgcggtcgaccgccagcccgacggcggttggcgggcggtgatcgatgggaagggagaatccgacgccgcgttcttcggcatgtccccgcgccaggccgccgcggtcgacccgcaacagcgcctgatgctcgaactcggctgggaggcactggagaacgcccgcatccggcccgccgacctgaagggctccgacactggcgtcttcgtggggctcaccgccgacgactacgccaccttgctgcgccgctccggcacgcccatcagcgggcacaccgcgacaggcctgaaccgtagcctcacggccaaccgtctctcgtacctgctgggtctgcgcggccccagcttcaccgtggactccgcgcagtcgtcatccctggtcgccgttcacctggcgtgcgaaagcctgctgcggggcgagagcgcggtcgccgtcgtcggcggggtgagcctcatcctggcagaggagagcaccgccgccatggcgcgtatgggggcactctctcctgacgggcgttgcttcaccttcgacgcccgggccaacggctacgtccgtggcgagggtggcgtggccatggtcctcaagccgctgatccgcgcgatcgaggacggcgaccaggtgcactgcgtcatccggggctgtgccgtcaacaacgacggcggtggccccagcctcacccatcccgaccgggaggcccaggaggcattgctgcgccgggcgtacgagcgggcgggggtggcccccgaacacgtcgactacgtcgagctgcacggcaccgggacgaaggccggcgaccccgtcgaggcggcggccctcggggcggtgctgggtgtcgcccgcggctgcgacaacccactcgcggtcggatcggtcaagaccaacgtcggccacctggagggggcggccggcatcacgggcctgctgaaggcggtgctgtgcgtacgtgagggggtgctgccgccgagcctcaacttccgtacgccgaacccggacatccgcctcgacgagctgaacctccgggttcagacggaactgcagccgtggccgggcgacgggacgggccgcccgcgtgtcgccggagtgagttccttcggcatgggcggtacgaatgcgcatctgattctcgagcaggctccggtggcggctgaggaaacggctgttaccgatgccggtgtcggttcggttcgggtggttccggtggtggtgtcgggtcgttcggtgggggctttgcgggcgtatgcgggtcggttgcgtgaggtgtgcgcggggttgtctgacggtggtggctccggtggtggttctggtctggtggatgtgggttggtcgttggtgtcgtcgcggtcggtgttcgagcatcgggcggtcgtgttcggtgggggtgtcgccgaggtggtggcgggtttggatgcggtggcttctggggcggtgagttcgggttcggtggtggtgggttcggtggcgtcgggtgttgctggtggtggtggtcgggtggtgtttgtgtttccgggtcagggttggcagtgggtgggtatgggtgcggctctgttggacgagtcggaggtgtttgctgagtcgatggtggagtgtgggcgggcgttgtcggggtttgtggattgggatttgttggaagtggtccgcggtggtgggggtgacggatcgtttggtcgggttgatgtggtgcagccggtgtcgtgggcggtgatggtgtcgttggcgcggttgtggatgtcggtgggtgtggtgccggatgcggtggtgggtcattcgcagggtgaggttgctgcgccggtggtggggggtgtgttgagtgtggctgatggggcgcgggtggtggcgttgcggtcgcgggtgatcggtgaggtgttggcgggtggtggtgcgatggtgtcggtggggttgccggtggcggttgtgttggatcggttggcggggtggggtggtcggttgggtgtggcggcggtgaatggtccgtcgttgacggtggtgtcgggggatgtggatgctgctgtggggtttgttggtgagtgtgagcgggatggggtgtgggtgcggcgggtggcggtggattatgcgtcgcattcggcgcatgtggaggcggtggaggggatgctgtcggggttgttgggtggtttgtgtccggggcggggtgtggtgccgttttattcgtcggtggtgggtggtgtggttgatggggtgggtttggatggtgggtattggtatcggaatctgcgtgagcgggtgttgttttcggatgtggtggggcggcttgttggggatgggttttcggggtttgtggagtgttcggggcatccggtgttggcgggtggggtgttggagtcggtggcggtggtggatccggatgtgcggccggtggtggtggggtcgctgcgccgtgatgatggtgggtggggccggtttttgacgtcggtgggtgaggcgttcgtcggcgggatgagtgttgactggaagggtgtgttcgcgggggcgggcgcgcggttggttgacctgccgacgtatccgttccaacgacgccactactgggcaccgaacaccgacggcgcgccagctccgatcctcgatgatcacgcggaggcggagaacgaaccagccgaatccgagccagggattcgggccgagcttctgacgttggccgagcccgagcaactgaaccgactcttggcgaccgttcgcgccagcaccgccgtcgttctgggcctcgactcggcgcaggcggtcgatccggagcgcacgttcaaggagcatggattcgaatcggtcaccgccgtcgagctctgtaaccacctgcaacgcggcactgggctgcgggttcccgcctcgcttgtatacaaccatcccaccccgatggccgctgcccggaagctgcaggaagaaattcagggccggcaaccggagaacgtccggcaggtcacctccgctgctgctgtggatgatccggtggtggtggtggggatgggttgtcgttttccgggtggggtggtgtgtgcggagggtttgtgggatttggtgttggggggtggggatgcggtgtcggggtttccggtggatcggggttgggatgtggaggggttgtttgatccggtgcggggtgtggtggggaagtcgtatgtgcgggagggggggtttgtgtatgacgcggggatgttcgatgcggagttttttggtgtgtcgccgcgtgaggcggtggcgatggatccgcagcagcgtttgtttttggaggtgtcgtgggaggcgttggagcgtgcggggattgatccgttgggtttgcggggttcgcggacgggtgtgtatgtgggggtgatgggtcaggagtatgggccgcggttggtggagtcgggtggtgggtttgagggttatttgttgacggggacgtcgccgagtgtggtgtcgggtcgtgtttcgtatgtgttggggttggagggtccgtcgatttcggttgatacggcgtgttcgtcgtcgttggtggcgttgcatttggcgtgtcaggggttgcggttgggtgagtgtgatgtggcgttggcgggtggggtgacggtgattgcggcgccggggttgtttgtggagttttctcggcagggtgggttgtcgggtgatgggcggtgtcgggcgtttgcgggtggtgcggatgggacggggtggggggagggtgcgggggtggtggtgttggagcggttgtcggtggcgcgggagcgtggtcatcgggtgttggcggtggtgcggggttctgcggtgaatcaggatggtgggtcgaatggtttgacggcgccgtcgggggtggcgcagcgtcgggtgattggtgcggcgttggtggcggcgggtttgggtgtgtcggatgtggatgtggtggaggcgcatgggacggggactcggttgggtgatccgattgaggctgaggcgttgttggggtcgtatgggcggggtcgtgtgggtggggcgttgttgttgggttcggtgaagtcgaatattggtcatacgcaggcggctgcgggtgtggcgggtgtgatcaagatggtgatggcgttgcgggcgggggtggtgccggcgacgttgcatgtggatgtgccgtcgccgttggtggattggtcttcgggtggggtggagttggtgacggaggcgcgggattggccggtggtgggtcgtgtgcgtcgtgcgggtgtgtcggcgtttggggtgtcggggacgaatgcgcatctgattttggagcaggcccccgaattcgacgatccggttgttaccgacaccgacaccgatgctggtgtgggtaggggtctatcggtggttccggtggtggtttcgggtcgttcgacggcggctttgcgcgcttatgcgggccggttgcgtgaggtgtgcgcgggtctttccgatggtgccggtctggtgaatgtgggttggtcgttggtgtcgtcgcggtcggtgttcgagcatcgggcggtcgtgtttggtgggggtgtcgccgaggtggtggcgggtttggatgcggtggtttccggggcggtggcttcgggttcggtggtggtgggttcggtggcgtcgggtgttgctggtggtggtggtcgggtggtgtttgtgtttccgggtcagggttggcagtgggtgggtatgggtgcggcgctgctggacgagtcggaggtgtttgctgagtcgatggtggagtgtggtcgggcgttgtcggggtttgtggattgggatttgttggaggtggtgcggggtggggcgggtgagggggtgtggggtcgggttgatgtggtgcagccggtgtcgtgggcggtgatggtgtcgttggcgcggttgtggatgtcggtgggtgtggtgccggatgcggtggtgggtcattcgcagggtgaggttgctgcggcggtggtggggggtgtgttgagtgtggctgatggggcgcgggtggtggcgttgcggtcgcgggtaattggtgaggtgttggccggtggtggtgcgatggtgtcggtcggactgccgatcgtggatgcgcaggaacggttggcggggtggggtggtcggttgggtgtggcggcggtgaatggtccgtcgttgacggtggtgtcgggggatgtggatgctgctgtggggtttgttggtgagtgtgagcgggatggggtgtgggtgcggcgggtggcggtggattatgcgtcgcattcggcgcatgtggaggcggtggaggggatgctgtcggggttgttgggtggtttgtgtccggggcggggtgtggtgccgttttattcgtcggtggtgggtggtgtggttgatggggtgggtttggatggtgggtattggtatcggaatctgcgtgagcgggtgttgttttcggatgtggtggggcggcttgttggggatgggttttcggggtttgtggagtgttcggggcatccggtgttggcgggtggggtgttggagtcggtggcggtggtggatccggatgtgcggccggtggtggtggggtcgctgcgccgtgatgatggtgggtggggccggtttttgacgtcggtgggtgaggcgttcgtcggcgggatgagtgttgactggaagggtgtgttcgcgggggcgggcgcgcggttggttgacctgccgacgtatccgttccaacgccgccactactgggcaccgactcccaccaaccccgccaccaaccccgccacgggcgacaccaccaccgccgacccggtgggtggcgtgcggtatcggatcacctggaaaccgttgccgacggacgacccccgacccctcaccaaccgctggctactcatcgccgacccggggaccgccggctcggagcttgccgcagacatcacagcagcgctcattcgcaggggcgccgaggtcgagttgctggccgtggacccgctcgcgggccgggcccggatcgccgaactgctcgccaccacgacggctgggccggtgccgctgtcgggcgccgtgtctcttctcgggcttgtgcaggacgcgcatcctcaacacccctccatcggaatgggcgtggtctcgtcgctggcgctggtgcaggccatcggtgacgcgggagccgagactcctttgtggagcgtcacgcagggggcggtcgctgtggtgccccaggaggcgccggatgtgttcggtgcgcaggtgtgggcgttcgggcgggtggccgccctggaactgccggaccgctggggcggcctggtcgaccttccgtccgtaccgaatgcccggatgctggaccagctcgccaacgccctcgccggagcggacggcgaggaccagatcgcggtacgcggctcggggatctacgggcgtcgggtgacgcgcgcggcgggcactgcgcgccgggaatggcgccctcgcgggaacatcctggtgaccggaggtacgggaagtctgggtggccgggtggcccggtggctcgctcgcaacggtgccgaacacctcgttctcaccagtcgtcggggtgccgacgccccgggggcggcagaactggaagctgatcttcgcgcgctcggtgtcgaggtgaccatggccgcctgcgatgtagcggaccgggctgcgctgtccgacgtcctggcggcgcatccgcccactgcggtcttccacaccgccggagtcctgcacgacggtgtgatcgacacgctcgccgccggacacatcgacgaggtcttccgtccgaagaccgctgccgcgctgctgctcgacgaactcacccagcaccaggagctggacgccttcgtcctcttctcatcggttaccggagtctggggcaacggcggccaggcggcgtacgcggcggcgaacgcatcgctggacgccctggcggagcgacgtcgtgccgcaggtcttcccgccacctccatagcttggggactgtggggcggcggtggcatggcggaggggatcggcgagcagaacctgaaccgccgtggcatcacggccttggacccggagctcggcatcgccgctctgcagcaggccctcgaccgcgatgacgtgtctgtcaccgtcgccgacgtcgactggacggttttcgctccgcgtcttgccgacctgcgctcggggcggctcttcgacggggtgcccgaggccaggagcgcgctcgatgcccggaaagtggacaccgagtcgccgagcgccggccttgcgcagcgggtggcggggatgcccgacgcggaacggcagcgggtcctcctcgaaacggtgcgggcggcggccgcggcggtcctgaggcacgagacggtggatgcggtcgcgcccacccgggccttcaaggacgccggcttcgactcgctcacggcgctcgaactgcgcaaccacctcaacagcacgaccggtctgagtctgcctccgacggtggtcttcgaccaccccaccccgtccacgttggcgaagttcctggagggcgtcctcgtcggcgcttctgccgaggaagtcccggtgactgccgcagccgtgcccgtcgacgagcctattgccatcgtcggcatggcctgccgctaccccggcggagccgacactcccgagaagctctgggacctcctgctggccggtgctgacgtcatcggcccagcccccgacgaccggggctgggacgtggactccttctttgatcccgtgccgggcgccgcggggaagtcgtatgcgcgggagggggggtttgtgtatgacgcggggatgttcgatgcggagttctttggtgtgtcgccgcgtgaggcggtggcgatggatccgcagcagcgcttgttgttggagacgtcgtgggaggcgttggagcgtgcgggaatcgatccggcgggtctgcggggtagccggaccggcgtgtactccggcctgacccaccaggagtatgccgcccgtctgcacgaggctccgcaggaactcgagggctatctgctcaccggcaagtcggtgagcgtcgcgtcgggtcgtgtttcgtatgtgttggggttggagggtccgtcgatttcggttgatacggcgtgttcgtcgtcgttggtggcgttgcatttggcgtgtcaggggttgcggttgggtgagtgtgatgtggcgttggcgggtggggtgacggtgattgcggcgccggggttgtttgtggagttttctcggcagggtgggttgtcgggtgatgggcggtgtcgggcgtttgcgggtggtgcggatgggacggggtggggggagggtgcgggggtggtggtgttggagcggttgtcggtggcgcgggagcgtggtcatcgggtgttggcggtggtgcggggttctgcggtgaatcaggatggtgggtcgaatggtttgacggcgccgtcgggggtggcgcagcgtcgggtgattggtgcggcgttggtggcggcgggtttgggtgtgtcggatgtggatgtggtggaggcgcatgggacggggactcggttgggtgatccgattgaggctgaggcgttgttggggtcgtatgggcggggtcgtgtgggtggggcgttgttgttgggttcggtgaagtcgaatattggtcatacgcaggcggctgcgggtgtggcgggtgtgatcaagatggtgatggcgttgcgggcgggggtggtgccggcgacgttgcatgtggatgtgccgtcgccgttggtggattggtcttcgggtggggtggagttggtgacggaggcgcgggattggccggtggtgggtcgtgtgcgtcgtgcgggtgtgtcggcgtttggggtgtcggggacgaatgcgcatctgattttggagcaggcccccgagttcgacgatcctgccgattccgattccgattccgattccgattccgatgccggtgtcgtggatggcggcgagggtggtgttggcaggagcttgtcggtggttccggtggtggtgtcgggtcgttcggtgggggctttgcgggcgtatgcgggtcggttgcgtgaggtgtgcgcggggttgtctgacggtggtggctccggtggtggttctggtttggtggatgtgggttggtcgttggtgtcgtcgcggtcggtgtttgagcatcgggcggtcgtgttcggtgggggtgtggaggaggttgttgctggtcttggtgcggtggcttctggggcggtggcttcgggttcggtggtggtgggttcggtggcgtcgggtgttgctggtggtggtggtcgggtggtgtttgtgtttccgggtcagggttggcagtgggtgggtatgggtgcggcgctgctggacgagtcggaggtgttcgccgagtcgatggtggagtgtggtcgggcgttgtcggggtttgtggattgggatttgttggaggtggtgcgcggcggggcgggtgagggggtgtggggtcgggttgatgtggtgcagccggtgtcgtgggcggtgatggtgtcgttggcgcggttgtggatgtcggtgggtgtggtgccggatgcggtggtgggtcattcgcagggtgaggttgctgcggcggtggtggggggtgtgttgagtgtggctgatggggcgcgggtggtggcgttgcggtcgcgggtgatcggtgaggtgttggccggtggtggtgcgatggtgtcggtcggactgccgatcgtggatgtgcaggaacggttggcggggtggggtggtcggttgggtgtggcggcggtgaatggtccgtcgttgacggtggtgtcgggggatgtggatgctgctgtggggtttgttggtgagtgtgagcgggatggggtgtgggtgcggcgggtggcggtggattatgcgtcgcattcggcgcatgtggaggcggtggaggggatgctgtcggggttgttgggtggtttgtgtccggggcggggtgtggtgccgttttattcgtcggtggtgggtggtgtggttgatggggtgggtttggatggtgggtattggtatcggaatctgcgtgagcgggtgttgttttcggatgtggtggggcggcttgttggggatgggttttcggggtttgtggagtgttcggggcatccggtgttggcgggtggggtgttggagtcggtggcggtggtggatccggatgtgcggccggtggtggtggggtcgctgcgccgtgatgatggtgggtggggccggtttctgacgtcggtgggtgaggcgttcgtcggcgggatgagtgttgactggaagggtgtgttcgcgggggcgggcgcgcggttggttgacctgccgacgtatccgttccaacgacgccactactgggcccagacctcgcccgctggcgtcgggacggccgcggcggcccggttcggcatggagtgggaggaccatcccctgctcggcggtgcgctgtcggtcgggggctccaggagcctgcttctggccgggcatctgtcgctcgcctcgcacgcctggctgaccgaccatgccgtctccggcaccgtgctgctgcccggtacggccttcgtggaactcgccctgcacgccgccgctgcggctggctgtccggaggtcgaggagctgcggctggaggctcccctggtggtgccggccaggggcggggtgcggctccaggtgctcgtggacgaccccgacgacggatccgaccgccgcgcggtaagcgtgttctcccgggacgatgcggcgccggccgagtccgcctggacgcggcacgcggtgggcgtcctggccgcgcggtcgcggcctgcaccggctgcgccctggcacaccgacgcctggccaccttcgggcacggagccggtcgacgtggccgacctgtatgagcggttcgcggcgctgggctacgagtacggggaggcgttcgccgggctccagggggtctggcggggggacggcgaggtgttcgccgaggtgcggctgcccgaccgggtcagcgcggaggccattcgcttcgggctgcatcccgcgctgctcgacgccgccctgcaggggtggttggcgggcgacctcgtcggcgtccccgagggcagtgtgctgctgcccttcgcctggcagggcgtcgtgctccacgccaccggcgccgacactctgcgggttcgcatcggccggtccggtgactcggccgtctgcctgcacgcggtggacccggccggtgctccggtcctctcgttggacgccctggccctgcgtccgctcgtccgggaacgcctcgggctgcccgccgatgccggagccggggcgttgtaccgggtcggctggcggcggcaggccgccgttgccggggcagccgaccggcggtgggcggtcgtggccccgaacggtgccgaggcggacggggccgccgagccgcaccggtggccggtcgccgccgtcgacgtgcacaccgacgtggactcgctgcgggcggccctggacgcgggcgcggaactgcccgccgtcgtcctcgccgacttccggagggccgccggctggagcgtcgacagttcgctggccgccggcccgtcgcccaacgacggcgcggtgggcgacggcgcggtgggcgacgcccgggccggggccgtccgggcggcgacccgggccgggctggatctgctgcaacgctggctggccgacgagcggttcatcgcggccaggctcgtggtggtcaccgaacgggccgtggccgccgggccggacgaggacgtgccgggcctcgtccacgcgggactgtggggcctgctccggtcggcccaatcggagcacccggaccgcttcgtgctggtggacgtcgacgcggacgacagctcgctcgcggcgctgccgtcggccctcgccatggacgcgccccaactggtggtgcgggccggtcagatcctgctgcccgagatcgagccggtgcggcccgtacccgagccggagcaggcggaacccgaaccgggggccgtcctggaccccgacggcacggtcctgctcaccggcgcgaccggcacgctcggcgggctgctcgcccggcacctggtgaccacccgtggtgcgcgccggctgctgctggtcagccgcagcggtccggacgcccccgatgccggccggctgaccgaggagctgaccgggctcggcgcccacgtgacgctggccgcctgcgacaccacggatcgcgccgcgctggccggcgtcctgggcggcatccccgccgagcatccgctgaccgccgtggtgcacgtggccggcgtactcgacgacggggcggtgcaggcgctcacccccgagcgggtcgacgcggtgctccggccgaaggtggacgcggcactgcacctgcacgaactgaccgcggggctgccgctggccgcgttcgtgctgttctccggggcggcggggatcctgggccggcccggccaggccaactacgcggcggcgaacaccttcctggacgccctggcgcagcaccgacgggcccggggcctgcccggcgtctccctcgcctggggcctgtgggggctggccagcgacatgacgggccacctgggcgagcaggacctgcggcggatgcggcgctccggcatcgccccgatgaccggcgaggagggcctcgcgctgttcgacctggccctcgacctggcccgggacgaaccggtgctcgtaccggcccgactggacccggcggcgctgcgccgggagtgggccgccaacggaccgggcgccgtcccggtcctgctgcggggtctggtgccggcggctccgctccgtcgcgcggccccgtcgggcgccgccggcggtgcgcccgtgcccgccgtcgccgcgccgcagcaggcggacgagctgcgcgggcaactggccgggaaggacgcgcaggcccaggtccggcagctgctggatctggtacgcgcccatgtcgccggggtgctcgccctccgggaagcggcggacgtggacccgggcagaccgttccgcgaggtcggattcgactcgttgaccgcagtcgaactgcgcaaccggctgggctcggcgaccggcctgcggttggcaccgagcctggtgttcgaccatccgaccccgtcggccgtggccgagcacctcgtggaccgcctcgccgccgagggggcggctgacgagggcgcggcggcactgaccgggctcgacgcagtggccgcggcgctcggcgggatgcggacggacgacgttcgccgggacatcgtccgcaggcggctggaggagatgctcgccctggtcggcgggccacggtccgggccggcaggtgacgggctggtggatgccacggtcgccgagcgactggactcggcttccgacgacgaactcttcgccctgatcgaggagcagctgtga

SEQ ID NO: 12 - protein, 1925 aa, micromonospora carbonacea subspecies aurantiaca
VTANEDRMREYLKRVTAELAGTRRRLRELEDSAREPIAIVGMSCRLPGGVSTPEDLWRLVEAGTDAISGFPDDRGWDVGRLYDPDPDSTGTSYVREGGFLYDCAEFDPEFFTVSPREALAMDPQQRLLLEAAWETFERAGIAPDSARGTRTGVYVGVMYDDYGSRLSEVPKDLEGYLVNGSAGSVASGRIAYTLGLQGPAVTVDTACSSSLVALHLAVQALRSGECELALAGGATVLATPTMFVDFARQRGLAEDGRCKAFADAADGTGFGEGVGMLLVERLSDAVRNRRQVLAVVRGSAVNQDGASNGLTAPNGTAQQLVIRQALTNAGLAADEVDAVEAHGTGTRLGDPIEAQALLATYGQGRPADRPLLLGSLKSNIGHTQAAAGVAGVIKTVLALRHARLPRTLHVDRPSTRVDWSSGAVRLLTEGRPWPDHGDRPRRAGVSSFGASGTNAHVILESAPGAAAGATGATDLSAPPASVAHHPATATATAPAATVPTAHEPAGTAGDDPVWVLSGRTEAALREQARRLHAHLTSRARPEPADAVARALARSRTAFAYRAAVLGRDDTARLDGLHALAAGRSAAGLVTGRAVPERRVAFLFTGQGSQRPGAGRELYARHPAFAQALDGVLAELDRHLDRPLRAVMLAEPGTEAAALLDDTAYTQPALFALEVALFRLVTSWGLRPDALLGHSVGEITAAYVAGVLTLPDAARLVAVRGRLMADLRAGGAMAALQAAESEVDPLLAGREGELSIAAVNGPQATVIAGDEAAVEEQVALWRDRGRRARRLRVGHAFHSVRMDGMLAEFEKAMGDLRAGEPTIPVVANVRGAIASGTDLRTAGYWIRHAREPVRFLDGMRALRAEGVDTFVELGPDGVLTAMARDCLADPADPVDLADAAEPAGAAEPDRSLLFLPTLRRDRDDAVAVREALASVHVHGLPVDPVAPLGDGPLATDLPTYPFQRSRYWLDPRPGARDLTAVGLDVAGHPLLAVAVDLPDGAGTVWSGQLCVRTHPWLADHSVWGRTVVPGTALLEIMHRVRAEVGCTRVAELTFEAPMVLADDGGVRVRVVVDGPDADGARQVRIHSAPVGPEPPHWTRHASGRVDSAAPGPAAGPPAWDAGPGSNWPPEGAEPVGVESEYERFADNGIGYGPAFRGLRAAWRRGNETFAEVRLPEGYAAEAGDYAVHPALLDAALHAIVFGDQFPGGAHGMLPFAFTDVRVFSSGADRLRVRIAPADADSVCVTVADGDGTPVLAAATLALRRVAADRIAATVTGQAPLYRLEWSAVRPAPVATGARFAVVGADAPLPSGALGAGVPVQAYPDLGALAGALATNGAPGHVLVDFRRRADGPAGRQPGDVGARTRRALAVVQEWLADDRFTGSRLVVLTSGAVDAGTAVTDPAAAGVWGLLRVAQTEHPDRFVLVDTDDHPDSLRALPGAIVAGEPQLALRAGTASVPGLVRVPAGTGAAPPWAAAGTVLVTGGTGMLGGAVARHLVRRHGVRRLLLVGRRGPDAPGAAALTRELEELGASVRVAACDVGDRGAVTRLLAGVPAAHPLTAVVHSAGLPDDGVLTAQTGERVAAVLRAKADAAVNLHELTRHLDLTAFVLFSSVAGTIGSAGQAGYAAANAFLDAFASWRQGQGLPATALAWGPLDGGMAAGLGTADVARLRRSGLVPLGVDDALVLFDAACSRPAAAYHPVRLDPAVLRSHAAADSAVPAVLLGPSRAHPRDGTPGKPAEAALAALLTGRSAAERTAILTDLVRTEAAAVLGHGEAAMLSTQRAFRDAGFDSLTAVDLRNRLGAATGLSLPAAVVFDHPTPAALAAYLRTELDRRSPTGQQFPTDAAGVLAMLDRLRDGIATVVRDDADRTRAADLLRVLLAEVGGPGTGPPRDTDGGSGGEVSDRLRTASDEELFDLLDSDFRLA

SEQ ID NO: 13 - DNA, 5778 bp, micromonospora carbonacea subspecies aurantiaca
gtgaccgcgaacgaggaccggatgcgtgagtacctcaagcgggtcaccgccgagctggccgggacgcggcgacgcctgcgcgagctggaggacagcgcgcgtgagcccatcgcgatcgtgggcatgagctgccggttgccgggcggggtgagcacgcccgaggacctgtggcggctggtcgaggccggtaccgacgcgatctccggcttccccgacgaccggggctgggatgtcgggaggctctacgacccggatccggactcgaccggaacgagctacgtgcgcgagggcggcttcctctacgactgcgccgagttcgacccggagttcttcaccgtctcgccccgcgaggcgctggccatggacccgcagcagcggctgctgctggaggccgcctgggagaccttcgaacgggcggggatcgcccccgactcggcccgcggcacccgcaccggggtctacgtcggggtgatgtacgacgactacggcagccggctgtcggaggtgccgaaggacctggagggctacctggtcaacggcagcgcgggcagtgtcgcgtcgggccggatcgcgtacacgctggggttgcaggggccggcggtgacggtcgacacggcctgctcgtcgtcgctggtcgcgttgcacctggccgtgcaggcgctgcggtcgggcgagtgtgagctggccctggcgggcggggcgacggtgctcgccacgccgacgatgttcgtcgacttcgcccggcagcgcggtctcgccgaggacggccgttgcaaggcgttcgcggacgccgccgacgggaccgggttcggcgagggcgtggggatgctgctggtggaacggctctcggacgcggtccgcaaccgtcgccaggtgctggccgtcgtgcggggcagcgcggtcaaccaggacggggcgagcaacggcctgaccgccccgaacggtacggcccagcaactggtcatccggcaggcgttgaccaacgcggggctggccgcggacgaggtggacgcggtggaggcacacggcaccggcacccggctgggcgatccgatcgaggcgcaggcgctgctggcgacgtacggccagggccggccggcggaccggccgctcctgctgggatccctgaagtccaacatcggccacacccaggccgccgcaggggtcgccggggtgatcaagaccgtgctggcgctgcgtcacgcgcggctgccccggaccctgcacgtcgatcgcccctcgacccgggtggactggtcgtcgggcgcggtgcggctgctgaccgaggggcggccctggcccgatcacggcgaccggccccgccgggccggggtctcctcgttcggcgcgagcggcaccaacgcgcacgtcatcctggagagcgcccccggtgcggcggcgggggcgaccggggcgacggacctctcggccccgccggcatccgtcgcccaccatccggccacggccacggccacggccccggcggcgacggtgcccactgcccacgaaccggcggggacggccggcgacgaccccgtctgggtcctgtccggccggaccgaggcggccctgcgcgagcaggcccggcggctacacgcccacctgacatcccgggcgcggcccgagcccgccgacgccgtggcccgcgcgctggcgcgctcccgcaccgcgttcgcgtaccgggccgccgtgctgggccgggacgacaccgcgcggctcgacggcctccacgcgctcgcggcgggtcgcagcgccgcggggctcgtcaccgggcgggccgtgccggagcggcgcgtggccttcctcttcaccgggcagggcagccagcgaccgggcgcgggccgggaactgtacgcccggcatcccgccttcgcacaggccctggacggcgtcctcgcggaactcgaccggcacctggaccggccgctgcgcgccgtcatgctcgccgagccgggcaccgaggcggcggcgctgctggacgacaccgcgtacacccagcccgccctgttcgcgctggaggtggcgctgttccggctggtcacgagctgggggctgcggcctgacgccctgctgggccactcggtcggggagatcaccgcggcgtacgtcgcgggcgtcctcaccctgccggacgccgcccggctggtggcggtgcgcggtcgactcatggcggacctgcgggccggcggtgcgatggccgcgctccaggccgccgagagcgaggtcgaccccctgttggcggggcgggagggcgaactgtcgatcgcagcggtcaacgggccgcaggcaaccgtgatcgcgggcgacgaggcggccgtcgaggagcaggtcgcgctgtggcgtgaccggggtcgccgggccaggcgactgcgggtcggccacgccttccactccgtacggatggacgggatgctcgccgagttcgagaaggcgatgggtgatctccgtgccggcgagccgacgatccccgtggtcgccaacgtcaggggggcgatcgcgtccggcaccgacctccgtacggccgggtactggatccggcacgcccgcgagccggtgcgtttcctcgacggcatgcgtgcgctgcgggccgagggcgtcgacacgttcgtggaactcggccccgacggagtgctcacggcgatggcgcgcgactgcctggcggatcccgccgacccggtggatctcgcggacgccgccgagcccgccggggccgcggagcccgaccgctccctgctgttcctgcccaccctgcgccgggaccgcgacgacgcagtggccgtgcgggaggccctggcatccgtccacgtgcacgggcttcccgtcgacccggtcgcgccgctcggcgacggcccgctcgccaccgacctgcccacctacccgttccagcggtcccgctactggctcgacccgcgtcccggggcacgcgacctgaccgccgtgggcctcgacgtggccgggcacccgctgctcgccgtcgccgtggacctgcccgacggcgccggcacggtctggagcggtcagctctgcgtgcggacgcatccgtggctcgccgaccacagcgtgtgggggcgcacggtggtgccggggaccgcgctgctggagatcatgcaccgagtgcgcgccgaggtgggctgcacccgggtcgcggaactgaccttcgaggcgccgatggtgctggccgacgacgggggcgtccgcgtgcgggtcgtcgtcgacggaccagacgccgacggggcccgccaggtccggatccactccgcaccggtggggcccgagcctccccactggacccggcacgcctcgggccgcgtcgacagcgccgcgccggggccggccgccggcccacccgcgtgggacgccggccctggcagcaactggccgcccgagggggcggagccggtgggcgtcgagagcgagtacgagcgcttcgccgacaacggcatcggatacggccccgccttccgagggctgcgcgccgcgtggcgtcgcgggaacgagacgttcgccgaggtccggctccccgaggggtacgccgccgaggcgggcgactacgccgtccatccggcactgctggacgcggccctgcacgcgatcgtcttcggtgaccagtttcccggtggggcacacgggatgctgccgttcgccttcaccgacgtgcgggtgttcagctccggcgccgaccggctccgggtgcgcatcgcgcccgccgatgccgactcggtctgcgtgaccgtcgccgacggcgacgggacgccggtcctcgccgcagccaccctggcgttgcgccgggtcgccgccgaccggatcgcggcgaccgtcaccggccaggcaccgctgtaccggttggagtggtccgccgtgcggcccgccccggtggccaccggggcgcggttcgccgtcgtcggcgcggacgccccgctgccgtccggtgcgctgggggccggggtgcccgtccaggcgtacccggacctgggcgcgctggccggcgcgttggccaccaacggggcaccgggccacgtgctcgtcgacttccgccgccgcgccgacggcccggcagggcggcagcccggtgacgtgggtgcacggacccgacgggcgctggccgtcgtccaggagtggctcgccgacgaccgtttcaccggctcacggctggtcgtgctcaccagcggagccgtggacgccggaacagccgtcaccgatccggccgccgccggggtgtggggcctgctgcgggtcgcccagaccgagcatccggaccggttcgtcctcgtggacaccgacgaccacccggattcgctgcgtgccctccccggggcgatcgttgcgggcgagccgcagctggcactgcgggccggcacggccagcgttccgggcctggtgcgggtgccggccggcaccggtgccgccccgccgtgggccgcagccggcaccgtcctcgtcaccgggggcaccggcatgctcggcggcgcggtggcccggcacctggtccgccggcacggggtccgccgcctgctgctggtcggccggcgcgggccggacgcacccggcgcggcggccctgacccgggaactggaggagctgggagcgtccgtccgcgtcgccgcctgcgacgtcggcgatcgtggcgcggtgacgcgcctgttggccggggttcccgccgcgcatccgctcaccgcggtggtgcactcggccggcctgcccgacgacggcgtgctgaccgcacagaccggcgagcgggtcgcggcggtgctccgcgccaaggcggacgcagcggtcaacctgcacgaactcacccggcatctcgacctcaccgccttcgtgctgttctcgtcggtagcggggacgatcggcagcgccgggcaggccgggtacgccgccgcgaacgccttcctcgacgcgttcgcgagctggcggcagggccaggggctgcccgccaccgccctggcgtgggggccgttggacggcgggatggccgccggcctcggcactgcggacgtggcacggctgcgccggtccgggctcgtgccgctcggcgtggacgacgcgctcgttctcttcgacgccgcctgctcccgaccggcggcggcgtaccaccccgtccgcctcgatccggcggtgctgcggtcccacgccgccgccgacagcgcggtgcccgccgtcctgctcggtccgagccgtgcgcacccgagggacggtacgccggggaagcctgccgaagccgccctcgccgcgctgctgaccggcaggtcggcggccgagcgtacggcgatcctgaccgacctggtgcggacggaggccgccgccgttctcgggcatggcgaggcggcgatgctgagcacgcagcgggccttccgcgacgccggcttcgactcgctcaccgccgtggacctccgcaaccggctcggcgcggccacgggcctcagcctgccggccgccgtcgtcttcgaccacccgaccccggcggccctggccgcctatctgcggaccgaactggaccgccggtcgcccaccgggcaacagttcccgacggacgccgccggtgttctggccatgctcgaccgcctgcgggacggaatcgcgacggtcgtcagggacgacgccgaccggacccgcgcagccgacctgttgcgtgtcctgctcgccgaggtcggcgggcccgggacgggcccgccccgcgacaccgacggcggctccggcggcgaggtcagcgaccgcctccggaccgcctccgacgaggaactgttcgacctgctcgacagcgatttccgactggcgtag

SEQ ID NO: 14 - protein, 3745 aa, micromonospora carbonacea subspecies aurantiaca
VSVNNEDKLREYLRRAMADLHESRERLRQYESAAAVDDPVVVVGMGCRFPGGVVCAEGLWDLVLGGGDAVSGFPVDRGWDVEGLFDPVRGVVGKSYVREGGFVYDAGMFDAEFFGVSPREAVAMDPQQRLFLEVSWEALERAGIDPLGLRGSRTGVYVGVMGQEYGPRLVESGGGFEGYLLTGTSPSVVSGRVSYVLGLEGPSISVDTACSSSLVALHLACQGLRLGECDVALAGGVTVIAAPGLFVEFSRQGGLSGDGRCRAFAGGADGTGWGEGAGVVVLERLSVARERGHRVLAVVRGSAVNQDGGSNGLTAPSGVAQRRVIGAALVAAGLGVSDVDVVEAHGTGTRLGDPIEAEALLGSYGRGRVGGALLLGSVKSNIGHTQAAAGVAGVIKMVMALRAGVVPATLHVDVPSPLVDWSSGGVELVTEARDWPVVGRVRRAGVSAFGVSGTNAHLILEQAPEFDDPADSDSDSDSDAGVVDGGEGGVGRSLSVVPVVVSGRSVGALRAYAGRLREVCAGLSDGGGSGGGSGLVDVGWSLVSSRSVFEHRAVVFGGGVEEVVAGLGAVASGAVASGSVVVGSVASGVAGGGGRVVFVFPGQGWQWVGMGAALLDESEVFAESMVECGRALSGFVDWDLLEVVRGGAGEGVWGRVDVVQPVSWAVMVSLARLWMSVGVVPDAVVGHSQGEVAAAVVGGVLSVADGARVVALRSRVIGEVLAGGGAMVSVGLPIVDVQERLAGWGGRLGVAAVNGPSLTVVSGDVDAAVGFVGECERDGVWVRRVAVDYASHSAHVEAVEGMLSGLLGGLCPGRGVVPFYSSVVGGVVDGVGLDGGYWYRNLRERVLFSDVVGRLVGDGFSGFVECSGHPVLAGGVLESVAVVDPDVRPVVVGSLRRDDGGWGRFLTSVGEAFVGGMSVDWKGVFAGAGARLVDLPTYPFQRRHYWAPTPTNPATNPATNPATNPATGDTTTADPAGDLRYRITWKPLPTDDPRPLTNRWLLMVPEALAGDGVVAGVRQALAARGASVELLTVGTADRAGLAALLTSAAPGDPEAAGPAGVVSLLALAEGADARHPAVPLGLTASLALIQALADAGTQARLWAVTRGAVAVSSGEVPDAGQAQVWGLGRVAALELPDRWGGLVDLPALTGERAFAQLADVVGGSNGEDQVAVRASGVYGRRLVRSRATVTSGDWPARGTILVVGDTGPVAALLAGRLLGDGAAHVVLAGPAAASTVGLTGGADRVALIDCDPSDRDALAGLLGAYRPTTIVVAPPAVALTALAETTPEDFVAAVAAKTTTAVHLDALAAEAELELDAFVVFSSVSGTWGGAGHGGYAAGTARLDALVEERRARGLPATAIAWTPWADATTAAGGQAPDASAGGHEPDTRAGGPDRELLRRGGLTPLDPGAALDVLRGAVARGEGLVTVADVDWARFVASYTAARPTTLFDELPELRATREAEHTPAEDSSAGGELVRALSGRPAADQHRTLLRLVRAHVAAVLGHDEAEAADPDRAFRELGFTSVTAVDLRNRLNAATGLNLPASVVFDHPSARVLAAYLRAELLGPEADEDTAEAVAPPSAPAGAGDDEPIAVIGMACRFPGGVDAPDDLWDLLAKGRDAISRFPTNRGWDVDGLYDPDPEAPGRTYVREGGFLHDAPDFDAAFFGISPREALAMDPQQRLLLETTWESLERAGLDPTALRGTRTGVFVGTNGQHYMPLLRDGADDFDGYLGTGNSASVMSGRLSYVFGLEGPAVTVDTACSASLVALHLAVQALRRGECTLALVGGATVMSTPDMLVEFSRQRAMSPDGRSKAFAAAADGVALSEGAAMMVVQRLADAEAAGHEILAVVKGSAVNQDGASNGLTAPNGPSQERVIRQALADAGLRPDQVDAVEAHGTGTALGDPIEAQALLATYGRDRPAGRPLWLGSLKSNIGHTQAAAGIAGVMKVILALRHDTLPRTLHVDRPTPRVDWASGAVSLLTEPVPWPQGDEPRRAAVSSFGISGTNAHVIVEQAPPVVREPIDHEADEVTVPLFLSARGSAALCAQAARLRARLIEEPDLDIAEVGYTLAATRARFEHRAVVIGESRAEVGDALAALARGEEHPSLLRGRAGASDRVAFVFPGQGSQWAEMADGLLDRSPAFRASASACDEALRAHLDWSVLDVLRRVPDAPALSRVDVVQPVLFTMMVSLAAAWRALGVHPSAVVGHSQGEIAAAHVAGGLSLDDAARIVALRSQAWLRLAGQGGMVAVSLPVDALRARLARFGDRLSVAAVNSPGTAAVSGYPDALAELVDELTAEGVHAKAIPGVDTAGHSAQVEVLKDHLMAALAPVSPRSSQIPFYSTVTGGLLDTALLDAAYWYRNMRDPVEFEQATRAMLADGHEGFLEPSPHPMLSVSLQGTAADAGVAATVLGTLRRGKGGARWFGMALGLAHAHGIEIDASVLFGTDSRRVDLPTYPFQRERFWYHPPAARGDVASAGLSGADHPLLGGAVELPDRGGHVYPARLGVRHHPWLGEHALLGAAILPGAAYAELALWAGRRDGAGRIEELTLDAPLVVADESAAQLRLVVGPADAEGRRQLTVHSRADGADADTAWTRHAQGTLVPADADAAGSGDPGAPWPPAGAEPVEVAGLYDRFADRGYQYGPSFRGVRAAWRAGDTVYAEVALPVPQPGSPRFGVHPALLDAAFQAMSLGAFFPEDGQVRMPFALRGVSSSGVGADRLRVTISPAGAEAVRIACVDERGNPVVVIDSLVARAVPVEALTPGTPGTGDGALHHVAWTARPEPGVAAVQRWAVVGAADPGLAGGLDRAGGLCGAYPDLAGLVAAVAEGAALPDVVAVPVPSGAPVGPDAVRATVLGALDLIRAWLAVEGRLGLARLAFVTTSAVAVGDGTEHVDPVSAALWGLVRSAQSEEPGRFVLVDLDADPASASALPAALAAGEPQLAVRAGAVHVPRLVRHRPRPDGPLTPPAGAAWRLAAGGQGTLEGLALVPAPDALAPLAPGQVRVAVRAAGVNFRDTLIALGMYPGTPVLGAEGAGVITEVAPDVAGFAPGDRVLGMWTGGLGPVAVADARMLARVPRGWSYAEAASVPAVFLTAHYALTRLAGIRPGQSLLVHAGAGGVGMATLQLARHLGVEVYATASRGKWDTLRGLGLDDAHIADSRSLDFAGRFLAATGGRGVDVVLNSLAGDFVDASLRLLPRGGHFLELGKADVRDPDRIAADHPGVGYRAFDLVEAGPELVGQLLGELMELFAAGVLSPLPLTVRDVRRAREAFRLISQARHVGKVVLTMPPAFGAYGTVLVTGGTGTLGGAVARHLVARHGVRHLVLTGRSGPAADGASALVDELTASGASVTVVACDAADRVALRRLLDGIPAAHPLTAVVHAAGVLDDATITALTAGQVDAVLRPKADAVINLHELTRDRELSAFVLFSSAAALFGSPGQGNYSAANGFVDAFAQYRRAQGLHAVSLAWGLWADSSRMAGHLDQEGMRRRMARGGVLPLTTDQGLALFDAAQLVDEALQVPIRLNVGALRAAGRVPALLADLVPAAASGAPAATPTRDDADRTLADRLAGLTVAEQRELVLESVRGHAAAVLGHADPQAVDADRAFRELGFDSLTAVELRNRLATASGLRLPATLVFDHPTPEALAEHLLAGLAPEQARAELPLLAELGRLEAALAATDGAALDGLDDLVRREVGVRIAALAARWGAAGDDVAGSDGGGTADALESADDDEIFAFIDERFRA

SEQ ID NO: 15 - DNA, 11238 bp, micromonospora carbonacea subspecies aurantiaca
gtgtctgtcaacaacgaagacaagcttcgcgagtatctgcgtcgtgccatggcggatctccatgagtcccgcgagcggttgcggcagtacgagtccgctgctgctgtggatgatccggtggtggtggtggggatgggttgtcgttttccgggtggggtggtgtgtgcggagggtttgtgggatttggtgttggggggtggggatgcggtgtcggggtttccggtggatcggggttgggatgtggaggggttgtttgatccggtgcggggtgtggtggggaagtcgtatgtgcgggagggggggtttgtgtatgacgcggggatgttcgatgcggagttttttggtgtgtcgccgcgtgaggcggtggcgatggatccgcagcagcgtttgtttttggaggtgtcgtgggaggcgttggagcgtgcggggattgatccgttgggtttgcggggttcgcggacgggtgtgtatgtgggggtgatgggtcaggagtatgggccgcggttggtggagtcgggtggtgggtttgagggttatttgttgacggggacgtcgccgagtgtggtgtcgggtcgtgtttcgtatgtgttggggttggagggtccgtcgatttcggttgatacggcgtgttcgtcgtcgttggtggcgttgcatttggcgtgtcaggggttgcggttgggtgagtgtgatgtggcgttggcgggtggggtgacggtgattgcggcgccggggttgtttgtggagttttctcggcagggtgggttgtcgggtgatgggcggtgtcgggcgtttgcgggtggtgcggatgggacggggtggggggagggtgcgggggtggtggtgttggagcggttgtcggtggcgcgggagcgtggtcatcgggtgttggcggtggtgcggggttctgcggtgaatcaggatggtgggtcgaatggtttgacggcgccgtcgggggtggcgcagcgtcgggtgattggtgcggcgttggtggcggcgggtttgggtgtgtcggatgtggatgtggtggaggcgcatgggacggggactcggttgggtgatccgattgaggctgaggcgttgttggggtcgtatgggcggggtcgtgtgggtggggcgttgttgttgggttcggtgaagtcgaatattggtcatacgcaggcggctgcgggtgtggcgggtgtgatcaagatggtgatggcgttgcgggcgggggtggtgccggcgacgttgcatgtggatgtgccgtcgccgttggtggattggtcttcgggtggggtggagttggtgacggaggcgcgggattggccggtggtgggtcgtgtgcgtcgtgcgggtgtgtcggcgtttggggtgtcggggacgaatgcgcatctgattttggagcaggcccccgagttcgacgatcctgccgattccgattccgattccgattccgatgccggtgtcgtggatggcggcgagggtggtgttggcaggagcttgtcggtggttccggtggtggtgtcgggtcgttcggtgggggctttgcgggcgtatgcgggtcggttgcgtgaggtgtgcgcggggttgtctgacggtggtggctccggtggtggttctggtttggtggatgtgggttggtcgttggtgtcgtcgcggtcggtgtttgagcatcgggcggtcgtgttcggtgggggtgtggaggaggttgttgctggtcttggtgcggtggcttctggggcggtggcttcgggttcggtggtggtgggttcggtggcgtcgggtgttgctggtggtggtggtcgggtggtgtttgtgtttccgggtcagggttggcagtgggtgggtatgggtgcggcgctgctggacgagtcggaggtgttcgccgagtcgatggtggagtgtggtcgggcgttgtcggggtttgtggattgggatttgttggaggtggtgcgcggcggggcgggtgagggggtgtggggtcgggttgatgtggtgcagccggtgtcgtgggcggtgatggtgtcgttggcgcggttgtggatgtcggtgggtgtggtgccggatgcggtggtgggtcattcgcagggtgaggttgctgcggcggtggtggggggtgtgttgagtgtggctgatggggcgcgggtggtggcgttgcggtcgcgggtgatcggtgaggtgttggccggtggtggtgcgatggtgtcggtcggactgccgatcgtggatgtgcaggaacggttggcggggtggggtggtcggttgggtgtggcggcggtgaatggtccgtcgttgacggtggtgtcgggggatgtggatgctgctgtggggtttgttggtgagtgtgagcgggatggggtgtgggtgcggcgggtggcggtggattatgcgtcgcattcggcgcatgtggaggcggtggaggggatgctgtcggggttgttgggtggtttgtgtccggggcggggtgtggtgccgttttattcgtcggtggtgggtggtgtggttgatggggtgggtttggatggtgggtattggtatcggaatctgcgtgagcgggtgttgttttcggatgtggtggggcggcttgttggggatgggttttcggggtttgtggagtgttcggggcatccggtgttggcgggtggggtgttggagtcggtggcggtggtggatccggatgtgcggccggtggtggtggggtcgctgcgccgtgatgatggtgggtggggccggtttctgacgtcggtgggtgaggcgttcgtcggcgggatgagtgttgactggaagggtgtgttcgcgggggcgggcgcgcggttggttgacctgccgacgtatccgttccaacgccgccactactgggcaccgactcccaccaaccccgccaccaaccccgccaccaaccccgccaccaaccccgccacgggcgacaccaccaccgccgacccggcgggtgacctgcggtatcggatcacctggaaaccgttgccgaccgacgacccccgacccctcaccaaccgctggctgctgatggtgcccgaggcgctggccggtgacggggtggtggcgggcgtacggcaggcgctggccgcgcgtggcgcctccgtcgaactgctgaccgtcggcaccgccgaccgggccggccttgccgcgctcctgacctccgccgcccccggcgacccggaggcggccggcccggcgggcgtggtctccctgctggcgctcgccgagggcgcggacgcgcgccacccggccgtaccgctcggcctgaccgcctcgctcgccctgatccaggcattggcggacgcggggacgcaggcccgcctctgggcggtcacccggggggccgtcgccgtgtcctccggcgaggtgccggacgccgggcaggcccaggtgtgggggctcggccgggtcgcggccctcgaactgccggaccgatggggcgggctggtggacctgccggcgctcaccggggagcgtgccttcgcgcagctcgccgatgtcgtgggcggctcgaacggcgaggaccaggtcgccgtacgggcctccggcgtctacggtcgacgcctcgtgcgttcccgcgccaccgtcacgtccggcgactggccggcccggggcaccatcctcgtcgtcggggacaccggcccggtcgccgcgctcctggccggccgcctcctcggcgacggggcggcgcacgtggtgctcgccggcccggccgccgcgtccaccgtcgggctcaccggcggggccgaccgggtggccctgatcgactgcgacccgagcgaccgggacgcgctcgccgggctgctcggcgcgtaccggcccacgacgatcgtggtggctccgcccgccgtcgcgctcaccgccctcgccgagaccacgccggaggacttcgtcgccgccgtcgccgcgaagacgacgacggcagtgcacctcgacgcccttgcggcggaggcggaactggagctcgacgcgttcgtcgtcttctcctcggtctccggcacctggggcggcgcggggcacggcggctacgcggcgggcaccgcccggctggacgcgctggtcgaggagaggcgggcccgtggcctgcccgccacggcgatcgcgtggacgccgtgggccgacgcgaccacagccgccggcgggcaggcacccgatgccagcgccggcgggcacgaacccgacacgagggccgggggccccgaccgcgaactgctgcgccggggtggcctcaccccgttggacccgggggccgcgctggacgtgctgcgcggggcggtggcgcggggcgagggcctggtgaccgtggccgacgtcgactgggcgcggttcgtcgcctcgtacaccgcggcccggcccaccacgctcttcgacgaactgcccgagctgcgggcgacccgggaggcggagcacaccccggccgaggactcgtcggccggcggcgaactggtccgtgccctcagcggccggcccgcggccgatcagcaccggacgctgctgcggctggtccgtgcgcacgtcgcggccgtcctggggcacgacgaggccgaggcggccgatccggaccgggcgttccgggaactcggcttcacctcggtgacggcggtggacctgcggaaccggctgaacgcggccaccgggctgaacctgccggcgtccgtcgtcttcgaccatcccagcgcccgggtgctggccgcgtacctgcgtgccgagctgctcgggccggaggccgacgaggacacggcggaggccgtcgccccgccgtccgcgccggccggggcgggcgacgacgagccgatcgcggtgatcgggatggcctgtcggttcccgggcggggtcgacgcccccgacgacctgtgggatctgctggcgaagggccgcgacgccatctccaggttccccacgaaccggggctgggacgtcgacggcctgtacgacccggacccggaggcgcccggccgcacctacgtccgcgagggcggcttcctgcacgacgcgcccgacttcgatgccgcgttcttcgggatctcgccccgcgaggccctcgccatggatccgcagcagcgcctgctgctggagaccacgtgggagtccctggaacgggccgggttggacccgaccgcgttgcgcggcacccggaccggggtgttcgtggggaccaacggccagcactacatgccgctgctgcgagacggcgcggacgacttcgacggctacctcggcaccggcaactcggccagcgtcatgtccggccggctctcctacgtcttcggcctggagggcccggcggtgaccgtggacacggcctgctccgcctccctcgtggcgctgcacctcgcggtgcaggcgctgcgccggggcgagtgcacgctggccctggtcggcggggccacggtgatgtcgacgccggacatgctggtggagttctcccggcagcgggcgatgtcgccggacggccggtcgaaggcgttcgccgccgccgccgacggggtggcgctcagcgagggcgccgccatgatggtggtgcagcggctcgccgacgcggaggccgccgggcacgagatcctggccgtggtcaagggctcggccgtcaaccaggacggggccagcaacggcctcaccgccccgaacgggccctcccaggaacgggtcatccggcaggcgctggccgacgccggcctgcggccggaccaggtggacgcggtcgaggcgcacggcaccggcaccgccctgggcgaccccatcgaggcgcaggcgctgctcgccacgtacggccgggaccggccggcgggccggccactgtggctcggctcgctgaagtccaacatcggtcacacccaggccgccgccggcatcgccggggtgatgaaggtgatcctggcgctgcggcacgacacgctgccgcgcacgctgcacgtggaccggccgacgccccgggtggactgggcttccggggcggtgtcgttgctgaccgagccggtgccgtggccgcagggcgacgaaccccgccgggcggcggtgtcctcgttcgggatcagcggcaccaacgcccacgtgatcgtcgagcaggcgccgccggtggtgcgggaaccgatcgaccacgaggcggacgaggtcaccgtcccgctgttcctgtcggcccgggggagcgccgcgctctgcgcccaggcggcacggctgcgggcccggttgatcgaggaacccgacctggacatcgccgaggtcggctacacgctggcggccacccgggcccgcttcgagcaccgggccgtggtgatcggggagagccgcgcggaggtcggcgacgcgctcgccgcgctggcccggggcgaggagcacccgtcgctgctgcgggggcgggccggcgcgagcgaccgggtcgcgttcgtctttcccggccagggctcgcagtgggccgagatggccgacggcctgctcgaccgctccccggccttccgggcgagcgcgtcggcgtgcgacgaggcgctgcgggcgcacctcgactggtccgtgctggacgtgctgcgtcgcgtgccggacgcgcctgcgctgagccgggtcgacgtggtccagccggtgctgttcacgatgatggtgtcgctggcggcggcctggcgggcgctgggcgtgcacccgtccgccgtggtcggccactcgcagggtgagatcgcggcggcccacgtggcgggcggcctctcgctggacgacgcggcgcgcatcgtcgccctgcgcagccaggcgtggctgcggctggccgggcagggcgggatggtggcggtgtcgctccccgtcgacgcgctccgcgcccgcctggcgcggttcggcgaccggctgtccgtcgccgcggtcaacagccccggtacggcggcggtgagcggctaccccgacgcgctcgccgaactcgtcgacgagctgaccgccgagggcgtgcacgccaaggcgatcccgggggtggacacggccgggcactccgcgcaggtggaggtgctgaaggaccacctgatggccgccctcgccccggtgtcgccccgcagctcgcagatccccttctactcgaccgtcacgggcggcctgctggacaccgcgctgctggacgccgcctactggtaccgcaacatgcgcgacccggtggagttcgagcaggcgacccgggcgatgctcgcggacgggcacgaggggttcctggagcccagcccgcacccgatgctgtcggtgtcgttgcagggcaccgcggccgatgccggggtcgccgcgacggtgctggggacactgcggcgcggcaagggcggcgcccgctggttcggcatggcgctcgggctcgcccacgcccacgggatcgagatcgacgcgagtgtgctcttcggaaccgactcgcgccgggtcgacctgccgacgtacccgttccagcgcgagcgcttctggtatcacccgccggccgcgcgcggggacgtggcctccgccgggctcagcggtgccgaccatccgctgctgggcggggcggtcgagctgcctgaccggggcggccacgtgtatccggcccggctcggcgtccgacaccacccgtggctcggcgagcatgccctgctgggcgcggcgatcctgcccggggccgcgtacgcggaactcgccctgtgggccgggcggcgtgacggggccggccggatcgaggagctgaccctcgacgcgccgctggtggtggccgacgagtcggcggcgcaactgcggctcgtggtgggcccggcggacgcggaggggcgccggcagctcaccgtccactcgcgcgccgacggcgcggacgcggacaccgcgtggacccggcacgcgcagggcaccctcgtgccggccgacgccgacgccgccgggagcggggacccgggcgcgccctggccgccggccggggccgagcccgtcgaggtggcgggcctgtacgaccggttcgccgaccggggctaccagtacgggccgtcgttccggggggtccgggccgcctggcgggccggcgacacggtgtacgccgaggtggccctgcccgtcccgcagcccgggagcccgcgcttcggtgtccacccggcgctgctcgacgcggcgttccaggcgatgagcctcggcgcgttcttccccgaggacgggcaggtccggatgccgttcgccctgcggggcgtgtcgtcgtccggggtcggggccgaccggctgcgggtcaccatcagcccggccggtgccgaggcggtccggatcgcctgcgtcgacgagcggggcaacccggtcgtggtgatcgactccctggtggcgcgcgcggtgccggtggaggcgctcacccccggcacccccggcaccggggacggcgcgctgcaccacgtcgcctggaccgcccggccggaaccgggggtcgccgccgtgcagcgctgggcggtcgtgggcgcggccgatcccgggctggccgggggcctggaccgggcgggcggcctctgcggggcgtaccccgatctcgccggtctggtcgcggcggtggccgaaggggcggcgctgcccgacgtggtcgcggtgccggtcccgtcgggcgcgccggtcgggcccgacgcggtgcgcgccaccgtgctcggcgccctggacctgatccgggcctggctcgcggtcgagggccggctggggctggccaggctggcgttcgtcaccacctcggcggtggcggtcggcgacggcaccgagcacgtggacccggtgtcggccgccctgtgggggctggtgcgttccgcccagtccgaggagcccggccggttcgtcctcgtcgacctggacgccgacccggccagcgcctcggccctgcccgccgcgctcgccgccggtgagccgcaactggccgttcgcgccggggcggtgcacgtgccccggctggttcggcaccgaccccgcccggacggcccgctgacgcccccggccggtgccgcgtggcggctcgccgccggtgggcagggcaccctggagggcctggcgctggtcccggccccggacgccttggcgccgctggcccccgggcaggtccgggtcgcggtgcgcgccgccggagtgaacttccgggacaccctcatcgcgctcggcatgtacccgggcacgccggtgctgggtgccgagggggccggggtgatcaccgaggtcgcgccggacgtggccggcttcgcccccggcgaccgggtgctgggcatgtggaccggcggcctggggccggtggcggtcgccgacgcccggatgctcgcccgggttccgcgcggctggtcgtacgccgaggccgcgtcggtgccggccgtcttcctcacggcccactacgcgctcaccaggctcgccgggatccgcccggggcagtcgctgctggtgcacgcgggggccggcggcgtcggcatggcgaccctccaactggcccggcacctgggcgtggaggtctacgccacggcgagccggggcaagtgggacaccctgcgtggcctcggcctggacgacgcgcacatcgccgactcccgcagcctcgacttcgccggacggttcctggccgccaccggggggcgcggcgtcgacgtggtgctgaactcccttgccggggacttcgtggacgcgtccctgcggctgctgccgcgcggcggccacttcctggaactgggcaaggccgacgtccgcgaccccgaccggatcgcggccgaccacccgggggtcggctaccgggcgttcgacctcgtcgaggctggtccggagctggtcgggcagctgctcggcgagctgatggagctgttcgccgccggggtgctcagcccgctgccgttgaccgtgcgggacgtccggcgggcccgggaggcgttccgcctgatcagccaggcccggcacgtcggcaaggtggtgctgaccatgccgcccgcgttcggcgcgtacggcaccgtcctggtcaccggcggcaccgggacgctcggcggcgccgtcgcccggcacctggtcgcccggcacggcgtacggcacctggtgctcaccggccgcagcggcccggcggcggacggggcgtccgcgctcgtcgacgagctgaccgcgtccggcgcgtcggtgaccgtcgtcgcctgcgacgccgccgaccgggtcgcgctgcgccggctgctcgacggcattccggccgcgcacccgctcaccgccgtcgtgcacgctgccggcgtcctcgacgacgccaccatcaccgcgctgaccgccgggcaggtggacgcggtgctgcggcccaaggccgacgcggtgatcaacctgcacgagttgacccgggaccgggagctgtccgcgttcgtgctgttctcctcggcggcggccctgttcggcagcccggggcagggcaactactcggcggccaacgggttcgtcgacgcgttcgcccagtaccgccgcgcgcaggggctccacgcggtgtcgctggcctggggcctgtgggccgacagcagccggatggccgggcacctcgaccaggaggggatgcggcgccggatggcgcgcggcggcgtcctgccgctcaccaccgaccagggcctcgccctgttcgacgccgcgcagctggtggacgaggcgctccaggtgccgatccggctcaacgtcggcgcgttgcgggccgccgggagggtccccgcgctcctcgccgacctggtgccggcggcggcgtcgggggccccggccgccaccccgacccgggacgacgcggaccgcacgctcgccgaccggctcgccgggctgaccgtggccgaacagcgggagctggtgctggagagcgtgcgcggacacgcggcggccgtcctcggacacgccgacccgcaggccgtcgacgccgaccgggccttccgggaactcggcttcgactcgctgacggcggtggagctgcgcaatcggctggccaccgcgtccgggctgcgcctgccggcgacgctggtcttcgaccaccccaccccggaagcgttggcggagcacctgctcgccgggctcgcgcccgagcaggcccgggccgagttgccgttgctggccgagctgggccggctggaggcggccctggccgccaccgacggggccgccctcgacgggctggacgacctggtgcgccgggaggtcggcgtccggatcgcggcgctggccgccaggtggggcgcggccggcgacgacgtggccggcagcgacggcggcgggacggccgacgcgctcgagtccgctgacgacgacgagatcttcgcgttcatcgacgagcggttccgcgcctga

SEQ ID NO: 16 - protein, 1574 aa, micromonospora carbonacea subspecies aurantiaca
MSNEQKLREYLRLTTTELARATDRLRAVEARAHEPIAIVGMACRYPGGVGSPEELWELVASGTDAISPFPDDHGWDGDALYDPDPEAAGRTYCREGGFLAGVGDFDAAFFGISPREALAMDPQQRLLLETSWEALERAGIPPDSLRGSRTGVCVGAWHGGYTDVVGQPPAELEGHLLTGGVVSFTSGRISYALGLEGPALTVDTACSSSLVALHLAVRALRQGECDLALAGGATVLASPAVFVQFSRQRGLAPDGRCKAFADSADGFGPAEGVGMLVVERLSDAVRHGRRVLALVTGTAVNQDGASNGLTAPSGPAQEKVLRQALVDARVTAADVDAVEAHGTGTRLGDPIEVRALMNVYGAGRPADRPLWLGSLKSNIGHTQAAAGVGGVIKTVLAMRHGVLPPTLHVDAPTTEVDWSAGQVALLRAETPWPDTGRPRRAGVSSFGVSGTNAHVVLEQAPGPAAAPAGDAPPAETRPVGDPPPVVPLVLSARSQPALAGQARRLRDLLAAAPETDLASAGLALATARSVFDHRAVVTAAGRPQALDALDLLAGGEPGPAVTTGVAAPTGRTVFVFPGQGTHWAGMGADLLDQSPVFAESMRRCEQALSAHTDWKLGEVIRGAAGSPPLDRVDVLQPVSWAVMVSLAQVWRSLGVEPDAVVGHSQGEIAAAVVCGALTLPDAARVVALRSQVIGRVLSGRGGMASVQLPAREVAGRLAAWAGRLDVAAVNGPQSTVVSGAADAVTELVEAFAAEDVRVRRIPVDYASHSTQVDRLRAELLTVLGPVDARPAQVPFYSTVQGGRVDTAGLDAGYWYRNLRGQVRFEETVRVLLDDGHRAFVEAAAHAVLVPAIQELGDSAGVRVVAVGSLRREAGGLDRLLASAAEAFTQGVAVDWSRALAGAARVAVDLPTYAFQRQRYWLEPAAQADSGPAGDGWRYRVGWRRLQRTGAAPADRWLLVTGPEQPAELVEAVRDALTARGAEVRLVTVEPTSTDRAACAALLTAAGAGGATRVLSLLGTDRRPHPDHPAVSVGAAATLLLTQAVADALPAARLWVVTRGAVSVGPGETADERQAQVWGFGRVAALELPRTWGGLVDLPADADGPVWEAFVDVLAGDEDQVALRGPVGYGRRLRRAPALPAKRRYRPRGTVLVTGGTGALGAHVARRLAAGGAAHLVLTSRRGADAPGAAGLVGELRALGAEVTVAVCDVADRAAVAALLAGLPADAPLSAVFHTAGVAHSMPIGETGLTDVAEVFAGKVAGARHLDELTRGHDLDAFVLYSSNAGVWGSSGQSAYGAANAALDALAERRRAAGLTATSVAWGLWGSGGMGEGDAEEYLSRRGLRPMPPERGVDALLAALDRDETFVAVADVDWTLFTAGFTAFRPSPLLGDLPEARATLADAGPAGSDLPAWHAAASPDERRRGLLDLVRRQVAAVLGHPGPEHVGPDAAFREIGFDSLTAVDLAKRLRAAVGVPLSATLVFDHPTATAVAEHLAGLLGPAPAGGDPREAEVRRALADLPLARLRDAGLLDGLLALAGLDADAVPDGPEPAPGDAIDELDPEELVRRVLDNASS

SEQ ID NO: 17 - DNA, 4725 bp, micromonospora carbonacea subspecies aurantiaca
atgtcgaacgagcagaagctccgcgagtacctgcggttgaccaccaccgagctggccagggccaccgaccggctgcgcgcggtcgaggcgcgggcgcacgagccgatcgcgatcgtcggcatggcctgccggtaccccggcggggtcggctcaccggaggaactgtgggagctggtcgcctcgggcacggacgcgatctccccgttccccgacgaccacggctgggacggcgacgcgctgtacgacccggacccggaggcggcgggccgcacctactgccgcgagggcgggttcctcgccggggtcggcgacttcgacgccgcgttcttcggcatctcgccccgcgaggcgctggccatggacccgcagcagcgcctgctgctggagacgtcctgggaggcgctggagcgggccgggatccccccggactcgctgcgcggcagccgtaccggggtgtgcgtcggggcgtggcacggcggctacaccgacgtcgtcgggcagcccccggcggaactggagggccacctgctgaccggcggggtggtcagcttcacctcggggcggatctcgtacgcgctgggcctggaggggcccgcgttgacggtggacaccgcctgctcgtcctcgctggtggccctgcacctggcggtgcgggccctgcggcagggcgagtgcgacctggcgttggccggcggggcgacggtgctggccagcccggcggtgttcgtgcagttctcgcggcagcgggggctggccccggacggccggtgcaaggcgttcgccgactcggcggacgggttcgggccggccgagggggtcggcatgctggtcgtggagcggctgtcggacgccgtccgccacgggcgccgggtgctggccctggtcaccggcacggcggtcaaccaggacggggcgagcaacggcctcaccgcccccagcggcccggcgcaggagaaggtgctgcgccaggcgctcgtggacgcccgggtgacggccgccgacgtcgacgcggtcgaggcgcacggcaccggcacccggctcggcgacccgatcgaggtgcgggccctgatgaacgtgtacggtgccggccggcccgccgaccgtccgctctggctcggttcgctgaagtccaacatcggccacacccaggcggcggccggggtcggcggggtcatcaagacggtgctggcgatgcggcacggcgtcctgccgcccaccctgcacgtggacgccccgaccaccgaggtcgactggtccgccggccaggtggccctgctgcgggcagagacaccgtggccggacacgggtcgcccgcgccgcgccggggtctcctccttcggggtgagcggcaccaacgcgcacgtggtgctggagcaggcccctgggcccgccgccgccccggcgggtgacgccccgcccgccgagacccggcccgtcggcgacccgccgccggtcgtaccgctggtgttgtccgccaggtcgcagccggcgctggccgggcaggcccgccggctgcgcgacctgctggccgcagcgccggagaccgacctcgccagcgccggactcgccctggccaccgcgcggtcggtgttcgaccaccgggcggtggtgacggccgccgggcgaccgcaggcgctcgacgcgctcgacctgctggccggcggcgaacccggaccggcggtcacgaccggcgtcgccgcccccaccgggcgcaccgtgttcgtctttcccgggcaggggacgcactgggccggcatgggtgccgacctgctcgaccagtcaccggtgttcgccgagtcgatgcgacggtgcgagcaggcgctgtcggcgcacaccgactggaagctcggcgaggtgatccggggcgcggccggcagcccgccgctggaccgcgtggacgtgctccagcccgtctcctgggcggtgatggtgtcgctggcgcaggtgtggcggtcgctcggcgtcgagccggacgcggtggtcggccattcccagggcgagatcgccgccgcggtggtctgcggcgcgctgaccctgccggacgcggcccgggtggtcgcgctgcggtcccaggtcatcggtcgggtgctctccggtcgcggcggcatggcgtccgtccagctgccggcccgggaggtcgcggggcggctggccgcctgggcgggccggctcgacgtcgcggccgtcaacgggccacagtcgaccgtcgtgtccggtgccgccgacgcggtcaccgaactggtcgaggcgttcgcggccgaggacgtccgggtgcggcggatcccggtggactacgcgtcccactcgacgcaggtggaccggctgcgcgccgagctgctcaccgtcctgggcccggtcgacgcccgtccggcgcaggtgcccttctactcgacggtgcagggcgggcgcgtcgacactgccggcctggacgccggctactggtaccgcaacctgcgggggcaggtccgcttcgaggagaccgtgcgggtgctgctcgacgacgggcaccgcgccttcgtcgaggccgccgcgcacgccgtcctcgtacccgcgatccaggagctgggggacagcgccggcgtccgggtggtggccgtggggtcgctgcgccgggaggcgggcggcctggaccggctcctggcctcggcggccgaggcgttcacccagggggtggccgtggactggtcccgggctctggccggggccgcgcgcgtcgccgtggacctgcccacgtacgcgttccagcggcaacgctactggctggagcccgccgcgcaggcggactccggcccggccggggacggctggcgctaccgggtcggctggcggcggcttcagcgcaccggcgccgcgccggccgaccggtggctgctggtgaccggcccggagcagccggcggagctggtcgaggcggtgcgcgacgcgctcaccgcgcggggcgccgaggtgcgcctggtgaccgtcgagccgaccagcaccgaccgggccgcgtgcgcggcgttgctcaccgcggccggtgcgggcggggcgacccgggtgctgtcgctgctcggcaccgatcgtcgcccgcaccccgaccacccggccgtgtccgtcggcgccgccgcgacgttgctgctgacccaggccgtcgccgacgccctgccggccgcccggctgtgggtcgtcacccggggcgcggtctccgtcgggcccggcgagaccgccgacgagcgccaggcgcaggtctgggggttcggccgggtcgcggccctcgaactgccccgcacgtggggcgggctcgtcgacctgcccgccgacgcggacggcccggtgtgggaggcgttcgtggacgtgctggccggggacgaggaccaggtcgcgctgcgcggcccggtcgggtacggtcgccggctccggcgcgcccccgcgctacccgcgaagcggcggtaccggcccaggggcaccgtcctggtcaccggcggcaccggcgcgctcggcgcgcacgtggcccggcggttggccgccggcggggccgcgcacctcgtgctcaccagccggcgcggggccgacgcccccggtgcggccgggctggtcggggaactccgggcgctgggcgccgaggtgaccgtcgcggtctgcgacgtcgccgaccgggccgccgtggcggcgctgctcgccgggctgcccgccgacgcgccgctgagcgcggtcttccacaccgcgggcgtggcgcactcgatgccgatcggcgagaccgggctcaccgacgtcgccgaggtgttcgccgggaaggtcgccggagcccgccacctcgacgaactcacccgggggcacgacctggacgcgttcgtcctgtactcgtcgaacgcgggcgtgtggggcagcagcgggcagagcgcgtacggggcggccaacgcggccctcgacgcgctcgccgaacggcggcgcgccgccgggctgaccgccacctccgtcgcctggggcctgtggggctccgggggcatgggcgagggcgacgccgaggagtacctgagccgccggggcctgcggccgatgcctcccgagcgtggcgtggacgccctcctggccgccctggaccgggacgagaccttcgtcgccgtcgccgacgtggactggacgctgttcacggccgggttcaccgcgttccggcccagcccgctgctcggcgacctcccggaggcccgcgcgacgctggccgacgccggacccgcgggctccgacctgccggcctggcacgccgccgcgagccccgacgaacgccgccggggcctgctcgacctggtacgccggcaggtcgccgccgtcctcggccacccggggcccgagcacgtcggccccgacgccgcgttccgggagatcggattcgactcgctgaccgccgtcgacctggccaagcggctcagggcggcggtcggcgtgccgctgtccgccaccctcgtcttcgaccaccccaccgcgacggcggtcgccgagcacctggccgggctgctcggtcccgcgccggccggcggcgacccgcgcgaggccgaggtgcgccgggccctggccgacctgccgctggcccggctgcgggacgccggcctactggacggcctgcttgcgcttgcggggctggacgccgacgcggtgccggacgggcccgagccggctcccggcgacgccatcgacgaactcgatccagaggagctggtgcgccgggtgctggacaacgccagctcctga

SEQ ID NO: 18 - protein, 1784 aa, micromonospora carbonacea subspecies aurantiaca
MVMPPDKVIEALRVSVKETERLRRQNHELLAALHGPIAVVGMACRYPGGVSSPEDLWRLVETGTDAIGGFPTDRGWDVDAVYDPDPESRNTTYCREGGFLAGAGDFDAAFFGVSPHEAVVMDPQQRLLLEVSWEALERSGTDPHSLRGSRTGVYVGAAHQGYAVDAGQVPEGAEGFRLTGSADAVLSGRISYLLGLEGPALTVETACSSSLVAVHLAVQALRRGECGLALAGGVAVMPDPAAFVEFSRQRGLAADGRCRAFGAGADGTGWAEGVGVLVLQRLSDAVRDGRWVLGVIRGSAVNQDGASNGLTAPSGPAQQRVIRQALTDARLGADQIDAVEAHGTGTRLGDPIEAQALIAAYGADRTPDRPLWLGSLKSNIGHAQAAAGVGGLIKMLLAMRAGTLPPTLHADVPTPLVDWSAGVVRLSTGVVPWPALPGAPRRAGISAFGVSGTNAHVIVEQPPPVPVDDPAPPTRTLPLVPWVLSGRTEAALRAQADRLRTHLAAHPDADPLDVGFSLATSRAALEHRAVLVAADRDGLLRLVDALAAGEPAAGLIRGTVRHDRRTGFLFAGQGGQRVGMARELYEAFPAFADALDQLAARLDRHLDRPLLRVLFAEPGSDDARLLDGTRYAQAALFAVEVALFRLVHGWGVRPDVLLGHSVGELAAAHVAGVLDVDDACELVAARGRLMGELPSGGAMVAVRATEEEVGPLLDGQRVAVAAVNGPRSVVVSGDEEAVLAVAARCAALGHRTRRLNVSHAFHSPHVEAMLEPFRRVARGLTYHAPTIPVVSNATGRLATADALRDPGYWVRHVRQPVRFRDGVRAARDQGATAFVGLGPDGVLCALAEECLGPTGDVLLLPVLRPGRPEPATLLAALAGAYAGGAEMDWSRVFAGTGARRVELPTYAFQHRRYWLAPGPPSARRDDAWRYRIAWRPLPTVPAAAGTETVAGAWLLVVPAHDGVASLADAAERAVHRGGATVTRLTVDAADVDRDTLAAVLTEAAADADGGPDGVLCLLGLDDRAHPRSASVPRGVLATLSLAQALTDLGASARLWCVTRGAVAVTPGESPSVAGAQLWGFGRVAALELPRSWGGLVDLPVDPDDRDWDLLRRALRGPEDQVAVRGAVGYARRLVPAPAPRAERAWRPRGTVLVTGGTGALGAHTARWLARNGATHLVLTSRRGGNAPGVAALRAELVTLGAEVTVVACDVADREAVAGLLAGIPRAAPLTAVFHAAGVPQVTPLHETTPELFAQVCAGKVAGAVHLHELAGDLDAFVTFASAAGVWGSGGQCAYAAANAALDALAERRRAAGLPATSVAWGVWGGPGMGAGAGEEYLRRRGVRAMPPAAALAALGRILDADETGVTVSDTEWGRFASGFAAARPAPLLAELPGGDVDPAGPAHRAQPPVPRPAPAATDRPGLLALVRAEAAGVLGHDGADDVPADAEFSALGFDSLAAVQLRRRLAEATGLSLSAPVLFDHRTPDALAAHLHGLLTGAAGGPPAPAAGSALVEMYRRAVATGRAAEAVEVLGTVATFRPVFRSPDELGEPPALVPLGTGAGGPALVCCAGTAAASGPREFTAFAAALAGLRDVTVLPQTGFLPGEPLPAGLDVLLDAQADAVLAHCAGGPFVLVGHSAGANMAHALTVRLEARGADPAALVLMDIYTPAAPGAMGVWREEMLAWVAERSVVPVDDTRLTAMGAYHRLLLDWAPRPTRAPVLHLYAGEPAGAWPDPRQDWRSRFDGAHTSAEVPGTHFSMMTEHAPVTAATVHKWLDEVCPPRVP

SEQ ID NO: 19 - DNA, 5355 bp, micromonospora carbonacea subspecies aurantiaca
atggtcatgccccccgacaaggtgatcgaggcgctgcgtgtctccgtcaaggagacggagcggctgcgccggcagaaccacgagctgctcgccgccctgcacgggccgatcgccgtcgtgggcatggcctgccgctacccgggcggggtgtcctctccggaggacctgtggcggctggtcgagacgggcacggacgcgatcggcggcttccccaccgaccgtggctgggacgtcgacgccgtgtacgacccggatcctgagtcgcggaacaccacctactgccgggagggcgggttcctggccggggcaggagacttcgacgccgcgttcttcggggtgtcgccgcacgaggccgtggtcatggacccccagcagcggctgcttctggaggtgtcctgggaggcgctggagcggtccgggaccgacccgcacagcctgcgcggctcgcgcaccggggtctacgtcggtgcggcccaccaggggtacgcggtcgacgccggtcaggtgccggagggcgcggaggggttccggctgaccggcagcgccgacgccgtcctgtccggacggatctcgtacctgctcgggctggagggtccggccctgaccgtcgagacggcctgctcgtcctcgctggtggcggtgcacctcgcggtgcaggcgctgcgccggggcgagtgcgggctggcactggccggcggggtcgccgtgatgcccgacccggcggcattcgtggagttctcccggcagcggggcctcgcggcggacgggcgctgccgggcgttcggggcgggcgcggacggcaccggctgggcggagggcgtcggtgtgctggtcctgcaacggctctccgacgcggtgcgcgacggccgctgggtgctgggcgtgatccggggttcggccgtcaaccaggacggggccagcaacgggctgaccgccccgagcggccccgcccagcagcgggtcatccggcaggcgctgaccgacgcccggctcggcgccgaccagatcgacgcggtcgaggcgcacggcacgggcacccggctcggcgacccgatcgaggcgcaggcgctgatcgccgcctacggcgccgaccggaccccggaccggccgctctggctcggctcgttgaagtcgaacatcgggcacgcccaggcggcggccggcgtcggcggcctgatcaagatgctcctggcgatgcgggccgggacgctcccacccaccctgcacgccgacgtcccgaccccgctggtcgactggtccgccggtgtcgtccggctgtcgaccggggtggtgccctggcccgcgttgcccggggcgccccgcagggccgggatctccgcgttcggggtgagcggcaccaacgcgcacgtgatcgtcgagcagccgccgccggtcccggtcgacgacccggcgccacccacgaggaccctgccgctggtgccgtgggtgctctccggccggacggaggcggcgctgcgcgcccaggcggaccggttgcgtacgcacctggcggcgcaccccgacgcggacccgctggacgtgggattctccctggccaccagccgggccgcgctggagcaccgggccgtgctggtggccgccgaccgcgacggcctgctccgcctcgtcgacgcgctggccgccggcgagccggcggcgggcctgatccggggcacggtacgtcacgatcgccggaccgggttcctcttcgccgggcagggcggccagcgcgtcgggatggcgcgcgaactgtacgaggcgttccccgccttcgccgacgccctggaccagctcgccgcccggctggaccggcacctcgatcgtccgctgctgcgggtgctgttcgccgagccggggtcggacgacgcccggctgctcgacggcacccggtacgcgcaggccgccctcttcgccgtcgaggtggcgttgttccgactggtccacggctggggggtccggcccgacgtgctgctcggccactcggtgggcgagctggcggccgcgcacgtggccggcgtactcgacgtggacgacgcgtgcgagctggtcgcggcgcggggccggctgatgggggagctgccgtcgggcggcgcgatggtggcggtccgggccaccgaggaggaggtcgggcccctgctcgacgggcagcgggtcgcggtggcggcggtcaacggcccgcgctcggtcgtggtctccggcgacgaggaggcggtgctggccgtggccgcccggtgcgccgccctcggccaccggacgcgacgcctcaacgtcagccacgcgttccactccccgcacgtggaggcgatgctggagccgttccggcgggtggcgcggggcctgacgtaccatgccccgacgatcccggtggtgtcgaacgcgacgggccggctcgccaccgccgacgcgctgcgcgaccccggttactgggtccggcacgtccgccagcccgtccggttccgggacggggtgcgggccgcccgcgaccagggggccaccgccttcgtcgggctcggcccggacggggtgctgtgcgcgttggccgaggagtgcctcgggcccaccggcgacgtgctgctgctgccggtgctgcgccccggtcggccggagcccgccaccctgctggccgccctggccggggcgtacgccggcggcgcggaaatggactggtcccgggtgttcgcgggcaccggcgcgcgcagggtcgagctgcccacgtacgccttccagcaccggcgctactggctggcgccgggcccgccgtcggcccgccgcgacgacgcctggcggtaccggatcgcctggcggcccctgccgaccgtgcccgccgccgccgggaccgagacggtggccggggcgtggttgctggtggtccccgcccacgacggcgtcgcgtcgctcgccgacgccgccgagcgggccgtgcaccggggcggggccacggtcacccggctgacggtggacgccgccgacgtggaccgggacaccctcgccgccgtgctgaccgaggccgccgccgacgcggacggcgggccggacggggtgctctgcctgctgggcctcgacgaccgggcacatccccggtccgcctcggtgccccgcggggtgctggcgaccctgtccctcgcccaggccctgaccgacctgggggcctccgcgcggctgtggtgcgtgacccggggggcggtcgccgtgacgcccggcgagtccccgtcggtcgccggagcccagttgtggggcttcggccgcgtggccgcgctcgaactcccccggtcctggggcggcctggtggacctgccggtcgacccggacgaccgggactgggacctgctgcggcgcgcgctgcgcggcccggaggaccaggtcgcggtccggggggcggtcgggtacgcccggcggctggtccccgcgcccgcgccccgggccgagcgggcctggcgtccgcgcggcacggtcctggtgaccggcggtacgggcgcgctcggcgcgcacacggcccgctggctggcgcgcaacggcgccacgcacctcgtcctcaccagccgccggggcgggaacgcccccggggtcgccgcgctgcgggcggaactggtcacgctcggtgccgaggtgaccgtggtcgcctgcgacgtcgccgaccgggaggccgtggccggcctgctcgccgggattccccgcgccgctccgctcaccgccgtgttccacgcggcgggcgtgccccaggtgacgccgcttcacgagacgaccccggagttgttcgcgcaggtctgcgcaggcaaggtcgccggggcggtgcacctgcacgagttggccggtgacctggacgccttcgtcaccttcgcctccgccgccggggtgtggggcagcggcgggcagtgcgcgtacgctgcggccaacgccgccctcgacgcgctcgccgagcgtcgtcgcgccgcagggctgcccgcgacctccgtcgcctggggggtctggggcgggcccggcatgggggcgggcgcgggggaggagtacctgcgccgccggggcgtccgggcgatgcccccggcagccgccctcgccgccctcgggcggatcctggacgccgacgagaccggggtgacggtctccgacaccgagtggggccggttcgcgtccggcttcgccgccgcgcgtcccgccccgctgctcgccgagctgccgggcggggacgtcgatccggccggcccggcgcaccgggcgcagccgcccgtgccccgaccggccccggcagccaccgaccgccccgggctgctggcgctggtccgcgccgaggccgccggggtgctggggcacgacggtgccgacgacgttccggccgacgcggagttctccgccctcggcttcgactcgctcgccgccgtccagctgcgccgccggctcgccgaggccaccggcctgagcctctcggccccggttctgttcgaccaccgcacccctgacgcgctcgccgcgcacctgcacggcctgctcaccggcgcggcgggcgggccacccgcgccggccgccgggagcgccctggtcgagatgtaccggcgggccgtcgccaccggccgcgccgccgaggcggtggaggtgctcggcaccgtcgccacgttccggccggtgttccggtccccggacgaactgggcgagccaccggccctcgtcccgctcggcaccggggcggggggacccgcgctggtctgctgcgcgggcacggccgcggcgtccggcccccgcgagttcacggcgttcgccgccgcgctggccggtctccgggacgtcaccgtccttccgcagaccggcttcctgcccggcgagccgctgcccgccgggctggacgtgctgctcgacgcccaggccgacgccgtcctggcccactgcgccgggggacccttcgtcctggtcggccactcggccggggcgaacatggcgcacgcgctgacggtccgcctggaggcgcggggcgcggaccccgccgcgctggtgctgatggacatctacacgcccgccgccccgggggcgatgggggtgtggcgcgaggagatgctggcctgggtcgccgagcggtccgtcgtccccgtcgacgacacgcggctgaccgcgatgggcgcctatcaccggctgctcctggactgggcgccccggccgacccgggcacccgtgctgcacctgtatgccggtgaaccggcgggcgcctggccggatccccggcaggactggcgttcgcgcttcgacggcgcgcacaccagcgccgaggtgcccggcacccacttctcgatgatgaccgagcacgcccccgtcaccgccgcgaccgtgcacaagtggctcgacgaggtgtgcccgccccgcgttccgtga

SEQ ID NO: 20 - protein, 464 aa, micromonospora carbonacea subspecies aurantiaca
VTRTPGPSRRVRRQQERKRMITVPPDGDPATWARRLQLTRAAQWFAGNHGDPYALILRAETDDPTPYEQRVAAQPLFRSEQLDTWVTGDAALAREVLTDDRFGWLTRAGQRPAERTLPLAGTALDHGPEARRRLDALAGFGGPVLRADAAGARTRVVETTAVLLDGIGERFDLAVLARRLVAAVLADLLGVPAARRGRFAEALAAAGRTLDSRLCPQTVATALATVAATAELTDLLGEVPPPPSLSPSAAGSGPPRPSAAGSWPPLPADDRTAAALALAVGTAEPAITLLCNAVGALLDRPGQWALLGGDLDRSAAVVEETLRCLPPVRLESRVAQQDVTLGGQFLPADSHLVVLVAMANRGPRAATAPSPDAFDPGGSRVPARDVVGLPQLAGAGPLIRLVVTTALRTLAEALPTLRRASGGVRWRRSPVLLGHARFPVARAESGEQRSDDRPALEEAIRCAS

SEQ ID NO: 21 - DNA, 1395 bp, micromonospora carbonacea subspecies aurantiaca
gtgacccgtacgccgggtccgtcccggcgagtccgacgacagcaggagaggaagcgcatgatcacagtcccgcccgacggggatcccgcgacctgggcccgccggctgcaactgacccgcgccgcgcagtggttcgccggcaaccacggcgacccgtacgcgctgatcctgcgcgcggagaccgacgacccgaccccgtacgagcagcgggtggccgcccagccgctgttccgcagcgagcagttggacacctgggtgaccggggacgccgcgctggcccgggaggtgttgaccgacgaccggttcggctggctgacccgggctgggcagcggcccgccgagcggaccctgccgctggccggcacggcactggaccacgggccggaggcccggcgtcggctggacgcgctcgccgggttcggcgggccggtcctgcgggccgacgccgcaggggcgcgtacccgggtcgtggagaccaccgcggtcctgctcgacgggatcggggagcggttcgacctggccgtgctcgcccggcggctggtcgctgcggtgctggccgacctgctgggggtgcccgccgcgcggcggggccgcttcgccgaggcactcgccgccgccggccgtacgctggacagccggctgtgcccgcagaccgtggcgaccgctctcgccaccgtcgccgccaccgccgagctgaccgacctgctgggcgaggtgccgcccccgccgtcgctgtccccgtccgccgccggctccgggccgccgcgtccgtccgcagccggttcctggccgccgctgccggctgacgaccggacggccgccgcgctcgcgctggcggtcggcacggccgaaccggcgatcaccctgctctgcaacgcggtcggtgcgctgctcgaccgccccgggcagtgggccctgctcggtggggacctcgaccggtccgccgccgtcgtcgaggagaccctgcgctgccttccgccggtgcgcctggagagccgcgtcgcgcagcaggacgtcaccctgggcgggcagttcctcccggcggacagccacctggtcgtgctggtcgccatggcgaaccggggtccgcgcgcggcgaccgccccgagcccggacgcgttcgaccctggcgggtcgcgcgtcccggcccgcgacgtggtgggcctgccgcagcttgccggcgccgggccgctgatcagactcgtcgtcacgaccgccctgcggaccctcgccgaggcgctgcccacgctgcggcgggcgtccggcggcgtccggtggcgacgctcgcccgtcctgctcggccacgcccgctttcccgtcgcacgggcggagagcggcgaacagcggtccgacgaccgcccggcgctggaggaggcgatccgatgcgcgtcctga

SEQ ID NO: 22 - protein, 429 aa, micromonospora carbonacea subspecies aurantiaca
MTSFAHNTHYYSLVPLAWALRAAGHEVRVASQPSLTDTIVRSGLTAVPVGDDQAIIDLLAEVGGDLVPYQRGLDFTEARPEVLTWEYLLGQQTMLTALCFAPLNGVSTMDDMVALARSWQPELVIWEPFTYAGPVAARVVGATHARLLWGPDVVGNARRLFTESLARQPDEQREDPMAEWLRCTLHRYGCELGDDEVETLVTGGWTIDPTADSTRLPVPGRRVAMRYTPYNSPSVVPEWVAKADRPRVCLTLGVSSRETYGRDVVSFQELLGALGDLDVEVVATLSDAQREDLGDLPDNVRVCDFVPLDVLLPTCAAIIHHGGAGTWSTAMLYGVPQIMIASLWDAPLKAQQAERLGTGISIPPERLDAPTLRAAVVRILDDPSIAAAARRQRDELRAAPSPAEVVRILERLVADDRPGRPAGTATDHS

SEQ ID NO: 23 - DNA, 1290 bp, micromonospora carbonacea subspecies aurantiaca
atgacgtccttcgcgcacaacacccactactacagcctggtgccgttggcctgggcgctgcgcgcggccggccacgaggtacgggtggcgagccagccctcgctcaccgacaccatcgtgcggtcggggctgaccgcggtgccggtcggcgacgaccaggcgatcatcgacctgctcgccgaggtcggcggcgacctggtgccgtaccagcggggactggacttcaccgaggcccgtcccgaagtgctgacctgggagtatctgctcgggcagcagaccatgctcaccgcgctgtgcttcgcgccgctcaacggcgtctccacgatggacgacatggtcgccctggcccggtcctggcagcccgagctggtgatctgggagccgttcacctacgccgggccggtcgcggcgcgggtcgtcggtgcgacgcacgcccggctgctctgggggccggacgtggtcggcaacgcccggcggctgttcaccgagagcctggcgcggcagccggatgagcagcgcgaggacccgatggccgagtggttgcgctgcaccctgcaccggtacggctgcgagctcggcgacgacgaggtggagaccctggtcaccggcgggtggaccatcgatcccaccgccgacagcacccggcttcccgtccccgggcgtcgggtggccatgcggtacaccccgtacaacagcccgtccgtggtgccggagtgggtggccaaggccgaccggccccgcgtctgcctcaccctcggcgtgtcgagccgggagacgtacggcagggacgtggtctccttccaggagctgctcggcgccctcggcgacctggacgtcgaggtcgtcgcgacgctcagcgacgcccagcgcgaggacctgggtgacctgccggacaacgtccgggtgtgcgacttcgtgccgctggacgtgctgctgccgacctgtgccgcgatcatccaccacggcggggcgggcacgtggtcgacggccatgctctacggggtgccgcagatcatgatcgcgtcgctgtgggacgccccgctcaaggcgcagcaggcggagcgactcggcacggggatctcgatcccgccggagcggctcgacgccccgacgctgcgggcggccgtcgtccggatcctcgacgacccgtcgatcgccgccgccgcccgccgtcagcgcgacgagctgcgtgccgcgccgtcgccggccgaggtggtccgcatcctggaacgcctcgtcgcggacgaccggcccggccggccggccggaaccgccaccgaccactcctga

SEQ ID NO: 24 - protein, 240 aa, micromonospora carbonacea subspecies aurantiaca
MSMMYADAIAEVYDLIYQGKGKDYAAEAAELEALARARRPHARTLLDVACGTGLHLRHLAGLFDDVGGIELAPDMLSIAQQRNPGAALHLGDMRTFDLGHRYDVITCMFSSVGHLATTAELDATLARFAAHLSPGGVAIVEPWWFPETFTPGYVGASLVEVDGRTISRVSHSVREGGATRITVHYLVASPGGGVRHFDESHLITLFERSDYERAFARAGFTTEYLTPGPSGRGLFVGVHP

SEQ ID NO: 25 - DNA, 723 bp, micromonospora carbonacea subspecies aurantiaca
atgtccatgatgtacgcggacgccatcgccgaggtctacgacctgatctaccagggcaagggcaaggactacgcggcggaggcggcggagctggaggcgctggcccgggcccgtcggccgcacgcccggacgctgctggacgtggcgtgcggcacggggctgcacctgcggcacctggcggggctcttcgacgacgtgggcggcatcgagctggcaccggacatgctgagcatcgcccagcagcgaaaccccggggcggccctgcacctcggcgacatgcggaccttcgacctggggcaccgctacgacgtcatcacctgcatgttcagttcggtgggccacctggccaccacggccgagctggacgcgacgttggcccggttcgccgcgcacctgtcccccgggggagtggcgatcgtcgagccgtggtggttcccggagaccttcacccccgggtacgtgggcgcgagcctggtggaggtcgacggccgtaccatctcgcgggtctcccattcggtgcgcgagggcggcgcgacccggatcaccgtgcactacctcgtggccagccccggcgggggagtccggcacttcgacgagagccacctgatcaccctcttcgaacggtccgactacgaacgtgccttcgcccgggcgggtttcacgacggagtacctgacgcccggcccgtccggccgcggtctgttcgtcggcgtccacccctga

SEQ ID NO: 26 - protein, 1811 aa, micromonospora carbonacea subspecies aurantiaca
MPDTPELNRILDAILAQETDARELAALPLPSSYRAVTVHKDETGMFLGLPRQEKDPRKSLHTEEVPVPELGPGEALVAVLASSVNYNTVWSSLFEPLPTFGFLERYGRLSELARRHDLPYHILGSDLAGVVLRVGPGVNRWRPGDEVVAHCLSVELESADGHGDTMLDPEQRIWGFETNFGGLAEIALVKANQLMPKPDHLTWEEAAAPGLVNSTAYRQLVSGNGARMKQGDNVLVWGASGGLGAFATQLVLAGGANPVCVVSSPRKADICRRMGAEAVIDRVAEDYRFWSDERTQNPREWKRFGARIRELTGGEDVDIVFEHPGRETFGASVYVTRKGGTVVTCASTSGFEHVYDNRYLWMSLKRIVGTHFANYREAWEANRLVVKGKIHPTLSRCYPLEEVGQAVYDVHHNLHQGKVGVLALAPREGLGVRNPELRECHLAAINRFRVPAATGCCAGACACCCCCGAGCTGAACCGGATACTCGACGCGATCCTCGCCCAGGAGACCGACGCGCGGGAGCTGGCGGCCCTGCCGCTGCCCTCCTCCTACCGGGCCGTGACGGTGCACAAGGACGAGACGGGGATGTTCCTGGGCCTTCCCCGCCAGGAGAAGGACCCGCGCAAGTCGCTGCACACGGAGGAGGTGCCGGTGCCCGAGCTGGGCCCCGGGGAGGCCCTCGTCGCGGTCCTGGCCAGCTCGGTCAACTACAACACGGTCTGGTCGTCGTTGTTCGAGCCGCTGCCCACCTTCGGCTTCCTGGAGCGCTACGGCCGGCTCTCCGAGCTGGCCCGGCGGCACGACCTGCCGTACCACATCCTCGGCTCGGACCTGGCCGGCGTGGTGCTGAGGGTCGGGCCCGGCGTCAACCGCTGGCGGCCGGGTGACGAGGTCGTGGCGCACTGCCTCTCGGTGGAGCTGGAGTCCGCCGACGGCCACGGCGACACCATGCTCGACCCGGAACAGCGGATCTGGGGCTTCGAGACCAACTTCGGCGGCCTCGCCGAGATCGCGTTGGTCAAGGCGAACCAGCTGATGCCCAAACCCGACCACCTGACCTGGGAGGAGGCCGCCGCGCCGGGACTGGTCAACTCCACCGCCTACCGCCAGCTGGTCTCCGGCAACGGGGCCCGGATGAAGCAGGGCGACAACGTCCTCGTCTGGGGGGCCAGCGGCGGTCTCGGCGCGTTCGCCACCCAGCTCGTGCTGGCCGGCGGGGCCAATCCCGTCTGCGTGGTCTCCAGCCCGCGCAAGGCCGACATCTGCCGTCGGATGGGCGCCGAGGCCGTCATCGACCGGGTCGCCGAGGACTACCGCTTCTGGTCCGACGAGCGCACCCAGAATCCCCGGGAGTGGAAGCGCTTCGGCGCACGCATTCGGGAGCTGACCGGAGGCGAGGACGTCGACATCGTCTTCGAGCACCCCGGCCGGGAGACGTTCGGCGCCTCGGTCTACGTGACCCGCAAAGGAGGCACCGTGGTCACCTGCGCCTCGACGAGCGGTTTCGAGCACGTCTACGACAACCGTTACCTGTGGATGTCCCTGAAGCGCATCGTCGGCACGCACTTCGCCAATTACCGGGAGGCGTGGGAAGCCAACCGGTTGGTGGTCAAGGGCAAGATCCACCCGACGCTGTCGCGCTGCTACCCGCTGGAGGAGGTCGGCCAGGCGGTCTACGACGTCCATCACAACCTGCACCAGGGCAAGGTCGGCGTGCTCGCGCTCGCGCCGCGCGAGGGGCTCGGGGTCCGGAACCCGGAGCTGCGGGAATGCCATCTTGCCGCGATCAACCGCTTCCGGGTGCCGGCCTGA

SEQ ID NO: 27 - DNA, 1359 bp, micromonospora carbonacea subspecies aurantiaca
atgccagacacccccgagctgaaccggatactcgacgcgatcctcgcccaggagaccgacgcgcgggagctggcggccctgccgctgccctcctcctaccgggccgtgacggtgcacaaggacgagacggggatgttcctgggccttccccgccaggagaaggacccgcgcaagtcgctgcacacggaggaggtgccggtgcccgagctgggccccggggaggccctcgtcgcggtcctggccagctcggtcaactacaacacggtctggtcgtcgttgttcgagccgctgcccaccttcggcttcctggagcgctacggccggctctccgagctggcccggcggcacgacctgccgtaccacatcctcggctcggacctggccggcgtggtgctgagggtcgggcccggcgtcaaccgctggcggccgggtgacgaggtcgtggcgcactgcctctcggtggagctggagtccgccgacggccacggcgacaccatgctcgacccggaacagcggatctggggcttcgagaccaacttcggcggcctcgccgagatcgcgttggtcaaggcgaaccagctgatgcccaaacccgaccacctgacctgggaggaggccgccgcgccgggactggtcaactccaccgcctaccgccagctggtctccggcaacggggcccggatgaagcagggcgacaacgtcctcgtctggggggccagcggcggtctcggcgcgttcgccacccagctcgtgctggccggcggggccaatcccgtctgcgtggtctccagcccgcgcaaggccgacatctgccgtcggatgggcgccgaggccgtcatcgaccgggtcgccgaggactaccgcttctggtccgacgagcgcacccagaatccccgggagtggaagcgcttcggcgcacgcattcgggagctgaccggaggcgaggacgtcgacatcgtcttcgagcaccccggccgggagacgttcggcgcctcggtctacgtgacccgcaaaggaggcaccgtggtcacctgcgcctcgacgagcggtttcgagcacgtctacgacaaccgttacctgtggatgtccctgaagcgcatcgtcggcacgcacttcgccaattaccgggaggcgtgggaagccaaccggttggtggtcaagggcaagatccacccgacgctgtcgcgctgctacccgctggaggaggtcggccaggcggtctacgacgtccatcacaacctgcaccagggcaaggtcggcgtgctcgcgctcgcgccgcgcgaggggctcggggtccggaacccggagctgcgggaatgccatcttgccgcgatcaaccgcttccgggtgccggcctga

SEQ ID NO: 28 - protein, 636 aa, micromonospora carbonacea subspecies aurantiaca
VHQAHRDGVDQATLDRVMIAKRLALELREVIGRRCQRQAELAALVDTARDLAGATNLEAGLQLVVRRTQLLLAGDVAFVSLVDDATGESYVASAVGAATALTSGYRLPWRDGLVVAAAPREPLSWTADHLADERLERHPAADGLVRAEGLHAVLSVVLSVEGRHLGNLHVGHRQVRHFAPDEVASLRLLADLAATAVERIMLLDDTWAELKQAQQEAARARAELNAVRMADRLQPELVQLILDGGELDDLVGSAVRRLGGALHVRDRANGVLAAAGEIPVPNERELARVRLNAHATGRPGRLTTGSWVVPLAARAGDLGCVLFHADEPSDDERMAALPAVAQTVALLMTRNGGSHGQPGDGLLEDLLGPWPDLERGGKRRRYTPVEFDRPYVVVVARPEGATSPRVFERAVSVAHGLNGMKAIRDGQAVLLLPGDDPGARARDVTRELSGLLGLPVTAGGAGPVRTADSVSRTYQEAARCVDALAALDAKGRAACSRDLGFLGLLVAGGHDVTGFVDRVIGPVLSYDARRLTNLRETLQTYFDSAGSRTRAAEMLHLHPNTVSRRLDRISQLLGRDWRQPDRALDTQLALRLHRIRGLLCQERGYPGPSQEPDQPARPIRRHRPPASAGRAPRTPR

SEQ ID NO: 29 - DNA, 1911 bp, micromonospora carbonacea subspecies aurantiaca
gtgcaccaggcgcaccgggacggagtggaccaggccacgctcgaccgggtgatgatcgccaagcgactcgcgttggagcttcgagaggtcatcgggaggcggtgtcagcggcaggcggagctggccgccctcgtcgacaccgcccgtgacctcgccggggcgacgaacctggaggccgggctgcagctggtggtgcggcggacccaactgctgctcgccggggacgtggcgttcgtcagcctcgtcgacgacgcgaccggcgaatcctacgtcgcctcggccgtcggggcggccaccgcgctgaccagcggctaccggctgccctggcgcgacgggctggtcgtggccgccgcaccgcgcgagccactctcctggacggcggaccacctcgccgacgagcgcctcgaacgacacccggccgccgacggcctggtccgcgcggaagggctgcacgcggtgctgtccgtggttctgagcgtcgagggccggcacctcggcaacctgcacgtcggccaccggcaggtccgccacttcgccccggacgaggtcgcgtcgctgcgcctgctcgccgatctcgcggcgacggcagtggagcggatcatgctgctcgacgacacgtgggccgaactcaagcaggcccagcaggaggcggccagggcccgagccgagctgaacgcggtccgcatggccgaccgcctgcaacccgaactcgtccagctcatcctcgacggcggcgaactcgacgacctggtgggcagcgccgtgcggcgactgggcggcgccctgcacgtgcgtgaccgggccaacggcgtgctggcggcggccggtgaaatccctgtcccgaacgagcgggaactggcccgagtgcggctgaacgcccacgccaccggccgacccggccgcctgaccaccggttcctgggtggtgcccctggcggcccgcgccggtgacctcggctgtgtgttgttccacgccgacgagccgtccgacgacgagcggatggcggccctgccggcggtcgcgcagaccgtggcgctgctgatgaccaggaacggcgggagccacggccagccgggcgacgggctcctggaggacctgctcggcccgtggccggacctggagcggggcgggaagcgccgtcggtacacacctgtcgagttcgaccggccctacgtcgtcgtggtcgcccgccccgagggcgccacctcgccccgggtgttcgaacgggcggtctccgtcgcccacggcctgaacggcatgaaggccatccgggacggccaggcggtgctgctgctgcccggtgacgacccgggggcccgggcccgggacgtgacgcgggaactgagcgggctgctcggcctaccggtcacggccggaggcgccggaccggtgcgcacggcggactcggtcagccgcacctaccaggaggcggcccggtgcgtcgacgccctggccgcgctggacgcgaaggggcgggcggcctgctcacgggacctgggcttcctcgggctgctggtcgccggcggccacgacgtcaccggtttcgtcgaccgggtcatcggacccgtgctgagctacgacgcgcgccggctcacgaatctcagggagaccctccagacctacttcgactcggcgggcagccgtacccgggcggcggagatgctgcatctgcatccgaacaccgtgtcccgccggctggaccgcatctcccagctgctcggccgggactggcggcagccggaccgggccctcgacacgcagctcgctctgcgcctgcaccggatccgtggcctgctctgccaggaacggggctacccgggcccatcgcaggagccggaccaacccgcgcggcctatccggcggcaccgccctccagcatccgcagggcgtgcgccacggacgccaaggtga

SEQ ID NO: 30 - protein, 403 aa, micromonospora carbonacea subspecies aurantiaca
MVPTLDVREEVTAARSDPDTVSRFCAALLASLPRADQRRKGELYVRGLLTASGRKTMRNLAAIADDPAAAQSMHHFISCSTWDWETVRAALAGHLDRTLSPRAWVVRSMLVPKTGRHSVGVERRYVPALGETVNSQQSYGLWLASETVAAPINWQLSIGKGWLQDNRARASVPADEDGTTSDGAAVQAVLKAAAWGIGPRPVVMDARHSALPPLIEAFTTAGLPFLLRINSGCTLLAAGPGPRENRVAAASAEHLLSLTRAQRRPVEWIDPASPGARRTSLVAPLQVYWPGLSGARPPGPSAPAPPGAARAAAPGLPLTLLGKWQTYERGVRQMWLTNMTDAGYGPLLRLSKLTRRVETDFSQVSLDVGIQDFEGRSYQGWHRHVTLASVAHALRMLEGGAAG

SEQ ID NO: 31 - DNA, 1212 bp, micromonospora carbonacea subspecies aurantiaca
atggtgccgacgctcgacgtccgcgaggaggtgaccgcggcaaggtccgatccggacaccgtgtcccggttctgcgccgccctgctggcctcgctgccccgcgccgaccagcgacgcaagggcgaactgtacgtccgggggctgctgaccgcctccggccgcaagaccatgcgcaacctggccgccatcgccgacgatccggcggcggcacagagcatgcaccacttcatcagttgctccacctgggactgggagaccgtccgtgccgcgctcgccggccacctggaccggacgctgtcgccccgggcctgggtggtgcggtcgatgctggtgccgaagaccggccggcactcggtcggcgtggaacgccggtacgtgcccgcgctgggcgagacggtcaacagccagcagagctacggcctctggctggcctcggagaccgtcgccgcgcccatcaactggcagttgtccatcggtaagggttggctccaggacaaccgcgcccgcgcgagcgtaccggcggacgaggacggcacgaccagcgacggcgcggcggtgcaggcggtgctgaaggccgcggcctggggaatcggccctcgcccggtggtaatggacgcccggcactcggcgctgcccccgctgatcgaggcgttcaccacggcgggtctgcccttcctgctacggatcaacagcggctgcaccctgctggccgccgggcccggcccgcgcgagaaccgggtcgcggcggcctccgccgagcacctgctcagcctgacgcgggcccagcgccgtccggtggagtggatcgacccggccagccccggcgcacggcgcacgagcctggtcgcaccgctacaggtctattggccgggcctgtccggtgcccgcccgcccggtccgtccgccccggccccgccgggggcggcgcgcgccgccgcgcccgggctgcccctgacactgctcggcaagtggcagacctacgagcgcggcgtacggcagatgtggctgaccaacatgaccgacgccgggtacggcccactgctgcggctgagcaagctcacccggcgggtcgagaccgacttctcccaggtcagcctcgacgtcggcatccaagacttcgagggtcggtcataccaaggctggcaccggcacgtcaccttggcgtccgtggcgcacgccctgcggatgctggagggcggtgccgccggatag

SEQ ID NO: 32 - protein, 481 aa, micromonospora carbonacea subspecies aurantiaca
MTSAAHHSPHPAKADALMDDAHADIGADAEADGRRLDRAALRRVAGLSTERADVTEVEYRQVRLERVVLVGVWTSGTADEAERSLAELAALAETAGAVVLDGVIQRRDRPDPATYIGSGKARELRDIVQEVGADTVICDGELSPAQLVRLEEVVDAKVVDRTALILDIFAQHATSREGKAQVALAQMQYMLPRLRGWGQSLSRQMGGGAGGGGMATRGPGETKIETDRRRIHERMARLRREIAEMKSGRELKRRDRRRNSVPSVAIAGYTNAGKSSLLNRLTGASVLVQNALFATLDPTVRRATTPSGRSYTITDTVGFVRHLPHHLVEAFRSTLEEVAEADLLLHVVDGAHPAPLEQLASVRAVIRDVDAAGVPELVVINKADAATPAALAALAEAEPHHVVVSARTGQGIDTLRQLLEAALPHREVRVDVLIPYVAGSLVARVHADGEVLAEEHTADGTLLQARVAPDLAAELSAYART

SEQ ID NO: 33 - DNA, 1446 bp, micromonospora carbonacea subspecies aurantiaca
atgacctccgcagcgcaccattccccgcatccggcgaaggccgacgccctgatggacgacgcccacgccgacatcggggccgatgccgaggccgacggtcgacggctcgaccgggccgccctgcggcgggtcgccgggctgtcgaccgagagggccgacgtcacggaggtcgagtaccggcaggtgcggctggagcgcgtcgtcctggtcggcgtgtggacctcgggcaccgccgacgaggccgaacggtccctcgccgagctggcggcactcgccgagaccgcgggagccgtggtgctcgacggggtgatccagcgccgcgaccggcccgacccggcgacgtacatcggctccggcaaggcgcgggagttgcgggacatcgtccaggaggtgggggccgacacggtgatctgcgacggtgagctgagcccggcccaactggtacgcctcgaagaggtcgtcgacgccaaggtggtggaccgcaccgcgctgatcctcgacatcttcgcccagcacgccacgtcccgcgaggggaaggcgcaggtggccctggcacagatgcaatacatgctgccgcggctgcgcggctggggccagtcgctctcccggcagatgggcggaggtgccggcggcggtggcatggccacccgggggcccggcgagaccaagatcgagaccgaccggcggcgcatccacgagaggatggcccggctccgacgggagatcgcggagatgaagtccggccgcgaactcaagcgccgcgatcggcggcgcaacagcgtcccgtcggtcgcgatcgccggttacaccaacgccggcaagtcctcgctgctcaaccggctcactggcgcgagcgtgctggtgcagaacgcgctgttcgccaccctcgacccgacggtgcgccgggccaccaccccgagcgggcgcagctacacgatcaccgacaccgtcggattcgtccggcacctgccgcaccacctggtggaggcgttccgctccaccctggaagaggtggccgaggccgacctcctgctgcacgtggtggacggcgcccaccccgccccgctggagcagctcgcctcggtgcgcgcggtcatccgggacgtggacgcggcgggagtgcccgaactcgtcgtgatcaacaaggccgacgccgccaccccggccgccctggccgcgttggcggaggccgagccgcaccacgtcgtcgtctcggcccgcaccggtcagggcatcgacacgcttcggcagttgctggaggccgcgctgccgcaccgggaggtccgggtcgacgtcctgatcccgtacgtcgcgggcagcctcgtggcccgggtgcacgccgacggcgaggtgctggccgaggagcacacggccgacggcaccctgctgcaggcgcgggtggcccccgacctggctgccgagctcagcgcgtacgccaggacctga

SEQ ID NO: 34 - protein, 408 aa, micromonospora carbonacea subspecies aurantiaca
MKRDLGDLALFGGHASFLQQIHVGRPNRIDRARLFDRLSWALDNEWLTNNGPLAREFEERVADMVGVGNCVATCNATVALQLLAHATELTGEVIMPSLTFAATAHAVRWLGLEPVFCDIDPRTGCLDHVAVAAAITPRTSAVFGVHLWGRPCDVNALEKVTADAGLRLFFDAAHAIGCTSQGRPVGRFGHAEVFSFHATKVVNAFEGGAIVTDDDDLAHRVRSLANFGFGLHSPSAAGGTNAKMSEASAAMGLTSLDAFPEVARHNQANYEQYCGELARIPGLSVIDFAPDERHNYQYVIVEIDPDVTGLHRDLLVDLLRAENVVAQRYFSPACHQLEPYRSRQQFQLPHTERLSARVLALPTGSAISREDIRRVCNIVRLAVSRGFELTARWQQQPGPDGQSVVAPG

SEQ ID NO: 35 - DNA, 1227 bp, micromonospora carbonacea subspecies aurantiaca
atgaagcgagatctcggggatctggcactcttcggaggacacgccagcttcctccagcagatccacgtcgggcgccccaaccggatcgatcgggccaggctgttcgaccggctgtcctgggcgctcgacaacgagtggttgaccaacaacgggccgctggcacgggagttcgaggagcgggtcgccgacatggtcggggtcggcaactgcgtggcgacgtgcaacgccacggtggccctccagctgctcgcgcacgccaccgagctgaccggtgaggtgatcatgccatcgctcaccttcgccgcgaccgcacacgcggtgcgctggctcgggctggagccggtcttctgcgacatcgacccgcgcaccggatgcctcgaccacgtggcggtcgccgcggccatcacgccgcgcacgtcggcggtcttcggcgtccacctctggggccgcccctgcgacgtcaacgcgctggagaaggtgaccgccgacgcgggcctgcgcctgttcttcgacgccgcccacgccatcgggtgcacctcacagggccgcccggtggggcggttcggccacgccgaggtgttcagcttccacgcgacgaaggtcgtcaacgccttcgagggcggggcgatcgtcaccgacgacgacgacctcgcccaccgcgtccgctccctggcgaacttcggcttcggcctgcacagccccagcgcggccggcggcaccaacgcgaagatgagcgaggcgtccgccgccatggggctcacctcgctcgacgcgttccccgaggtggcccgccacaaccaggccaactacgagcagtactgcggtgagctggcccggattcccggcctcagcgtgatcgacttcgcccccgacgagcggcacaactaccagtacgtgatcgtcgagatcgacccggacgtcaccgggttgcaccgcgacctgctcgtcgacctgctccgggccgagaacgtcgtggcgcagcgctacttctcgccggcctgtcaccaattggagccctaccggtcccggcagcagttccagctgccgcacaccgagcggctctcggcgcgcgtcctggcgctgccgaccggctccgccatctcccgggaagacatccgcagggtgtgcaacatcgtgcggttggcggtctcccggggattcgaattgaccgctcggtggcagcagcagcccgggcccgacggacagagcgtggtggcacccggttga

SEQ ID NO: 36 - protein, 488 aa, micromonospora carbonacea subspecies aurantiaca
VGGPVTMEISASNPVATCAVPGSDPTAAARVLYDEVAGSGIVPPAEIGAAAQGLVALARIYGTTPFLPLEQARREIGLDRAGFGRLLDLFARIPGLRTAVENGPSGRYWTNTVLGLERAGVFDAVLDRRPAFPHLVGLYPGPTCMFRCHFCVRVTGARYQASALDDGNAMFASVIDEVPAHNRDAVYVSGGLEPLTNPGLGALVSRAAERGFRIILYTNSFALTEQKLKGERGLWSLHAIRTSLYGLNDEEYRATTGKQGAFTRVRANLTRFQQLRAERGEPVRLGLSYIVLPGRAGRLSALIDFVAELNEAAPDRPLDYINLREDYSGRPDGKLSLDERAELQAELHRFRERAMQRTPTLHIDYGYALHSLMTGSDVELVRIRPETMRPAAHPQVSVQVDILGDVYLYREAAFPGLAGADRYRIGTVSPGTTLAQVVETFVTSGGSVVAKPGDEYFLDGFDQAVTARLNQMETDVADGWGDRRGFLR

SEQ ID NO: 37 - DNA, 1467 bp, micromonospora carbonacea subspecies aurantiaca
gtgggagggcccgtgaccatggagatctccgcctcgaatcccgtggcgacctgcgctgtccccggcagcgacccgaccgcggcggcgcgcgtgctgtacgacgaggtcgccgggtcaggaatcgtgccgccggcagagatcggggccgccgcccaggggttggtggcattggcacgcatctacgggaccacaccttttctgccgcttgagcaggcccgccgcgaaatcggcctggaccgggccgggttcgggcggctgctggacctgttcgcccggattcccgggttgcgcaccgcagtggagaacggaccgtccggtcgctactggaccaacacggtgctcggcctcgaaagggccggcgtcttcgacgccgtgctcgaccggaggccggcgtttccgcatctcgtcgggctctacccgggccccacgtgcatgttccgctgtcacttctgcgtaagggtcaccggggcccgctaccaggcctcggcgctggacgacgggaacgccatgttcgcctctgtcatcgacgaggtccccgcgcacaaccgcgacgcggtgtacgtctccggtggcctcgagccactcaccaaccccgggctcggtgcactggtcagccgggcggccgagcggggatttcggatcatcctctacaccaactcgttcgccctcacggagcagaagctcaagggtgagcggggattgtggagcctgcacgccatccgcacgtcgctgtacgggttgaacgacgaggaataccgggcgaccaccggcaagcagggggccttcacccgggtacgggcgaacctcacgcggttccagcagctgcgtgccgagcggggcgagccggtgcggctcggcctcagctacatcgtcctgcccggccgcgccgggcggctgagcgcgctgatcgacttcgtcgccgagctcaacgaggcggcaccggaccgcccgctggactacatcaacctgcgggaggactacagcgggcggccggacgggaagctctccctggacgagcgcgccgagctccaggccgagctgcaccggttccgggagagggcaatgcagcggacgccgaccctgcacatcgactacggctacgccctgcacagcctgatgacgggaagcgacgtggagctcgtgcgtatccggccggagacgatgcgccctgcggcccacccgcaggtgtcggtgcaggtggatatcctcggtgatgtctacctctatcgggaggcggcgtttccgggcctggccggtgccgaccgctatcgcatcggcacggtatctcccggcacgacgttggcgcaggtggtggagacgttcgtgaccagcggcggatcggtggtcgcgaagcctggcgacgaatacttcctggacggattcgaccaggcggtgaccgcgcggctgaaccagatggagaccgacgtcgccgatggctggggagaccgacggggtttcctccgctga

SEQ ID NO: 38 - protein, 277 aa, micromonospora carbonacea subspecies aurantiaca
MPYIQHAGRHEFGQNFLVDRSVIDDFVELVARTDGPIVEIGAGDGALTLPLSRQGRELTAVEIDSKRSKRLSRQTPDNVTVVCADVLSFRFPQHPHVVVGNIPFHVTTPIVRALLAADHWHTAVLLVQWEVARRRAGVGGATLLTASWWPWYDFELHSRVPARAFRPVPSVDGGLFSMVRRGTPLVDDRRGYQEFVRLVFTGKGHGLPEILQRTGRIARKDQQDWQRANRVGPQHLPKDLTAHQWASLWHLVAPARPAGPRRPAPRRPGSPASARRR

SEQ ID NO: 39 - DNA, 834 bp, micromonospora carbonacea subspecies aurantiaca
atgccctacatccagcacgccgggcgacatgaattcggccagaatttcctggtcgaccgctcggtgatcgacgatttcgtcgaactcgtcgcccggaccgacggccctatcgtggagatcggcgccggcgacggtgcgctgaccctacccctgagccggcagggaagggagttgaccgcagtggagatcgactccaagcgttccaagcggctcagccggcagacacccgacaacgtcaccgtggtctgcgcggatgtcctgagcttccggttcccccagcatccgcacgtggtcgtcgggaacatccccttccacgtgaccacccccatcgtgcgggctctcctcgccgcggaccactggcacacggcggtgctgctggtgcagtgggaggtggcccgcaggcgggccggcgtcggcggcgcgacgctgctgaccgcgagctggtggccctggtacgacttcgaactgcactcccgggttccggcccgcgccttccggcctgtcccttccgtcgacggcgggctgttctccatggtccgtcgcgggaccccgctggtcgacgaccggaggggttaccaggaattcgtccggctggtgttcaccggcaaggggcacggattgccggagatccttcagcggaccgggcggatcgcccgcaaggaccagcaggactggcaacgggccaaccgggtggggccgcagcacctgcccaaggacctgaccgcccaccagtgggcctccctgtggcacctggtggcacccgcccggccggccggcccccgccgtccggcaccgcgccggccaggaagccccgcttcggcgcgccggcgctga

(19) United States
(12) Patent Application Publication
(10) Pub. No.: US 2003/0113874 A1
(43) Pub. Date: Jun. 19, 2003

(54) Genes and proteins for the biosynthesis of rosaramicin

(76) Inventors: Chris M. Farnet, Outremont (CA); Alfredo Staffa, Saint-Laurent (CA); Xianshu Yang, Belmont, MA (US)

Correspondence Address: Ywe J. Looper, Ecopia Biosciences Inc., 7290 Frederick-Banting, Saint-Laurent, QC, H4S 2A1, CA

(21) Appl. No.: 10/205,032

(22) Filed: Jul. 26, 2002

Related U.S. Application Data
(60) Provisional application No. 60/307,629, filed on Jul. 26, 2001.

(51) Int. Cl. C12Q 1/68; C07H 21/04; C12P 19/60; C12N 1/20; C12N 9/10; C12N 15/74

(52) U.S. Cl. 435/75; 435/69.1; 435/193; 435/320.1; 435/252.3; 536/23.2; 435/6

(57) ABSTRACT

Genes and proteins involved in the biosynthesis of macrolides by microorganisms, in particular the nucleic acids forming the biosynthetic locus for the 16-member macrolide rosaramicin from Micromonospora carbonacea. These nucleic acids can be used to make expression constructs and transformed host cells for the production of rosaramicin. The genes and proteins allow direct manipulation of macrolides and related chemical structures via chemical engineering of the proteins involved in the biosynthesis of rosaramicin.

### CROSS-REFERENCING TO RELATED APPLICATION:

This application claims benefit under 35 USC §119 of provisional application U.S. Ser. No. 60/307,629 filed on Jul. 26, 2001 which is hereby incorporated by reference in its entirety for all purposes.

### FIELD OF INVENTION

The present invention relates to nucleic acid molecules that encode proteins that direct the synthesis of macrolides, in particular the 16-member macrolide rosaramicin. The present invention also is directed to the use of nucleic acids and proteins to produce compounds exhibiting antibiotic activity based on the rosaramicin structure.

### BACKGROUND

Rosaramicin is a 16-member macrolide antibiotic. Macrolides consitute a group of antibiotics mainly active against Gram-positive bacteria. They have clinical applications in the treatment of bacterial infections. Macrolides compounds are structurally characterized by a macrolide lactone ring to which one or several deoxy-sugars moieties are attached.

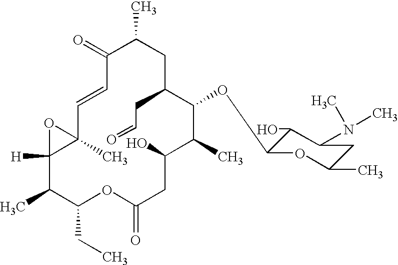

The carbohydrate ligands and macrolide lactone ring serve as molecular recognition elements critical for biological activity. Variations in the sugar composition of a macrolide or in the structure of the macrolide lactone ring may vary the biological activity of the molecule. Elucidation of gene clusters involved in the biosynthesis of rosaramicin expands the repertoire of genes and proteins useful to macrolides via combinatorial biosynthesis.

The increasing number of microbial strains that have acquired resistance to the currently available antibiotic compounds is recognized as a dangerous threat to public health. The genes and proteins involved in the biosynthesis of rosaramicin may be used to generate new unnatural compounds having desirable biological activity. The genes and proteins from the rosaramicin locus may also be used as probes to identify new rosaramicin-like natural products.

The genome of many microorganisms contains multiple natural product biosynthetic loci that are not normally expressed in nature or under conventional experimental conditions. For example, twenty-five secondary metabolic gene clusters in the genome of the actinomycete Streptomyces avermitilis were identified by whole genome shotgun sequencing of the genome despite the fact that the organism was known to produce only two antimicrobial natural products (Osura et al. PNAS, vol. 98, no. 21 12215-12220). An important new source of antimicrobial compounds lies in the products of cryptic biosynthetic loci. It is desirable to discover and characterize a biosynthetic locus producing an antimicrobial product and present in the genome of organisms not known to product the antimicrobial product of the locus.

### SUMMARY OF THE INVENTION

Micromonospora carbonacea is known to produce the antimicrobial orthosomycin natural product everninomicin. Micromonospora carbonacea was not previously reported to produce other natural products. We have surprisingly discovered, in the Micromonospora carbonacea genome, a type I polyketide biosynthetic gene cluster directed to the production of a rosaramicin-type polyketide.

The invention provides polynucleotides and polypeptides useful in the production and engineering of macrolides. In one embodiment, the polynucleotide molecules are selected from the contiguous DNA sequence SEQ ID NO: 1. Other embodiments of the polynucleotides and polypeptides are provided in the accompanying sequence listing. SEQ ID NOS: 3, 5, 7, 9, 11, 13, 15, 17, 19, 21, 23, 25, 27, 29, 31, 33, 35, 37, 39 provide nucleic acids responsible for biosynthesis of the 16-member macrolide rosaramicin. SEQ ID NOS: 2, 4, 6, 8, 10, 12, 14, 16, 18, 20, 22, 24, 26, 28, 30, 32, 34, 36, 38 provide amino acid sequences for proteins responsible for biosynthesis of the 16-member macrolide rosaramicin. Certain embodiments of the invention specifically exclude one or more of open reading frames of the rosaramicin biosynthetic locus, most notably any one or more of ORFs 3, 11, 13, 16, 17 and 18 (SEQ ID NOS: 7, 23, 27, 33, 35 and 37) and the corresponding gene products (SEQ ID NOS: 6, 22, 26, 32, 34 and 36) deduced therefrom, although other ORFs and polypeptides listed in the sequence listing can be excluded from certain embodiments without departing from the scope of the invention.

The polynucleotides and polypeptides of the invention provide the machinery for producing novel compounds based on the structure of rosaramicin. The invention allows direct manipulation of rosaramicin and related chemical structures via chemical engineering of the enzymes involved in the biosynthesis of rosaramicin, modifications which may not be presently possible by chemical methodology because of the complexity of the structures. The invention can also be used to introduce “chemical handles” into normally inert positions that permit subsequence chemical modifications. Several general approaches to achieve the development of novel macrolides are facilitated by the methods and compositions of the present invention. For example, tylosin is structurally related to rosaramicin but, unlike rosaramicin, it does not contain an epoxide. Accordingly, genes and proteins disclosed herein may be used to enzymatically create a tylosin derivative that contains an epoxide modification.

Various macrolide structures can be generated by genetic manipulation of the rosaramicin gene cluster or use of various genes from the rosaramicin gene cluster in accordance with the methods of the invention. The invention can be used to generate a focused library of analogs around a macrolide lead candidate to fine-tune the compound for optimal properties. Genetic engineering methods of the invention can be directed to modify positions of the molecule previously inert to chemical modifications. Known techniques allow one to manipulate a known macrolide gene cluster either to produce the macrolide compound synthesized by that gene cluster at higher levels than occur in nature or in hosts that otherwise do not produce the macrolide. Known techniques allow one to produce molecules that are structurally related to, but distinct from, the macrolide compounds produced from known macrolide gene clusters. Cloning, analysis, and manipulation by recombinant DNA technology of genes that encode rosaramicin gene products can be performed according to known techniques.

Thus, in a first aspect the invention provides an isolated, purified or enriched nucleic acid comprising a sequence selected from the group consisting of SEQ ID NO: 1; the sequences complementary to SEQ ID NO: 1; fragments comprising at least 100, 200, 300, 500, 1000, 2000 or more consecutive nucleotides of SEQ ID NO: 1; and fragments comprising at least 100, 200, 300, 500, 1000, 2000 or more consecutive nucleotides of the sequences complementary to SEQ ID NO: 1. Preferred embodiments of this aspect include isolated, purified or enriched nucleic acids capable of hybridizing to the above sequences under conditions of moderate or high stringency; isolated, purified or enriched nucleic acid comprising at least 100, 200, 300, 500, 1000, 2000 or more consecutive bases of the above sequences; and isolated, purified or enriched nucleic acid having at least 70%, 75%, 80%, 85%, 90%, 95%, 97% or 99% homology to the above sequences as determined by analysis with BLASTN version 2.0 with the default parameters.

Further embodiments of this aspect of the invention include an isolated, purified or enriched nucleic acid comprising a sequence selected from the group consisting of SEQ ID NOS: 3, 5, 7, 9, 11, 13, 15, 17, 19, 21, 23, 25, 27, 29, 31, 33, 35, 37, 39 and the sequences complementary thereto; an isolated, purified or enriched nucleic acid comprising at least 50, 75, 100, 200, 500, 800 or more consecutive bases of a sequence selected from the group consisting of SEQ ID NOS: 3, 5, 7, 9, 11, 13, 15, 17, 19, 21, 23, 25, 27, 29, 31, 33, 35, 37, 39 and the sequences complementary thereto; and an isolated, purified or enriched nucleic acid capable of hybridizing to the above listed nucleic acids under conditions of moderate or high stringency, and isolated, purified or enriched nucleic acid having at least 70%, 75%, 80%, 85%, 90%, 95%, 97% or 99% homology to the nucleic acid of SEQ ID NOS: 3, 5, 7, 9, 11, 13, 15, 17, 19, 21, 23, 25, 27, 29, 31, 33, 35, 37, 39 as determined by analysis with BLASTN version 2.0 with the default parameters.

In a second embodiment, the invention provides an isolated or purified polypeptide comprising a sequence selected from the group consisting of SEQ ID NOS: 2, 4, 6, 8,10, 12, 14, 16, 18, 20, 22, 24, 26, 28, 30, 32, 34, 36, 38; an isolated or purified polypeptide comprising at least 50, 75, 100, 200, 300 or more consecutive amino acids of the polypeptides of SEQ ID NOS: 2, 4, 6, 8, 10, 12, 14, 16, 18, 20, 22, 24, 26, 28, 30, 32, 34, 36, 38; and an isolated or purified polypeptide having at least 70%, 75%, 80%, 85%, 90%, 95%, 97%, or 99% homology to the polypeptide of SEQ ID NOS: 2, 4, 6, 8, 10, 12, 14, 16, 18, 20, 22, 24, 26, 28, 30, 32, 34, 36, 38 as determined by analysis with BLASTP version 2.2.2 with the default parameters. In a further aspect, the invention provides a polypeptide comprising one or two or three or five or more or the above polypeptide sequences.

The invention also provides recombinant DNA expression vectors containing the above nucleic acids. The polynucleotides and the methods of the invention enable one skilled in the art to create recombinant host cells with the ability to produce macrolides. Thus, the invention provides a method of preparing a macrolide compound, said method comprising transforming a heterologous host cell with a recombinant DNA vector that encodes at least one of the above nucleic acids, and culturing said host cell under conditions such that a macrolide is produced. In one aspect, the method is practiced with a Streptomyces host cell. In another aspect, the macrolide produced is rosoramicin. In another aspect, the macrolide produced is a compound related in structure to rosaramicin. The invention also provides a method for producing a rosaramicin compound by culturing Micromonospora carbonacea under conditions allowing for expression of its endogenous rosaramicin biosynthetic locus.

The invention also encompasses a method of invention for detecting by, in silico hybridization or traditional hybridization, putative macrolide gene clusters or macrolide-producing microorganisms using compositions of the invention. In one embodiment, a polypeptide encoding one or more of the polyketide synthase proteins (SEQ ID NOS: 10, 12, 14, 16 and 18) or fragments thereof are used as probes to detect putative macrolide gene clusters by in silico hybridization.

### BRIEF DESCRIPTION OF THE DRAWINGS

The present invention will be further understood from the following description with reference to the following figures:

FIG. 1 is a block diagram of a computer system which implements and executes software tools for the purpose of comparing a query to a subject, wherein the subject is selected from the reference sequences of the invention.

FIGS. 2A, 2B, 2C and 2D are flow diagrams of a sequence comparison software that can be employed for the purpose of comparing a query to a subject, wherein the subject is selected from the reference sequences of the invention, wherein FIG. 2A is the query initialization subprocess of the sequence comparison software, FIG. 2B is the subject datasource initialization subprocess of the sequence comparison software, FIG. 2C illustrates the comparison subprocess and the analysis subprocess of the sequence comparison software, and FIG. 2D is the Display/Report subprocess of the sequence comparison software.

FIG. 3 is a flow diagram of the comparator algorithm (238) of FIG. 2C which is one embodiment of a comparator algorithm that can be used for pairwise determination of similarity between a query/subject pair.

FIG. 4 is a flow diagram of the analyzer algorithm (244) of FIG. 2C which is one embodiment of an analyzer algorithm that can be used to assign identity to a query sequence, based on similarity to a subject sequence, where the subject sequence is a reference sequence of the invention.

FIG. 5 is a graphical depiction of the rosaramicin biosynthetic locus showing, at the top of the figure, the regions covered by the three deposited cosmid clones 010CK, 010CF and 010CJ; a scale in kilobase pairs; the positioning of the open reading frames on a continuous black line representing the continuous DNA sequence (SEQ ID NO: 1); and the relative position and orientation of 19 ORFs referred to by number at the bottom of figure.

FIG. 6 illustrates the construction of the rosaramicin backbone by the Type 1 polyketide synthase enzymes (PKS) in the rosaramicin biosynthetic locus.

FIG. 7 illustrates a mechanism for the biosynthesis of rosaramicin.

FIGS. 8A and 8B represent a Clustal amino acid alignment of the eight ketosynthase (KS) domains found in the rosaramicin PKS enzyme complex. Key residues are highlighted.

FIGS. 9A and 9B represent a Clustal amino acid alignment of the eight acyl transferase (AT) domains in the rosaramicin PKS enzyme complex. Key residues are highlighted. Regions important in substrate recognition are indicated by “s” above the alignment.

FIG. 10 represents a Clustal amino acid alignment of the 3 DH domains in the rosaramicin PKS enzyme complex. Key residues are highlighted.

FIG. 11 represents a Clustal amino acid alignment comparing the single enoyl reductase (ER) domain in the rosaramicin PKS enzyme complex to a prototypical ER domain of the erythromycin PKS, i.e. 6-deoxyerythronolide B synthase (DEBS), key residues are highlighted.

FIG. 12 represents a Clustal amino acid alignment of the 7 KR domains in the rosaramicin PKS enzyme complex. Key residues are highlighted.

FIG. 13 represents a Clustal amino acid alignment of the 8 ACP domains in the rosaramicin PKS enzyme complex. The key active site serine residue is highlighted.

FIG. 14 represents a Clustal amino acid alignment comparing the single thioesterase (Te) domain in the rosaramicin PKS enzyme complex to a prototypical Te domain of the erythromycin PKS, DEBS.

FIG. 15 represents a Clustal amino acid alignment that demonstrates the overall high degree of homology between the second AT domain of ORF7 with two other ethylmalonyl-CoA-specific AT domains from the tylosin and niddamycin PKS complexes.

FIG. 16 is a LCMS graph showing the production of a compound of the molecular weight of rosaramicin.

### DETAILED DESCRIPTION OF THE INVENTION:

Throughout the description and the figures, the biosynthetic locus for rosaramicin from Micromonospora carbonacea is sometimes referred to as ROSA. The ORFs in ROSA are assigned a putative function sometimes referred to throughout the description and figures by reference to a four-letter designation, as indicated in Table I.

TABLE 1

| Families | ORF # | Function |
|---|---|---|
| ABCC | 1 | ABC transporter; contains repeated domain |
| DATF | 17 | dehydratase/aminotransferase; SMAT family |
| | | (secondary metabolism aminotransferase); |
| | | transaminase |
| GTFA | 11 | glycosyl transferase |
| MTFA | 12 | methyltransferase, SAM-dependent; N,N-dimethyl- |
| | | transferases |
| MTRA | 19 | resistance methyltransferase; 23S ribosomal |
| NBPA | 16 | unknown, nucleotide (ATP/GTP) binding protein; |
| | | may be involved in regulated proteolysis |
| OXRB | 10 | oxidoreductase; similar to NDP-hexose-3,4- |
| | | isomerases (tautomerase) |
| OXRC | 3, 4 | oxidoreductase; cytP450 monooxygenase, |
| | | hydroxylase; oxygen-binding site motif: |
| | | LLxAGx (D,E); heme-binding pocket motif: |
| | | GxGxHxCxGxxLxR, the cysteine is invariable and |
| | | coordinates the heme |
| OXRH | 13 | oxidoreductase, NAD(P)-dependent; similar to |
| | | crotonyl CoA reductases (CCR); similarity to some |
| | | quinone oxidoreductases, zinc-containing alcohol |
| | | dehydrogenases |
| PKSH | 5-9 | polyketide synthase, type I |
| REGM | 15 | regulator; similar to TyIR global activator of the |
| | | tylosin locus and the carbomycin AcyB2 positive |
| | | regulator |
| REGS | 14 | regulator, may be positive regulator; similar to |
| | | spiramycin SrmR, which specifically activates the |
| | | production of spiramycin |
| SURA | 18 | sugar reductase; iron-sulfur (4Fe-4S) protein; may |
| | | be involved in 1,2-migration of the amino group |
| | | from C4 to C3 via the Schiff's base intermediate |
| TESA | 2 | thioesterase |

The terms “macrolide producer” and “macrolide-producing organism” refer to a microorganism that carries the genetic information necessary to produce a macrolide compound, whether or not the organism is known to produce a macrolide compound. The terms “rosaramicin producer” and “rosaramicin-producing organism” refer to a microorganism that carries the genetic information necessary to produce a rosaramicin compound, whether or not the organism is known to produce a rosaramicin product. The terms apply equally to organisms in which the genetic information to produce the macrolide or rosaramicin compound is found in the organism as it exists in its natural environment, and to organisms in which the genetic information is introduced by recombinant techniques. For the sake of particularity, specific organisms contemplated herein include organisms of the family Micromonosporaceae, of which preferred genera include Micromonospora, Actinoplanes and Dactylosporangium; the family Streptomycetaceae, of which preferred genera include Streptomyces and Kitasatospora; the family Pseudonocardiaceae, of which preferred genera are Amycolatopsis and Saccharopolyspora; and the family Actinosynnemataceae, of which preferred genera include Saccharothrix and Actinosynnema; however the terms are intended to encompass all organisms containing genetic information necessary to produce a macrolide compound.

The term rosaramicin biosynthetic gene product refers to any enzyme or polypeptide involved in the biosynthesis of rosaramicin. The term “rosaramicin” is intended to encompass the compounds sometimes referred to as 4′-deoxycirramycin A1, rosamicin, izenamicin A1, juvenimicin A3, 6108A3, M 4365A2, Sch 14947, antibiotic 6108A3, antibiotic M 4365A2 and antibiotic Sch 14947. For the sake of particularity, the rosaramicin biosynthetic pathway is associated with Micromonospora carbonacea. However, it should be understood that this term encompasses rosaramicin biosynthetic enzymes (and genes encoding such enzymes) isolated from any microorganism of the genus Micromonospora or Streptomyces, and furthermore that these genes may have novel homologues in related actinomycete microorganisms or non-actinomycete microorganisms that fall within the scope of the invention. Representative rosaramicin biosynthetic gene products include the polypeptides listed in SEQ ID NOS: 2, 4, 6, 8, 10, 12, 14, 16, 18, 20, 22, 24, 26, 28, 30, 32, 34, 36, 38 or homologues thereof.

The term “isolated” means that the material is removed from its original environment, e.g. the natural environment if it is naturally-occurring. For example, a naturally-occurring polynucleotide or polypeptide present in a living organism is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

The term “purified” does not require absolute purity; rather, it is intended as a relative definition. Individual nucleic acids obtained from a library have been conventionally purified to electrophoretic homogeneity. The purified nucleic acids of the present invention have been purified from the remainder of the genomic DNA in the organism by at least 10⁴ to 10⁶ fold. However, the term “purified” also includes nucleic acids which have been purified from the remainder of the genomic DNA or from other sequences in a library or other environment by at least one order of magnitude, preferably two or three orders of magnitude, and more preferably four or five orders of magnitude.

“Recombinant” means that the nucleic acid is adjacent to “backbone” nucleic acid to which it is not adjacent in its natural environment. “Enriched” nucleic acids represent 5% or more of the number of nucleic acid inserts in a population of nucleic acid backbone molecules. “Backbone” molecules include nucleic acids such as expression vectors, self-replicating nucleic acids, viruses, integrating nucleic acids, and other vectors or nucleic acids used to maintain or manipulate a nucleic acid of interest. Preferably, the enriched nucleic acids represent 15% or more, more preferably 50% or more, and most preferably 90% or more, of the number of nucleic acid inserts in the population of recombinant backbone molecules.

“Recombinant” polypeptides or proteins refer to polypeptides or proteins produced by recombinant DNA techniques, i.e. produced from cells transformed by an exogenous DNA construct encoding the desired polypeptide or protein. “Synthetic” polypeptides or proteins are those prepared by chemical synthesis.

The term “gene” means the segment of DNA involved in producing a polypeptide chain; it includes regions preceding and following the coding region (leader and trailer) as well as, where applicable, intervening regions (introns) between individual coding segments (exons).

A DNA or nucleotide “coding sequence” or “sequence encoding” a particular polypeptide or protein, is a DNA sequence which is transcribed and translated into a polypeptide or protein when placed under the control of appropriate regulatory sequences.

“Oligonucleotide” refers to a nucleic acid, generally of at least 10, preferably 15 and more preferably at least 20 nucleotides, preferably no more than 100 nucleotides, that are hybridizable to a genomic DNA molecule, a cDNA molecule, or an mRNA molecule encoding a gene, mRNA, cDNA or other nucleic acid of interest.

A promoter sequence is “operably linked to” a coding sequence recognized by RNA polymerase which initiates transcription at the promoter and transcribes the coding sequence into mRNA.

“Plasmids” are designated herein by a lowercase p preceded or followed by capital letters and/or numbers. The starting plasmids herein are commercially available, publicly available on an unrestricted basis, or can be constructed from available plasmids in accord with published procedures. In addition, equivalent plasmids to those described herein are known in the art and will be apparent to the skilled artisan.

“Digestion” of DNA refers to enzymatic cleavage of the DNA with a restriction enzyme that acts only at certain sequences in the DNA. The various restriction enzymes used herein are commercially available and their reaction conditions, cofactors and other requirements were used as would be known to the ordinary skilled artisan. For analytical purposes, typically 1 μg of plasmid or DNA fragment is used with about 2 units of enzyme in about 20 μl of buffer solution. For the purpose of isolating DNA fragments for plasmid construction, typically 5 to 50 μg of DNA are digested with 20 to 250 units of enzyme in a larger volume. Appropriate buffers and substrate amounts for particular enzymes are specified by the manufacturer. Incubation times of about 1 hour at 37° C. are ordinarily used, but may vary in accordance with the supplier's instructions. After digestion the gel electrophoresis may be performed to isolate the desired fragment.

We have now discovered the genes and proteins involved in the biosynthesis of the 16-member macrolide rosaramicin. Nucleic acid sequences encoding proteins involved in the biosynthesis of rosaramicin are provided in the accompanying sequence listing as SEQ ID NOS: 3, 5, 7, 9, 11, 13, 15, 17, 19, 21, 23, 25, 27, 29, 31, 33, 35, 37, 39. Polypeptides involved in the biosynthesis of rosaramicin are provided in the accompanying sequence listing as SEQ ID NOS: 2, 4, 6, 8, 10, 12, 14, 16, 18, 20, 22, 24, 26, 28, 30, 32, 34, 36, 38.

One aspect of the present invention is an isolated, purified, or enriched nucleic acid comprising one of the sequences of SEQ ID NOS: 3, 5, 7, 9, 11, 13, 15, 17, 19, 21, 23, 25, 27, 29, 31, 33, 35, 37, 39, the sequences complementary thereto, or a fragment comprising at least 100, 200, 300, 400, 500, 600, 700, 800 or more consecutive bases of one of the sequences of SEQ ID NOS: 3, 5, 7, 9, 11, 13, 15, 17, 19, 21, 23, 25, 27, 29, 31, 33, 35, 37, 39 or the sequences complementary thereto. The isolated, purified or enriched nucleic acids may comprise DNA, including cDNA, genomic DNA, and synthetic DNA. The DNA may be double stranded or single stranded, and if single stranded may be the coding (sense) or non-coding (anti-sense) strand. Alternatively, the isolated, purified or enriched nucleic acids may comprise RNA.

As discussed in more detail below, the isolated, purified or enriched nucleic acids of one of SEQ ID NOS: 3, 5, 7, 9, 11, 13, 15, 17, 19, 21, 23, 25, 27, 29, 31, 33, 35, 37, 39 may be used to prepare one of the polypeptides of SEQ ID NOS: 2, 4, 6, 8, 10, 12, 14, 16, 18, 20, 22, 24, 26, 28, 30, 32, 34, 36, 38 respectively or fragments comprising at least 50, 75, 100, 200, 300, 500 or more consecutive amino acids of one of the polypeptides of SEQ ID NO: 2, 4, 6, 8, 10, 12, 14, 16, 18, 20, 22, 24, 26, 28, 30, 32, 34, 36, 38.

Accordingly, another aspect of the present invention is an isolated, purified or enriched nucleic acid which encodes one of the polypeptides of SEQ ID NOS: 2, 4, 6, 8, 10, 12, 14, 16, 18, 20, 22, 24, 26, 28, 30, 32, 34, 36, 38 or fragments comprising at least 50, 75, 100, 150, 200, 300 or more consecutive amino acids of one of the polypeptides of SEQ ID NOS: 2, 4, 6, 8, 10, 12, 14, 16, 18, 20, 22, 24, 26, 28, 30, 32, 34, 36, 38. The coding sequences of these nucleic acids may be identical to one of the coding sequences of one of the nucleic acids of SEQ ID NOS: 3, 5, 7, 9, 11, 13, 15, 17, 19, 21, 23, 25, 27, 29, 31, 33, 35, 37, 39 or a fragment thereof or may be different coding sequences which encode one of the polypeptides of SEQ ID NOS: 2, 4, 6, 8,10,12, 14, 16, 18, 20, 22, 24, 26, 28, 30, 32, 34, 36, 38 or fragments comprising at least 50, 75, 100, 150, 200, 300 consecutive amino acids of one of the polypeptides of SEQ ID NOS: 2, 4, 6, 8, 10, 12, 14, 16, 18, 20, 22, 24, 26, 28, 30, 32, 34, 36, 38 as a result of the redundancy or degeneracy of the genetic code. The genetic code is well known to those of skill in the art and can be obtained, for example, from Stryer, Biochemistry, 3^{rd} edition, W. H. Freeman & Co., New York.

The isolated, purified or enriched nucleic acid which encodes one of the polypeptides of SEQ ID NOS: 2, 4, 6, 8, 10, 12, 14, 16, 18, 20, 22, 24, 26, 28, 30, 32, 34, 36, 38, may include, but is not limited to: (1) only the coding sequences of one of SEQ ID NOS: 3, 5, 7, 9, 11, 13, 15, 17, 19, 21, 23, 25, 27, 29, 31, 33, 35, 37, 39; (2) the coding sequences of SEQ ID NOS: 3, 5, 7, 9, 11, 13, 15, 17, 19, 21, 23, 25, 27, 29, 31, 33, 35, 37, 39 and additional coding sequences, such as leader sequences or proprotein; and (3) the coding sequences of SEQ IDNOS: 3, 5, 7, 9, 11, 13, 15, 17, 19, 21, 23, 25, 27, 29, 31, 33, 35, 37, 39 and non-coding sequences, such as introns or non-coding sequences 5′ and/or 3′ of the coding sequence. Thus, as used herein, the term “polynucleotide encoding a polypeptide” encompasses a polynucleotide that includes only coding sequence for the polypeptide as well as a polynucleotide that includes additional coding and/or non-coding sequence.

The invention relates to polynucleotides based on SEQ ID NOS: 3, 5, 7, 9, 11, 13, 15, 17, 19, 21, 23, 25, 27, 29, 31, 33, 35, 37, 39 but having polynucleotide changes that are “silent”, for example changes which do not alter the amino acid sequence encoded by the polynucleotides of SEQ ID NOS: 3, 5, 7, 9, 11, 13, 15, 17, 19, 21, 23, 25, 27, 29, 31, 33, 35, 37, 39. The invention also relates to polynucleotides which have nucleotide changes which result in amino acid substitutions, additions, deletions, fusions and truncations of the polypeptides of SEQ ID NOS: 2, 4, 6, 8, 10, 12, 14, 16, 18, 20, 22, 24, 26, 28, 30, 32, 34, 36, 38. Such nucleotide changes may be introduced using techniques such as site directed mutagenesis, random chemical mutagenesis, exonuclease III deletion, and other recombinant DNA techniques.

The isolated, purified or enriched nucleic acids of SEQ ID NOS: 3, 5, 7, 9, 11, 13,15, 17, 19, 21, 23, 25, 27, 29, 31, 33, 35, 37, 39, the sequences complementary thereto, or a fragment comprising at least 10, 15, 20, 25, 30, 35, 40, 50, 75, 100, 150, 200, 300, 400 or 500 consecutive bases of one of the sequence of SEQ ID NOS: 3, 5, 7, 9, 11, 13, 15, 17, 19, 21, 23, 25, 27, 29, 31, 33, 35, 37, 39, or the sequences complementary thereto may be used as probes to identify and isolate DNAs encoding the polypeptides of SEQ ID NOS: 2, 4, 6, 8, 10, 12, 14, 16, 18, 20, 22, 24, 26, 28, 30, 32, 34, 36, 38 respectively. In such procedures, a genomic DNA library is constructed from a sample microorganism or a sample containing a microorganism capable of producing a macrolide. The genomic DNA library is then contacted with a probe comprising a coding sequence or a fragment of the coding sequence, encoding one of the polypeptides of SEQ ID NOS: 2, 4, 6, 8, 10, 12, 14, 16, 18, 20, 22, 24, 26, 28, 30, 32, 34, 36, 38, or a fragment thereof under conditions which permit the probe to specifically hybridize to sequences complementary thereto. In a preferred embodiment, the probe is an oligonucleotide of about 10 to about 30 nucleotides in length designed based on a nucleic acid of SEQ ID NOS: 3, 5, 7, 9, 11, 13, 15, 17, 19, 21, 23, 25, 27, 29, 31, 33, 35, 37, 39. Genomic DNA clones which hybridize to the probe are then detected and isolated. Procedures for preparing and identifying DNA clones of interest are disclosed in Ausubel et al., Current Protocols in Molecular Biology, John Wiley 503 Sons, Inc. 1997; and Sambrook et al., Molecular Cloning: A Laboratory Manual 2d Ed., Cold Spring Harbor Laboratory Press, 1989. In another embodiment, the probe is a restriction fragment or a PCR amplified nucleic acid derived from SEQ ID NOS: 3, 5, 7, 9, 11, 13, 15, 17, 19, 21, 23, 25, 27, 29, 31, 33, 35, 37, 39.

The isolated, purified or enriched nucleic acids of SEQ ID NOS: 3, 5, 7, 9, 11, 13, 15, 17, 19, 21, 23, 25, 27, 29, 31, 33, 35, 37, 39, the sequences complementary thereto, or a fragment comprising at least 10, 15, 20, 25, 30, 35, 40, 50, 75, 100, 150, 200, 300, 400 or 500 consecutive bases of one of the sequences of SEQ ID NOS: 3, 5, 7, 9, 11, 13, 15, 17, 19, 21, 23, 25, 27, 29, 31, 33, 35, 37, 39, or the sequences complementary thereto may be used as probes to identify and isolate related nucleic acids. In some embodiments, the related nucleic acids may be genomic DNAs (or cDNAs) from potential macrolide producers. In such procedures, a nucleic acid sample containing nucleic acids from a potential macrolide-producer or rosaramicin-producer is contacted with the probe under conditions that permit the probe to specifically hybridize to related sequences. The nucleic acid sample may be a genomic DNA (or cDNA) library from the potential macrolide-producer. Hybridization of the probe to nucleic acids is then detected using any of the methods described above.

Hybridization may be carried out under conditions of low stringency, moderate stringency or high stringency. As an example of nucleic acid hybridization, a polymer membrane containing immobilized denatured nucleic acids is first prehybridized for 30 minutes at 45° C. in a solution consisting of 0.9 M NaCl, 50 mM NaH₂PO₄, pH 7.0, 5.0 mM Na₂EDTA, 0.5% SDS, 10× Denhardt's, and 0.5 mg/ml polyriboadenylic acid. Approximately 2×10⁷ cpm (specific activity 4-9×10⁸ cpm/ug) of ³²p end-labeled oligonucleotide probe are then added to the solution. After 12-16 hours of incubation, the membrane is washed for 30 minutes at room temperature in 1× SET (150 mM NaCl, 20 mM Tris hydrochloride, pH 7.8, 1 mM Na₂EDTA) containing 0.5% SDS, followed by a 30 minute wash in fresh 1× SET at Tm10° C. for the oligonucleotide probe where Tm is the melting temperature. The membrane is then exposed to autoradiographic film for detection of hybridization signals.

By varying the stringency of the hybridization conditions used to identify nucleic acids, such as genomic DNAs or cDNAs, which hybridize to the detectable probe, nucleic acids having different levels of homology to the probe can be identified and isolated. Stringency may be varied by conducting the hybridization at varying temperatures below the melting temperatures of the probes. The melting temperature of the probe may be calculated using the following formulas:

For oligonucleotide probes between 14 and 70 nucleotides in length the melting temperature (Tm) in degrees Celcius may be calculated using the formula: Tm=81.5+16.6(log [Na+])+0.41 (fraction G+C)−(600/N) where N is the length of the oligonucleotide.

If the hybridization is carried out in a solution containing formamide, the melting temperature may be calculated using the equation Tm=81.5+16.6(log [Na +])+0.41 (fraction G+C)−(0.63% formamide)−(600/N) where N is the length of the probe.

Prehybridization may be carried out in 6× SSC, 5× Denhardt's reagent, 0.5% SDS, 0.1 mg/ml denatured fragmented salmon sperm DNA or 6× SSC, 5× Denhardt's reagent, 0.5% SDS, 0.1 mg/ml denatured fragmented salmon sperm DNA, 50% formamide. The composition of the SSC and Denhardt's solutions are listed in Sambrook et al., supra.

Hybridization is conducted by adding the detectable probe to the hybridization solutions listed above. Where the probe comprises double stranded DNA, it is denatured by incubating at elevated temperatures and quickly cooling before addition to the hybridization solution. It may also be desirable to similarly denature single stranded probes to eliminate or diminish formation of secondary structures or oligomerization. The filter is contacted with the hybridization solution for a sufficient period of time to allow the probe to hybridize to cDNAs or genomic DNAs containing sequences complementary thereto or homologous thereto. For probes over 200 nucleotides in length, the hybridization may be carried out at 15-25° C. below the Tm. For shorter probes, such as oligonucleotide probes, the hybridization may be conducted at 5-10 ° C. below the Tm. Preferably, the hybridization is conducted in 6× SSC, for shorter probes. Preferably, the hybridization is conducted in 50% formamide containing solutions, for longer probes.

All the foregoing hybridizations would be considered to be examples of hybridization performed under conditions of high stringency.

Following hybridization, the filter is washed for at least 15 minutes in 2× SSC, 0.1% SDS at room temperature or higher, depending on the desired stringency. The filter is then washed with 0.1× SSC, 0.5% SDS at room temperature (again) for 30 minutes to 1 hour.

Nucleic acids which have hybridized to the probe are identified by conventional autoradiography and non-radioactive detection methods.

The above procedure may be modified to identify nucleic acids having decreasing levels of homology to the probe sequence. For example, to obtain nucleic acids of decreasing homology to the detectable probe, less stringent conditions may be used. For example, the hybridization temperature may be decreased in increments of 5° C. from 68° C. to 42° C. in a hybridization buffer having a Na+ concentration of approximately 1M. Following hybridization, the filter may be washed with 2× SSC, 0.5% SDS at the temperature of hybridization. These conditions are considered to be “moderate stringency” conditions above 50° C. and “low stringency” conditions below 50° C. A specific example of “moderate stringency” hybridization conditions is when the above hybridization is conducted at 55° C. A specific example of “low stringency” hybridization conditions is when the above hybridization is conducted at 45° C.

Alternatively, the hybridization may be carried out in buffers, such as 6× SSC, containing formamide at a temperature of 42° C. In this case, the concentration of formamide in the hybridization buffer may be reduced in 5% increments from 50% to 0% to identify clones having decreasing levels of homology to the probe. Following hybridization, the filter may be washed with 6× SSC, 0.5% SDS at 50° C. These conditions are considered to be “moderate stringency” conditions above 25% formamide and “low stringency” conditions below 25% formamide. A specific example of “moderate stringency” hybridization conditions is when the above hybridization is conducted at 30% formamide. A specific example of “low stringency” hybridization conditions is when the above hybridization is conducted at 10% formamide.

Nucleic acids which have hybridized to the probe are identified by conventional autoradiography and non-radioactive detection methods.

For example, the preceding methods may be used to isolate nucleic acids having a sequence with at least 97%, at least 95%, at least 90%, at least 85%, at least 80%, or at least 70% homology to a nucleic acid sequence selected from the group consisting of the sequences of SEQ ID NOS: 3, 5, 7, 9, 11, 13, 15, 17, 19, 21, 23, 25, 27, 29, 31, 33, 35, 37, 39, fragments comprising at least 10, 15, 20, 25, 30, 35, 40, 50, 75, 100, 150, 200, 300, 400, or 500 consecutive bases thereof, and the sequences complementary thereto. Homology may be measured using BLASTN version 2.0 with the default parameters. For example, the homologous polynucleotides may have a coding sequence that is a naturally occurring allelic variant of one of the coding sequences described herein. Such allelic variant may have a substitution, deletion or addition of one or more nucleotides when compared to the nucleic acids of SEQ ID NOS: 3, 5, 7, 9, 11, 13, 15, 17, 19, 21, 23, 25, 27, 29, 31, 33, 35, 37, 39, or the sequences complementary thereto.

Additionally, the above procedures may be used to isolate nucleic acids which encode polypeptides having at least 99%, 95%, at least 90%, at least 85%, at least 80%, or at least 70% homology to a polypeptide having the sequence of one of SEQ ID NOS: 2, 4, 6, 8, 10, 12, 14, 16, 18, 20, 22, 24, 26, 28, 30, 32, 34, 36, 38, or fragments comprising at least 50, 75, 100, 150, 200, 300 consecutive amino acids thereof as determined using the BLASTP version 2.2.2 algorithm with default parameters.

Another aspect of the present invention is an isolated or purified polypeptide comprising the sequence of one of SEQ ID NOS: 2, 4, 6, 8, 10, 12, 14, 16, 18, 20, 22, 24, 26, 28, 30, 32, 34, 36, 38 or fragments comprising at least 50, 75, 100, 150, 200 or 300 consecutive amino acids thereof. As discussed herein, such polypeptides may be obtained by inserting a nucleic acid encoding the polypeptide into a vector such that the coding sequence is operably linked to a sequence capable of driving the expression of the encoded polypeptide in a suitable host cell. For example, the expression vector may comprise a promoter, a ribosome binding site for translation initiation and a transcription terminator. The vector may also include appropriate sequences for modulating expression levels, an origin of replication and a selectable marker.

Promoters suitable for expressing the polypeptide or fragment thereof in bacteria include the E.coli lac or trp promoters, the lacl promoter, the lacZ promoter, the T3 promoter, the T7 promoter, the gpt promoter, the lambda P_{R} promoter, the lambda P_{L} promoter, promoters from operons encoding glycolytic enzymes such as 3-phosphoglycerate kinase (PGK), and the acid phosphatase promoter. Fungal promoters include the α factor promoter. Eukaryotic promoters include the CMV immediate early promoter, the HSV thymidine kinase promoter, heat shock promoters, the early and late SV40 promoter, LTRs from retroviruses, and the mouse metallothionein-l promoter. Other promoters known to control expression of genes in prokaryotic or eukaryotic cells or their viruses may also be used.

Mammalian expression vectors may also comprise an origin of replication, any necessary ribosome binding sites, a polyadenylation site, splice donors and acceptor sites, transcriptional termination sequences, and 5′ flanking nontranscribed sequences. In some embodiments, DNA sequences derived from the SV40 splice and polyadenylation sites may be used to provide the required nontranscribed genetic elements.

Vectors for expressing the polypeptide or fragment thereof in eukaryotic cells may also contain enhancers to increase expression levels. Enhancers are cis-acting elements of DNA, usually from about 10 to about 300 bp in length that act on a promoter to increase its transcription. Examples include the SV40 enhancer on the late side of the replication origin bp 100 to 270, the cytomegalovirus early promoter enhancer, the polyoma enhancer on the late side of the replication origin, and the adenovirus enhancers.

In addition, the expression vectors preferably contain one or more selectable marker genes to permit selection of host cells containing the vector. Examples of selectable markers that may be used include genes encoding dihydrofolate reductase or genes conferring neomycin resistance for eukaryotic cell culture, genes conferring tetracycline or ampicillin resistance in E. coli, and the S. cerevisiae TRP1 gene.

In some embodiments, the nucleic acid encoding one of the polypeptides of SEQ ID NOS: 2, 4, 6, 8, 10, 12, 14, 16, 18, 20, 22, 24, 26, 28, 30, 32, 34, 36, 38, or fragments comprising at least 50, 75, 100, 150, 200 or 300 consecutive amino acids thereof is assembled in appropriate phase with a leader sequence capable of directing secretion of the translated polypeptides or fragments thereof. Optionally, the nucleic acid can encode a fusion polypeptide in which one of the polypeptide of SEQ ID NOS: 2, 4, 6, 8, 10, 12, 14, 16, 18, 20, 22, 24, 26, 28, 30, 32, 34, 36, 38 or fragments comprising at least 5, 10, 15, 20, 25, 30, 35, 40, 50, 75, 100, or 150 consecutive amino acids thereof is fused to heterologous peptides or polypeptides, such as N-terminal identification peptides which impart desired characteristics such as increased stability or simplified purification or detection.

The appropriate DNA sequence may be inserted into the vector by a variety of procedures. In general, the DNA sequence is ligated to the desired position in the vector following digestion of the insert and the vector with appropriate restriction endonucleases. Alternatively, appropriate restriction enzyme sites can be engineered into a DNA sequence by PCR. A variety of cloning techniques are disclosed in Ausbel et al. Current Protocols in Molecular Biology, John Wiley 503 Sons, Inc. 1997 and Sambrook et al., Molecular Cloning: A Laboratory Manual 2d Ed., Cold Spring Harbour Laboratory Press, 1989. Such procedures and others are deemed to be within the scope of those skilled in the art.

The vector may be, for example, in the form of a plasmid, a viral particle, or a phage. Other vectors include derivatives of chromosomal, nonchromosomal and synthetic DNA sequences, viruses, bacterial plasmids, phage DNA, baculovirus, yeast plasmids, vectors derived from combinations of plasmids and phage DNA, viral DNA such as vaccinia, adenovirus, fowl pox virus, and pseudorabies. A variety of cloning and expression vectors for use with prokaryotic and eukaryotic hosts are described by Sambrook, et al., Molecular Cloning: A Laboratory Manual, Second Edition, Cold Spring Harbor, N.Y., (1989).

Particular bacterial vectors which may be used include the commercially available plasmids comprising genetic elements of the well known cloning vector pBR322 (ATCC 37017), pKK223-3 (Pharmacia Fine Chemicals, Uppsala, Sweden), pGEM1 (Promega Biotec, Madison, Wis., U.S.A.) pQE70, pQE60, pQE-9 (Qiagen), pD10, phiX174, pBluescript II KS, pNH8A, pNH16a, pNH18A, pNH46A (Stratagene), ptrc99a, pKK223-3, pKK233-3, pDR540, pRIT5 (Pharmacia), pKK232-8 and pCM7. Particular eukaryotic vectors include pSV2CAT, pOG44, pXT1, pSG (Stratagene) pSVK3, pBPV, pMSG, and pSVL (Pharmacia). However, any other vector may be used as long as it is replicable and stable in the host cell.

The host cell may be any of the host cells familiar to those skilled in the art, including prokaryotic cells or eukaryotic cells. As representative examples of appropriate hosts, there may be mentioned: bacteria cells, such as E. coli, Streptomyces, Bacillus subtilis, Salmonella typhimurium and various species within the genera Pseudomonas, Streptomyces, and Staphylococcus, fungal cells, such as yeast, insect cells such as Drosophila S2 and Spodoptera Sf9, animal cells such as CHO, COS or Bowes melanoma, and adenoviruses. The selection of an appropriate host is within the abilities of those skilled in the art.

The vector may be introduced into the host cells using any of a variety of techniques, including electroporation transformation, transfection, transduction, viral infection, gene guns, or Ti-mediated gene transfer. Where appropriate, the engineered host cells can be cultured in conventional nutrient media modified as appropriate for activating promoters, selecting transformants or amplifying the genes of the present invention. Following transformation of a suitable host strain and growth of the host strain to an appropriate cell density, the selected promoter may be induced by appropriate means (e.g., temperature shift or chemical induction) and the cells may be cultured for an additional period to allow them to produce the desired polypeptide or fragment thereof.

Cells are typically harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment thereof can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts (described by Gluzman, Cell, 23:175(1981)), and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptide produced by host cells containing the vector may be glycosylated or may be non-glycosylated. Polypeptides of the invention may or may not also include an initial methionine amino acid residue.

Alternatively, the polypeptides of SEQ ID NOS: 2, 4, 6, 8, 10, 12, 14, 16, 18, 20, 22, 24, 26, 28, 30, 32, 34, 36, 38, or fragments comprising at least 50, 75, 100, 150, 200 or 300 consecutive amino acids thereof can be synthetically produced by conventional peptide synthesizers. In other embodiments, fragments or portions of the polynucleotides may be employed for producing the corresponding full-length polypeptide by peptide synthesis; therefore, the fragments may be employed as intermediates for producing the full-length polypeptides.

Cell-free translation systems can also be employed to produce one of the polypeptides of SEQ ID NOS: 2, 4, 6, 8, 10, 12, 14, 16, 18, 20, 22, 24, 26, 28, 30, 32, 34, 36, 38, or fragments comprising at least 50, 75, 100, 150, 200 or 300 consecutive amino acids thereof using mRNAs transcribed from a DNA construct comprising a promoter operably linked to a nucleic acid encoding the polypeptide or fragment thereof. In some embodiments, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof.

The present invention also relates to variants of the polypeptides of SEQ ID NOS: 2, 4, 6, 8, 10, 12, 14, 16, 18, 20, 22, 24, 26, 28, 30, 32, 34, 36, 38, or fragments comprising at least 50, 75, 100, 150, 200 or 300 consecutive amino acids thereof. The term “variant” includes derivatives or analogs of these polypeptides. In particular, the variants may differ in amino acid sequence from the polypeptides of SEQ ID NOS: 2, 4, 6, 8, 10, 12, 14, 16, 18, 20, 22, 24, 26, 28, 30, 32, 34, 36, 38, by one or more substitutions, additions, deletions, fusions and truncations, which may be present in any combination.

The variants may be naturally occurring or created in vitro. In particular, such variants may be created using genetic engineering techniques such as site directed mutagenesis, random chemical mutagenesis, Exonuclease III deletion procedures, and standard cloning techniques. Alternatively, such variants, fragments, analogs, or derivatives may be created using chemical synthesis or modification procedures.

Other methods of making variants are also familiar to those skilled in the art. These include procedures in which nucleic acid sequences obtained from natural isolates are modified to generate nucleic acids that encode polypeptides having characteristics which enhance their value in industrial or laboratory applications. In such procedures, a large number of variant sequences having one or more nucleotide differences with respect to the sequence obtained from the natural isolate are generated and characterized. Preferably, these nucleotide differences result in amino acid changes with respect to the polypeptides encoded by the nucleic acids from the natural isolates.

For example, variants may be created using error prone PCR. In error prone PCR, DNA amplification is performed under conditions where the fidelity of the DNA polymerase is low, such that a high rate of point mutation is obtained along the entire length of the PCR product. Error prone PCR is described in Leung, D.W., et al., Technique, 1:11-15 (1989) and Caldwell, R. C. & Joyce G. F., PCR Methods Applic., 2:28-33 (1992). Variants may also be created using site directed mutagenesis to generate site-specific mutations in any cloned DNA segment of interest. Oligonucleotide mutagenesis is described in Reidhaar-Olson, J. F. & Sauer, R. T., et al., Science, 241:53-57 (1988). Variants may also be created using directed evolution strategies such as those described in U.S. Pat. Nos. 6,361,974 and 6,372,497. The variants of the polypeptides of SEQ ID NOS: 2, 4, 6, 8, 10, 12, 14, 16, 18, 20, 22, 24, 26, 28, 30, 32, 34, 36, 38, may be (i) variants in which one or more of the amino acid residues of the polypeptides of SEQ ID NOS: 2, 4, 6, 8, 10, 12,14, 16, 18, 20, 22, 24, 26, 28, 30, 32, 34, 36, 38, are substituted with a conserved or non-conserved amino acid residue (preferably a conserved amino acid residue) and such substituted amino acid residue may or may not be one encoded by the genetic code.

Conservative substitutions are those that substitute a given amino acid in a polypeptide by another amino acid of like characteristics. Typically seen as conservative substitutions are the following replacements: replacements of an aliphatic amino acid such as Ala, Val, Leu and lle with another aliphatic amino acid; replacement of a Ser with a Thr or vice versa; replacement of an acidic residue such as Asp or Glu with another acidic residue; replacement of a residue bearing an amide group, such as Asn or Gln, with another residue bearing an amide group; exchange of a basic residue such as Lys or Arg with another basic residue; and replacement of an aromatic residue such as Phe or Tyr with another aromatic residue.

Other variants are those in which one or more of the amino acid residues of the polypeptides of SEQ ID NOS: 2, 4, 6, 8, 10, 12, 14, 16, 18, 20, 22, 24, 26, 28, 30, 32, 34, 36, 38 includes a substituent group.

Still other variants are those in which the polypeptide is associated with another compound, such as a compound to increase the half-life of the polypeptide (for example, polyethylene glycol).

Additional variants are those in which additional amino acids are fused to the polypeptide, such as leader sequence, a secretory sequence, a proprotein sequence or a sequence which facilitates purification, enrichment, or stabilization of the polypeptide.

In some embodiments, the fragments, derivatives and analogs retain the same biological function or activity as the polypeptides of SEQ ID NOS: 2, 4, 6, 8, 10, 12, 14, 16, 18, 20, 22, 24, 26, 28, 30, 32, 34, 36, 38. In other embodiments, the fragment, derivative or analogue includes a fused heterologous sequence which facilitates purification, enrichment, detection, stabilization or secretion of the polypeptide that can be enzymatically cleaved, in whole or in part, away from the fragment, derivative or analogue.

Another aspect of the present invention are polypeptides or fragments thereof which have at least 70%, at least 80%, at least 85%, at least 90%, or more than 95% homology to one of the polypeptides of SEQ ID NOS: 2, 4, 6, 8, 10, 12, 14, 16, 18, 20, 22, 24, 26, 28, 30, 32, 34, 36, 38, or a fragment comprising at least 50, 75, 100, 150, 200 or 300 consecutive amino acids thereof. Homology may be determined using a program, such as BLASTP version 2.2.2 with the default parameters, which aligns the polypeptides or fragments being compared and determines the extent of amino acid identity or similarity between them. It will be appreciated that amino acid “homology” includes conservative substitutions such as those described above.

The polypeptides or fragments having homology to one of the polypeptides of SEQ ID NOS: 2, 4, 6, 8, 10, 12, 14, 16, 18, 20, 22, 24, 26, 28, 30, 32, 34, 36, 38, or a fragment comprising at least 50, 75, 100, 150, 200 or 300 consecutive amino acids thereof may be obtained by isolating the nucleic acids encoding them using the techniques described above.

Alternatively, the homologous polypeptides or fragments may be obtained through biochemical enrichment or purification procedures. The sequence of potentially homologous polypeptides or fragments may be determined by proteolytic digestion, gel electrophoresis and/or microsequencing. The sequence of the prospective homologous polypeptide or fragment can be compared to one of the polypeptides of SEQ ID NOS: 2, 4, 6, 8, 10, 12, 14, 16, 18, 20, 22, 24, 26, 28, 30, 32, 34, 36, 38, or a fragment comprising at least 5, 10, 15, 20, 25, 30, 35, 40, 50, 75, 100, or 150 consecutive amino acids thereof using a program such as BLASTP version 2.2.2 with the default parameters.

The polypeptides of SEQ ID NOS: 2, 4, 6, 8, 10, 12, 14, 16, 18, 20, 22, 24, 26, 28, 30, 32, 34, 36, 38, or fragments, derivatives or analogs thereof comprising at least 40, 50, 75, 100, 150, 200 or 300 consecutive amino acids thereof invention may be used in a variety of applications. For example, the polypeptides or fragments, derivatives or analogs thereof may be used to catalyze certain biochemical reactions. In particular, the polypeptides of the TESA family, namely SEQ ID NO: 4 or fragments, derivatives or analogs thereof; the PKSH family, namely SEQ ID NOS: 10, 12, 14, 16, 18 or fragments, derivatives or analogs thereof; the OXRH family, namely SEQ ID NO: 26 or fragments, derivatives or analogs thereof may be used in any combination, in vitro or in vivo, to direct or enhance the synthesis or modification of a polyketide, polyketide substructure, or precursor thereof. Polypeptides of the MTFA family, namely SEQ ID NO: 24 or fragments, derivatives or analogs thereof may be used, in vitro or in vivo, to catalyze methylation reactions that modify compounds that are either endogenously produced by the host, supplemented to the growth medium, or are added to a cell-free, purified or enriched preparation of MTFA polypeptide. Polypeptides of the OXRC family, namely SEQ ID NOS: 6, 8 or fragments, derivatives or analogs thereof; the OXRB family, namely SEQ ID NO: 20 or fragments, derivatives or analogs thereof; the OXRH family, namely SEQ ID NO: 26 or fragments, derivatives or analogs thereof may be used, in vitro or in vivo, to catalyze oxidation reactions that modify compounds that are either endogenously produced by the host, supplemented to the growth medium, or are added to a cell-free, purified or enriched preparation of said polypeptide. Polypeptides of the NBPA family, namely SEQ ID NO: 32 or fragments, derivatives or analogs thereof; the OXRB family, namely SEQ ID NO: 20 or fragments, derivatives or analogs thereof; the DATF family, namely SEQ ID NO: 34 or fragments, derivatives or analogs thereof; the SURA family, namely SEQ ID NO: 36 or fragments, derivatives or analogs thereof; the MTFA family, namely SEQ ID NO: 24 or fragments, derivatives or analogs thereof; the GTFA family, namely SEQ ID NO: 22 or fragments, derivatives or analogs thereof may be used, in vitro or in vivo, to catalyze biochemical reactions involved in activating, modifying, or transferring sugar moieties. Polypeptides of the ABCC family, namely SEQ ID NO: 2 or fragments, derivatives or analogs thereof; the MTRA family, namely SEQ ID NO: 38 or fragments, derivatives or analogs thereof may be used to confer to microorganisms or eukaryotic cells resistance to polyketides, macrolides, rosaramicin, or compounds related to rosaramicin. Polypeptides of the REGS family, namely SEQ ID NO: 28 or fragments, derivatives or analogs thereof; the REGM family, namely SEQ ID NO: 30 or fragments, derivatives or analogs thereof may be used to increase the yield of polyketides, macrolides, rosaramicin, or compounds related to rosaramicin in either naturally producing organisms or heterologously producing recombinant organisms.

The polypeptides of SEQ ID NOS: 2, 4, 6, 8, 10, 12, 14, 16, 18, 20, 22, 24, 26, 28, 30, 32, 34, 36, 38, or fragments, derivatives or analogues thereof comprising at least 5, 10, 15, 20, 25, 30, 35, 40, 50, 75, 100, or 150 consecutive amino acids thereof, may also be used to generate antibodies which bind specifically to the polypeptides or fragments, derivatives or analogues. The antibodies generated from SEQ ID NOS: 2, 4, 6, 8, 10, 12, 14, 16, 18, 20, 22, 24, 26, 28, 30, 32, 34, 36, 38 may be used to determine whether a biological sample contains Micromonospora carbonacea or a related microorganism.

In such procedures, a biological sample is contacted with an antibody capable of specifically binding to one of the polypeptides of SEQ ID NOS: 2, 4, 6, 8, 10, 12, 14, 16, 18, 20, 22, 24, 26, 28, 30, 32, 34, 36, 38, or fragments comprising at least 5, 10, 15, 20, 25, 30, 35, 40, 50, 75, 100, or 150 consecutive amino acids thereof. The ability of the biological sample to bind to the antibody is then determined. For example, binding may be determined by labeling the antibody with a detectable label such as a fluorescent agent, an enzymatic label, or a radioisotope. Alternatively, binding of the antibody to the sample may be detected using a secondary antibody having such a detectable label thereon. A variety of assay protocols which may be used to detect the presence of an rosaramicin-producer or of Micromonospora carbonacea or of polypeptides related to SEQ ID NOS: 2, 4, 6, 8, 10, 12, 14, 16, 18, 20, 22, 24, 26, 28, 30, 32, 34, 36, 38, in a sample are familiar to those skilled in the art. Particular assays include ELISA assays, sandwich assays, radioimmunoassays, and Western Blots. Alternatively, antibodies generated from SEQ ID NOS: 2, 4, 6, 8, 10, 12, 14, 16, 18, 20, 22, 24, 26, 28, 30, 32, 34, 36, 38, may be used to determine whether a biological . sample contains related polypeptides that may be involved in the biosynthesis of natural products of the rosaramicin class or other macrolides.

Polyclonal antibodies generated against the polypeptides of SEQ ID NOS: 2, 4, 6, 8, 10, 12, 14, 16, 18, 20, 22, 24, 26, 28, 30, 32, 34, 36, 38, or fragments comprising at least 5, 10, 15, 20, 25, 30, 35, 40, 50, 75, 100, or 150 consecutive amino acids thereof can be obtained by direct injection of the polypeptides into an animal or by administering the polypeptides to an animal, preferably a nonhuman. The antibody so obtained will then bind the polypeptide itself. In this manner, even a sequence encoding only a fragment of the polypeptide can be used to generate antibodies that may bind to the whole native polypeptide. Such antibodies can then be used to isolate the polypeptide from cells expressing that polypeptide.

For preparation of monoclonal antibodies, any technique which provides antibodies produced by continuous cell line cultures can be used. Examples include the hybridoma technique (Kholer and Milstein, 1975, Nature, 256:495-497), the trioma technique, the human B-cell hybridoma technique (Kozbor et al., 1983, Immunology Today 4:72), and the EBV-hybridoma technique (Cole, et al., 1985, in Monoclonal Antibodies and Cancer Therapy, Alan R. Liss, Inc., pp. 77-96).

Techniques described for the production of single chain antibodies (U.S. Pat. No. 4,946,778) can be adapted to produce single chain antibodies to the polypeptides of SEQ ID NOS: 2, 4, 6, 8, 10, 12, 14, 16, 18, 20, 22, 24, 26, 28, 30, 32, 34, 36, 38, or fragments comprising at least 5, 10, 15, 20, 25, 30, 35, 40, 50, 75, 100, or 150 consecutive amino acids thereof. Alternatively, transgenic mice may be used to express humanized antibodies to these polypeptides or fragments thereof.

Antibodies generated against the polypeptides of SEQ ID NOS: 2, 4, 6, 8, 10, 12, 14, 16, 18, 20, 22, 24, 26, 28, 30, 32, 34, 36, 38, or fragments comprising at least 5, 10, 15, 20, 25, 30, 35, 40, 50, 75, 100, or 150 consecutive amino acids thereof may be used in screening for similar polypeptides from a sample containing organisms or cell-free extracts thereof. In such techniques, polypeptides from the sample is contacted with the antibodies and those polypeptides which specifically bind the antibody are detected. Any of the procedures described above may be used to detect antibody binding. One such screening assay is described in “Methods for measuring Cellulase Activities”, Methods in Enzymology, Vol 160, pp. 87-116.

As used herein, the term “nucleic acid codes of SEQ ID NOS: 3, 5, 7, 9, 11, 13, 15, 17, 19, 21, 23, 25, 27, 29, 31, 33, 35, 37, 39” encompass the nucleotide sequences of SEQ ID NOS: 3, 5, 7, 9, 11, 13, 15, 17, 19, 21, 23, 25, 27, 29, 31, 33, 35, 37, 39, fragments of SEQ ID NOS: 3, 5, 7, 9, 11, 13, 15, 17, 19, 21, 23, 25, 27, 29, 31, 33, 35, 37, 39, nucleotide sequences homologous to SEQ ID NOS: 3, 5, 7, 9, 11, 13, 15, 17, 19, 21, 23, 25, 27, 29, 31, 33, 35, 37, 39, or homologous to fragments of SEQ ID NOS: 3, 5, 7, 9, 11, 13, 15, 17, 19, 21, 23, 25, 27, 29, 31, 33 ,35, 37, 39, and sequences complementary to all of the preceding sequences. The fragments include portions of SEQ ID NOS: 3, 5, 7, 9, 11, 13, 15, 17, 19, 21, 23, 25, 27, 29, 31, 33, 35, 37, 39, comprising at least 10, 15, 20, 25, 30, 35, 40, 50, 75, 100, 150, 200, 300, 400 or 500 consecutive nucleotides of SEQ ID NOS: 3, 5, 7, 9, 11, 13, 15, 17, 19, 21, 23, 25, 27, 29, 31, 33, 35, 37, 39. Preferably, the fragments are novel fragments. Homologous sequences and fragments of SEQ ID NOS: 3, 5, 7, 9, 11, 13, 15, 17, 19, 21, 23, 25, 27, 29, 31, 33, 35, 37, 39 refer to a sequence having at least 99%, 98%, 97%, 96%, 95%, 90%, 80%, 75% or 70% identity to these sequences. Homology may be determined using any of the computer programs and parameters described herein, including BLASTN and TBLASTX with the default parameters. Homologous sequences also include RNA sequences in which uridines replace the thymines in the nucleic acid codes of SEQ ID NOS: 3, 5, 7, 9, 11, 13, 15, 17, 19, 21, 23, 25, 27, 29, 31, 33, 35, 37, 39.

The homologous sequences may be obtained using any of the procedures described herein or may result from the correction of a sequencing error. It will be appreciated that the nucleic acid codes of SEQ ID NOS: 3, 5, 7, 9, 11, 13, 15, 17, 19, 21, 23, 25, 27, 29, 31, 33, 35, 37, 39 can be represented in the traditional single character format in which G, A, T and C denote the guanine, adenine, thymine and cytosine bases of the deoxyribonucleic acid (DNA) sequence respectively, or in which G, A, U and C denote the guanine, adenine, uracil and cytosine bases of the ribonucleic acid (RNA) sequence (see the inside back cover of Stryer, Biochemistry, 3^{rd} edition, W. H. Freeman & Co., New York) or in any other format which records the identity of the nucleotides in a sequence.

“Polypeptide codes of SEQ ID NOS: 2, 4, 6, 8, 10, 12, 14, 16, 18, 20, 22, 24, 26, 28, 30, 32, 34, 36, 38” encompass the polypeptide sequences of SEQ ID NOS: 2, 4, 6, 8, 10, 12, 14, 16, 18, 20, 22, 24, 26, 28, 30, 32, 34, 36, 38 which are encoded by the nucleic acid sequences of SEQ ID NOS: 3, 5, 7, 9, 11, 13, 15, 17, 19, 21, 23, 25, 27, 29, 31, 33, 35, 37, 39, polypeptide sequences homologous to the polypeptides of SEQ ID NOS: 2, 4, 6, 8, 10, 12, 14, 16, 18, 20, 22, 24, 26, 28, 30, 32, 34, 36, 38, or fragments of any of the preceding sequences. Homologous polypeptide sequences refer to a polypeptide sequence having at least 99%, 98%, 97%, 96%, 95%, 90%, 85%, 80%, 75% or 70% identity to one of the polypeptide sequences of SEQ ID NOS: 2, 4, 6, 8, 10, 12, 14, 16, 18, 20, 22, 24, 26, 28, 30, 32, 34, 36, 38. Polypeptide sequence homology may be determined using any of the computer programs and parameters described herein, including BLASTP version 2.2.1 with the default parameters or with any user-specified parameters. The homologous sequences may be obtained using any of the procedures described herein or may result from the correction of a sequencing error. The polypeptide fragments comprise at least 5, 10, 15, 20, 25, 30, 35, 40, 50, 75, 100 or 150 consecutive amino acids of the polypeptides of SEQ ID NOS: 2, 4, 6, 8, 10, 12, 14, 16, 18, 20, 22, 24, 26, 28, 30, 32, 34, 36, 38. Preferably the fragments are novel fragments. It will be appreciated that the polypeptide codes of the SEQ ID NOS: 2, 4, 6, 8, 10, 12, 14, 16, 18, 20, 22, 24, 26, 28, 30, 32, 34, 36, 38 can be represented in the traditional single character format or three letter format (see the inside back cover of Stryer, Biochemistry, 3^{rd} edition, W.H. Freeman & Co., New York) or in any other format which relates the identity of the polypeptides in a sequence.

It will be readily appreciated by those skilled in the art that the nucleic acid codes of SEQ ID NOS: 3, 5, 7, 9, 11, 13, 15, 17, 19, 21, 23, 25, 27, 29, 31, 33, 35, 37, 39, and the polypeptide codes of SEQ ID NOS: 2, 4, 6, 8, 10, 12, 14, 16, 18, 20, 22, 24, 26, 28, 30, 32, 34, 36, 38 can be stored, recorded and manipulated on any medium which can be read and accessed by a computer. As used herein, the words “recorded” and “stored” refer to a process for storing information on a computer medium. A skilled artisan can readily adopt any of the presently known methods for recording information on a computer readable medium to generate manufactures comprising one or more of the nucleic acid codes of SEQ ID NOS: 3, 5, 7, 9, 11, 13, 15, 17, 19, 21, 23, 25, 27, 29, 31, 33, 35, 37, 39, and the polypeptide codes of SEQ ID NOS: 2, 4, 6, 8, 10, 12, 14, 16, 18, 20, 22, 24, 26, 28, 30, 32, 34, 36, 38.

Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of media known to those skilled in the art.

The nucleic acid codes of SEQ ID NOS: 3, 5, 7, 9, 11, 13, 15, 17, 19, 21, 23, 25, 27, 29, 31, 33, 35, 37, 39, a subset thereof, the polypeptide codes of SEQ ID NOS: 2, 4, 6, 8, 10, 12, 14, 16, 18, 20, 22, 24, 26, 28, 30, 32, 34, 36, 38, and a subset thereof may be stored and manipulated in a variety of data processor programs in a variety of formats. For example, one or more of the nucleic acid codes of SEQ ID NOS: 3, 5, 7, 9, 11, 13, 15, 17, 19, 21, 23, 25, 27, 29, 31, 33, 35, 37, 39, and one or more of the polypeptide codes of SEQ ID NOS: 2, 4, 6, 8,10, 12,14, 16, 18, 20, 22, 24, 26, 28, 30, 32, 34, 36, 38 may be stored as ASCII or text in a word processing file, such as MicrosoftWORD or WORDPERFECT in a variety of database programs familiar to those of skill in the art, such as DB2 or ORACLE. In addition, many computer programs and databases may be used as sequence comparers, identifiers or sources of query nucleotide sequences or query polypeptide sequences to be compared to one or more of the nucleic acid codes of SEQ ID NOS: 3, 5, 7, 9, 11, 13, 15, 17, 19, 21, 23, 25, 27, 29, 31, 33, 35, 37, 39, and one or more of the polypeptide codes of SEQ ID NOS: 2, 4, 6, 8, 10, 12, 14, 16, 18, 20, 22, 24, 26, 28, 30, 32, 34, 36, 38.

The following list is intended not to limit the invention but to provide guidance to programs and databases useful with one or more of the nucleic acid codes of SEQ ID NOS: 3, 5, 7, 9, 11, 13, 15, 17, 19, 21, 23, 25, 27, 29, 31, 33, 35, 37, 39, and the polypeptide codes of SEQ ID NOS: 2, 4, 6, 8, 10, 12, 14, 16, 18, 20, 22, 24, 26, 28, 30, 32, 34, 36, 38. The program and databases which may be used include, but are not limited to: MacPattern (EMBL), DiscoveryBase (Molecular Applications Group), GeneMine (Molecular Applications Group) Look (Molecular Applications Group), MacLook (Molecular Applications Group), BLAST and BLAST2 (NCBI), BLASTN and BLASTX (Altschul et al, J. Mol. Biol. 215:403 (1990)), FASTA (Person and Lipman, Proc. Nalt. Acad. Sci. USA, 85:2444 (1988)), FASTDB (Brutlag et al. Comp. App. Biosci. 6-237-245,1990), Catalyst (Molecular Simulations Inc.), Catalyst/SHAPE (Molecular Simulations Inc.), Cerius².DBAccess (Molecular Simulations Inc.), HypoGen (Molecular Simulations Inc.), Insight II (Molecular Simulations Inc.), Discover (Molecular Simulations Inc.), CHARMm (Molecular Simulations Inc.), Felix (Molecular Simulations Inc.), DelPhi (Molecular Simulations Inc.), QuanteMM (Molecular Simulations Inc.), Homology (Molecular Simulations Inc.), Modeler (Molecular Simulations Inc.), ISIS (Molecular Simulations Inc.), Quanta/Protein Design (Molecular Simulations Inc.), WetLab (Molecular Simulations Inc.), WetLab Diversity Explorer (Molecular Simulations Inc.), Gene Explorer (Molecular Simulations Inc.), SeqFold (Molecular Simulations Inc.), the MDL Available Chemicals Directory database, the MDL Drug Data Report data base, the Comprehensive Medicinal Chemistry database, Derwents' World Drug Index database, the BioByteMasterFile database, the Genbank database, and the Gensyqn database. Many other programs and databases would be apparent to one of skill in the art given the present disclosure.

Embodiments of the present invention include systems, particularly computer systems that store and manipulate the sequence information described herein. As used herein, “a computer system”, refers to the hardware components, software components, and data storage components used to analyze one or more of the nucleic acid codes of SEQ ID NOS: 3, 5, 7, 9, 11, 13, 15, 17, 19, 21, 23, 25, 27, 29, 31, 33, 35, 37, 39, and the polypeptide codes of SEQ ID NOS: 2, 4, 6, 8, 10, 12, 14, 16, 18, 20, 22, 24, 26, 28, 30, 32, 34, 36, 38.

Preferably, the computer system is a general purpose system that comprises a processor and one or more internal data storage components for storing data, and one or more data retrieving devices for retrieving the data stored on the data storage components. A skilled artisan can readily appreciate that any one of the currently available computer systems are suitable.

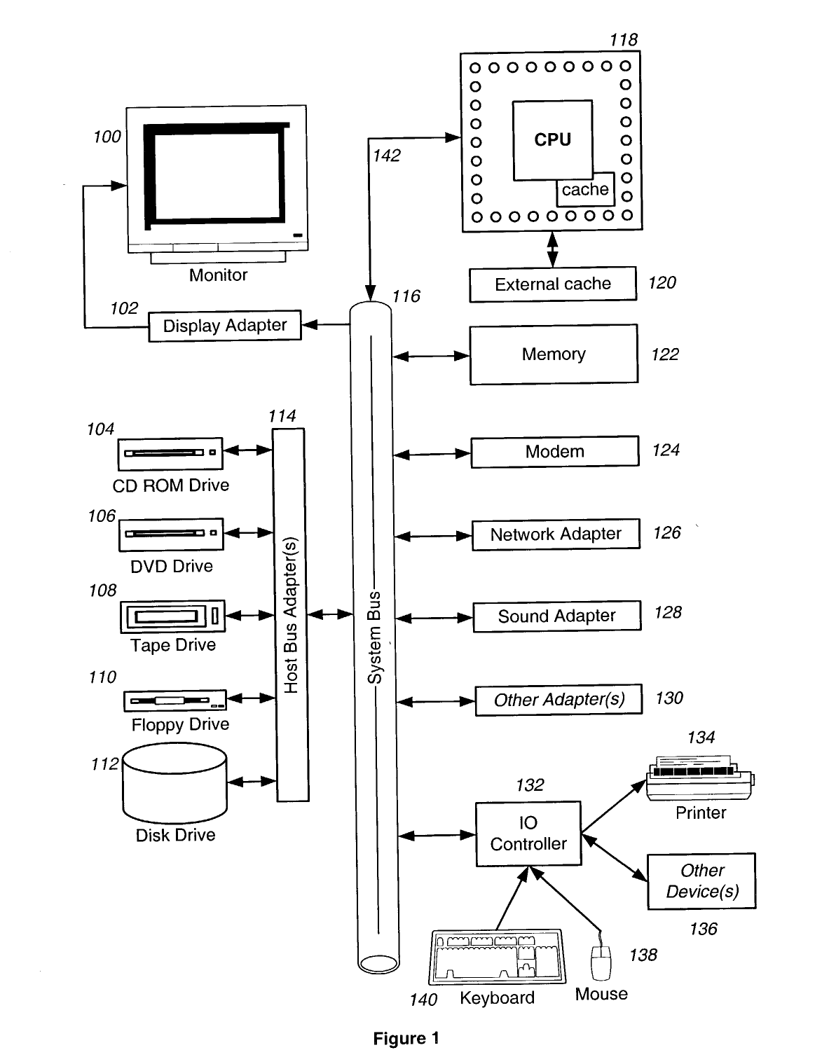

The computer system of FIG. 1 illustrates components that may be present in a conventional computer system. One skilled in the art will readily appreciate that not all components illustrated in FIG. 1 are required to practice the invention and, likewise, additional components not illustrated in FIG. 1 may be present in a computer system contemplated for use with the invention. Referring to the computer system of FIG. 1, the components are connected to a central system bus 116. The components include a central processing unit 118 with internal 118 and/or external cache memory 120, system memory 122, display adapter 102 connected to a monitor 100, network adapter 126 which may also be referred to as a network interface, internal modem 124, sound adapter 128, IO controller 132 to which may be connected a keyboard 140 and mouse 138, or other suitable input device such as a trackball or tablet, as well as external printer 134, and/or any number of external devices such as external modems, tape storage drives, or disk drives 136. One or more host bus adapters 114 may be connected to the system bus 116. To host bus adapter 114 may optionally be connected one or more storage devices such as disk drives 112 (removable or fixed), floppy drives 110, tape drives 108, digital versatile disk DVD drives 106, and compact disk CD ROM drives 104. The storage devices may operate in read-only mode and / or in read-write mode. The computer system may optionally include multiple central processing units 118, or multiple banks of memory 122. Arrows 142 in FIG. 1 indicate the interconnection of internal components of the computer system. The arrows are illustrative only and do not specify exact connection architecture.

Software for accessing and processing the one or more of the nucleic acid codes of SEQ ID NOS: 3, 5, 7, 9, 11, 13, 15, 17, 19, 21, 23, 25, 27, 29, 31, 33, 35, 37, 39, and the polypeptide codes of SEQ ID NOS: 2, 4, 6, 8, 10, 12, 14, 16, 18, 20, 22, 24, 26, 28, 30, 32, 34, 36, 38 (such as sequence comparison software, analysis software as well as search tools, annotation tools, and modeling tools etc.) may reside in main memory 122 during execution.

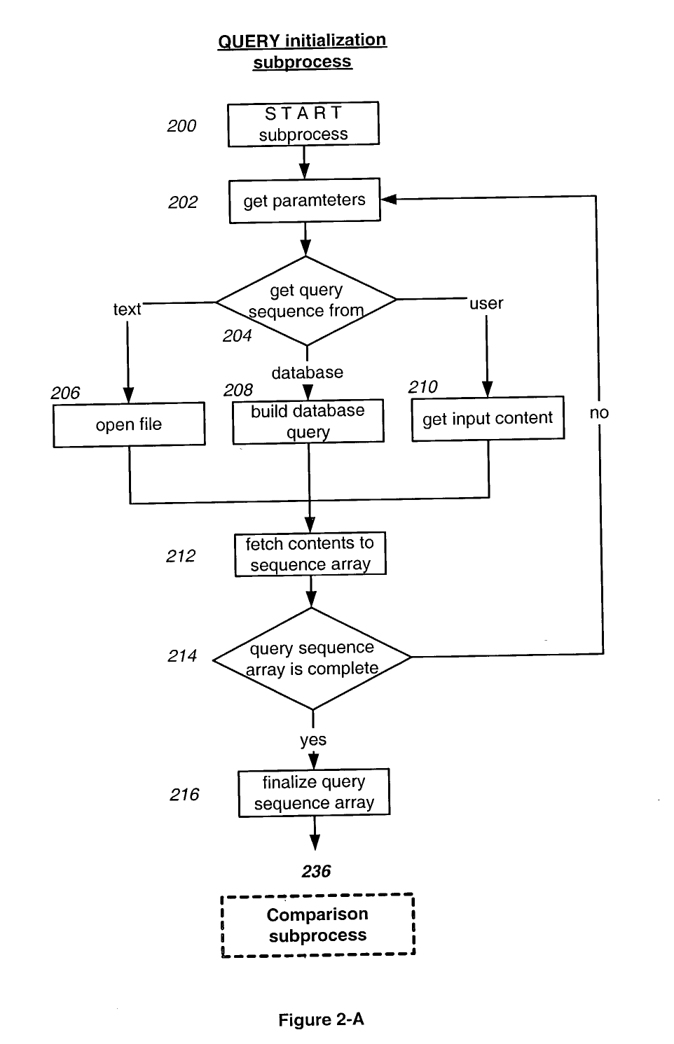

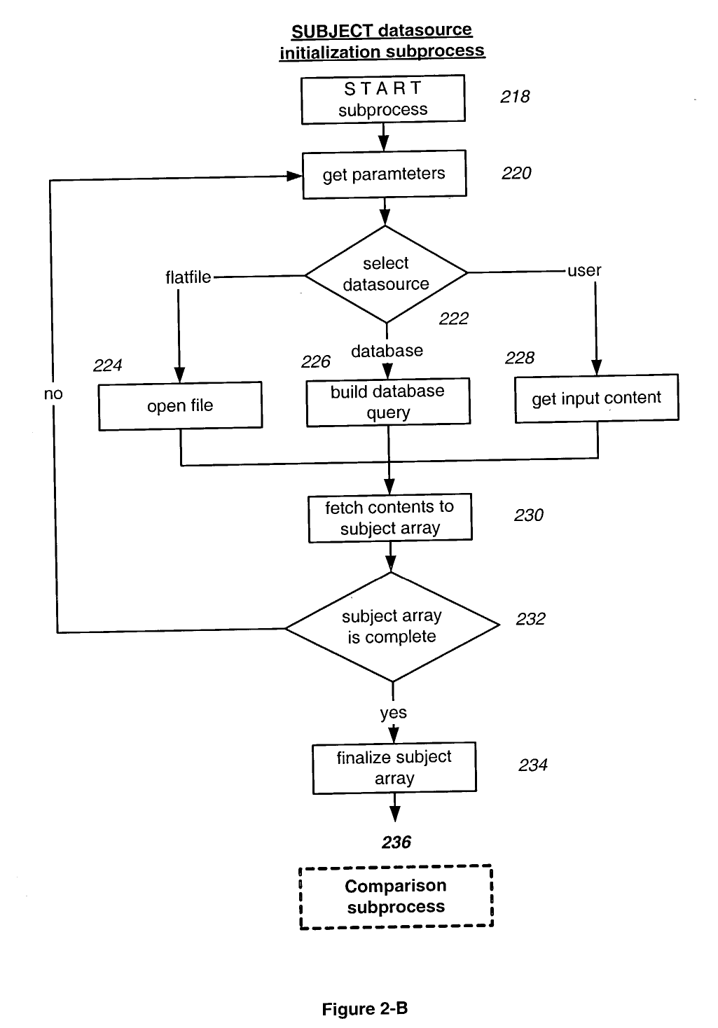

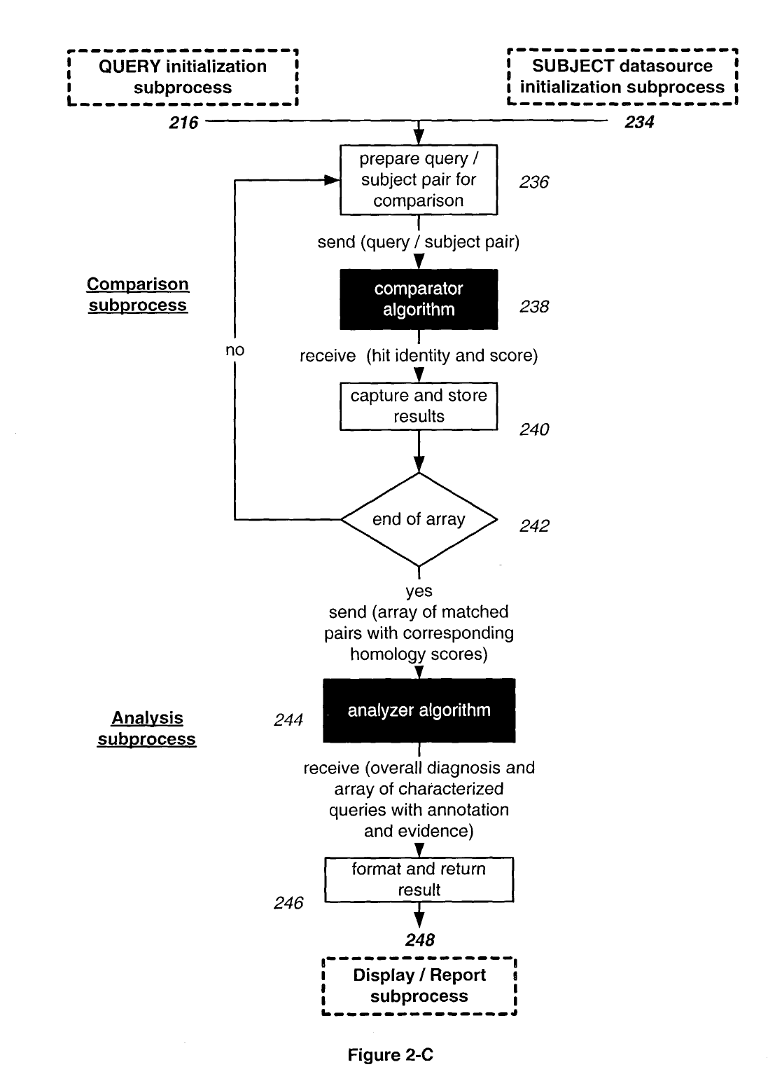

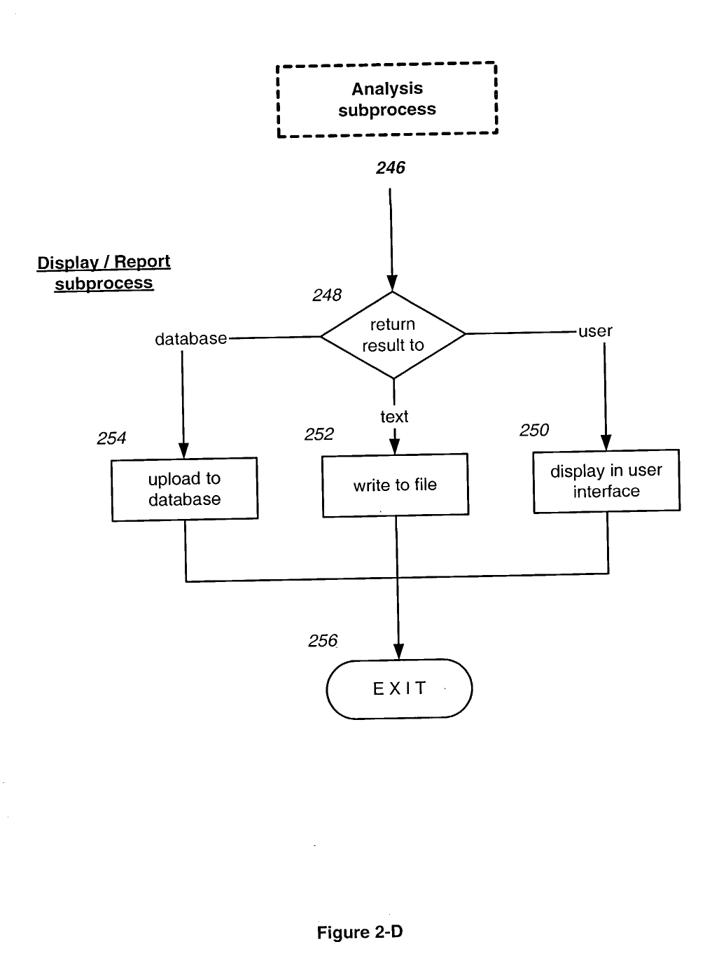

In one embodiment, the computer system further comprises a sequence comparison software for comparing the nucleic acid codes of a query sequence stored on a computer readable medium to a subject sequence which is also stored on a computer readable medium; or for comparing the polypeptide code of a query sequence stored on a computer readable medium to a subject sequence which is also stored on computer readable medium. A “sequence comparison software” refers to one or more programs that are implemented on the computer system to compare nucleotide and/or protein sequences with other nucleotide and/or sequences stored within the data storage means. The design of one example of a sequence comparison software is provided in FIGS. 2A, 2B, 2C and 2D.

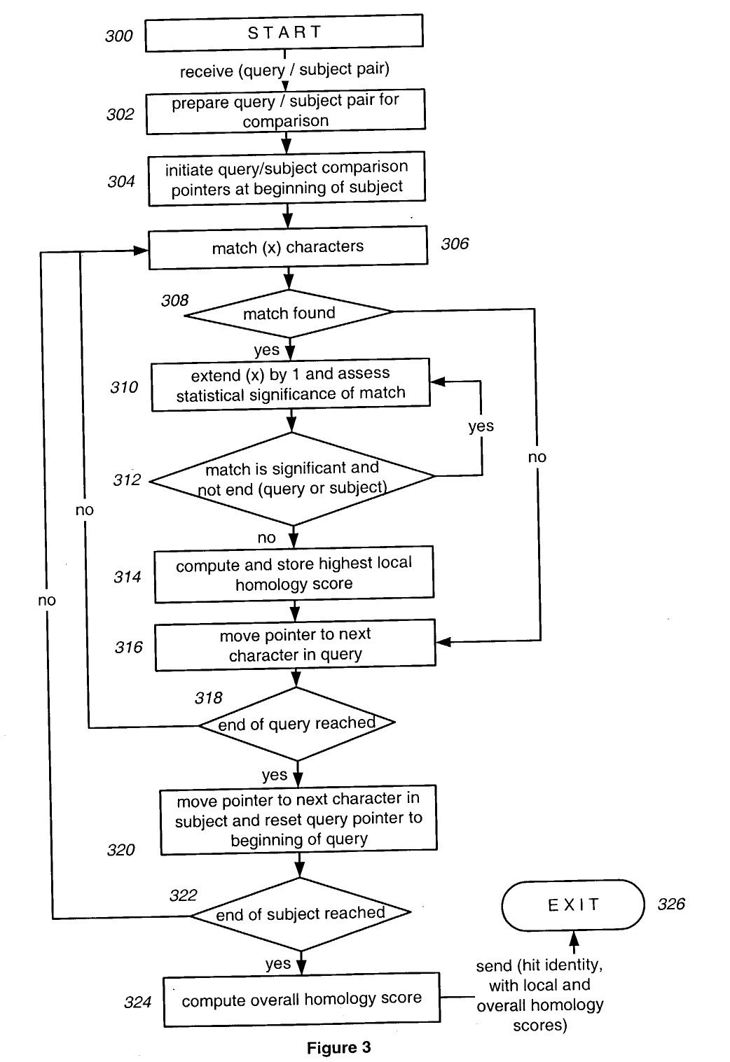

The sequence comparison software will typically employ one or more specialized comparator algorithms. Protein and/or nucleic acid sequence similarities may be evaluated using any of the variety of sequence comparator algorithms and programs known in the art. Such algorithms and programs include, but are no way limited to, TBLASTN, BLASTN, BLASTP, FASTA, TFASTA, CLUSTAL, HMMER, MAST, or other suitable algorithm known to those skilled in the art. (Pearson and Lipman, 1988, Proc. Natl. Acad. Sci USA 85(8): 2444-2448; Altschul et al., 1990, J. Mol. Biol. 215(3):403-410; Thompson et al., 1994, Nucleic Acids Res. 22(2):4673-4680; Higgins et al., 1996, Methods Enzymol. 266:383-402; Altschul et al., 1990, J. Mol. Biol. 215(3):403-410; Altschul et al., 1993, Nature Genetics 3:266-272; Eddy S. R., Bioinformatics 14:755-763, 1998; Bailey T L et al, J Steroid Biochem Mol Biol 1997 May; 62(1):29-44). One example of a comparator algorithm is illustrated in FIG. 3. Sequence comparator algorithms identified in this specification are particularly contemplated for use in this aspect of the invention.

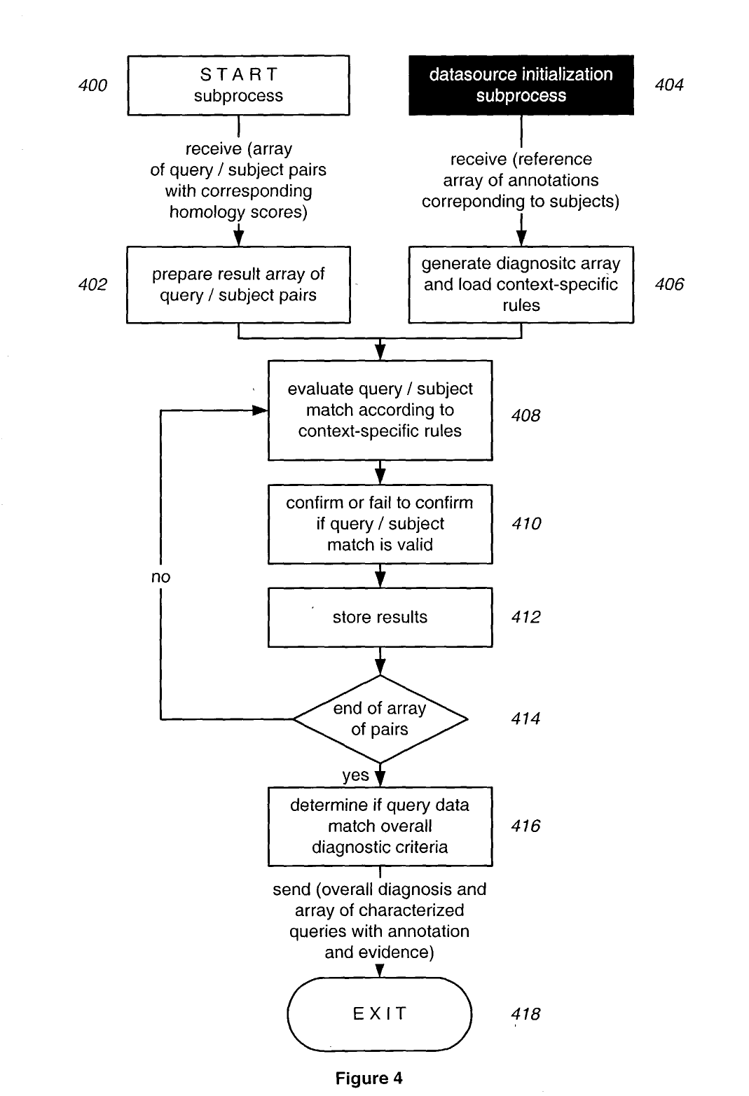

The sequence comparison software will typically employ one or more specialized analyzer algorithms. One example of an analyzer algorithm is illustrated in FIG. 4. Any appropriate analyzer algorithm can be used to evaluate similarities, determined by the comparator algorithm, between a query sequence and a subject sequence (referred to herein as a query/subject pair). Based on context specific rules, the annotation of a subject sequence may be assigned to the query sequence. A skilled artisan can readily determine the selection of an appropriate analyzer algorithm and appropriate context specific rules. Analyzer algorithms identified elsewhere in this specification are particularly contemplated for use in this aspect of the invention.

FIGS. 2A, 2B, 2C and 2D together provide a flowchart of one example of a sequence comparison software for comparing query sequences to a subject sequence. The software determines if a gene or set of genes represented by their nucleotide sequence, polypeptide sequence or other representation (the query sequence) is significantly similar to the one or more of the nucleic acid codes of SEQ ID NOS: 3, 5, 7, 9, 11, 13, 15, 17, 19, 21, 23, 25, 27, 29, 31, 33, 35, 37, 39 and the corresponding polypeptide codes of SEQ ID NOS: 2, 4, 6, 8, 10, 12, 14, 16, 18, 20, 22, 24, 26, 28, 30, 32, 34, 36, 38 of the invention (the subject sequence). The software may be implemented in the C or C++ programming language, Java, Perl or other suitable programming language known to a person skilled in the art.

One or more query sequence(s) are accessed by the program by means of input from the user 210, accessing a database 208 or opening a text file 206 as illustrated in the query initialization subprocess (FIG. 2A). The query initialization subprocess allows one or more query sequence(s) to be loaded into computer memory 122, or under control of the program stored on a disk drive 112 or other storage device in the form of a query sequence array 216. The query array 216 is one or more query nucleotide or polypeptide sequences accompanied by some appropriate identifiers.

A dataset is accessed by the program by means of input from the user 228, accessing a database 226, or opening a text file 224 as illustrated in the subject datasource initialization subprocess (FIG. 2B). The subject data source initialization process refers to the method by which a reference dataset containing one or more sequence selected from the nucleic acid codes of SEQ ID NOS: 3, 5, 7, 9, 11, 13, 15, 17, 19, 21, 23, 25, 27, 29, 31, 33, 35, 37, 39 and the corresponding polypeptide codes of SEQ ID NOS: 2, 4, 6, 8, 10, 12, 14, 16, 18, 20, 22, 24, 26, 28, 30, 32, 34, 36, 38 is loaded into computer memory 122, or under control of the program stored on a disk drive 112 or other storage device in the form of a subject array 234. The subject array 234 comprises one or more subject nucleotide or polypeptide sequences accompanied by some appropriate identifiers.

The comparison subprocess of FIG. 2C illustrates a process by which the comparator algorithm 238 is invoked by the software for pairwise comparisons between query elements in the query sequence array 216, and subject elements in the subject array 234. The “comparator algorithm” of FIG. 2C refers to the pair-wise comparisons between a query sequence and subject sequence, i.e. a query/subject pair from their respective arrays 216, 234. Comparator algorithm 238 may be any algorithm that acts on a query/subject pair, including but not limited to homology algorithms such as BLAST, Smith Waterman, Fasta, or statistical representation/probabilistic algorithms such as Markov models exemplified by HMMER, or other suitable algorithm known to one skilled in the art. Suitable algorithms would generally require a query/subject pair as input and return a score (an indication of likeness between the query and subject), usually through the use of appropriate statistical methods such as Karlin Altschul statistics used in BLAST, Forward or Viterbi algorithms used in Markov models, or other suitable statistics known to those skilled in the art.

The sequence comparison software of FIG. 2C also comprises a means of analysis of the results of the pair-wise comparisons performed by the comparator algorithm 238. The “analysis subprocess” of FIG. 2C is a process by which the analyzer algorithm 244 is invoked by the software. The “analyzer algorithm” refers to a process by which annotation of a subject is assigned to the query based on query/subject similarity as determined by the comparator algorithm 238 according to context-specific rules coded into the program or dynamically loaded at runtime. Context-specific rules are what the program uses to determine if the annotation of the subject can be assigned to the query given the context of the comparison. These rules allow the software to qualify the overall meaning of the results of the comparator algorithm 238.

In one embodiment, context-specific rules may state that for a set of query sequences to be considered representative of a rosaramicin biosynthetic locus, the comparator algorithm 238 must determine that the set of query sequences contains at least five query sequences that show a statistical similarity to a subject sequence corresponding to the polypeptide codes of SEQ ID NOS: 2, 4, 6, 8, 10, 12, 14, 16, 18, 20, 22, 24, 26, 28, 30, 32, 34, 36, 38. Of course preferred context specific rules may specify a wide variety of thresholds for identifying rosaramicin biosynthetic genes or rosaramicin-producing organisms without departing from the scope of the invention. Some thresholds contemplate that at least one query sequence in the set of query sequences show a statistical similarity to the nucleic acid code corresponding to 5, 6, 7, 8 or more of the polypeptide codes of SEQ ID NOS: 2, 4, 6, 8, 10, 12, 14, 16, 18, 20, 22, 24, 26, 28, 30, 32, 34, 36, 38. Other context specific rules set the level of homology required in each of the group and may be set at 70%, 80%, 85%, 90%, 95% or 98% in regards to any one or more of the subject sequences.

In another embodiment context-specific rules may state that for a query sequence to be considered indicative of a macrolide, the comparator algorithm 238 must determine that the query sequence shows a statistical similarity to subject sequences corresponding to a nucleic acid sequence code for a polypeptide of SEQ ID NO: 10, 12, 14, 16 and 18, polypeptides having at least 75% homology to a polypeptide of SEQ ID NOS: 10, 12, 14, 16 and 18 and fragment comprising at least 400 consecutive amino acids of the polypeptides of SEQ ID NOS: 10, 12, 14, 16 and 18. Of course preferred context specific rules may specify a wide variety of thresholds for identifying a macrolide protein without departing from the scope of the invention. Some context specific rules set level of homology required of the query sequence at 70%, 80%, 85%, 90%, 95% or 98%.

Thus, the analysis subprocess may be employed in conjunction with any other context specific rules and may be adapted to suit different embodiments. The principal function of the analyzer algorithm 244 is to assign meaning or a diagnosis to a query or set of queries based on context specific rules that are application specific and may be changed without altering the overall role of the analyzer algorithm 244.

Finally the sequence comparison software of FIG. 2 comprises a means of returning of the results of the comparisons by the comparator algorithm 238 and analyzed by the analyzer algorithm 244 to the user or process that requested the comparison or comparisons. The “display / report subprocess” of FIG. 2D is the process by which the results of the comparisons by the comparator algorithm 238 and analyses by the analyzer algorithm 244 are returned to the user or process that requested the comparison or comparisons. The results 240, 246 may be written to a file 252, displayed in some user interface such as a console, custom graphical interface, web interface, or other suitable implementation specific interface, or uploaded to some database such as a relational database, or other suitable implementation specific database. Once the results have been returned to the user or process that requested the comparison or comparisons the program exits.

The principle of the sequence comparison software of FIG. 2 is to receive or load a query or queries, receive or load a reference dataset, then run a pair-wise comparison by means of the comparator algorithm 238, then evaluate the results using an analyzer algorithm 244 to arrive at a determination if the query or queries bear significant similarity to the reference sequences, and finally return the results to the user or calling program or process.

FIG. 3 is a flow diagram illustrating one embodiment of comparator algorithm 238 process in a computer for determining whether two sequences are homologous. The comparator algorithm receives a query/subject pair for comparison, performs an appropriate comparison, and returns the pair along with a calculated degree of similarity.

Referring to FIG. 3, the comparison is initiated at the beginning of sequences 304. A match of (x) characters is attempted 306 where (x) is a user specified number. If a match is not found the query sequence is advanced 316 by one character with respect to the subject, and if the end of the query has not been reached 318 another match of (x) characters is attempted 306. Thus if no match has been found the query is incrementally advanced in entirety past the initial position of the subject. Once the end of the query is reached 318, the subject pointer is advanced by 1 character and the query pointer is set to the beginning of the query 320. If the end of the subject has been reached and still no matches have been found a null homology result score is assigned 324 and the algorithm returns the pair of sequences along with a null score to the calling process or program. The algorithm then exits 326. If instead a match is found 308, an extension of the matched region is attempted 310 and the match is analyzed statistically 312. The extension may be unidirectional or bidirectional. The algorithm continues in a loop extending the matched region and computing the homology score, giving penalties for mismatches taking into consideration that given the chemical properties of the amino acid side chains (in the case of comparisons) not all mismatches are equal. For example a mismatch of a lysine with an arginine both of which have basic side chains receive a lesser penalty than a mismatch between lysine and glutamate which has an acidic side chain. The extension loop stops once the accumulated penalty exceeds some user specified value, or of the end of either sequence is reached 312. The maximal score is stored 314, and the query sequence is advanced 316 by one character with respect to the subject, and if the end of the query has not been reached 318 another match of (x) characters is attempted 306. The process continues until the entire length of the subject has been evaluated for matches to the entire length of the query. All individual scores and alignments are stored 314 by the algorithm and an overall score is computed 324 and stored. The algorithm returns the pair of sequences along with local and global scores to the calling process or program. The algorithm then exits 326.

One example of comparator algorithm 238 algorithm may be represented in pseudocode as follows:

| | | |
|---|---|---|
| INPUT: | Q[m]: | query, m is the length |
| | S[n]: | subject, n is the length |
| | x: | x is the size of a segment |

START:
for each i in [1,n] do

for each j in [1,m] do

if ( j + x − 1 ) <= m and ( i + x −1 ) <= n then

if Q(j, j+x−1) = S(i, i+x−1) then

k=1;
while Q(j, j+x−1+k ) = S(i, i+x−1 + k) do

k++;

Store highest local homology

Compute overall homology score
Return local and overall homology scores
END.

The comparator algorithm 238 may be written for use on nucleotide sequences, in which case the scoring scheme would be implemented so as to calculate scores and apply penalties based on the chemical nature of nucleotides. The comparator algorithm 238 may also provide for the presence of gaps in the scoring method for nucleotide or polypeptide sequences.

BLAST is one implementation of the comparator algorithm 238. HMMER is another implementation of the comparator algorithm 238 based on Markov model analysis. In a HMMER implementation a query sequence would be compared to a mathematical model representative of a subject sequence or sequences rather than using sequence homology.

FIG. 4 is a flow diagram illustrating an analyzer algorithm 244 process for detecting the presence of a rosaramicin biosynthetic locus. The analyzer algorithm of FIG. 4 may be used in the process by which the annotation of a subject is assigned to the query based on their similarity as determined by the comparator algorithm 238 and according to context-specific rules coded into the program or dynamically loaded at runtime. Context sensitive rules are what determines if the annotation of the subject can be assigned to the query given the context of the comparison. Context specific rules set the thresholds for determining the level and quality of similarity that would be accepted in the process of evaluating matched pairs.

The analyzer algorithm 244 receives as its input an array of pairs that had been matched by the comparator algorithm 238. The array consists of at least a query identifier, a subject identifier and the associated value of the measure of their similarity. To determine if a group of query sequences includes sequences diagnostic of a rosaramicin biosynthetic gene cluster, a reference or diagnostic array 406 is generated by accessing a data source and retrieving rosaramicin specific information 404 relating to nucleic acid codes of SEQ ID NOS: 3, 5, 7, 9, 11, 13, 15, 17, 19, 21, 23, 25, 27, 29, 31, 33, 35, 37, 39 and the corresponding polypeptide codes of SEQ ID NOS: 2, 4, 6, 8, 10, 12, 14, 16, 18, 20, 22, 24, 26, 28, 30, 32, 34, 36, 38. Diagnostic array 406 consists at least of subject identifiers and their associated annotation. Annotation may include reference to the protein families ABCC, DATF, GTFA, MTFA, MTRA, NBPA, OXRB, OXRC, OXRH, PKSH, REGM, REGS, SURA and TESA. Annotation may also include information regarding presence in loci of a specific structural class or may include previously computed matches to other databases, for example databases of motifs.

Once the algorithm has successfully generated or received the two necessary arrays 402, 406, and holds in memory any context specific rules, each matched pair as determined by the comparator algorithm 238 can be evaluated. The algorithm will perform an evaluation 408 of each matched pair and based on the context specific rules confirm or fail to confirm the match as valid 410. In cases of successful confirmation of the match 410 the annotation of the subject is assigned to the query. Results of each comparison are stored 412. The loop ends when the end of the query/subject array is reached. Once all query/subject pairs have been evaluated against one or more of the nucleic acid codes of SEQ ID NOS: 3, 5, 7, 9, 11, 13, 15, 17, 19, 21, 23, 25, 27, 29, 31, 33, 35, 37, 39 and the polypeptide codes of SEQ ID NOS: 2, 4, 6, 8, 10, 12, 14, 16, 18, 20, 22, 24, 26, 28, 30, 32, 34, 36, 38 in the subject array, a final determination can be made if the query set of ORFs represents a rosaramicin locus 416. The algorithm then returns the overall diagnosis and an array of characterized query/subject pairs along with supporting evidence to the calling program or process and then terminates 418.

The analyzer algorithm 244 may be configured to dynamically load different diagnostic arrays and context specific rules. It may be used for example in the comparison of query/subject pairs with diagnostic subjects for other biosynthetic pathways, such as macrolide biosynthetic pathways.

Thus one embodiment of the present invention is a computer readable medium having stored thereon a sequence selected from the group consisting of a nucleic acid code of SEQ ID NOS: 3, 5, 7, 9, 11, 13, 15, 17, 19, 21, 23, 25, 27, 29, 31, 33, 35, 37, 39 and a polypeptide code of SEQ ID NOS: 2, 4, 6, 8, 10, 12, 14, 16, 18, 20, 22, 24, 26, 28, 30, 32, 34, 36, 38. Another aspect of the present invention is a computer readable medium having recorded thereon one or more nucleic acid codes of SEQ ID NOS: 3, 5, 7, 9, 11, 13, 15, 17, 19, 21, 23, 25, 27, 29, 31, 33, 35, 37, 39, preferably at least 2, 5, 10, 15, or 20 nucleic acid codes of SEQ ID NOS: 3, 5, 7, 9, 11, 13, 15, 17, 19, 21, 23, 25, 27, 29, 31, 33, 35, 37, 39. Another aspect of the invention is a computer readable medium having recorded thereon one or more of the polypeptide codes of SEQ ID NOS: 2, 4, 6, 8, 10, 12, 14, 16, 18, 20, 22, 24, 26, 28, 30, 32, 34, 36, 38, preferably at least 2, 5, 10, 15 or 20 polypeptide codes of SEQ ID NOS: 2, 4, 6, 8, 10, 12, 14, 16, 18, 20, 22, 24, 26, 28, 30, 32, 34, 36, 38.

Another embodiment of the present invention is a computer system comprising a processor and a data storage device wherein said data storage device has stored thereon a reference sequence selected from the group consisting of a nucleic acid code of SEQ ID NOS: 3, 5, 7, 9, 11, 13, 15, 17, 19, 21, 23, 25, 27, 29, 31, 33, 35, 37, 39 and a polypeptide code of SEQ ID NOS: 2, 4, 6, 8, 10, 12, 14, 16, 18, 20, 22, 24, 26, 28, 30, 32, 34, 36, 38.

Computer readable media include magnetically readable media, optically readable media, electronically readable media and magnetic/optical media. For example, the computer readable media may be a hard disk, a floppy disk, a magnetic tape, CD-ROM, Digital Versatile Disk (DVD), Random Access Memory (RAM), or Read Only Memory (ROM) as well as other types of media known to those skilled in the art.

The present invention will be further described with reference to the following examples; however, it is to be understood that the present invention is not limited to such examples.

### EXAMLPE 1

### Identification and Sequencing of a Rosaramicin Biosynthetic Locus in Micromonospora carbonacea var. Aurantiaca NRRL 2997

Micromonospora carbonacea var. aurantiaca NRRL 2997 was obtained from the Agricultural Research Service collection (National Center for Agricultural Utilization Research, 1815 N. University Street, Peoria, Ill. 61604) and cultured using standard microbiological techniques (Kieser et al., supra). This organism was propagated on oatmeal agar medium at 28 degrees Celsius for several days. For isolation of high molecular weight genomic DNA, cell mass from three freshly grown, near confluent 100 mm petri dishes was used. The cell mass was collected by gentle scraping with a plastic spatula. Residual agar medium was removed by repeated washes with STE buffer (75 mM NaCl; 20 mM Tris-HCl, pH 8.0; 25 mM EDTA). High molecular weight DNA was isolated by established protocols (Kieser et al. supra) and its integrity was verified by field inversion gel electrophoresis (FIGE) using the preset program number 6 of the FIGE MAPPER™ power supply (BIORAD). This high molecular weight genomic DNA served for the preparation of a small size fragment genomic sampling library (GSL), as well as a large size fragment cluster identification library (CIL). Both libraries contained randomly generated M. carbonacea genomic DNA fragments and, therefore, are representative of the entire genome of this organism.

For the generation of the GSL library, genomic DNA was randomly sheared by sonication. DNA fragments having a size range between 1.5 and 3 kb were fractionated on a agarose gel and isolated using standard molecular biology techniques (Sambrook et al., supra). The ends of the obtained DNA fragments were repaired using T4 DNA polymerase (Roche) as described by the supplier. This enzyme creates DNA fragments with blunt ends that can be subsequently cloned into an appropriate vector. The repaired DNA fragments were subcloned into a derivative of pBluescript SK+ vector (Stratagene) which does not allow transcription of cloned DNA fragments. This vector was selected as it contains a convenient polylinker region surrounded by sequences corresponding to universal sequencing primers such as T3, T7, SK, and KS (Stratagene). The unique EcoRV restriction site found in the polylinker region was used as it allows insertion of blunt-end DNA fragments. Ligation of the inserts, use of the ligation products to transform E. coli DH10B (Invitrogen) host and selection for recombinant clones were performed as previously described (Sambrook et al., supra). Plasmid DNA carrying the M. carbonacea genomic DNA fragments was extracted by the alkaline lysis method (Sambrook et al., supra) and the insert size of 1.5 to 3 kb was confirmed by electrophoresis on agarose gels. Using this procedure, a library of small size random genomic DNA fragments is generated that covers the entire genome of the studied microorganism. The number of individual clones that can be generated is infinite but only a small number is further analyzed to sample the microorganism's genome.

A CIL library was constructed from the M. carbonacea high molecular weight genomic DNA using the SuperCos-1 cosmid vector (Stratagene™). The cosmid arms were prepared as specified by the manufacturer. The high molecular weight DNA was subjected to partial digestion at 37 degrees Celsius with approximately one unit of Sau3Al restriction enzyme (New England Biolabs) per 100 micrograms of DNA in the buffer supplied by the manufacturer. This procedure generates random fragments of DNA ranging from the initial undigested size of the DNA to short fragments of which the length is dependent upon the frequency of the enzyme DNA recognition site in the genome and the extent of the DNA digestion by the enzyme. At various timepoints, aliquots of the digestion were transferred to new microfuge tubes and the enzyme was inactivated by adding a final concentration of 10 mM EDTA and 0.1% SDS. Aliquots judged by FIGE analysis to contain a significant fraction of DNA in the desired size range (30-50 kb) were pooled, extracted with phenol/chloroform (1:1 vol:vol), and pelletted by ethanol precipitation. The 5′ ends of Sau3Al DNA fragments were dephosphorylated using alkaline phosphatase (Roche) according to the manufacturer's specifications at 37 degrees Celcius for 30 min. The phosphatase was heat inactivated at 70 degrees Celcius for 10 min and the DNA was extracted with phenol/chloroform (1:1 vol:vol), pelletted by ethanol precipitation, and resuspended in sterile water. The dephosphorylated Sau3Al DNA fragments were then ligated overnight at room temperature to the SuperCos-1 cosmid arms in a reaction containing approximately four-fold molar excess SuperCos-1 cosmid arms. The ligation products were packaged using Gigapack® III XL packaging extracts (Stratagene™) according to the manufacturer's specifications. The CIL library consisted of 864 isolated cosmid clones in E. coli DH10B (Invitrogen). These clones were picked and inoculated into nine 96-well microtiter plates containing LB broth (per liter of water: 10.0 g NaCl; 10.0 g tryptone; 5.0 g yeast extract) which were grown overnight and then adjusted to contain a final concentration of 25% glycerol. These microtiter plates were stored at −80 degrees Celcius and served as glycerol stocks of the CIL library. Duplicate microtiter plates were arrayed onto nylon membranes as follows. Cultures grown on microtiter plates were concentrated by pelleting and resuspending in a small volume of LB broth. A 3×3 grid (96-pin) was arrayed onto nylon membranes. These membranes representing the complete CIL library were then layered onto LB agar and incubated ovenight at 37 degrees Celcius to allow the colonies to grow. The membranes were layered onto filter paper pre-soaked with 0.5 N NaOH/1.5 M NaCl for 10 min to denature the DNA and then neutralized by transferring onto filter paper pre-soaked with 0.5 M Tris (pH 8)/1.5 M NaCl for 10 min. Cell debris was gently scraped off with a plastic spatula and the DNA was crosslinked onto the membranes by UV irradiation using a GS GENE LINKER™ UV Chamber (BIORAD). Considering an average size of 8 Mb for an actinomycete genome and an average size of 35 kb of genomic insert in the CIL library, this library represents roughly a 4-fold coverage of the microorganism's entire genome.

The GSL library was analyzed by sequence determination of the cloned genomic DNA inserts. The universal primers KS or T7, referred to as forward (F) primers, were used to initiate polymerization of labeled DNA. Extension of at least 700 bp from the priming site can be routinely achieved using the TF, BDT v2.0 sequencing kit as specified by the supplier (Applied Biosystems). Sequence analysis of the small genomic DNA fragments (Genomic Sequence Tags, GSTs) was performed using a 3700 ABI capillary electrophoresis DNA sequencer (Applied Biosystems). The average length of the DNA sequence reads was ˜700 bp. Further analysis of the obtained GSTs was performed by sequence homology comparison to various protein sequence databases. The DNA sequences of the obtained GSTs were translated into amino acid sequences and compared to the National Center for Biotechnology Information (NCBI) nonredundant protein database and the proprietary Ecopia natural product biosynthetic gene Decipher™ database using previously described algorithms (Altschul et al., supra). Sequence similarity with known proteins of defined function in the database enables one to make predictions on the function of the partial protein that is encoded by the translated GST.

A total of 437 M. carbonacea GSTs were generated using the forward sequencing primer and analyzed by sequence comparison using the Blast algorithm (Altschul et al., supra). Sequence alignments displaying an E value of at least e-5 were considered as significantly homologous and retained for further evaluation. GSTs showing similarity to a gene of interest can be at this point selected and used to identify larger segments of genomic DNA from the CIL library that include the gene(s) of interest. Polyketide natural products are often synthesized by type I polyketide synthases (PKSs). Several forward GST reads were identified as portions of PKS genes. For example, one such GST encoded an internal portion of a PKS acyl transferase (AT) domain in the antisense orientation relative to the sequencing primer. The GSL clone from which this GST was obtained was also sequenced using the reverse sequencing primer and was found to encode the N-terminal portion of a PKS ketosynthase (KS) domain in the sense orientation relative to the sequencing primer. Based on the sequence of the forward read of this GSL clone, a 20mer oligonucleotide was designed for use as a probe to identify and isolate CIL clones which harbored the sequences of interest.

Hybridization oligonucleotide probes were radiolabeled with P³² using T4 polynucleotide kinase (New England Biolabs) in 15 microliter reactions containing 5 picomoles of oligonucleotide and 6.6 picomoles of [γ-P³²]ATP in the kinase reaction buffer supplied by the manufacturer. After 1 hour at 37 degrees Celcius, the kinase reaction was terminated by the addition of EDTA to a final concentration of 5 mM. The specific activity of the radiolabeled oligonucleotide probes was estimated using a Model 3 Geiger counter (Ludlum Measurements Inc., Sweetwater, Tex.) with a built-in integrator feature. The radiolabeled oligonucleotide probes were heat-denatured by incubation at 85 degrees Celcius for 10 minutes and quick-cooled in an ice bath immediately prior to use.

The CIL library membranes were pretreated by incubation for at least 2 hours at 42 degrees Celcius in Prehyb Solution (6× SSC; 20 mM NaH₂PO₄; 5× Denhardt's; 0.4% SDS; 0.1 mg/ml sonicated, denatured salmon sperm DNA) using a hybridization oven with gentle rotation. The membranes were then placed in Hyb Solution (6× SSC; 20 mM NaH₂PO₄; 0.4% SDS; 0.1 mg/ml sonicated, denatured salmon sperm DNA) containing 1×10⁶ cpm/ml of radiolabeled oligonucleotide probe and incubated overnight at 42 degrees Celcius using a hybridization oven with gentle rotation. The next day, the membranes were washed with Wash Buffer (6×SSC, 0.1% SDS) for 45 minutes each at 46, 48, and 50 degrees Celcius using a hybridization oven with gentle rotation. The membranes were then exposed to X-ray film to visualize and identify the positive cosmid clones. Positive clones were identified, cosmid DNA was extracted from 30 ml cultures using the alkaline lysis method (Sambrook et al., supra) and the inserts were entirely sequenced using a shotgun sequencing approach (Fleischmann et al., Science, 269:496-512).

Sequencing reads were assembled using the Phred-Phrap™ algorithm (University of Washington, Seattle, U.S.A.) recreating the entire DNA sequence of the cosmid insert. Reiterations of hybridizations of the CIL library with probes derived from the ends of the original cosmid allow indefinite extension of sequence information on both sides of the original cosmid sequence until the complete sought-after gene cluster is obtained. Three overlapping cosmid clones that were either directly identified by the original oligonucleotide probe (derived from the GSL clone) or by probes derived from the ends of the original cosmids have been completely sequenced to provide over 60 Kb of genetic information. Subsequently, the forward and reverse reads of the GSL clone from which the original oligonucleotide probe was derived were mapped to a region of the rosaramicin biosynthetic locus that encodes a portion of the PKS gene identified herein as ORF 7, more specifically nucleotides encoding amino acids 1531 kb to 2416 approximately. This corresponds to a GSL clone with an insert size of approximately 2.6 kb, in good agreement with the selected size range of 1.5-3 kb described above. The sequence of these cosmids and analysis of the proteins encoded by them undoubtedly demonstrated that the gene cluster obtained was indeed responsible for the production of a glycosylated macrolide consistent with the known structure of rosaramicin, which was not previously reported to be produced by M. carbonacea var aurantiaca NRRL 2997.

### EXAMPLE 2

### Genes and Proteins Involved in Biosynthesis of Rosaramicin

The rosaramicin locus includes the 60196 base pairs provided in SEQ ID NO: 1 and contains the 19 ORFs provided SEQ ID NOS: 3, 5, 7, 9, 11, 13, 15, 17, 19, 21, 23, 25, 27, 29, 31, 33, 35, 37, 39. More than 19 kilobases of DNA sequence were analyzed on each side of the rosaramicin locus and these regions contain primary metabolic genes. The accompanying sequence listing provides the nucleotide sequence of the 19 ORFs regulating the biosynthesis of rosaramicin and the corresponding deduced polypeptides, wherein ORF 1 (SEQ ID NO: 3) represents the polynucleotide drawn from residues 1 to 1683 (sense strand) of SEQ ID NO: 1; QRF 2 (SEQ ID NO: 5) represents the polynucleotide drawn from residues 2522 to 1728 (antisense strand) of SEQ ID NO: 1; ORF 3 (SEQ ID NO: 7) represents the polynucleotide drawn from residues 3861 to 2629 (antisense strand) of SEQ ID NO: 1; ORF 4 (SEQ ID NO: 9) represents the polynucleotide drawn from residues 4365 to 5573 (sense strand) of SEQ ID NO: 1; ORF 5 (SEQ ID NO: 11) represents the polynucleotide drawn from residues 5702 to 19117 (sense strand) of SEQ ID NO: 1; ORF 6 (SEQ ID NO: 13) represents the polynucleotide drawn from residues 19144 to 24921 (sense strand) of SEQ ID NO: 1; ORF 7 (SEQ ID NO: 15) represents the polynucleotide drawn from residues 24993 to 36230 (sense strand) of SEQ ID NO: 1; ORF 8 (SEQ ID NO: 17) represents the polynucleotide drawn from residues 36292 to 41016 (sense strand) of SEQ ID NO: 1; ORF 9 (SEQ ID NO: 19) represents the polynucleotide drawn from residues 41049 to 46403 (sense strand) of SEQ ID NO: 1; ORF 10 (SEQ ID NO: 21) represents the polynucleotide drawn from residues 46400 to 47794 (sense strand) of SEQ ID NO: 1; ORF 11 (SEQ ID NO: 23) represents the polynucleotide drawn from residues 47794 to 49083 (sense strand) of SEQ ID NO: 1; ORF 12 (SEQ ID NO: 25) represents the polynucleotide drawn from residues 49092 to 49814 (sense strand) of SEQ ID NO: 1; ORF 13 (SEQ ID NO: 27) represents the polynucleotide drawn from residues 49868 to 51226 (sense strand) of SEQ ID NO: 1; ORF 14 (SEQ ID NO: 29) represents the polynucleotide drawn from residues 51506 to 53416 (sense strand) of SEQ ID NO: 1; ORF 15 (SEQ ID NO: 31) represents the polynucleotide drawn from residues 54569 to 53358 (antisense strand) of SEQ ID NO: 1; ORF 16 (SEQ ID NO: 33) represents the polynucleotide drawn from residues 54897 to 56342 (sense strand) of SEQ ID NO: 33; ORF 17 (SEQ ID NO: 35) represents the polynucleotide drawn from residues 56408 to 57634 (sense strand) of SEQ ID NO: 1; ORF 18 (SEQ ID NO: 37) represents the polynucleotide drawn from residues 57657 to 59123 (sense strand) of SEQ ID NO: 1; ORF 19 (SEQ ID NO: 39) represents the polynucleotide drawn from residues 59363 to 60196 (sense strand) of SEQ ID NO: 1.

Some open reading frames listed herein initiate with non-standard initiation codons (e.g. GTG-Valine or CTG-Leucine) rather than the standard initiation codon ATG, namely ORFs 1, 6, 7, 10, 14 and 18. All ORFs are listed with the appropriate M, V or L amino acids at the amino-terminal position to indicate the specificity of the first codon of the ORF. It is expected, however, that in all cases the biosynthesized protein will contain a methionine residue, and more specifically a formylmethionine residue, at the amino terminal position, in keeping with the widely accepted principle that protein synthesis in bacteria initiates with methionine (formylmethionine) even when the encoding gene specifies a non-standard initiation codon (e.g. Stryer, Biochemistry 3^{rd} edition, 1998, W.H. Freeman and Co., New York, pp. 752-754).

Three deposits, namely E. coli DH10B (O10CK) strain, E. coli DH10B (O10CF) strain and E. coli DH10B (O10CJ) strain each harbouring a cosmid clone of a partial biosynthetic locus for rosaramicin from Micromonospora carbonacea subsp. aurantiaca have been deposited with the International Depositary Authority of Canada, Bureau of Microbiology, Health Canada, 1015 Arlington Street, Winnipeg, Manitoba, Canada R3E 3R2 on Jul. 10, 2002 and were assigned deposit accession number IDAC 100702-1, 100702-2 and 100702-3 respectively. The E. coli strain deposits are referred to herein as “the deposited strains”.

The cosmids harbored in the deposited strains comprise a complete biosynthetic locus for rosaramicin. The sequence of the polynucleotides comprised in the deposited strains, as well as the amino acid sequence of any polypeptide encoded thereby are controlling in the event of any conflict with any description of sequences herein.

The deposit of the deposited strains has been made under the terms of the Budapest Treaty on the International Recognition of the Deposit of Micro-organisms for Purposes of Patent Procedure. The deposited strains will be irrevocably and without restriction or condition released to the public upon the issuance of a patent. The deposited strains are provided merely as convenience to those skilled in the art and are not an admission that a deposit is required for enablement, such as that required under 35 U.S.C. §112. A license may be required to make, use or sell the deposited strains, and compounds derived therefrom, and no such license is hereby granted.

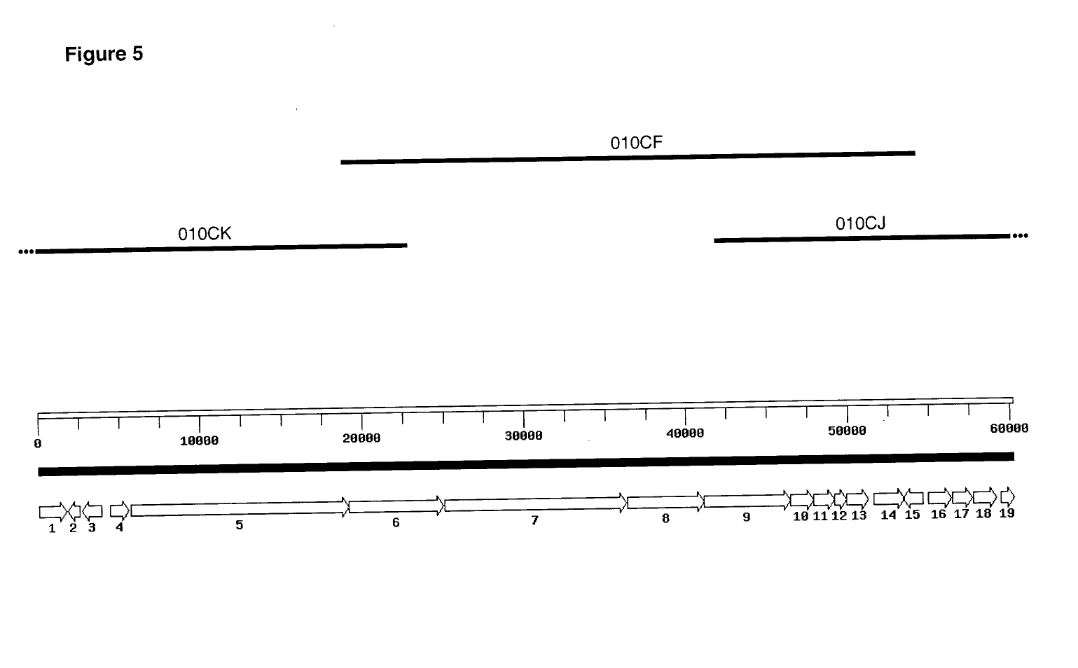

The order and relative position of the 19 open reading frames and the corresponding polypeptides of the biosynthetic locus for rosaramicin are provided in FIG. 5. The arrows represent the orientatation of the ORFs of the rosaramicin biosynthetic locus. The top line in FIG. 5 provides a scale in kilobase pairs. The black bars depict the part of the locus covered by each of the deposited cosmids O10CK, O10CF and O10CJ.

In order to identify the function of the genes in the rosaramicin locus, SEQ ID NOS: 2, 4, 6, 8, 10, 12, 14, 16, 18, 20, 22, 24, 26, 28, 30, 32, 34, 36, 38 were compared, using the BLASTP version 2.2.1 algorithm with the default parameters, to sequences in the National Center for Biotechnology Information (NCBI) nonredundant protein database and the DECIPHER™ database of microbial genes, pathways and natural products (Ecopia BioSciences Inc. St.-Laurent, QC, Canada).

The accession numbers of the top GenBank hits of this BLAST analysis are presented in Table 2 along with the corresponding E value. The E value relates the expected number of chance alignments with an alignment score at least equal to the observed alignment score. An E value of 0.00 indicates a perfect homolog or nearly perfect homolog. The E values are calculated as described in Altschul et al. J. Mol. Biol., October 5; 215(3) 403-10. The E value assists in the determination of whether two sequences display sufficient similarity to justify an inference of homology.

TABLE 2

| ORF | | | | | | | |
|---|---|---|---|---|---|---|---|
| no. | Family | #aa | GenBank homology | probability | % identity | % similarity | proposed function of GenBank match |
| 1 | ABCC | 560 | S25202, 550aa | 1e-144 | 284/542 (52.4%) | 337/542 (62.18%) | spiramycin-resistance protein, Streptomyces |
| | | | | | | | ambofaciens |
| | | | AAC32027.1, 551aa | 1e-142 | 275/543 (50.64%) | 341/543 (62.8%) | carbomycin resistance protein, Streptomyces |
| | | | | | | | thermotolerans |
| | | | S67863, 569aa | 1e-126 | 259/553 (46.84%) | 323/553 (58.41%) | oleandomycin resistance protein, Streptomyces |
| | | | | | | | antibioticus |
| 2 | TESA | 264 | BAB69315.1, 255aa | 3e-52 | 109/232 (46.98%) | 131/232 (56.47%) | thioesterase, Streptomyces avermitilis |
| | | | S49055, 253aa | 9e-48 | 110/238 (46.22%) | 129/238 (54.2%) | thioesterase, Streptomyces fradiae |
| | | | AAC01736.1, 254aa | 8e-47 | 101/229 (44.1%) | 125/229 (54.59%) | thioesterase, Amycolatopsis mediterranei |
| 3 | OXRC | 410 | S49051, 417aa | 1e-168 | 282/394 (71.57%) | 335/394 (85.03%) | cytochrome P450, Streptomyces fradiae |
| | | | BAB83674.1, 402aa | 2e-73 | 161/384 (41.93%) | 222/384 (57.81%) | cytochrome P450 monooxygenase, |
| | | | | | | | Streptomyces virginiae |
| | | | B40634, 412aa | 3e-73 | 161/393 (40.97%) | 218/393 (55.47%) | erythromycin monooxygenase, |
| | | | | | | | Saccharopolyspora erythraea |
| 4 | OXRC | 402 | AAA92553.1, 407aa | 4e-95 | 187/400 (46.75%) | 242/400 (60.5%) | cytochrome P450, Streptomyces antibioticus |
| | | | AAD28449.1, 410aa | 8e-85 | 166/399 (41.6%) | 229/399 (57.39%) | cytochrome P450, Streptomyces lavendulae |
| | | | S51594, 397aa | 9e-81 | 161/398 (40.45%) | 223/398 (56.03%) | cytochrome P450, Micromonospora |
| | | | | | | | griseorubida |
| 5 | PKSH | 4471 | BAA76543.1, 4290aa | 0.0 | 1488/2961 (50.25%) | 1726/2961 (58.29%) | polyketide synthase, Streptomyces griseorubida |
| | | | T17409, 4613aa | 0.0 | 1405/3027 (46.42%) | 1696/3027 (56.03%) | polyketide synthase, Streptomyces venezuelae |
| | | | AAB66504.1, 4472aa | 0.0 | 1275/2459 (51.85%) | 1483/2459 (60.31%) | tylactone polyketide synthase, Streptomyces |
| | | | | | | | fradiae |
| 6 | PKSH | 1925 | AAB66505.1, 1864aa | 0.0 | 923/1883 (49.02%) | 1087/1883 (57.73%) | tylactone polyketide synthase, Streptomyces |
| | | | | | | | fradiae |
| | | | AAC46025.1, 1839aa | 0.0 | 847/1897 (44.65%) | 1027/1897 (54.14%) | polyketide synthase, Streptomyces caelestis |
| | | | AAK73514.1, 10917aa | 0.0 | 834/1810 (46.08%) | 1015/1810 (56.08%) | AmphC polyketide synthase, Streptomyces |
| | | | | | | | nodosus |
| 7 | PKSH | 3745 | AAB66506.1, 3729aa | 0.0 | 2004/3782 (52.99%) | 2283/3782 (60.36%) | tylactone polyketide synthase, Streptomyces |
| | | | | | | | fradiae |
| | | | T17410, 3739aa | 0.0 | 1417/3144 (45.07%) | 1730/3144 (55.03%) | polyketide synthase, Streptomyces venezuelae |
| | | | AAG13918.1, 3562aa | 0.0 | 1269/3054 (41.55%) | 1588/3054 (52%) | 6-deoxyerythronolide B synthase, |
| | | | | | | | Micromonospora megalomicea |
| 8 | PKSH | 1574 | AAB66507.1, 1611aa | 0.0 | 756/1521 (49.7%) | 869/1521 (57.13%) | tylactone polyketide synthase, Streptomyces |
| | | | | | | | fradiae |
| | | | BAB69192.1, 6146aa | 0.0 | 723/1527 (47.35%) | 839/1527 (54.94%) | polyketide synthase, Streptomyces avermitilis |
| | | | AAG2366.1, 3170aa | 0.0 | 721/1556 (46.34%) | 847/1556 (54.43%) | polyketide synthase, Saccharopolyspora |
| | | | | | | | spinosa |
| 9 | PKSH | 1784 | AAB66508.1, 1841aa | 0.0 | 956/1832 (52.18%) | 1079/1832 (58.9%) | tylactone polyketide synthase, Streptomyces |
| | | | | | | | fradiae |
| | | | BAB69307.1, 3352aa | 0.0 | 726/1546 (46.96%) | 855/1546 (55.3%) | polyketide synthase, Streptomyces avermitilis |
| | | | AAF71766.1, 9477aa | 0.0 | 725/1614 (44.92%) | 869/1614 (53.84%) | Nysl polyketide synthase, Streptomyces noursei |
| 10 | OXRB | 464 | CAA57471.1, 423aa | 6e-55 | 161/422 (38.15%) | 182/422 (43.13%) | NDP hexose 3,4 isomerase, Streptomyces |
| | | | | | | | fradiae |
| | | | AAF73456.1, 443aa | 3e-36 | 138/430 (32.09%) | 166/430 (38.6%) | AknT, Streptomyces galilaeus |
| | | | AAD15266.1, 438aa | 1e-27 | 127/421 (30.17%) | 150/421 (35.63%) | dnQ, Streptomyces peucetius |
| 11 | GTFA | 429 | CAA57472.2, 452aa | 1e-148 | 258/421 (61.28%) | 315/421 (74.82%) | glycosyltransferase, Streptomyces fradiae |
| | | | AAC68677.1, 426aa | 1e-126 | 237/426 (55.63%) | 289/426 (67.84%) | glycosyl transferase, Streptomyces venezuelae |
| | | | CAA05642.1, 426aa | 1e-125 | 230/424 (54.25%) | 293/424 (69.1%) | glycosyltransferase, Streptomyces antibioticus |
| 12 | MTFA | 240 | CAA57473.2, 255aa | 8e-74 | 132/234 (56.41%) | 155/234 (66.24%) | N-methyltransferase, Streptomyces fradiae |
| | | | CAA05643.1, 246aa | 2e-72 | 130/233 (55.79%) | 150/233 (64.38%) | methyltransferase, Streptomyces antibioticus |
| | | | AAC68678.1, 237aa | 1e-67 | 125/235 (53.19%) | 147/235 (62.55%) | N,N-dimethyltransferase, Streptomyces |
| | | | | | | | venezuelae |
| 13 | OXRH | 452 | NP_630556.1, 447aa | 0.0 | 344/440 (78.18%) | 386/440 (87.73%) | crotonyl CoA reductase, Streptomyces |
| | | | | | | | coelicolor |
| | | | S72400, 447aa | 0.0 | 342/440 (77.73%) | 381/440 (86.59%) | trans-2-enoyl-CoA reductase, Streptomyces |
| | | | | | | | collinus |
| | | | CAA57474.2, 449aa | 0.0 | 344/427 (80.56%) | 375/427 (87.82%) | crotonyl CoA reductase, Streptomyces fradiae |
| 14 | REGS | 636 | S25203, 604aa | 1e-92 | 219/583 (37.56%) | 290/583 (49.74%) | smR regulator, Streptomyces ambofaciens |
| | | | NP_625307.1, 634aa | 1e-63 | 189/612 (30.88%) | 264/612 (43.14%) | hypothetical protein, Streptomyces coelicolor |
| | | | NP_630273.1, 569aa | 4e-12 | 69/211 (32.7%) | 93/211 (44.08%) | putative regulatory protein, Streptomyces |
| | | | | | | | coelicolor |
| 15 | REGM | 403 | AAF29380.1, 430aa | 1e-99 | 192/386 (49.74%) | 236/386 (61.14%) | TylR regulator, Streptomyces fradiae |
| | | | JC2032, 387aa | 1e-78 | 170/387 (43 93%) | 219/387 (56 59%) | regulatory protein, Streptomyces sp |
| 16 | NBPA | 481 | NP_629920.1, 497aa | 1e-160 | 293/431 (67.98%) | 331/431 (76.8%) | hypothetical protein, Streptomyces coelicolor |
| | | | NP_217241.1, 495aa | 1e-135 | 261/438 (59.59%) | 303/438 (69.18%) | putative HflX GTP-binding protein, |
| | | | | | | | Mycobacterium tuberculosis |
| | | | NP_337300.1, 556aa | 1e-135 | 261/438 (59.59%) | 303/438 (69.18%) | GTP-binding protein, Mycobacterium |
| | | | | | | | tuberculosis |
| 17 | DATF | 408 | T51108, 393aa | 1e-149 | 259/392 (66.07%) | 304/392 (77.55%) | dehydratase, Streptomyces antibioticus |
| | | | AAC68684.1, 415aa | 1e-144 | 259/392 (66.07%) | 303/392 (77.3%) | 4-dehydrase, Streptomyces venezuelae |
| | | | AAB84075.1, 401aa | 1e-140 | 245/396 (61.87%) | 298/396 (75.25%) | EryCIV, Saccharopolyspora erythraea |
| 18 | SURA | 488 | AAC68683.1, 485aa | 0.0 | 313/480 (65.21%) | 367/480 (76.46%) | putative reductase, Streptomyces venezuelae |
| | | | T51109, 485aa | 1e-179 | 310/479 (64.72%) | 369/479 (77.04%) | probable reductase, Streptomyces antibioticus |
| | | | CAA72085.1, 489aa | 1e-168 | 294/474 (62.03%) | 352/474 (74.26%) | EryCV, Saccharopolyspora erythraea |
| 19 | MTRA | 277 | T17407, 322aa | 5e-95 | 167/251 (66.53%) | 198/251 (78.88%) | rRNA methyltransferase, Streptomyces |
| | | | | | | | venezuelae |
| | | | S28985, 278aa | 2e-73 | 143/249 (57.43%) | 173/249 (69.48%) | lincomycin resistance protein, Streptomyces |
| | | | | | | | lincolnensis |
| | | | P43433, 311aa | 4e-66 | 130/247 (52.63%) | 164/247 (66.4%) | mycinamycin resistance protein, |
| | | | | | | | Micromonospora griseorubida |

### EXAMPLE 3

### Formation of Rosaramicin

The chemical structure of rosaramicin is a 16-membered macrolide having an epoxide, an aldehyde and a deoxyamino sugar. The rosaramicin locus includes five polyketide synthase (PKS) Type I genes. ORF 5 represents a PKS Type I gene having a domain arrangement of KS-AT-ACP-KS-AT-KR-ACP-KS-AT-DH-KR-ACP. ORF 6 represents a PKS Type 1 gene having a domain arrangement of KS-AT-DH-KR-ACP. ORF 7 represents a PKS Type I gene having a domain arrangement of KS-AT-KR-ACP-KS-AT-DH-ER-KR-ACP. ORF 8 represents a PKS Type I gene having a domain arrangement of KS-AT-KR-ACP. ORF 9 represents a PKS Type I gene having a domain arrangement of KS-AT-KR-ACP-Te.

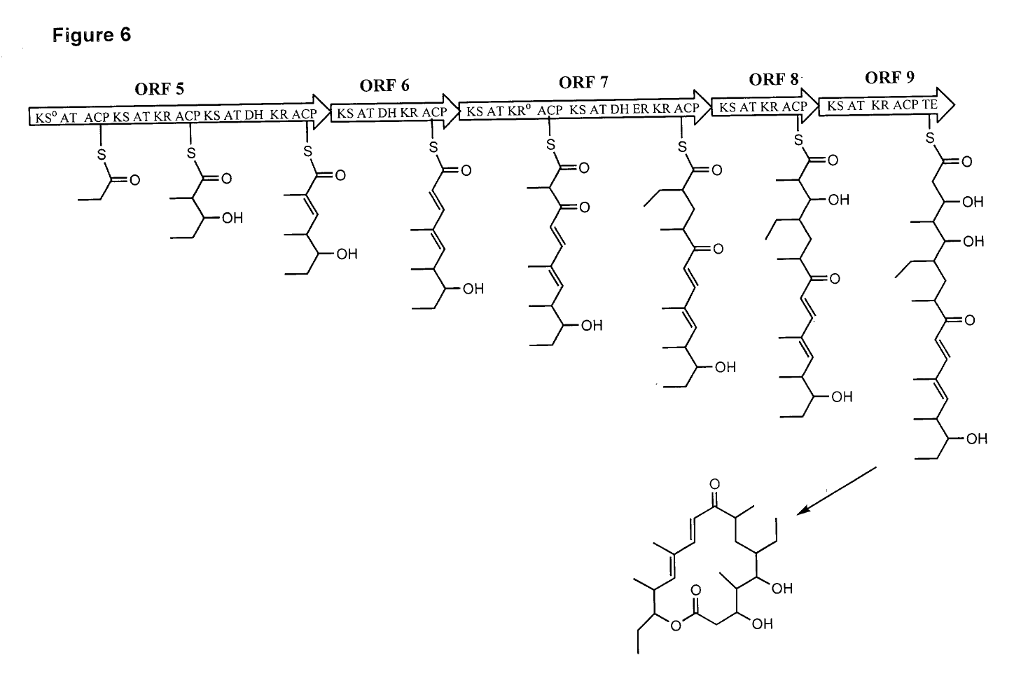

While not intending to be limited to any particular mode of action or biosynthetic scheme, the gene products of the invention can explain the synthesis of rosaramicin. ORFs 5, 6, 7, 8, and 9 constitute a polyketide synthase system that assembles the core polyketide precursor of rosaramicin. FIG. 6 highlights schematically the series of reactions catalyzed by this polyketide synthase system based on the correlation between the deduced domain architecture and the polyketide core of rosaramicin. Type I PKS domains and the reactions they carry out are well known to those skilled in the art and well documented in the literature, see for example, Hopwood (1997) Chem. Rev. Vol 97 pp. 2465-2497.

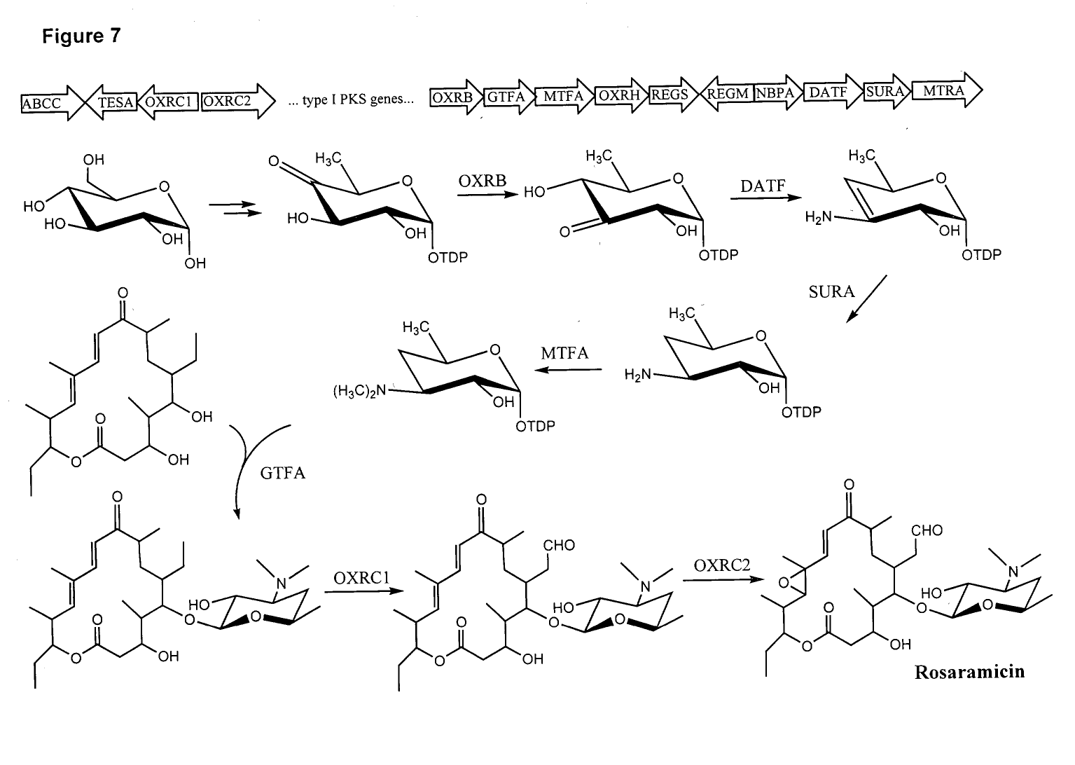

FIG. 7 depicts a proposed biochemical pathway involving the OXRB, DATF, SURA, MTFA gene products for the formation of the deoxyamino sugar. This sugar is transferred to the core polyketide precursor of rosaramicin by the GTFA gene product. Also depicted in FIG. 7 are the oxidation reactions carried out by two cytochrome P450 monooxygenases OXRC1 and OXRC2, referring to ORFs 3 and 4, respectively. OXRC1 is expected to catalyze the formation of an aldehyde while OXRC2 is expected to catalyze the formation of an epoxide. While FIG. 7 proposes one scheme in regard to timing of the glycosylation and oxidation reactions catalyzed by the GTFA, OXRC1 and OXRC2, the invention does not reside in the actual timing and order of the reactions, which may be different then that depicted in FIG. 7.

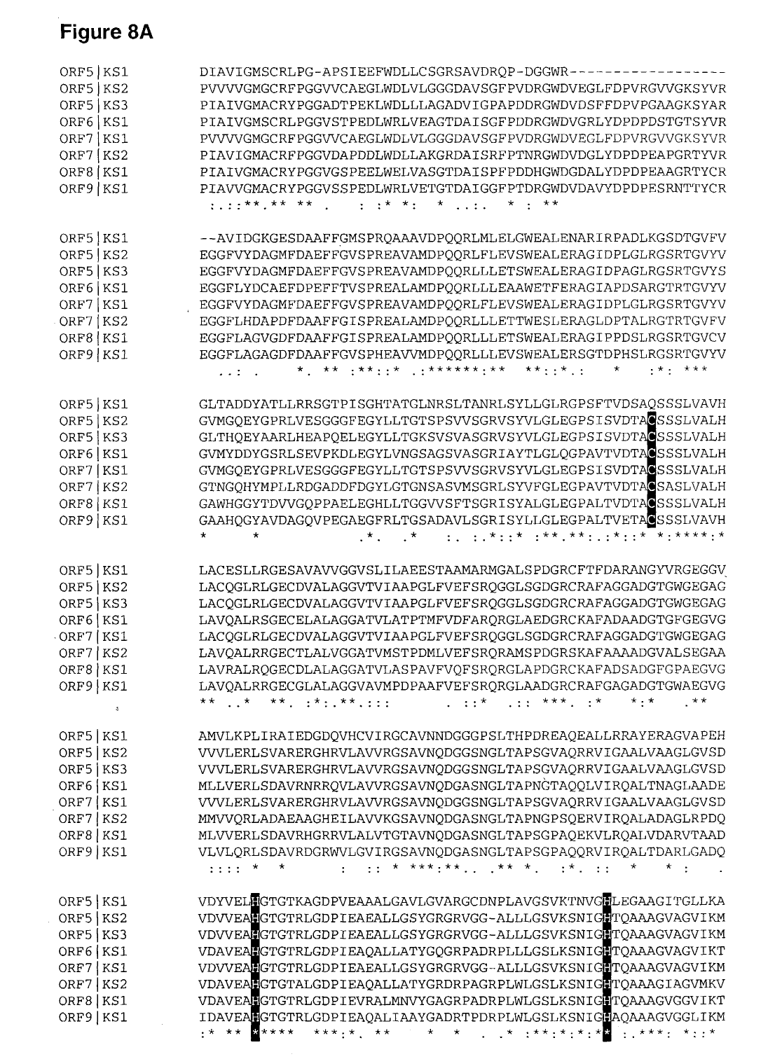

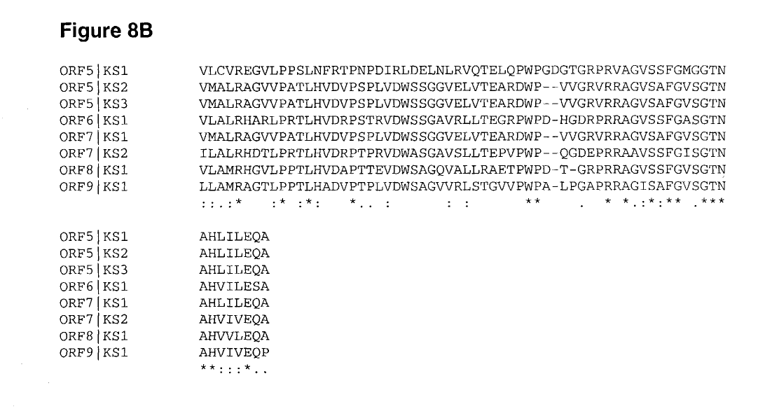

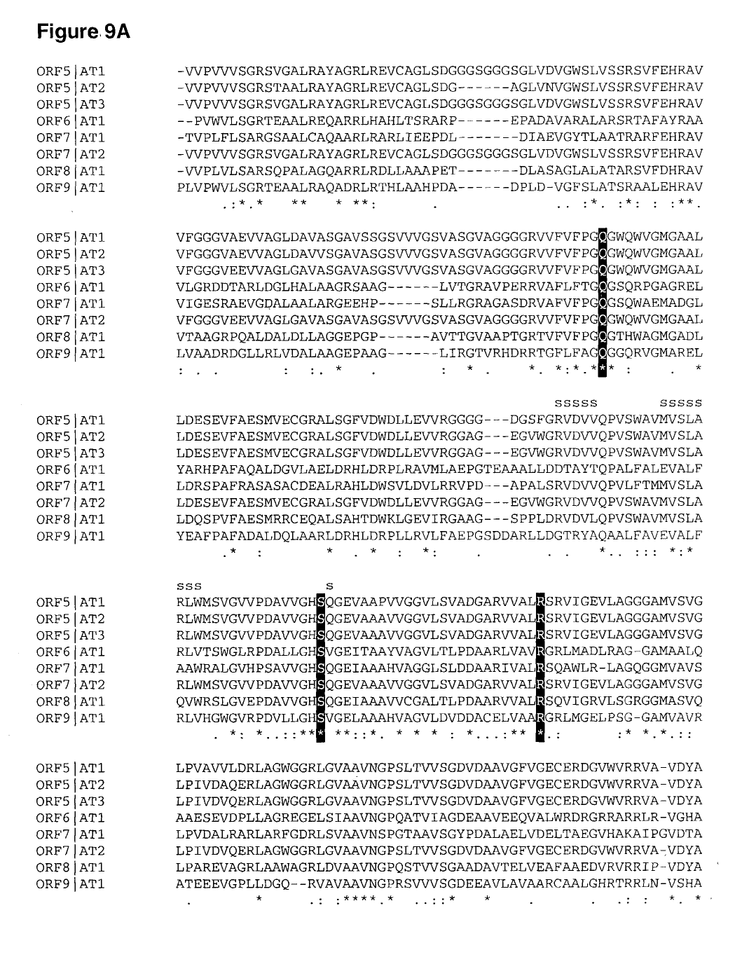

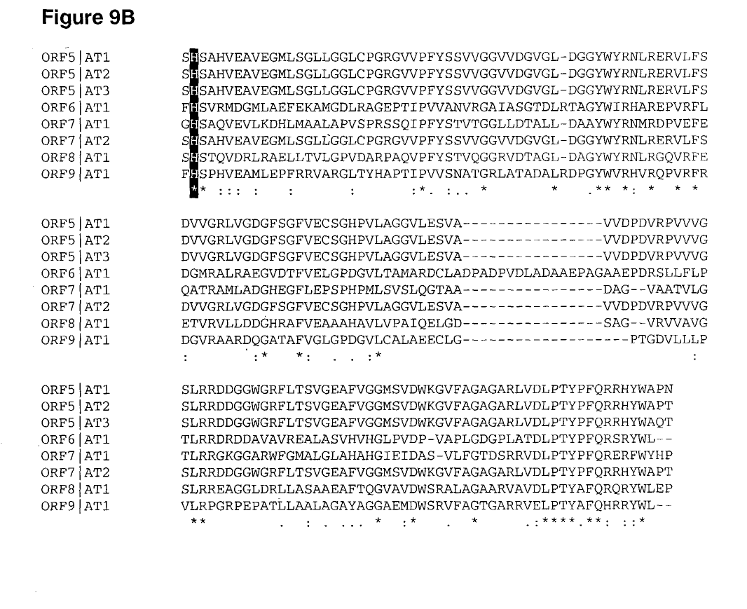

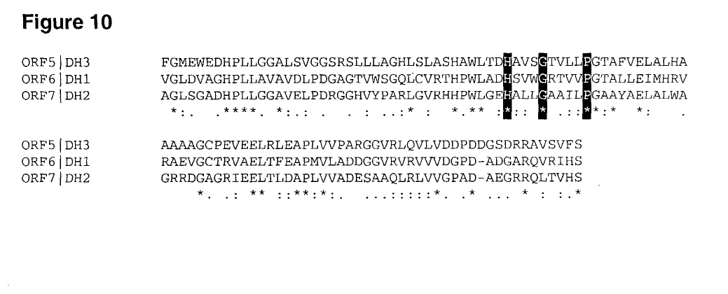

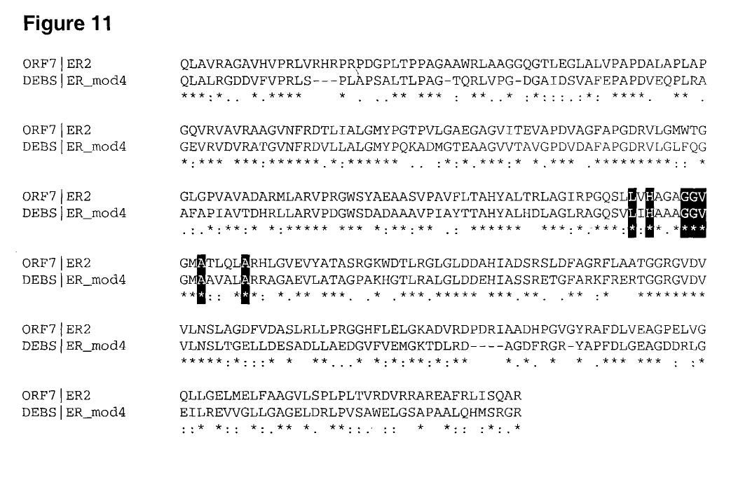

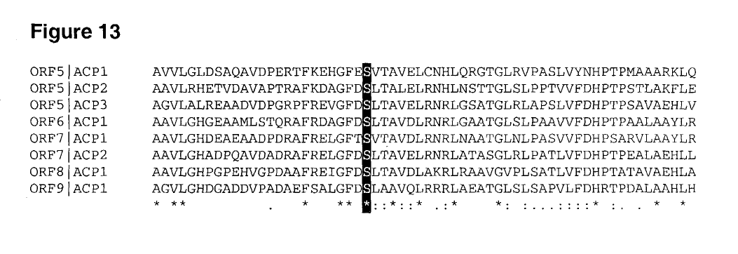

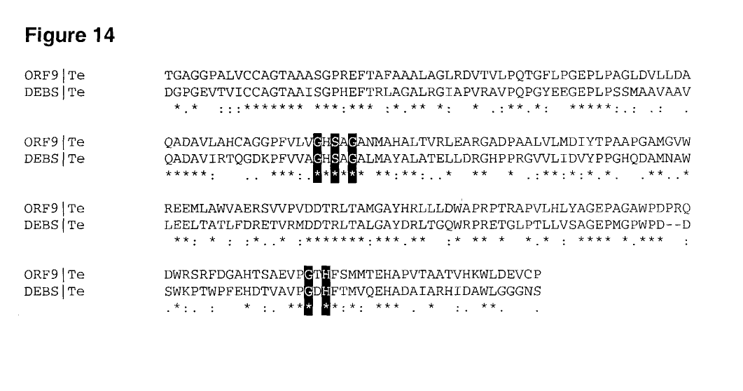

FIGS. 8 to 10 are amino acid alignments comparing the rosaramicin PKS domains. The domains which occur only once in the rosaramicin PKS, namely the enoylreductase (ER) and thioesterase (Te) domains, are compared to prototypical domains from the erythromycin PKS system (DEBS). Where applicable, key active site residues and motifs for the various polyketide synthase domains as described in Kakavas et al. (1997) J. Bacteriol. Vol 179 pp. 7515-7522 are indicated in FIGS. 8 to 14. In each of the clustal alignments a line above the alignement is used to mark strongly conserved positions. In addition, three characters, namely * (asterisk), : (colon) and . (period) are used, wherein “*” indicates positions which have a single, fully conserved residue; “:” indicates that one of the following strong groups is fully conserved: STA, NEQK, NHQK, NDEQ, QHRK, MILV, MILF, HY, and FYW; and “.” Indicates that one of the following weaker groups is fully conserved: CSA, ATV, SAG, STNK, STPA, SGND, SNDEQK, NDEQHK, NEQHRK, FVLIM, and HFY.

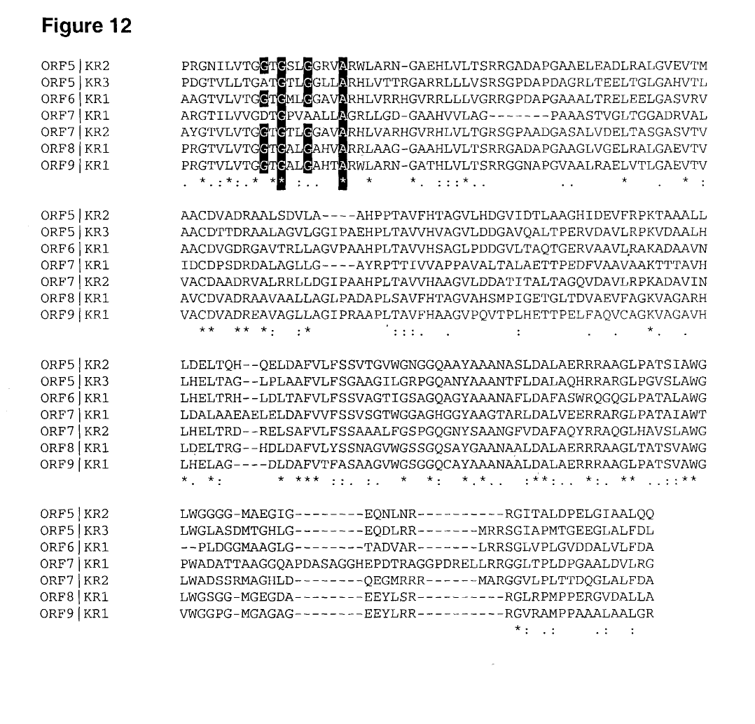

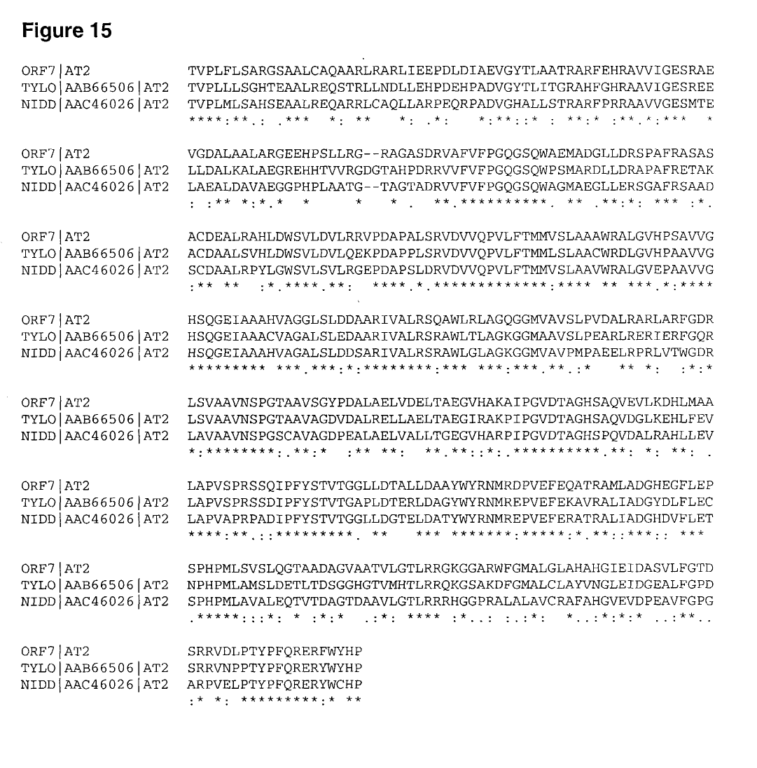

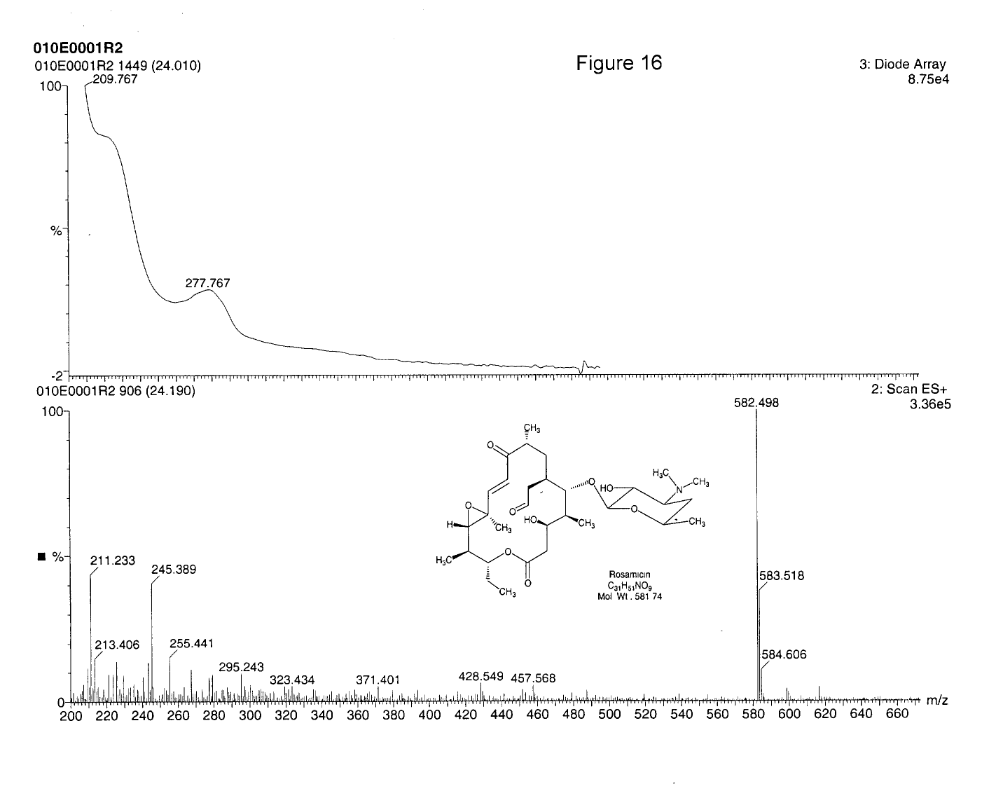

Of particular relevance with respect to PKS domain function, the KS domain in the loading module (ORF5|KS1) contains a Gln (Q) in place of the active site Cys (C) residue (FIG. 8) and that the KR domain of the first module of ORF7 (ORF7|KR1) contains several amino acid substitutions in the key cofactor-binding motif (FIG. 12). FIG. 15 shows the high degree of overall homology between ethylmalonyl-CoA-specific AT domains from the tylosin PKS (TYLO) and the niddamycin PKS (NIDD) and the second AT domain of rosaramicin ORF 7. This high degree of homology is indicative of their shared substrate specificity.

REGS and REGM are involved in regulation of gene expression. ABCC, a membrane transport protein and MTRA, a rRNA methyltransferase, are involved in resistance to and/or export of rosaramicin. The TESA gene product represents a free-standing thioesterase enzyme that is expected to play a “proofreading” role in the assembly of the rosaramicin core polyketide precursor. The OXRH gene product represents a crotonyl CoA reductase that is involved in the formation of the acyl-CoA precursor used by the loading module of ORF 5 and/or the second module of ORF 7. The step involving crotonyl CoA reductase, ie. the OXRH gene product, is expected to be a rate-limiting step in the biosynthesis of rosaramicin (Stassi D. L. et al., Proc Natl Acad Sci 95(13), 7305-9, Jun. 23, 1998) and it is expected that increasing the levels of the OXRH enzyme will have a beneficial effect on the yield of rosaramicin. The NBPA gene product is a nucleotide binding protein (i.e., contains a GTP/ATP binding motif) and is expected to activate a sugar by tethering it to a nucleotide, usually TTP. Therefore, the NBPA gene product is expected to be involved in the first step in the pathway leading to the formation of the deoxyamino sugar of rosaramicin.

### EXAMPLE 4

### Fermentation of Micronomospora carbonacea aurantiaca and Detection of Rosaramicin

Micromonospora carbonacea aurantiaca NRRL 2997 was cultured on a 30 ml media A plate (glucose 1.0%, dextrin 4.0%. sucrose 1.5%, casein enzymatic hydrolysate 1.0%, MgSO₄ 0.1%, CaCO₃ 0.2%, and agar 2.2 g/100 ml) at 30° C. for 14 days. The cells and agar were added to 25 ml of 95% ethanol and incubated at room temperature for 2 h under agitation. The ethanol phase was collected and the extraction step was repeated under the same conditions. The ethanol was evaporated from the pooled extracts and the residue was freeze-dried. The residue was then resuspended in 1.0 ml of water.

SPE of Extracts

The C-18 solid phase column (Burdick & Jackson) was conditioned before use by sequential washing with 3 ml of distilled water, 3 ml of methanol, and finally 3 ml of distilled water. The residue previously resuspended in 1.0 ml of water was loaded on the conditioned solid phase extraction system (SPE). Following passage of the sample though the SPE column washes were performed first, with 5 ml of water to remove polar materials, and then with 70% acetone and 30% methanol to elute a secondary metabolite-containing fraction which was then freeze-dried. This organic fraction was dissolved in 300 ul of 50% acetonitrile-distilled water.

Chemical Analysis

Chemical analysis of the organic fraction from the SPE column was performed by HPLC-ES-MS (Waters, ZQ systems). The extracts (50.0 ul) were separated on a C18 symmetry analytical column (2.1×150 mm) with HPLC 2690 system (Waters) using a 60-min linear gradient from 30% acetonitrile-5 mM ammonium acetate to 95% acetonitrile-5 mM ammonium acetate at a flow rate of 150 ul min⁻¹. UV and visible light absorption spectra (220 to 500 nm) were acquired with a PDA (Waters) by using the column effluents prior to their analysis by ES-MS. The electrospray source was switched between positive ion mode and negative ion mode at 0.3 s intervals to acquire both positive and negative ion spectra. The cone voltage was 25.0 V. The capillary was maintained at 3.0 V. The source temperature was kept at 100° C. The desolvation temperature was kept at 400° C. and the desolvation gas flow was 479 litre.h⁼¹. The data collection and analysis were performed with MassLynx V3.5 program (Waters).

FIG. 8 is a HPLC-ES-MS analysis of rosaramicin showing a UV spectra at a retention time of 24.4 minutes and a MS spectra showing a molecular ion consistent with rosaramicin at retention time 24.4 minutes (mass of 582.57 [M+H]^{+).}

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all sizes and all molecular weight or mass values are approximate, and are provided for description.

Patents, patent publications, procedures and publications cited throughout this application are incorporated herein in their entirety for all purposes.

1. An isolated, purified or enriched nucleic acid comprising a nucleic acid sequence selected from the group consisting of:
(a) a nucleic acid of SEQ ID NOS: 3, 5, 7, 9, 11, 13, 15, 17, 19, 21, 23, 25, 27, 29, 31, 33, 35, 37, 39;
(b) a nucleic acid encoding a polypeptide of SEQ ID NOS: 2, 4, 6, 8, 10, 12, 14, 16, 18, 20, 22, 24, 26, 28, 30, 32, 34, 36, 38;
(c) a nucleic acid having at least 75% homology to a nucleic acid of (a) or (b) as determined by analysis with BLASTN version 2.0 with the default parameters;
(d) a nucleic acid complementary to a nucleic acid of (a), (b) or (c).

2. An isolated, purified or enriched nucleic acid capable of hybridizing to a nucleic acid of claim 1 under conditions of high stringency.

3. An isolated, purified or enriched nucleic acid capable of hybridizing to the nucleic acid of claim 1 under conditions of moderate stringency.

4. An isolated, purified or enriched nucleic acid comprising the sequence of at least two nucleic acids of claim 1.

5. An isolated, purified or enriched nucleic acid comprising the sequence of at least three nucleic acids of claim 1.

6. An isolated, purified or enriched nucleic acid comprising a nucleic acid that hybridizes under stringent conditions to any one of rosaramicin open reading frames (ORFs) 1 to 19 (SEQ ID NOS: 3, 5, 7, 9, 11, 13, 15, 17, 19, 21, 23, 25, 27, 29, 31, 33, 35, 37, 39) and can substitute for the ORF to which it specifically hybridizes to direct the synthesis of a rosaramicin compound or analogue.

7. An isolated, purified or enriched nucleic acid that hybridizes under stringent conditions to any one of rosaramicin ORFs 1, 2, 4, 5, 6, 7, 8, 9, 10, 12, 14 or 15 (SEQ ID NOS: 3, 5, 9, 11, 13, 15, 17, 19, 21, 25, 29, and 31) and can substitute for the ORF to which it specifically hybridizes to direct the synthesis of a rosaramicin compound or analogue.

8. An isolated nucleic acid of claim 1 that hybridizes under stringent conditions to a nucleic acid encoding a polypeptide selected from the group comprising SEQ ID NOS: 2, 4, 6, 8, 10, 12, 14, 16,18, 20.

9. The isolated nucleic acid of claim 1 that hybridizes under stringent conditions to a nucleic acid encoding a polypeptide selected from the group consisting of SEQ ID NOS: 22, 24, 26, 28, 30, 32, 34, 36, 38.

10. An isolated gene cluster comprising ORFs encoding polypeptides sufficient to direct the synthesis of a rosaramicin compound or analogue.

11. The isolated gene cluster of claim 10 wherein the gene cluster is present in a bacterium.

12. The isolated gene cluster of claim 10 wherein the gene cluster contains a nucleic acid of any one of rosaramicin ORFs 1 to 19 (SEQ ID NOS: 3, 5, 7, 9, 11, 13, 15, 17, 19, 21, 23, 25, 27, 29, 31, 33, 35, 37, 39) present in the E. coli strains DH10B having accession nos. IDAC 100702-1, 100702-2 and 100702-3.

13. An isolated polypeptide comprising a polypeptide sequence selected from any one of:
(a) a polypeptide of any one of SEQ ID NOS: 2, 4, 6, 8, 10, 12, 14, 16, 18, 20, 22, 24, 26, 28, 30, 32, 34, 36, 38; and
(b) a polypeptide which is at least 75% identical in amino acid sequence to a polypeptide of any one of SEQ ID NOS: 2, 4, 6, 8, 10, 12, 14, 16, 18, 20, 22, 24, 26, 28, 30, 32, 34, 36, 38 as determined by analysis with BLASTP with the default parameters.

14. The isolated polypeptide of claim 13 wherein the polypeptide sequence selected from any one of:
a) a polypeptide of any one of rosaramicin ORFs 1, 2, 4, 5, 6, 7, 8, 9, 10, 12, 14 or 15 (SEQ ID NOS: 2, 4, 8, 10, 12, 14, 16, 18, 20, 24, 28 and 30); and
b) a polypeptide which is at least 75% identical in amino acid sequence to a pelypeptide of any one of rosaramicin ORFs 1, 2, 4, 5, 6, 7, 8, 9, 10, 12, 14 or 15 (SEQ ID NOS: 2, 4, 8, 10, 12, 14, 16, 18, 20, 24, 28 and 30) as determined by analysis with BLASTP with the default parameters.

15. A polypeptide comprising at least two polypeptides of claim 14.

16. A polypeptide comprising at least three polypeptides of claim 14.

17. A polypeptide comprising at least five or more polypeptides of claim 14.

18. An expression vector comprising a nucleic acid of claim 1.

19. A host cell transformed with an expression vector of claim 18.

20. The host cell of claim 19, wherein the cell is transformed with an exogenous nucleic acid comprising a gene cluster encoding polypeptides sufficient to direct the assembly of a rosaramicin compound or analogue.

21. A method of chemically modifying a biological molecule that is a substrate for a polypeptide encoded by a rosaramicin biosynthesis gene cluster, said method comprising contacting the biological molecule with a polypeptide of claim 13, wherein said polypeptide chemically modifies said biological molecule.

22. The method of chemically modifying a biological molecule that is a substrate for a polypeptide encoded by a rosaramicin biosynthesis gene cluster, said method comprising contacting the biological molecule with at least two different polypeptides of claim 13.

23. An isolated or purified antibody capable of specifically binding to a polypeptide having a sequence selected from the group consisting of SEQ ID NOS: 2, 4, 6, 8, 10, 12, 14, 16, 18, 20, 22, 24, 26, 28, 30, 32, 34, 36, 38.

24. A method of making a polypeptide having a sequence selected from the group consisting of SEQ ID NOS: 2, 4, 6, 8, 10, 12, 14, 16, 18, 20, 22, 24, 26, 28, 30, 32, 34, 36, 38 comprising introducing a nucleic acid encoding said polypeptide, said nucleic acid being operably linked to a promoter, into a host cell.

25. A method of making a rosaramicin compound or analog comprising the step of providing a bacterium containing a gene cluster with sufficient genes to produce a rosaramicin compound of analogue and culturing the bacterium under conditions allowing for expression of the sufficient genes to produce a rosaramicin compound, wherein the gene cluster contains at least one nucleic acid of claim 1.

26. A method of making a rosaramicin compound or analog comprising culturing a Micromonospora carbonacea bacterium under conditions allowing for expression of rosaramicin ORFs 1 to 19 (SEQ ID NOS: 3, 5, 7, 9, 11, 13, 15, 17, 19, 21, 23, 25, 27, 29, 31, 33, 35, 37, 39) present in the E. coli strains DH10B having accession nos. IDAC 100702-1, 100702-2 and 100702-3.

27. A computer readable medium having stored thereon a sequence selected from the group consisting of a nucleic acid code of SEQ ID NOS: 3, 5, 7, 9, 11, 13, 15, 17, 19, 21, 23, 25, 27, 29, 31, 33, 35, 37, 39, 41, 43, 45, 47, 49, 51 and a polypeptide code of SEQ ID NOS: 2, 4, 6, 8, 10, 12, 14, 16, 18, 20, 22, 24, 26, 28, 30, 32, 34, 36, 38, 40, 42, 44, 46, 48, 50.

28. A computer system comprising a processor and a data storage device wherein said data storage device has stored thereon a sequence selected from the group consisting of a nucleic acid code of SEQ ID NOS: 3, 5, 7, 9, 11, 13, 15, 17, 19, 21, 23, 25, 27, 29, 31, 33, 35, 37, 39, 41, 43, 45, 47, 49, 51 and a polypeptide code of SEQ ID NOS: 2, 4, 6, 8, 10, 12, 14, 16, 18, 20, 22, 24, 26, 28, 30, 32, 34, 36, 38, 40, 42, 44, 46, 48, 50.
